SEQUENCE LISTING (266 sequences)

SEQ ID NO: 1-104 - skipped (104 entries)

SEQ ID NO: 105 - protein, 16 aa, synthetic construct
  source 1..16: note = SARS-CoV-2 S309-v1 mAb VH
SARSCOVSVMABVLVK

SEQ ID NO: 106 - protein, 8 aa, synthetic construct
  source 1..8: note = SARS-CoV-2 S309-v1 (Sotrovimab and VIR-7832) mAb CDRH1
GYPFTSYG

SEQ ID NO: 107 - protein, 8 aa, synthetic construct
  source 1..8: note = SARS-CoV-2 S309-v1 mAb CDRH2
ISTYNGNT

SEQ ID NO: 108 - protein, 20 aa, synthetic construct
  source 1..20: note = SARS-CoV-2 S309-v1 (Sotrovimab and VIR-7832) mAb CDRH3
ARDYTRGAWFGESLIGGFDN

SEQ ID NO: 109-112 - skipped (4 entries)

SEQ ID NO: 113 - protein, 127 aa, synthetic construct
  source 1..127: note = SARS-CoV-2 S309-v1.1 (Sotrovimab and VIR-7832)mAb VH
QVQLVQSGAEVKKPGASVKVSCKASGYPFTSYGISWVRQAPGQGLEWMGWISTYQGNTNYAQKFQGRVTMTTDTSTTTGYMELRRLRSDDTAVYYCARDYTRGAWFGESLIGGFDNWGQGTLVTVSS

SEQ ID NO: 114-118 - skipped (5 entries)

SEQ ID NO: 119 - protein, 127 aa, synthetic construct
  source 1..127: note = SARS-CoV-2 S309-v1.7 mAb VH
QVQLVQSGAEVKKPGASVKVSCKASGYPFTSYGISWVRQAPGQGLEWMGWISTYNGNTNYAQKFQGRVTMTTDTSTTTGYMELRRLRSDDTAVYYCARDYTRGAFFGESLIGGFDNWGQGTLVTVSS

SEQ ID NO: 120 - protein, 127 aa, synthetic construct
  source 1..127: note = SARS-CoV-2 S309-v1.8 mAb VH
QVQLVQSGAEVKKPGASVKVSCKASGYPFTSYGISWVRQAPGQGLEWMGWISTYNGNTNYAQKFQGRVTMTTDTSTTTGYMELRRLRSDDTAVYYCARDYTRGAYFGESLIGGFDNWGQGTLVTVSS

SEQ ID NO: 121 - protein, 8 aa, synthetic construct
  source 1..8: note = SARS-CoV-2 S309-v1.1 (Sotrovimab and VIR-7832) mAb CDRH2
ISTYQGNT

SEQ ID NO: 122-164 - skipped (43 entries)

SEQ ID NO: 165 - protein, 1273 aa, SARS-CoV-2 betacoronavirus
  source 1..1273: note = surface glycoprotein [Wuhan seafood market pneumonia virus]; GenBank: QHD43416.1; January 23, 2020
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 166 - protein, 220 aa, SARS-CoV-2 betacoronavirus
  source 1..220: note = surface glycoprotein RBD [Wuhan seafood market pneumonia virus]; GenBank: QHD43416.1; January 23, 2020
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTG

SEQ ID NO: 167 - protein, 72 aa, SARS-CoV-2 betacoronavirus
  source 1..72: note = Receptor Binding Motif (RBM) in surface glycoprotein RBD [Wuhan seafood market pneumonia virus]; GenBank: QHD43416.1; January 23, 2020
NSNNLDSKVGGNYNYLYRLFRKSNLKPFERDISTEIYQAGSTPCNGVEGFNCYFPLQSYGFQPTNGVGYQPY

SEQ ID NO: 168 - protein, 107 aa, synthetic construct [sequence omitted]

SEQ ID NO: 169 - protein, 7 aa, synthetic construct
  source 1..7: note = SARS-CoV-2 S309-v13 (Sotrovimab and VIR-7832) mAb CDRL1
QTVSSTS

SEQ ID NO: 170 - skipped

SEQ ID NO: 171 - protein, 8 aa, synthetic construct
  source 1..8: note = SARS-CoV-2 S309-v13 (Sotrovimab and VIR-7832) mAb CDRL3
QQHDTSLT

SEQ ID NO: 172 - skipped

SEQ ID NO: 173 - protein, 330 aa, synthetic construct
  source 1..330: note = SARS-CoV-2 CH1-CH3 G1m17; IgG1*01 LS (aa) (present in Sotrovimab)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 174 - protein, 107 aa, synthetic construct [sequence omitted]

SEQ ID NO: 175 - protein, 330 aa, synthetic construct
  source 1..330: note = SARS-CoV-2 CH1-CH3 G1m17; IgG1*01 LS GAALIE (aa) (present in VIR-7832)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 176-192 - skipped (17 entries)

SEQ ID NO: 193 - protein, 106 aa, synthetic construct
  source 1..106: note = SARS-CoV-2 mAb CL IgLC*01
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 194-205 - skipped (12 entries)

SEQ ID NO: 206 - protein, 18 aa, synthetic construct
  source 1..18: note = Linker
GSTSGSGKPGSGEGSTKG

SEQ ID NO: 207 - protein, 10 aa, synthetic construct
  source 1..10: note = Linker
GSGKPGSGEG

SEQ ID NO: 208 - protein, 8 aa, synthetic construct
  source 1..8: note = Linker
GKPGSGEG

SEQ ID NO: 209 - protein, 8 aa, synthetic construct
  source 1..8: note = Linker
SGKPGSGE

SEQ ID NO: 210 - skipped

SEQ ID NO: 211 - protein, 110 aa, synthetic construct
  source 1..110: note = Linker
GGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGS

SEQ ID NO: 212 - protein, 15 aa, synthetic construct
  source 1..15: note = Linker
GGGGSGGGGSGGGGS

SEQ ID NO: 213 - protein, 50 aa, synthetic construct
  source 1..50: note = Linker
GGGGSGGGGSGGGGSGGGGSGGGGSGGGGSGGGGSGGGGSGGGGSGGGGS

SEQ ID NO: 214 - protein, 18 aa, synthetic construct
  source 1..18: note = Linker
GSTSGGGSGGGSGGGGSS

SEQ ID NO: 215 - protein, 14 aa, synthetic construct
  source 1..14: note = Linker
EGKSSGSGSESKVD

SEQ ID NO: 216 - protein, 18 aa, synthetic construct
  source 1..18: note = Linker
KESGSVSSEQLAQFRSLD

SEQ ID NO: 217 - protein, 5 aa, synthetic construct
  source 1..5: note = Linker
GGGGS

SEQ ID NO: 218 - protein, 494 aa, synthetic construct
  source 1..494: note = SARS-CoV-2 S309-scFab (H-L)
QVQLVQSGAEVKKPGASVKVSCKASGYPFTSYGISWVRQAPGQGLEWMGWISTYNGNTNYAQKFQGRVTMTTDTSTTTGYMELRRLRSDDTAVYYCARDYTRGAWFGESLIGGFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSGGGGSGGGGSGGGGSGGGGSGGGGSGGGGSGGGGSGGGGSGGGGSEIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 219 - protein, 494 aa, synthetic construct
  source 1..494: note = SARS-CoV-2 S309-scFab (L-H)
EIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSGGGGSGGGGSQVQLVQSGAEVKKPGASVKVSCKASGYPFTSYGISWVRQAPGQGLEWMGWISTYNGNTNYAQKFQGRVTMTTDTSTTTGYMELRRLRSDDTAVYYCARDYTRGAWFGESLIGGFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 220 - protein, 249 aa, synthetic construct
  source 1..249: note = SARS-CoV-2 S309-scFv (VH-VL)
QVQLVQSGAEVKKPGASVKVSCKASGYPFTSYGISWVRQAPGQGLEWMGWISTYNGNTNYAQKFQGRVTMTTDTSTTTGYMELRRLRSDDTAVYYCARDYTRGAWFGESLIGGFDNWGQGTLVTVSSGGGGSGGGGSGGGGSEIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIK

SEQ ID NO: 221 - protein, 249 aa, synthetic construct
  source 1..249: note = SARS-CoV-2 S309-scFv (VL-VH)
EIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIKGGGGSGGGGSGGGGSQVQLVQSGAEVKKPGASVKVSCKASGYPFTSYGISWVRQAPGQGLEWMGWISTYNGNTNYAQKFQGRVTMTTDTSTTTGYMELRRLRSDDTAVYYCARDYTRGAWFGESLIGGFDNWGQGTLVTVSS

SEQ ID NO: 222 - protein, 518 aa, synthetic construct
  source 1..518: note = SARS-CoV-2 S309-scFv (VH-VL)-(VH-VL)
QVQLVQSGAEVKKPGASVKVSCKASGYPFTSYGISWVRQAPGQGLEWMGWISTYNGNTNYAQKFQGRVTMTTDTSTTTGYMELRRLRSDDTAVYYCARDYTRGAWFGESLIGGFDNWGQGTLVTVSSGGGGSGGGGSGGGGSEIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIKGGGGSGGGGSGGGGSGGGGSQVQLVQSGAEVKKPGASVKVSCKASGYPFTSYGISWVRQAPGQGLEWMGWISTYNGNTNYAQKFQGRVTMTTDTSTTTGYMELRRLRSDDTAVYYCARDYTRGAWFGESLIGGFDNWGQGTLVTVSSGGGGSGGGGSGGGGSEIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIK

SEQ ID NO: 223 - protein, 518 aa, synthetic construct
  source 1..518: note = SARS-CoV-2 S309-scFv-(VH-VL)-(VL-VH)
QVQLVQSGAEVKKPGASVKVSCKASGYPFTSYGISWVRQAPGQGLEWMGWISTYNGNTNYAQKFQGRVTMTTDTSTTTGYMELRRLRSDDTAVYYCARDYTRGAWFGESLIGGFDNWGQGTLVTVSSGGGGSGGGGSGGGGSEIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIKGGGGSGGGGSGGGGSGGGGSEIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIKGGGGSGGGGSGGGGSQVQLVQSGAEVKKPGASVKVSCKASGYPFTSYGISWVRQAPGQGLEWMGWISTYNGNTNYAQKFQGRVTMTTDTSTTTGYMELRRLRSDDTAVYYCARDYTRGAWFGESLIGGFDNWGQGTLVTVSS

SEQ ID NO: 224 - protein, 518 aa, synthetic construct
  source 1..518: note = SARS-CoV-2 S309-scFv-(VL-VH)-(VH-VL)
EIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIKGGGGSGGGGSGGGGSQVQLVQSGAEVKKPGASVKVSCKASGYPFTSYGISWVRQAPGQGLEWMGWISTYNGNTNYAQKFQGRVTMTTDTSTTTGYMELRRLRSDDTAVYYCARDYTRGAWFGESLIGGFDNWGQGTLVTVSSGGGGSGGGGSGGGGSGGGGSQVQLVQSGAEVKKPGASVKVSCKASGYPFTSYGISWVRQAPGQGLEWMGWISTYNGNTNYAQKFQGRVTMTTDTSTTTGYMELRRLRSDDTAVYYCARDYTRGAWFGESLIGGFDNWGQGTLVTVSSGGGGSGGGGSGGGGSEIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIK

SEQ ID NO: 225 - protein, 518 aa, synthetic construct
  source 1..518: note = SARS-CoV-2 S309-scFv-(VL-VH)-(VL-VH)
EIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIKGGGGSGGGGSGGGGSQVQLVQSGAEVKKPGASVKVSCKASGYPFTSYGISWVRQAPGQGLEWMGWISTYNGNTNYAQKFQGRVTMTTDTSTTTGYMELRRLRSDDTAVYYCARDYTRGAWFGESLIGGFDNWGQGTLVTVSSGGGGSGGGGSGGGGSGGGGSEIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIKGGGGSGGGGSGGGGSQVQLVQSGAEVKKPGASVKVSCKASGYPFTSYGISWVRQAPGQGLEWMGWISTYNGNTNYAQKFQGRVTMTTDTSTTTGYMELRRLRSDDTAVYYCARDYTRGAWFGESLIGGFDNWGQGTLVTVSS

SEQ ID NO: 226 - protein, 494 aa, synthetic construct
  source 1..494: note = SARS-CoV-2 S309-scFab-(H-L) v1.1
QVQLVQSGAEVKKPGASVKVSCKASGYPFTSYGISWVRQAPGQGLEWMGWISTYQGNTNYAQKFQGRVTMTTDTSTTTGYMELRRLRSDDTAVYYCARDYTRGAWFGESLIGGFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSGGGGSGGGGSGGGGSGGGGSGGGGSGGGGSGGGGSGGGGSGGGGSEIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 227 - protein, 494 aa, synthetic construct
  source 1..494: note = SARS-CoV-2 S309-scFab-(L-H) v1.1
EIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSGGGGSGGGGSQVQLVQSGAEVKKPGASVKVSCKASGYPFTSYGISWVRQAPGQGLEWMGWISTYQGNTNYAQKFQGRVTMTTDTSTTTGYMELRRLRSDDTAVYYCARDYTRGAWFGESLIGGFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 228 - protein, 249 aa, synthetic construct
  source 1..249: note = SARS-CoV-2 S309-scFv-(VH-VL) v1.1
QVQLVQSGAEVKKPGASVKVSCKASGYPFTSYGISWVRQAPGQGLEWMGWISTYQGNTNYAQKFQGRVTMTTDTSTTTGYMELRRLRSDDTAVYYCARDYTRGAWFGESLIGGFDNWGQGTLVTVSSGGGGSGGGGSGGGGSEIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIK

SEQ ID NO: 229 - protein, 249 aa, synthetic construct
  source 1..249: note = SARS-CoV-2 S309-scFv-(VL-VH) v1.1
EIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIKGGGGSGGGGSGGGGSQVQLVQSGAEVKKPGASVKVSCKASGYPFTSYGISWVRQAPGQGLEWMGWISTYQGNTNYAQKFQGRVTMTTDTSTTTGYMELRRLRSDDTAVYYCARDYTRGAWFGESLIGGFDNWGQGTLVTVSS

SEQ ID NO: 230 - protein, 518 aa, synthetic construct
  source 1..518: note = SARS-CoV-2 S309-scFv-(VH-VL)-(VH-VL) v1.1
QVQLVQSGAEVKKPGASVKVSCKASGYPFTSYGISWVRQAPGQGLEWMGWISTYQGNTNYAQKFQGRVTMTTDTSTTTGYMELRRLRSDDTAVYYCARDYTRGAWFGESLIGGFDNWGQGTLVTVSSGGGGSGGGGSGGGGSEIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIKGGGGSGGGGSGGGGSGGGGSQVQLVQSGAEVKKPGASVKVSCKASGYPFTSYGISWVRQAPGQGLEWMGWISTYQGNTNYAQKFQGRVTMTTDTSTTTGYMELRRLRSDDTAVYYCARDYTRGAWFGESLIGGFDNWGQGTLVTVSSGGGGSGGGGSGGGGSEIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIK

SEQ ID NO: 231 - protein, 518 aa, synthetic construct
  source 1..518: note = SARS-CoV-2 S309-scFv-(VH-VL)-(VL-VH) v1.1
QVQLVQSGAEVKKPGASVKVSCKASGYPFTSYGISWVRQAPGQGLEWMGWISTYQGNTNYAQKFQGRVTMTTDTSTTTGYMELRRLRSDDTAVYYCARDYTRGAWFGESLIGGFDNWGQGTLVTVSSGGGGSGGGGSGGGGSEIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIKGGGGSGGGGSGGGGSGGGGSEIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIKGGGGSGGGGSGGGGSQVQLVQSGAEVKKPGASVKVSCKASGYPFTSYGISWVRQAPGQGLEWMGWISTYQGNTNYAQKFQGRVTMTTDTSTTTGYMELRRLRSDDTAVYYCARDYTRGAWFGESLIGGFDNWGQGTLVTVSS

SEQ ID NO: 232 - protein, 518 aa, synthetic construct
  source 1..518: note = SARS-CoV-2 S309-scFv-(VL-VH)-(VH-VL) v1.1
EIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIKGGGGSGGGGSGGGGSQVQLVQSGAEVKKPGASVKVSCKASGYPFTSYGISWVRQAPGQGLEWMGWISTYQGNTNYAQKFQGRVTMTTDTSTTTGYMELRRLRSDDTAVYYCARDYTRGAWFGESLIGGFDNWGQGTLVTVSSGGGGSGGGGSGGGGSGGGGSQVQLVQSGAEVKKPGASVKVSCKASGYPFTSYGISWVRQAPGQGLEWMGWISTYQGNTNYAQKFQGRVTMTTDTSTTTGYMELRRLRSDDTAVYYCARDYTRGAWFGESLIGGFDNWGQGTLVTVSSGGGGSGGGGSGGGGSEIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIK

SEQ ID NO: 233 - protein, 518 aa, synthetic construct
  source 1..518: note = SARS-CoV-2 S309-scFv-(VL-VH)-(VL-VH) v1.1
EIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIKGGGGSGGGGSGGGGSQVQLVQSGAEVKKPGASVKVSCKASGYPFTSYGISWVRQAPGQGLEWMGWISTYQGNTNYAQKFQGRVTMTTDTSTTTGYMELRRLRSDDTAVYYCARDYTRGAWFGESLIGGFDNWGQGTLVTVSSGGGGSGGGGSGGGGSGGGGSEIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIKGGGGSGGGGSGGGGSQVQLVQSGAEVKKPGASVKVSCKASGYPFTSYGISWVRQAPGQGLEWMGWISTYQGNTNYAQKFQGRVTMTTDTSTTTGYMELRRLRSDDTAVYYCARDYTRGAWFGESLIGGFDNWGQGTLVTVSS

SEQ ID NO: 234-250 - skipped (17 entries)

SEQ ID NO: 251 - DNA, 767 bp, synthetic construct
  source 1..767: mol_type = other DNA; note = CMV promoter
gacattgattattgactagttattaatagtaatcaattacggggtcattagttcatagcccatatatggagttccgcgttacataacttacggtaaatggcccgcctggctgaccgcccaacgacccccgcccatgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcgttag

SEQ ID NO: 252 - DNA, 50 bp, synthetic construct
  source 1..50: mol_type = other DNA; note = signal peptide
atgggatggtcatgtatcatcctttttctagtagcaactgcaaccggtgt

SEQ ID NO: 253 - DNA, 135 bp, synthetic construct
  source 1..135: mol_type = other DNA; note = Poly-adenylation signal sequence
aacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 254-255 - skipped (2 entries)

SEQ ID NO: 256 - protein, 16 aa, synthetic construct
  source 1..16: note = Signal peptide
MGWSCIILFLVATATG

SEQ ID NO: 257-262 - skipped (6 entries)

SEQ ID NO: 263 - DNA, 57 bp, synthetic construct
  source 1..57: mating_type = Signal peptide; mol_type = other DNA
atgggctggtcctgcatcatcctgttcctggtggccacagccaccggcgtgcacagc

SEQ ID NO: 264 - protein, 19 aa, synthetic construct
  source 1..19: note = Signal peptide
MGWSCIILFLVATATGVHS

SEQ ID NO: 265 - protein, 329 aa, synthetic construct
  source 1..329: note = SARS-CoV-2 CH1-CH3 G1m17; IgG1*01 LS no C-term Lys (aa) (may be used in Sotrovimab or VIR-7832)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 266 - protein, 329 aa, synthetic construct
  source 1..329: note = SARS-CoV-2 CH1-CH3 G1m17; IgG1*01 LS GAALIE no C-term Lys (aa) (may be used in Sotrovimab or VIR-7832)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

(19) United States
(12) Patent Application Publication
(10) Pub. No.: US 2024/0400652 A1
(43) Pub. Date: Dec. 5, 2024

(54) ANTIBODY THERAPIES FOR SARS-COV-2 INFECTION IN PEDIATRIC SUBJECTS

(71) Applicants: VIR BIOTECHNOLOGY, INC., San Francisco, CA (US); GLAXO WELLCOME UK LIMITED, Brentford (GB)

(72) Inventors: Daren J. AUSTIN, Brentford (GB); Elizabeth ALEXANDER, San Francisco, CA (US)

(21) Appl. No.: 18/688,305

(22) PCT Filed: Aug. 31, 2022
(86) PCT No.: PCT/US2022/075766

§ 371 Date: Feb. 29, 2024

Related U.S. Application Data
(60) Provisional application No. 63/239,888, filed on Sep. 1, 2021.
(60) Provisional application No. 63/286,966, filed on Dec. 7, 2021.

(51) Int. Cl. C07K 16/10; A61K 39/00

(52) U.S. Cl. CPC C07K 16/1003; A61K 2039/505; A61K 2039/55; C07K 2317/33; C07K 2317/732; C07K 2317/76; C07K 2317/92

(57) ABSTRACT

The instant disclosure provides methods of treating or preventing a SARS-CoV-2 infection in a pediatric subject, e.g., in a pediatric subject having or at risk for developing COVID-19, wherein the methods include administering an antibody, antigen-binding fragment, or composition comprising the same to a pediatric subject. Disclosed methods include prophylactic administration for preventing SARS-CoV-2 infection or transmission, as well as treatment of a pediatric subject having a SARS-CoV-2 infection. A SARS-CoV-2 infection (e.g., causing COVID-19) to be treated can be at any stage of infection and/or can result in any stage of disease, for example, mild, mild-to-moderate, severe, or critical.

### STATEMENT REGARDING SEQUENCE LISTING

The Sequence Listing associated with this application is provided in XML format in lieu of a paper copy, and is hereby incorporated by reference into the specification. The name of the XML file containing the Sequence Listing is 930585_428WO_SequenceListing.xml. The XML file is 111,330 bytes, was created on Aug. 30, 2022, and is being submitted electronically via EFS-Web.

### BACKGROUND

A novel betacoronavirus emerged in Wuhan, China, in late 2019. As of Jun. 9, 2021, approximately 174 million cases of infection by this virus (termed, among other names, SARS-CoV-2 and Wuhan coronavirus) were confirmed worldwide, and had resulted in approximately 3.7 million deaths. Therapies for preventing or treating SARS-CoV-2 infection and COVID-19 are needed.

### BRIEF DESCRIPTION OF THE DRAWINGS

FIG. 1 shows the design of an adult clinical study of recombinant monoclonal IgG1 antibody sotrovimab (also called, e.g., S309 N55Q LS or VIR-7831 herein) for treatment of mild to moderate COVID-19 disease.

FIGS. 2A-2E show a timeline of events for an adult clinical study of sotrovimab for treatment of mild to moderate COVID-19 disease.

FIG. 3 shows the study design for an adult clinical study of sotrovimab for treatment of severe to critical COVID-19 disease.

FIG. 4 shows the study design for an adult clinical study of sotrovimab for post-exposure prophylaxis of COVID-19 disease.

FIG. 5 shows the binding site of sotrovimab on the spike protein of SARS-CoV-2. The SARS-CoV-2 receptor-binding domain is shown, with the ACE2 receptor-binding motif in green and the sotrovimab epitope in orange. ACE2 denotes angiotensin-converting enzyme 2.

FIG. 6 shows the design of an adult clinical study using sotrovimab described herein. R denotes randomization. *Patients were stratified by age (≤70 vs. >70 years), symptom duration (≤3 days vs. 4-5 days), and region. †Study pharmacists reconstituted and dispensed all study medications within equal time frames to maintain blinding.

FIG. 7 shows patient disposition in the intent-to-treat (ITT) population of patients in an adult clinical study of sotrovimab.

FIG. 8 shows the design of a lead-in phase of an adult pharmacokinetics (PK) clinical study using sotrovimab.

FIG. 9A is a linear scale plot of sotrovimab serum concentration at Day 141 after a single injection according to an adult clinical study.

FIG. 9B is a semi-logarithmic scale plot of the same data. Note: Lower limit of quantification (LLQ)=0.1 μg/mL. Excludes anomalous concentrations identified as sampling errors by the clinical pharmacologist. Data is based on 1-hour infusion time.

FIG. 9C is a linear scale plot of sotrovimab serum concentration at Day 169 after a single injection according to an adult clinical study.

FIG. 9D is a semi-logarithmic scale plot of the same data. Note: Lower limit of quantification (LLQ)=0.1 μg/mL. Excludes anomalous concentrations identified as sampling errors by the clinical pharmacologist. Data is based on 1-hour infusion time.

FIG. 10 shows the design of a pediatric pharmacokinetics clinical study of sotrovimab.

FIGS. 11A-11B shows an alternative timeline of events for a pediatric pharmacokinetics clinical study of sotrovimab for patients 2 years of age or older.

FIGS. 11C-11D shows an alternative timeline of events for a pediatric pharmacokinetics clinical study of sotrovimab for patients less than 2 years of age.

### DETAILED DESCRIPTION

Provided herein are methods of treating or preventing a SARS-CoV-2 infection in a pediatric subject, e.g., in a subject having or at risk for developing COVID-19, using an antibody, an antigen-binding fragment, or composition that comprises the same. Certain antibodies and antigen-binding fragments for use in the methods recognize a conserved epitope in SARS-CoV-2 S glycoprotein and potently neutralize SARS-CoV-2 in vitro and in vivo. Non-limiting examples of antibodies include S309 and engineered variants of S309 (e.g., sotrovimab, VIR-7832). In some embodiments, a variant of S309 comprises a N55Q substitution in the VH region. In some embodiments, an antibody or antigen-binding fragment comprises an Fc polypeptide comprising one or more amino acid mutations that, for example, can extend in vivo half-life of the antibody or antigen-binding fragment and/or can promote a vaccinal effect of the antibody or antigen-binding fragment.

Presently disclosed methods include prophylaxis against SARS-CoV-2 infection or transmission, as well as treatment of a subject having a SARS-CoV-2 infection. A SARS-CoV-2 infection (e.g., causing COVID-19) can be at any stage of infection and/or can result in any stage of disease, for example, mild, mild-to-moderate, severe, or critical. For example, as described further herein, a single dose of an antibody of the present disclosure can be sufficient to reduce hospitalization or death in subjects with mild-to-moderate COVID-19.

Administration of the antibody or antigen-binding fragment can be performed using any method, such as for example, intravenous injection and intramuscular injection. In some contexts, a single dose of the antibody or antigen-binding fragment (or composition comprising the same) is administered to a subject. Pediatric subjects may be characterized in accordance with one or more criteria, and/or possess can one or more characteristics, as provided herein. Also provided are antibodies, antigen-binding fragments, and compositions for use in methods of treating or preventing a SARS-CoV-2 infection (or COVID-19) in a pediatric subject, as well as in the preparation of a medicament for the treatment or treating or prevention of a SARS-CoV-2 infection in a pediatric subject.

Also provided are kits that include the antibodies or antigen-binding fragments thereof described herein or compositions containing the antibodies or antigen-binding fragments thereof described herein or a nucleic acid, host cell, or vector as described herein that provides an antibody or antigen-binding fratment thereof as described herein to a pediatric subject. Kits for use in the methods described herein are also provided. Prior to setting forth this disclosure in more detail, it may be helpful to an understanding thereof to provide definitions of certain terms to be used herein. Additional definitions are set forth throughout this disclosure.

As used herein, “pediatric subject” refers to a human subject who is aged less than 18 years. Certain medical criteria may be different from that for adult subjects in the case of pediatric subjects. For example, the clinical definition of obesity differs between adult and pediatric subjects. Medical criteria described herein in the context of adult subjects may be adapted by one of ordinary skill in the art to the pediatric context using applicable medial standards. In clinical studies, a pediatric subject may be referred to as a “pediatric participant.” Any reference to a “child,” or “children” herein also refers to pediatric subject(s).

As used herein, “SARS-CoV-2”, also originally referred to as “Wuhan coronavirus”, “Wuhan seafood market pneumonia virus”, or “Wuhan CoV”, “novel CoV”, or “nCoV”, or “2019 nCoV”, or “Wuhan nCoV”, or a variant thereof, is a betacoronavirus of lineage B (sarbecovirus). SARS-CoV-2 was first identified in Wuhan, Hubei province, China, in late 2019 and spread within China and to other parts of the world by early 2020. SARS CoV-2 infection can result in a disease known as COVID-19; symptoms of COVID-19 include fever or chills, dry cough, dyspnea, fatigue, body aches, headache, new loss of taste or smell, sore throat, congestions or runny nose, nausea or vomiting, diarrhea, persistent pressure or pain in the chest, new confusion, inability to wake or stay awake, and bluish lips or face.

To date, infection with SARS-CoV-2 has often manifested as mild disease in children. As many as 20% of the infected children in Wuhan were asymptomatic and levels as high as 35% have been reported. When present, the most common symptoms experienced by children have included fever, mild cough, and pharyngitis, while sore throat, nasal congestion, rhinorrhea, and diarrhea have also been observed. Approximately 10% present with gastrointestinal symptoms. Unlike other viral respiratory diseases of children, wheeze has been uncommon. CT scans of the lungs have most often revealed a consolidation with a surrounding halo sign on CT scan of the lungs.

The genomic sequence of SARS-CoV-2 isolate Wuhan-Hu-1 is provided at GenBank MN908947.3, Jan. 23, 2020), and the amino acid translation of the genome is provided in GenBank QHD43416.1, Jan. 23, 2020. Like other coronaviruses (e.g., SARS CoV), SARS-CoV-2 comprises a “spike” or surface (“S”) type I transmembrane glycoprotein containing a receptor binding domain (RBD). RBD is believed to mediate entry of the lineage B SARS coronavirus to respiratory epithelial cells by binding to the cell surface receptor angiotensin-converting enzyme 2 (ACE2). In particular, a receptor binding motif (RBM) in the virus RBD is believed to interact with ACE2.

The amino acid sequence of the SARS-CoV-2 Wuhan-Hu-1 surface glycoprotein is provided in SEQ ID NO.:165. The amino acid sequence of SARS-CoV-2 Wuhan coronavirus RBD is provided in SEQ ID NO.:166. Wuhan coronavirus S protein has approximately 73% amino acid sequence identity with SARS-CoV. The amino acid sequence of Wuhan coronavirus RBM is provided in SEQ ID NO.:167. Wuhan coronavirus RBD has approximately 75% to 77% amino acid sequence similarity to SARS coronavirus RBD, and Wuhan coronavirus RBM has approximately 50% amino acid sequence similarity to SARS coronavirus RBM.

Unless otherwise indicated herein, SARS-CoV-2 Wu-Hu-1 refers to a virus comprising the amino acid sequence set forth in any one or more of GenBank QHD43416.1, Jan. 23, 2020 or SEQ ID NOs.:165, and 166, optionally with the genomic sequence set forth in GenBank MN908947.3, Jan. 23, 2020.

There have been a number of emerging SARS-CoV-2 variants. Some SARS-CoV-2 variants contain an N439K mutation, which has enhanced binding affinity to the human ACE2 receptor (Thomson, E. C., et al., The circulating SARS-CoV-2 spike variant N439K maintains fitness while evading antibody-mediated immunity. bioRxiv, 2020). Some SARS-CoV-2 variants contain an N501Y mutation, which is associated with increased transmissibility, including the lineages B.1.1.7 (also known as 20I/501Y.V1 and VOC 202012/01) and B.1.351 (also known as 20H/501Y.V2), which were discovered in the United Kingdom and South Africa, respectively (Tegally, H., et al., Emergence and rapid spread of a new severe acute respiratory syndrome-related coronavirus 2 (SARS-CoV-2) lineage with multiple spike mutations in South Africa. medRxiv, 2020: p. 2020.12.21.20248640; Leung, K., et al., Early empirical assessment of the N501Y mutant strains of SARS-CoV-2 in the United Kingdom, October to November 2020. medRxiv, 2020: p. 2020.12.20.20248581). B.1.351 also include two other mutations in the RBD domain of SARS-CoV2 spike protein, K417N and E484K (Tegally, H., et al., Emergence and rapid spread of a new severe acute respiratory syndrome-related coronavirus 2 (SARS-CoV-2) lineage with multiple spike mutations in South Africa. medRxiv, 2020: p. 2020.12.21.20248640).

Lineage B.1.617.2 (also known as the delta variant) contains multiple spike protein mutations: T19R, Δ157-158, L452R, T478K, D614G, P681R, and D950N. Of particular concern the P681R mutation appears to be linked to the higher viral loads and increased transmission of lineage B.1.617.2. (Bernal J L, Andrews N, Gower C, et al. Effectiveness of COVID-19 Vaccines against the B.1.617.2 (Delta) Variant. N Engl J Med. 385:585-594 (2021).) Although current vaccine remain effective against the lineage, vaccine breakthrough is a concern, as are infections in pediatric patients who are younger than approved vaccination ages.

Other SARS-CoV-2 variants include the Lineage B.1.1.28, which was first reported in Brazil; the Variant P.1, lineage B.1.1.28 (also known as 20J/501Y.V3), which was first reported in Japan; Variant L452R, which was first reported in California in the United States (Pan American Health Organization, Epidemiological update: Occurrence of variants of SARS-CoV-2 in the Americas, Jan. 20, 2021, available at https://reliefweb.int/sites/reliefweb.int/files/resources/2021-jan-20-phe-epi-update-SARS-CoV-2.pdf). Other SARS-CoV-2 variants include a SARS CoV-2 of clade 19A; SARS CoV-2 of clade 19B; a SARS CoV-2 of clade 20A; a SARS CoV-2 of clade 20B; a SARS CoV-2 of clade 20C; a SARS CoV-2 of clade 20D; a SARS CoV-2 of clade 20E (EU1); a SARS CoV-2 of clade 20F; a SARS CoV-2 of clade 20G; and SARS CoV-2 B1.1.207; and other SARS CoV-2 lineages described in Rambaut, A., et al., A dynamic nomenclature proposal for SARS-CoV-2 lineages to assist genomic epidemiology. Nat Microbiol 5, 1403-1407 (2020).

A SARS CoV-2 infection in accordance with the present disclosure includes infection by any one or more of the aforementioned SARS-CoV-2 viruses and variants thereof.

“Multisystem Inflammatory Syndrome” or “MIS-C,” MIS-C (also known as Pediatric Multisystem Inflammatory Syndrome temporally associated with COVID-19, PIMS-TS or PIMS for short) represents a (to-date) rare complication of SARS-CoV-2 infection observed in children and recently a similar syndrome in adults Multisystem Inflammatory Syndrome Adults (MIS A) has been observed. MIS C is similar in presentation to Kawasaki disease (KD) or Toxic Shock Syndrome (TSS), and the condition is characterized by a severe auto-inflammatory disorder involving two or more multiple organ systems (cardiac, renal, respiratory, hematologic, gastrointestinal, dermatologic, or neurological).

As of 3 May 2021, the US Centers for Disease Control and Prevention reported 3742 MIS-C cases meeting the case definition had been reported with 35 deaths meeting the case definition. The median age of children with MIS-C was 9 years, with half of the cases occurring in children between the ages of 5 and 13. As well, cases were disproportionately Hispanic or Latino or non-Hispanic Black (63%). Obesity was the most commonly reported comorbidity, and at least 60% of cases in all studies required ICU admission.

This condition is most often diagnosed with the use of SARS-CoV-2 serology as the majority of children presenting with the condition no longer have detectable virus by PCR. This is consistent with reports that MIS-C generally presents between three to four weeks following SARS-CoV-2 infection. It has been suggested that this three- to four-week lag coincides with the timing of acquired immunity and that MIS-C is likely to represent a post-infectious complication thought to be driven by formation of pathogenic autoantibodies.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives. As used herein, the terms “include,” “have,” and “comprise” are used synonymously, which terms and variants thereof are intended to be construed as non-limiting.

“Optional” or “optionally” means that the subsequently described element, component, event, or circumstance may or may not occur, and that the description includes instances in which the element, component, event, or circumstance occurs and instances in which they do not.

In addition, it should be understood that the individual constructs, or groups of constructs, derived from the various combinations of the structures and subunits described herein, are disclosed by the present application to the same extent as if each construct or group of constructs was set forth individually. Thus, selection of particular structures or particular subunits is within the scope of the present disclosure.

The term “consisting essentially of” is not equivalent to “comprising” and refers to the specified materials or steps of a claim, or to those that do not materially affect the basic characteristics of a claimed subject matter. For example, a protein domain, region, or module (e.g., a binding domain) or a protein “consists essentially of” a particular amino acid sequence when the amino acid sequence of a domain, region, module, or protein includes extensions, deletions, mutations, or a combination thereof (e.g., amino acids at the amino- or carboxy-terminus or between domains) that, in combination, contribute to at most 20% (e.g., at most 15%, 10%, 8%, 6%, 5%, 4%, 3%, 2% or 1%) of the length of a domain, region, module, or protein and do not substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity of a binding protein).

“Treat,” “treatment,” or “ameliorate” refers to medical management of a disease, disorder, or condition of a subject (e.g., a human or non-human mammal, such as a primate, horse, cat, dog, goat, mouse, or rat). In general, an appropriate dose or treatment regimen comprising an antibody, antigen-binding fragment, or composition of the present disclosure is administered in an amount sufficient to elicit a therapeutic or prophylactic benefit. Therapeutic or prophylactic/preventive benefit includes, for example, improved clinical outcome; lessening or alleviation of symptoms associated with a disease; decreased occurrence of symptoms; improved quality of life; longer disease-free status; diminishment of extent of disease, stabilization of disease state; delay or prevention of disease progression; remission; survival; prolonged survival; or any combination thereof. In certain embodiments, therapeutic or prophylactic/preventive benefit includes reduction or prevention of hospitalization for treatment of a SARS-CoV-2 infection or COVID-19 (i.e., in a statistically significant manner). In certain embodiments, therapeutic or prophylactic/preventive benefit includes a reduced duration of hospitalization for treatment of a SARS-CoV-2 infection or COVID-19 (i.e., in a statistically significant manner). In certain embodiments, therapeutic or prophylactic/preventive benefit includes a reduced or abrogated need for respiratory intervention, such as intubation and/or the use of a respirator device. In certain embodiments, therapeutic or prophylactic/preventive benefit includes reversing a late-stage disease pathology and/or reducing mortality. In certain embodiments, therapeutic and/or prophylactic benefit comprises a reduction in viral load and/or viral shedding in, e.g., a respiratory sample (lung tissue, nasal swab, sputum, or the like) from the subject. In certain embodiments, therapeutic and/or prophylactic benefit comprises preventing progression of COVID-19, e.g., from mild-to-moderate to severe, or from severe to critical, as described herein. In certain embodiments, therapeutic and/or prophylactic comprises preventing contraction and/or transmission of a SARS-CoV-2 infection, e.g., which can be symptomatic or asymptomatic.

A “therapeutically effective amount” or “effective amount” of an antibody, antigen-binding fragment, or composition of this disclosure refers to an amount of the composition or molecule sufficient to result in a therapeutic effect, including improved clinical outcome; lessening or alleviation of symptoms associated with a disease; decreased occurrence of symptoms; improved quality of life; longer disease-free status; diminishment of extent of disease, stabilization of disease state; delay of disease progression; remission; survival; or prolonged survival in a statistically significant manner. When referring to an individual active ingredient, administered alone, a therapeutically effective amount refers to the effects of that ingredient or cell expressing that ingredient alone. When referring to a combination, a therapeutically effective amount refers to the combined amounts of active ingredients or combined adjunctive active ingredient with a cell expressing an active ingredient that results in a therapeutic effect, whether administered serially, sequentially, or simultaneously. A combination may comprise, for example, two different antibodies that specifically bind a SARS-CoV-2 antigen, which in certain embodiments, may be the same or different SARS-CoV-2 antigen, and/or can comprise the same or different epitopes.

As used herein, “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that functions in a manner similar to a naturally occurring amino acid.

As used herein, “mutation” refers to a change in the sequence of a nucleic acid molecule or polypeptide molecule as compared to a reference or wild-type nucleic acid molecule or polypeptide molecule, respectively. A mutation can result in several different types of change in sequence, including substitution, insertion or deletion of nucleotide(s) or amino acid(s).

A “conservative substitution” refers to amino acid substitutions that do not significantly affect or alter binding characteristics of a particular protein. Generally, conservative substitutions are ones in which a substituted amino acid residue is replaced with an amino acid residue having a similar side chain. Conservative substitutions include a substitution found in one of the following groups: Group 1: Alanine (Ala or A), Glycine (Gly or G), Serine (Ser or S), Threonine (Thr or T); Group 2: Aspartic acid (Asp or D), Glutamic acid (Glu or Z); Group 3: Asparagine (Asn or N), Glutamine (Gln or Q); Group 4: Arginine (Arg or R), Lysine (Lys or K), Histidine (His or H); Group 5: Isoleucine (Ile or I), Leucine (Leu or L), Methionine (Met or M), Valine (Val or V); and Group 6: Phenylalanine (Phe or F), Tyrosine (Tyr or Y), Tryptophan (Trp or W). Additionally or alternatively, amino acids can be grouped into conservative substitution groups by similar function, chemical structure, or composition (e.g., acidic, basic, aliphatic, aromatic, or sulfur-containing). For example, an aliphatic grouping may include, for purposes of substitution, Gly, Ala, Val, Leu, and Ile. Other conservative substitutions groups include: sulfur-containing: Met and Cysteine (Cys or C); acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information can be found in Creighton (1984) Proteins, W.H. Freeman and Company.

As used herein, “protein” or “polypeptide” refers to a polymer of amino acid residues. Proteins apply to naturally occurring amino acid polymers, as well as to amino acid polymers in which one or more amino acid residue is an artificial chemical mimetic of a corresponding naturally occurring amino acid, and non-naturally occurring amino acid polymers. Variants of proteins, peptides, and polypeptides of this disclosure are also contemplated. In certain embodiments, variant proteins, peptides, and polypeptides comprise or consist of an amino acid sequence that is at least 70%, 75%, 80%, 85%, 90%, 91%, 92%, 93%, 94%, 95%, 96%, 97%, 98%, 99%, or 99.9% identical to an amino acid sequence of a defined or reference amino acid sequence as described herein.

“Nucleic acid molecule” or “polynucleotide” or “polynucleic acid” refers to a polymeric compound including covalently linked nucleotides, which can be made up of natural subunits (e.g., purine or pyrimidine bases) or non-natural subunits (e.g., morpholine ring). Purine bases include adenine, guanine, hypoxanthine, and xanthine, and pyrimidine bases include uracil, thymine, and cytosine. Nucleic acid molecules include polyribonucleic acid (RNA), which includes mRNA, microRNA, siRNA, viral genomic RNA, and synthetic RNA, and polydeoxyribonucleic acid (DNA), which includes cDNA, genomic DNA, and synthetic DNA, either of which may be single or double stranded. If single-stranded, the nucleic acid molecule may be the coding strand or non-coding (anti-sense) strand. A nucleic acid molecule encoding an amino acid sequence includes all nucleotide sequences that encode the same amino acid sequence. Some versions of the nucleotide sequences may also include intron(s) to the extent that the intron(s) would be removed through co- or post-transcriptional mechanisms. In other words, different nucleotide sequences may encode the same amino acid sequence as the result of the redundancy or degeneracy of the genetic code, or by splicing.

Variants of nucleic acid molecules of this disclosure are also contemplated. Variant nucleic acid molecules are at least 70%, 75%, 80%, 85%, 90%, and are preferably 95%, 96%, 97%, 98%, 99%, or 99.9% identical a nucleic acid molecule of a defined or reference polynucleotide as described herein, or that hybridize to a polynucleotide under stringent hybridization conditions of 0.015M sodium chloride, 0.0015M sodium citrate at about 65-68° C. or 0.015M sodium chloride, 0.0015M sodium citrate, and 50% formamide at about 42° C. Nucleic acid molecule variants retain the capacity to encode a binding domain thereof having a functionality described herein, such as binding a target molecule.

“Percent sequence identity” refers to a relationship between two or more sequences, as determined by comparing the sequences. Preferred methods to determine sequence identity are designed to give the best match between the sequences being compared. For example, the sequences are aligned for optimal comparison purposes (e.g., gaps can be introduced in one or both of a first and a second amino acid or nucleic acid sequence for optimal alignment). Further, non-homologous sequences may be disregarded for comparison purposes. The percent sequence identity referenced herein is calculated over the length of the reference sequence, unless indicated otherwise. Methods to determine sequence identity and similarity can be found in publicly available computer programs. Sequence alignments and percent identity calculations may be performed using a BLAST program (e.g., BLAST 2.0, BLASTP, BLASTN, or BLASTX). The mathematical algorithm used in the BLAST programs can be found in Altschul et al., Nucleic Acids Res. 25:3389-3402, 1997. Within the context of this disclosure, it will be understood that where sequence analysis software is used for analysis, the results of the analysis are based on the “default values” of the program referenced. “Default values” mean any set of values or parameters which originally load with the software when first initialized, however, filtering may not be used in connection with low complexity regions.

The term “isolated” means that the material is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such nucleic acid could be part of a vector and/or such nucleic acid or polypeptide could be part of a composition (e.g., a cell lysate), and still be isolated in that such vector or composition is not part of the natural environment for the nucleic acid or polypeptide.

The term “gene” means the segment of DNA or RNA involved in producing a polypeptide chain; in certain contexts, it includes regions preceding and following the coding region (e.g., 5′ untranslated region (UTR) and 3′ UTR) as well as intervening sequences (introns) between individual coding segments (exons).

A “functional variant” refers to a polypeptide or polynucleotide that is structurally similar or substantially structurally similar to a parent or reference compound of this disclosure, but differs slightly in composition (e.g., one base, atom or functional group is different, added, or removed), such that the polypeptide or encoded polypeptide is capable of performing at least one function of the parent polypeptide with at least 50% efficiency (i.e. level of activity of the parent polypeptide), preferably at least 55%, 60%, 70%, 75%, 80%, 85%, 90%, 95%, 96%, 97%, 98%, 99%, 99.9%, or 100% efficiency. In other words, a functional variant of a polypeptide or encoded polypeptide of this disclosure has “similar binding,” “similar affinity” or “similar activity” when the functional variant displays no more than a 50% reduction in performance in a selected assay as compared to the parent or reference polypeptide, such as an assay for measuring binding affinity (e.g., Biacore™ (Cytiva) or tetramer staining measuring an association (Ka) or a dissociation (K_{D}) constant).

As used herein, a “functional portion” or “functional fragment” refers to a polypeptide or polynucleotide that comprises only a domain, portion or fragment of a parent or reference compound, and the polypeptide or encoded polypeptide retains at least 50% efficiency (i.e. level of activity of the parent polypeptide) associated with the domain, portion or fragment of the parent or reference compound, preferably at least 55%, 60%, 70%, 75%, 80%, 85%, 90%, 95%, 96%, 97%, 98%, 99%, 99.9%, or 100% efficiency of the parent polypeptide, or provides a biological benefit (e.g., effector function). A “functional portion” or “functional fragment” of a polypeptide or encoded polypeptide of this disclosure has “similar binding” or “similar activity” when the functional portion or fragment displays no more than a 50% reduction in performance in a selected assay as compared to the parent or reference polypeptide (preferably no more than 20% or 10%, or no more than a log difference as compared to the parent or reference with regard to affinity).

As used herein, the term “engineered,” “recombinant,” or “non-natural” refers to an organism, microorganism, cell, nucleic acid molecule, or vector that includes at least one genetic alteration or has been modified by introduction of an exogenous or heterologous nucleic acid molecule, wherein such alterations or modifications are introduced by genetic engineering (i.e., human intervention). Genetic alterations include, for example, modifications introducing expressible nucleic acid molecules encoding functional RNA, proteins, fusion proteins or enzymes, or other nucleic acid molecule additions, deletions, substitutions, or other functional disruption of a cell's genetic material. Additional modifications include, for example, non-coding regulatory regions in which the modifications alter expression of a polynucleotide, gene, or operon.

As used herein, “heterologous” or “non-endogenous” or “exogenous” refers to any gene, protein, compound, nucleic acid molecule, or activity that is not native to a host cell or a subject, or any gene, protein, compound, nucleic acid molecule, or activity native to a host cell or a subject that has been altered. Heterologous, non-endogenous, or exogenous includes genes, proteins, compounds, or nucleic acid molecules that have been mutated or otherwise altered such that the structure, activity, or both is different as between the native and altered genes, proteins, compounds, or nucleic acid molecules. In certain embodiments, heterologous, non-endogenous, or exogenous genes, proteins, or nucleic acid molecules (e.g., receptors, ligands, etc.) may not be endogenous to a host cell or a subject, but instead nucleic acids encoding such genes, proteins, or nucleic acid molecules may have been added to a host cell by conjugation, transformation, transfection, electroporation, or the like, wherein the added nucleic acid molecule may integrate into a host cell genome or can exist as extra-chromosomal genetic material (e.g., as a plasmid or other self-replicating vector). The term “homologous” or “homolog” refers to a gene, protein, compound, nucleic acid molecule, or activity found in or derived from a host cell, species, or strain. For example, a heterologous or exogenous polynucleotide or gene encoding a polypeptide may be homologous to a native polynucleotide or gene and encode a homologous polypeptide or activity, but the polynucleotide or polypeptide may have an altered structure, sequence, expression level, or any combination thereof. A non-endogenous polynucleotide or gene, as well as the encoded polypeptide or activity, may be from the same species, a different species, or a combination thereof.

In certain embodiments, a nucleic acid molecule or portion thereof native to a host cell will be considered heterologous to the host cell if it has been altered or mutated, or a nucleic acid molecule native to a host cell may be considered heterologous if it has been altered with a heterologous expression control sequence or has been altered with an endogenous expression control sequence not normally associated with the nucleic acid molecule native to a host cell. In addition, the term “heterologous” can refer to a biological activity that is different, altered, or not endogenous to a host cell. As described herein, more than one heterologous nucleic acid molecule can be introduced into a host cell as separate nucleic acid molecules, as a plurality of individually controlled genes, as a polycistronic nucleic acid molecule, as a single nucleic acid molecule encoding a fusion protein, or any combination thereof.

As used herein, the term “endogenous” or “native” refers to a polynucleotide, gene, protein, compound, molecule, or activity that is normally present in a host cell or a subject.

The term “expression”, as used herein, refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof Δn expressed nucleic acid molecule is typically operably linked to an expression control sequence (e.g., a promoter).

The term “operably linked” refers to the association of two or more nucleic acid molecules on a single nucleic acid fragment so that the function of one is affected by the other. For example, a promoter is operably linked with a coding sequence when it is capable of affecting the expression of that coding sequence (i.e., the coding sequence is under the transcriptional control of the promoter). “Unlinked” means that the associated genetic elements are not closely associated with one another and the function of one does not affect the other.

As described herein, more than one heterologous nucleic acid molecule can be introduced into a host cell as separate nucleic acid molecules, as a plurality of individually controlled genes, as a polycistronic nucleic acid molecule, as a single nucleic acid molecule encoding a protein (e.g., a heavy chain of an antibody), or any combination thereof. When two or more heterologous nucleic acid molecules are introduced into a host cell, it is understood that the two or more heterologous nucleic acid molecules can be introduced as a single nucleic acid molecule (e.g., on a single vector), on separate vectors, integrated into the host chromosome at a single site or multiple sites, or any combination thereof. The number of referenced heterologous nucleic acid molecules or protein activities refers to the number of encoding nucleic acid molecules or the number of protein activities, not the number of separate nucleic acid molecules introduced into a host cell.

The term “construct” refers to any polynucleotide that contains a recombinant nucleic acid molecule (or, when the context clearly indicates, a fusion protein of the present disclosure). A (polynucleotide) construct may be present in a vector (e.g., a bacterial vector, a viral vector) or may be integrated into a genome. A “vector” is a nucleic acid molecule that is capable of transporting another nucleic acid molecule. Vectors may be, for example, plasmids, cosmids, viruses, a RNA vector or a linear or circular DNA or RNA molecule that may include chromosomal, non-chromosomal, semi-synthetic or synthetic nucleic acid molecules. Vectors of the present disclosure also include transposon systems (e.g., Sleeping Beauty, see, e.g., Geurts et al., Mol. Ther. 8:108, 2003: Mátés et al., Nat. Genet. 41:753, 2009). Exemplary vectors are those capable of autonomous replication (episomal vector), capable of delivering a polynucleotide to a cell genome (e.g., viral vector), or capable of expressing nucleic acid molecules to which they are linked (expression vectors).

As used herein, “expression vector” or “vector” refers to a DNA construct containing a nucleic acid molecule that is operably linked to a suitable control sequence capable of effecting the expression of the nucleic acid molecule in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host, the vector may replicate and function independently of the host genome, or may, in some instances, integrate into the genome itself or deliver the polynucleotide contained in the vector into the genome without the vector sequence. In the present specification, “plasmid,” “expression plasmid,” “virus,” and “vector” are often used interchangeably.

The term “introduced” in the context of inserting a nucleic acid molecule into a cell, means “transfection”, “transformation,” or “transduction” and includes reference to the incorporation of a nucleic acid molecule into a eukaryotic or prokaryotic cell wherein the nucleic acid molecule may be incorporated into the genome of a cell (e.g., chromosome, plasmid, plastid, or mitochondrial DNA), converted into an autonomous replicon, or transiently expressed (e.g., transfected mRNA).

In certain embodiments, polynucleotides of the present disclosure may be operatively linked to certain elements of a vector. For example, polynucleotide sequences that are needed to effect the expression and processing of coding sequences to which they are ligated may be operatively linked. Expression control sequences may include appropriate transcription initiation, termination, promoter, and enhancer sequences; efficient RNA processing signals such as splicing and polyadenylation signals; sequences that stabilize cytoplasmic mRNA; sequences that enhance translation efficiency (i.e., Kozak consensus sequences); sequences that enhance protein stability; and possibly sequences that enhance protein secretion. Expression control sequences may be operatively linked if they are contiguous with the gene of interest and expression control sequences that act in trans or at a distance to control the gene of interest.

In certain embodiments, the vector comprises a plasmid vector or a viral vector (e.g., a lentiviral vector or a γ-retroviral vector). Viral vectors include retrovirus, adenovirus, parvovirus (e.g., adeno-associated viruses), coronavirus, negative strand RNA viruses such as ortho-myxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox, and canarypox). Other viruses include, for example, Norwalk virus, togavirus, flavivirus, reoviruses, papovavirus, hepadnavirus, and hepatitis virus. Examples of retroviruses include avian leukosis-sarcoma, mammalian C-type, B-type viruses, D type viruses, HTLV-BLV group, lentivirus, and spumavirus (Coffin, J. M., Retroviridae: The viruses and their replication, In Fundamental Virology, Third Edition, B. N. Fields et al., Eds., Lippincott-Raven Publishers, Philadelphia, 1996).

“Retroviruses” are viruses having an RNA genome, which is reverse-transcribed into DNA using a reverse transcriptase enzyme, the reverse-transcribed DNA is then incorporated into the host cell genome. “Gammaretrovirus” refers to a genus of the retroviridae family. Examples of gammaretroviruses include mouse stem cell virus, murine leukemia virus, feline leukemia virus, feline sarcoma virus, and avian reticuloendotheliosis viruses.

“Lentiviral vectors” include HIV-based lentiviral vectors for gene delivery, which can be integrative or non-integrative, have relatively large packaging capacity, and can transduce a range of different cell types. Lentiviral vectors are usually generated following transient transfection of three (packaging, envelope, and transfer) or more plasmids into producer cells. Like HIV, lentiviral vectors enter the target cell through the interaction of viral surface glycoproteins with receptors on the cell surface. On entry, the viral RNA undergoes reverse transcription, which is mediated by the viral reverse transcriptase complex. The product of reverse transcription is a double-stranded linear viral DNA, which is the substrate for viral integration into the DNA of infected cells.

In certain embodiments, the viral vector can be a gammaretrovirus, e.g., Moloney murine leukemia virus (MLV)-derived vectors. In other embodiments, the viral vector can be a more complex retrovirus-derived vector, e.g., a lentivirus-derived vector. HIV-1-derived vectors belong to this category. Other examples include lentivirus vectors derived from HIV-2, FIV, equine infectious anemia virus, SIV, and Maedi-Visna virus (ovine lentivirus). Methods of using retroviral and lentiviral viral vectors and packaging cells for transducing mammalian host cells with viral particles containing transgenes are known in the art and have been previously described, for example, in: U.S. Pat. No. 8,119,772; Walchli et al., PLoS One 6:327930, 2011; Zhao et al., J. Immunol. 174:4415, 2005; Engels et al., Hum. Gene Ther. 14:1155, 2003; Frecha et al., Mol. Ther. 18:1748, 2010; and Verhoeyen et al., Methods Mol. Biol. 506:97, 2009. Retroviral and lentiviral vector constructs and expression systems are also commercially available. Other viral vectors also can be used for polynucleotide delivery including DNA viral vectors, including, for example adenovirus-based vectors and adeno-associated virus (AAV)-based vectors; vectors derived from herpes simplex viruses (HSVs), including amplicon vectors, replication-defective HSV and attenuated HSV (Krisky et al., Gene Ther. 5:1517, 1998).

Other vectors that can be used with the compositions and methods of this disclosure include those derived from baculoviruses and α-viruses. (Jolly, D J. 1999. Emerging Viral Vectors. pp 209-40 in Friedmann T. ed. The Development of Human Gene Therapy. New York: Cold Spring Harbor Lab), or plasmid vectors (such as Sleeping Beauty or other transposon vectors).

When a viral vector genome comprises a plurality of polynucleotides to be expressed in a host cell as separate transcripts, the viral vector may also comprise additional sequences between the two (or more) transcripts allowing for bicistronic or multicistronic expression. Examples of such sequences used in viral vectors include internal ribosome entry sites (IRES), furin cleavage sites, viral 2A peptide, or any combination thereof.

Plasmid vectors, including DNA-based antibody or antigen-binding fragment-encoding plasmid vectors for direct administration to a subject, are described further herein.

As used herein, the term “host” refers to a cell or microorganism targeted for genetic modification with a heterologous nucleic acid molecule to produce a polypeptide of interest (e.g., an antibody of the present disclosure).

A host cell may include any individual cell or cell culture which may receive a vector or the incorporation of nucleic acids or express proteins. The term also encompasses progeny of the host cell, whether genetically or phenotypically the same or different. Suitable host cells may depend on the vector and may include mammalian cells, animal cells, human cells, simian cells, insect cells, yeast cells, and bacterial cells. These cells may be induced to incorporate the vector or other material by use of a viral vector, transformation via calcium phosphate precipitation, DEAE-dextran, electroporation, microinjection, or other methods. See, for example, Sambrook et al., Molecular Cloning: A Laboratory Manual 2d ed. (Cold Spring Harbor Laboratory, 1989).

In the context of a SARS-CoV-2 infection, a “host” refers to a cell or a subject infected with the SARS-CoV-2.

“Antigen” or “Ag”, as used herein, refers to an immunogenic molecule that provokes an immune response. This immune response may involve antibody production, activation of specific immunologically-competent cells, activation of complement, antibody dependent cytotoxicity, or any combination thereof. An antigen (immunogenic molecule) may be, for example, a peptide, glycopeptide, polypeptide, glycopolypeptide, polynucleotide, polysaccharide, lipid, or the like. It is readily apparent that an antigen can be synthesized, produced recombinantly, or derived from a biological sample. Exemplary biological samples that can contain one or more antigens include tissue samples, stool samples, cells, biological fluids, or combinations thereof. Antigens can be produced by cells that have been modified or genetically engineered to express an antigen. Antigens can also be present in a SARS-CoV-2 (e.g., a surface glycoprotein or portion thereof), such as present in a virion, or expressed or presented on the surface of a cell infected by the SARS-CoV-2.

The term “epitope” or “antigenic epitope” includes any molecule, structure, amino acid sequence, or protein determinant that is recognized and specifically bound by a cognate binding molecule, such as an immunoglobulin, or other binding molecule, domain, or protein. Epitopic determinants generally contain chemically active surface groupings of molecules, such as amino acids or sugar side chains, and can have specific three-dimensional structural characteristics, as well as specific charge characteristics. Where an antigen is or comprises a peptide or protein, the epitope can be comprised of consecutive amino acids (e.g., a linear epitope), or can be comprised of amino acids from different parts or regions of the protein that are brought into proximity by protein folding (e.g., a discontinuous or conformational epitope), or non-contiguous amino acids that are in close proximity irrespective of protein folding.

### Antibodies, Antigen-Binding Fragments, and Compositions

Certain presently disclosed methods and uses comprise administering to a subject antibody, or an antigen-binding fragment thereof, that comprises a heavy chain variable domain (VH) comprising a CDRH1, a CDRH2, and a CDRH3, and a light chain variable domain (VL) comprising a CDRL1, a CDRL2, and a CDRL3, and is capable of binding to a surface glycoprotein of a SARS-CoV-2 (e.g. as expressed on a cell surface of a host cell and/or on a SARS-CoV-2 virion).

In certain preferred embodiments, described further herein, an antibody or antigen-binding fragment thereof used in a method comprises the CDRH1, CDRH2, CDRH3, CDRL1, CDRL2, and CDRL3 amino acid sequences set forth in SEQ ID NOs.:106, 121, 108, 169, 170, and 171, respectively, or set forth in SEQ ID NOs.:106, 107, 108, 169, 170, and 171, respectively. In certain embodiments, an antibody or antigen-binding fragment thereof used in a method comprises the VH amino acid sequence set forth in SEQ ID NO.:113 or 105 and the VL amino acid sequence set forth in SEQ ID NO.:168.

In certain embodiments, an antibody or antigen-binding fragment of used in a method associates with or unites with a SARS-CoV-2 surface glycoprotein epitope or antigen comprising the epitope, while not significantly associating or uniting with any other molecules or components in a sample.

In certain embodiments, an antibody or antigen-binding fragment of the present disclosure associates with or unites (e.g., binds) to a SARS-CoV-2 surface glycoprotein epitope, and can also associate with or unite with an epitope from another coronavirus (e.g., SARS CoV) present in the sample, but not significantly associating or uniting with any other molecules or components in the sample. In other words, in certain embodiments, an antibody or antigen binding fragment of the present disclosure is cross-reactive for SARS-CoV-2 and one or more additional coronavirus.

In certain embodiments, an antibody or antigen-binding fragment of the present disclosure specifically binds to a SARS-CoV-2 surface glycoprotein. As used herein, “specifically binds” refers to an association or union of an antibody or antigen-binding fragment to an antigen with an affinity or Kₐ (i.e., an equilibrium association constant of a particular binding interaction with units of 1/M) equal to or greater than 10⁵ M⁻¹ (which equals the ratio of the on-rate [Kₒₙ] to the off rate [K_{off}] for this association reaction), while not significantly associating or uniting with any other molecules or components in a sample. Alternatively, affinity may be defined as an equilibrium dissociation constant (K_{d}) of a particular binding interaction with units of M (e.g., 10⁻⁵ M to 10⁻¹³ M).

A variety of assays are known for identifying antibodies of the present disclosure that bind a particular target, as well as determining binding domain or binding protein affinities, such as Western blot, ELISA (e.g., direct, indirect, or sandwich), analytical ultracentrifugation, spectroscopy, and surface plasmon resonance (Biacore™) analysis (see, e.g., Scatchard et al., Ann. N.Y. Acad. Sci. 51:660, 1949; Wilson, Science 295:2103, 2002; Wolff et al., Cancer Res. 53:2560, 1993; and U.S. Pat. Nos. 5,283,173, 5,468,614, or the equivalent). Assays for assessing affinity or apparent affinity or relative affinity are also known.

Binding can be determined by, for example, recombinantly expressing a SARS-CoV-2 antigen in a host cell (e.g., by transfection) and immunostaining the (e.g., fixed, or fixed and permeabilized) host cell with antibody and analyzing binding by flow cytometery (e.g., using a ZE5 Cell Analyzer (BioRad®) and FlowJo™ software (TreeStar)). In some embodiments, positive binding can be defined by differential staining by antibody of SARS-CoV-2-expressing cells versus control (e.g., mock) cells.

In certain embodiments, an antibody or antigen-binding fragment is capable of neutralizing infection by SARS-CoV-2. As used herein, a “neutralizing antibody” is one that can neutralize, i.e., prevent, inhibit, reduce, impede, or interfere with, the ability of a pathogen to initiate and/or perpetuate an infection in a host. The terms “neutralizing antibody” and “an antibody that neutralizes” or “antibodies that neutralize” are used interchangeably herein. In any of the presently disclosed embodiments, the antibody or antigen-binding fragment is capable of preventing and/or neutralizing a SARS-CoV-2 infection in an in vitro model of infection and/or in an in vivo animal model of infection and/or in a human.

Terms understood by those in the art of antibody technology are each given the meaning acquired in the art, unless expressly defined differently herein. For example, the term “antibody” refers to an intact antibody comprising at least two heavy (H) chains and two light (L) chains inter-connected by disulfide bonds, as well as any antigen-binding portion or fragment of an intact antibody that has or retains the ability to bind to the antigen target molecule recognized by the intact antibody, such as an scFv, Fab, or Fab′2 fragment. Thus, the term “antibody” herein is used in the broadest sense and includes polyclonal and monoclonal antibodies, including intact antibodies and functional (antigen-binding) antibody fragments thereof, including fragment antigen binding (Fab) fragments, F(ab′)2 fragments, Fab′ fragments, Fv fragments, recombinant IgG (rIgG) fragments, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific antibodies, diabodies, triabodies, tetrabodies, tandem di-scFv, and tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof (IgG1, IgG2, IgG3, IgG4), IgM, IgE, IgA, and IgD. In particular embodiments, an antibody is an IgG1 isotype.

The terms “V_{L}” or “VL” and “V_{H}” or “VH” refer to the variable binding region from an antibody light chain and an antibody heavy chain, respectively. In certain embodiments, a VL is a kappa (κ) class (also “VK” herein). In certain embodiments, a VL is a lambda (λ) class. The variable binding regions comprise discrete, well-defined sub-regions known as “complementarity determining regions” (CDRs) and “framework regions” (FRs). The terms “complementarity determining region,” and “CDR,” are synonymous with “hypervariable region” or “HVR,” and refer to sequences of amino acids within antibody variable regions, which, in general, together confer the antigen specificity and/or binding affinity of the antibody, wherein consecutive CDRs (i.e., CDR1 and CDR2, CDR2 and CDR3) are separated from one another in primary structure by a framework region. There are three CDRs in each variable region (HCDR1, HCDR2, HCDR3; LCDR1, LCDR2, LCDR3; also referred to as CDRHs and CDRLs, respectively). In certain embodiments, an antibody VH comprises four FRs and three CDRs as follows: FR1-HCDR1-FR2-HCDR2-FR3-HCDR3-FR4; and an antibody VL comprises four FRs and three CDRs as follows: FR1-LCDR1-FR2-LCDR2-FR3-LCDR3-FR4. In general, the VH and the VL together form the antigen-binding site through their respective CDRs.

Numbering of CDR and framework regions may be according to any known method or scheme, such as the Kabat, Chothia, EU, IMGT, and AHo numbering schemes (see, e.g., Kabat et al., “Sequences of Proteins of Immunological Interest, US Dept. Health and Human Services, Public Health Service National Institutes of Health, 1991, 5^{th} ed.; Chothia and Lesk, J. Mol. Biol. 196:901-917 (1987)); Lefranc et al., Dev. Comp. Immunol. 27:55, 2003; Honegger and Pluckthun, J. Mol. Bio. 309:657-670 (2001)). Equivalent residue positions can be annotated and for different molecules to be compared using Antigen receptor Numbering And Receptor Classification (ANARCI) software tool (2016, Bioinformatics 15:298-300).

In certain embodiments, CDRs are according to the IMGT numbering method.

In certain embodiments, the antibody or antigen-binding fragment comprises CDRH1, CDRH2, CDRH3, CDRL1, CDRL2, and CDRL3 amino acid sequences according to SEQ ID NOs.:106, 121 or 107, 108, 169, 170, and 171, respectively.

In any of the presently disclosed embodiments, the antibody or antigen-binding fragment is capable of preventing and/or neutralizing a SARS-CoV-2 infection in an in vitro model of infection and/or in an in vivo animal model of infection and/or in a human.

The term “CL” refers to an “immunoglobulin light chain constant region” or a “light chain constant region,” i.e., a constant region from an antibody light chain. The term “CH” refers to an “immunoglobulin heavy chain constant region” or a “heavy chain constant region,” which is further divisible, depending on the antibody isotype into CH1, CH2, and CH3 (IgA, IgD, IgG), or CH1, CH2, CH3, and CH4 domains (IgE, IgM). The Fc region of an antibody heavy chain is described further herein. In any of the presently disclosed embodiments, an antibody or antigen-binding fragment of the present disclosure comprises any one or more of CL, a CH1, a CH2, and a CH3. In certain embodiments, a CL comprises an amino acid sequence having 90%, 91%, 92%, 93%, 94%, 95%, 96%, 97%, 98%, 99%, or 100% identity to the amino acid sequence of SEQ ID NO.:174 or SEQ ID NO.: 193. In certain embodiments, a CH1-CH2-CH3 comprises an amino acid sequence having 90%, 91%, 92%, 93%, 94%, 95%, 96%, 97%, 98%, 99%, or 100% identity to the amino acid sequence of SEQ ID NO.:173 or SEQ ID NO.:175.

It will be understood that, for example, production in a mammalian cell line can remove one or more C-terminal lysine of an antibody heavy chain (see, e.g., Liu et al. mAbs 6(5):1145-1154 (2014)). Accordingly, an antibody or antigen-binding fragment of the present disclosure can comprise a heavy chain, a CH1-CH3, a CH3, or an Fc polypeptide wherein a C-terminal lysine residue is present or is absent; in other words, encompassed are embodiments where the C-terminal residue of a heavy chain, a CH1-CH3, or an Fc polypeptide is not a lysine, and embodiments where a lysine is the C-terminal residue. Examples of CH1-CH3 amino acid sequences that lack a C-terminal lysine are provided in SEQ ID NOs.:265 and 266.

In certain embodiments, a composition comprises a plurality of an antibody and/or an antigen-binding fragment of the present disclosure, wherein one or more antibody or antigen-binding fragment does not comprise a lysine residue at the C-terminal end of the heavy chain, CH1-CH3, or Fc polypeptide, and wherein one or more antibody or antigen-binding fragment comprises a lysine residue at the C-terminal end of the heavy chain, CH1-CH3, or Fc polypeptide.

A “Fab” (fragment antigen binding) is the part of an antibody that binds to antigens and includes the variable region and CH1 of the heavy chain linked to the light chain via an inter-chain disulfide bond. Each Fab fragment is monovalent with respect to antigen binding, i.e., it has a single antigen-binding site. Pepsin treatment of an antibody yields a single large F(ab′)2 fragment that roughly corresponds to two disulfide linked Fab fragments having divalent antigen-binding activity and is still capable of cross-linking antigen. Both the Fab and F(ab′)2 are examples of “antigen-binding fragments.” Fab′ fragments differ from Fab fragments by having additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments that have hinge cysteines between them. Other chemical couplings of antibody fragments are also known.

Fab fragments may be joined, e.g., by a peptide linker, to form a single chain Fab, also referred to herein as “scFab.” In these embodiments, an inter-chain disulfide bond that is present in a native Fab may not be present, and the linker serves in full or in part to link or connect the Fab fragments in a single polypeptide chain. A heavy chain-derived Fab fragment (e.g., comprising, consisting of, or consisting essentially of VH+CH1, or “Fd”) and a light chain-derived Fab fragment (e.g., comprising, consisting of, or consisting essentially of VL+CL) may be linked in any arrangement to form a scFab. For example, a scFab may be arranged, in N-terminal to C-terminal direction, according to (heavy chain Fab fragment-linker-light chain Fab fragment) or (light chain Fab fragment-linker-heavy chain Fab fragment). Peptide linkers and exemplary linker sequences for use in scFabs are discussed in further detail herein.

A scFab can be comprise any combination of VH and VL sequences or any combination of the CDRH1, CDRH2, CDRH3, CDRL1, CDRL2, and CDRL3 sequences disclosed herein. In certain embodiments, a scFab comprises the VH sequence as provided in SEQ ID NO: 105 or SEQ ID NO: 113 and the VL sequence as provided in SEQ ID NO: 168. In certain embodiments, a scFab comprises a CDRH1 sequence as provided in SEQ ID NO: 106, a CDRH2 sequence as provided in SEQ ID NO: 107 or 121, a CDRH3 sequence as provided in SEQ ID NO: 108, a CDRL1 sequence as provided in SEQ ID NO: 169, a CDRL2 sequence as provided in SEQ ID NO: 170, and a CDRL3 sequence as provided in SEQ ID NO: 171. In certain embodiments, a scFab comprises an amino acid sequence having at least 90%, 91%, 92%, 93%, 94%, 95%, 96%, 97%, 98%, 99%, or 100% identity to the amino acid sequence provided in any one of SEQ ID NOs.: 218-219 or 226-227.

“Fv” is a small antibody fragment that contains a complete antigen-recognition and antigen-binding site. This fragment generally consists of a dimer of one heavy- and one light-chain variable region domain in tight, non-covalent association. However, even a single variable domain (or half of an Fv comprising only three CDRs specific for an antigen) has the ability to recognize and bind antigen, although typically at a lower affinity than the entire binding site.

“Single-chain Fv” also abbreviated as “sFv” or “scFv”, are antibody fragments that comprise the V_{H} and V_{L} antibody domains connected into a single polypeptide chain. In some embodiments, the scFv polypeptide comprises a polypeptide linker disposed between and linking the V_{H} and V_{L} domains that enables the scFv to retain or form the desired structure for antigen binding. Such a peptide linker can be incorporated into a fusion polypeptide using standard techniques well known in the art. For a review of scFv, see Pluckthun in The Pharmacology of Monoclonal Antibodies, vol. 113, Rosenburg and Moore eds., Springer-Verlag, New York, pp. 269-315 (1994); Borrebaeck 1995, infra. In certain embodiments, the antibody or antigen-binding fragment comprises a scFv comprising a VH domain, a VL domain, and a peptide linker linking the VH domain to the VL domain. In particular embodiments, a scFv comprises a VH domain linked to a VL domain by a peptide linker, which can be in a VH-linker-VL orientation or in a VL-linker-VH orientation. Any scFv of the present disclosure may be engineered so that the C-terminal end of the VL domain is linked by a short peptide sequence to the N-terminal end of the VH domain, or vice versa (i.e., (N)VL(C)-linker-(N)VH(C) or (N)VH(C)-linker-(N)VL(C). Alternatively, in some embodiments, a linker may be linked to an N-terminal portion or end of the VH domain, the VL domain, or both.

Peptide linker sequences may be chosen, for example, based on: (1) their ability to adopt a flexible extended conformation; (2) their inability or lack of ability to adopt a secondary structure that could interact with functional epitopes on the first and second polypeptides and/or on a target molecule; and/or (3) the lack or relative lack of hydrophobic or charged residues that might react with the polypeptides and/or target molecule. Other considerations regarding linker design (e.g., length) can include the conformation or range of conformations in which the VH and VL can form a functional antigen-binding site. In certain embodiments, peptide linker sequences contain, for example, Gly, Asn and Ser residues. Other near neutral amino acids, such as Thr and Ala, may also be included in a linker sequence. Other amino acid sequences which may be usefully employed as linker include those disclosed in Maratea et al., Gene 40:39 46 (1985); Murphy et al., Proc. Natl. Acad. Sci. USA 83:8258 8262 (1986); U.S. Pat. Nos. 4,935,233, and 4,751,180. Other illustrative and non-limiting examples of linkers may include, for example, Glu-Gly-Lys-Ser-Ser-Gly-Ser-Gly-Ser-Glu-Ser-Lys-Val-Asp (SEQ ID NO: 215) (Chaudhary et al., Proc. Natl. Acad. Sci. USA 87:1066-1070 (1990)) and Lys-Glu-Ser-Gly-Ser-Val-Ser-Ser-Glu-Gln-Leu-Ala-Gln-Phe-Arg-Ser-Leu-Asp (SEQ ID NO: 216) (Bird et al., Science 242:423-426 (1988)) and the pentamer Gly-Gly-Gly-Gly-Ser (SEQ ID NO: 217) when present in a single iteration or repeated 1 to 5 or more times, or more; see, e.g., SEQ ID NO: 213. Any suitable linker may be used, and in general can be about 3, 4, 5, 6, 7, 8, 9, 10, 11, 12, 13, 14, 15, 16, 17, 18, 19, 20, 21, 22, 15 23, 24, 25, 26, 27, 28, 29, 30, 40, 50, 60, 70, 80, 90, 100 amino acids in length, or less than about 200 amino acids in length, and will preferably comprise a flexible structure (can provide flexibility and room for conformational movement between two regions, domains, motifs, fragments, or modules connected by the linker), and will preferably be biologically inert and/or have a low risk of immunogenicity in a human. Exemplary linkers include those comprising or consisting of the amino acid sequence set forth in any one or more of SEQ ID NOs: 206-217. In certain embodiments, the linker comprises or consists of an amino acid sequence having at least 75% (i.e., at least about 75%, 80%, 85%, 90%, 91%, 92%, 93%, 94%, 95%, 96%, 97%, 98%, 99%, or more) identity to the amino acid sequence set forth in any one of SEQ ID NOs: 206-217.

scFv can be constructed using any combination of the VH and VL sequences or any combination of the CDRH1, CDRH2, CDRH3, CDRL1, CDRL2, and CDRL3 sequences disclosed herein. In certain embodiments, a scFv comprises the VH sequence provided in SEQ ID NO: 105 or SEQ ID NO: 113 and the VL sequence provided in SEQ ID NO: 168. In certain embodiments, a scFab comprises a CDRH1 sequence as provided in SEQ ID NO: 106, a CDRH2 sequence as provided in SEQ ID NO: 107 or 121, a CDRH3 sequence as provided in SEQ ID NO: 108, a CDRL1 sequence as provided in SEQ ID NO: 169, a CDRL2 sequence as provided in SEQ ID NO: 170, and a CDRL3 sequence as provided in SEQ ID NO: 171. In certain embodiments, a scFv can comprise the amino acid sequence as provided in SEQ ID NO: 220-221 or SEQ ID NO: 228-229.

In some embodiments, linker sequences are not required; for example, when the first and second polypeptides have non-essential N-terminal amino acid regions that can be used to separate the functional domains and prevent steric interference.

Also provided herein are variant antibodies that comprise one or more amino acid alterations in a variable region (e.g., VH, VL, framework or CDR) as compared to a presently disclosed (“parent”) antibody, wherein the variant antibody is capable of binding to a SARS-CoV-2 antigen.

In certain embodiments, the VH comprises or consists of an amino acid sequence having at least 85% (i.e., 85%, 86, 87, 88, 89, 90, 91, 92, 93, 94, 95, 96, 97, 98, 99, or 100%) identity to the amino acid sequence according to any one of SEQ ID NOs.: 105 or 113, wherein the variation is optionally limited to one or more framework regions and/or the variation comprises one or more substitution to a germline-encoded amino acid; and/or (ii) the VL comprises or consists of an amino acid sequence having at least 85% (i.e., 85%, 86, 87, 88, 89, 90, 91, 92, 93, 94, 95, 96, 97, 98, 99, or 100%) identity to the amino acid sequence according to any one of SEQ ID NOs.: 168, wherein the variation is optionally limited to one or more framework regions and/or the variation comprises one or more substitution to a germline-encoded amino acid.

In certain embodiments, an antibody or an antigen-binding fragment of the present disclosure comprises a VH comprising or consisting of the amino acid sequence according to SEQ ID NO: 113 or 105 and a VL comprising or consisting of the amino acid sequence according to SEQ ID NO: 168.

In certain embodiments, an antibody or antigen-binding fragment of the present disclosure is monospecific (e.g., binds to a single epitope) or is multispecific (e.g., binds to multiple epitopes and/or target molecules). Antibodies and antigen binding fragments may be constructed in various formats. Exemplary antibody formats disclosed in Spiess et al., Mol. Immunol. 67(2):95 (2015), and in Brinkmann and Kontermann, mAbs 9(2):182-212 (2017), which formats and methods of making the same are incorporated herein by reference and include, for example, Bispecific T cell Engagers (BiTEs), DARTs, Knobs-Into-Holes (KIH) assemblies, scFv-CH3-KIH assemblies, KIH Common Light-Chain antibodies, TandAbs, Triple Bodies, TriBi Minibodies, Fab-scFv, scFv-CH-CL-scFv, F(ab′)2-scFv2, tetravalent HCabs, Intrabodies, CrossMabs, Dual Action Fabs (DAFs) (two-in-one or four-in-one), DutaMabs, DT-IgG, Charge Pairs, Fab-arm Exchange, SEEDbodies, Triomabs, LUZ-Y assemblies, Fcabs, κλ-bodies, orthogonal Fabs, DVD-Igs (e.g., U.S. Pat. No. 8,258,268, which formats are incorporated herein by reference in their entirety), IgG(H)-scFv, scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig, Zybody, and DVI-IgG (four-in-one), as well as so-called FIT-Ig (e.g., PCT Publication No. WO 2015/103072, which formats are incorporated herein by reference in their entirety), so-called WuxiBody formats (e.g., PCT Publication No. WO 2019/057122, which formats are incorporated herein by reference in their entirety), and so-called In-Elbow-Insert Ig formats (IEI-Ig; e.g., PCT Publication Nos. WO 2019/024979 and WO 2019/025391, which formats are incorporated herein by reference in their entirety).

In certain embodiments, the antibody or antigen-binding fragment comprises two or more of VH domains, two or more VL domains, or both (i.e., two or more VH domains and two or more VL domains). In particular embodiments, an antigen-binding fragment comprises the format (N-terminal to C-terminal direction) VH-linker-VL-linker-VH-linker-VL, wherein the two VH sequences can be the same or different and the two VL sequences can be the same or different. Such linked scFvs can include any combination of VH and VL domains arranged to bind to a given target, and in formats comprising two or more VH and/or two or more VL, one, two, or more different epitopes or antigens may be bound. It will be appreciated that formats incorporating multiple antigen-binding domains may include VH and/or VL sequences in any combination or orientation. For example, the antigen-binding fragment can comprise the format VL-linker-VH-linker-VL-linker-VH, VH-linker-VL-linker-VL-linker-VH, or VL-linker-VH-linker-VH-linker-VL.

Monospecific or multispecific antibodies or antigen-binding fragments of the present disclosure can comprise any combination of the VH and VL sequences and/or any combination of the CDRH1, CDRH2, CDRH3, CDRL1, CDRL2, and CDRL3 sequences disclosed herein. In certain embodiments, an antibody or antigen-binding fragment comprises the VH sequence provided in SEQ ID NO: 105 or SEQ ID NO: 113 and the VL sequence provided in SEQ ID NO: 168. In certain embodiments, an antibody or antigen-binding fragment comprises a CDRH1 sequence as provided in SEQ ID NO: 106, a CDRH2 sequence as provided in SEQ ID NO: 107 or 121, a CDRH3 sequence as provided in SEQ ID NO: 108, a CDRL1 sequence as provided in SEQ ID NO: 169, a CDRL2 sequence as provided in SEQ ID NO: 170, and a CDRL3 sequence as provided in SEQ ID NO: 171. In certain embodiments, an antibody or antigen-binding fragment comprises the amino acid sequence as provided in SEQ ID NO: 222-225 or SEQ ID NO: 230-233.A bispecific or multispecific antibody or antigen-binding fragment may, in some embodiments, comprise one, two, or more antigen-binding domains (e.g., a VH and a VL) of the instant disclosure. Two or more binding domains may be present that bind to the same or a different SARS-CoV-2 epitope, and a bispecific or multispecific antibody or antigen-binding fragment as provided herein can, in some embodiments, comprise a further SARS-CoV-2 binding domain, and/or can comprise a binding domain that binds to a different antigen or pathogen altogether.

In any of the presently disclosed embodiments, the antibody or antigen-binding fragment can be multispecific; e.g., bispecific, trispecific, or the like.

In certain embodiments, the antibody or antigen-binding fragment comprises a Fc polypeptide, or a fragment thereof. The “Fc” fragment or Fc polypeptide comprises the carboxy-terminal portions (i.e., the CH2 and CH3 domains of IgG) of both antibody H chains held together by disulfides. Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptor); and B cell activation. As discussed herein, modifications (e.g., amino acid substitutions) may be made to an Fc domain in order to modify (e.g., improve, reduce, or ablate) one or more functionality of an Fc-containing polypeptide (e.g., an antibody of the present disclosure). Such functions include, for example, Fc receptor (FcR) binding, antibody half-life modulation (e.g., by binding to FcRn), ADCC function, protein A binding, protein G binding, and complement binding. Amino acid modifications that modify (e.g., improve, reduce, or ablate) Fc functionalities include, for example, the T250Q/M428L, M252Y/S254T/T256E, H433K/N434F, M428L/N434S, E233P/L234V/L235A/G236+A327G/A330S/P331S, E333A, S239D/A330L/1332E, P257I/Q311, K326W/E333S, S239D/1332E/G236A, N297Q, K322A, S228P, L235E+E318A/K320A/K322A, L234A/L235A (also referred to herein as “LALA”), and L234A/L235A/P329G mutations, which mutations are summarized and annotated in “Engineered Fc Regions”, published by InvivoGen (2011) and available online at invivogen.com/PDF/review/review-Engineered-Fc-Regions-invivogen.pdf?utm_source=review&utm_medium=pdf&utm_campaign=review&utm_content=Engineered-Fc-Regions, and are incorporated herein by reference.

For example, to activate the complement cascade, the C1q protein complex can bind to at least two molecules of IgG1 or one molecule of IgM when the immunoglobulin molecule(s) is attached to the antigenic target (Ward, E. S., and Ghetie, V., Ther. Immunol. 2 (1995) 77-94). Burton, D. R., described (Mol. Immunol. 22 (1985) 161-206) that the heavy chain region comprising amino acid residues 318 to 337 is involved in complement fixation. Duncan, A. R., and Winter, G. (Nature 332 (1988) 738-740), using site directed mutagenesis, reported that Glu318, Lys320 and Lys322 form the binding site to C1q. The role of Glu318, Lys320 and Lys 322 residues in the binding of C1q was confirmed by the ability of a short synthetic peptide containing these residues to inhibit complement mediated lysis.

For example, FcR binding can be mediated by the interaction of the Fc moiety (of an antibody) with Fc receptors (FcRs), which are specialized cell surface receptors on cells including hematopoietic cells. Fc receptors belong to the immunoglobulin superfamily, and shown to mediate both the removal of antibody-coated pathogens by phagocytosis of immune complexes, and the lysis of erythrocytes and various other cellular targets (e.g. tumor cells) coated with the corresponding antibody, via antibody dependent cell mediated cytotoxicity (ADCC; Van de Winkel, J. G., and Anderson, C. L., J. Leukoc. Biol. 49 (1991) 511-524). FcRs are defined by their specificity for immunoglobulin classes; Fc receptors for IgG antibodies are referred to as FcTR, for IgE as FcR, for IgA as FcαR and so on and neonatal Fc receptors are referred to as FcRn. Fc receptor binding is described for example in Ravetch, J. V., and Kinet, J. P., Annu. Rev. Immunol. 9 (1991) 457-492; Capel, P. J., et al., Immunomethods 4 (1994) 25-34; de Haas, M., et al., J Lab. Clin. Med. 126 (1995) 330-341; and Gessner, J. E., et al., Ann. Hematol. 76 (1998) 231-248.

Cross-linking of receptors by the Fc domain of native IgG antibodies (FcTR) triggers a wide variety of effector functions including phagocytosis, antibody-dependent cellular cytotoxicity, and release of inflammatory mediators, as well as immune complex clearance and regulation of antibody production. Fc moieties providing cross-linking of receptors (e.g., FcTR) are contemplated herein. In humans, three classes of FcTR have been characterized to-date, which are: (i) FcγRI (CD64), which binds monomeric IgG with high affinity and is expressed on macrophages, monocytes, neutrophils and eosinophils; (ii) FcγRII (CD32), which binds complexed IgG with medium to low affinity, is widely expressed, in particular on leukocytes, is believed to be a central player in antibody-mediated immunity, and which can be divided into FcγRIIA, FcγRIIB and FcγRIIC, which perform different functions in the immune system, but bind with similar low affinity to the IgG-Fc, and the ectodomains of these receptors are highly homologous; and (iii) FcγRIII (CD16), which binds IgG with medium to low affinity and has been found in two forms: FcγRIIIA, which has been found on NK cells, macrophages, eosinophils, and some monocytes and T cells, and is believed to mediate ADCC; and FcγRIIIB, which is highly expressed on neutrophils.

FcγRIIA is found on many cells involved in killing (e.g. macrophages, monocytes, neutrophils) and seems able to activate the killing process. FcγRIIB seems to play a role in inhibitory processes and is found on B-cells, macrophages and on mast cells and eosinophils. Importantly, it has been shown that 75% of all FcγRIIB is found in the liver (Ganesan, L. P. et al., 2012: “FcγRIIb on liver sinusoidal endothelium clears small immune complexes,” Journal of Immunology 189: 4981-4988). FcγRIIB is abundantly expressed on Liver Sinusoidal Endothelium, called LSEC, and in Kupffer cells in the liver and LSEC are the major site of small immune complexes clearance (Ganesan, L. P. et al., 2012: FcγRIIb on liver sinusoidal endothelium clears small immune complexes. Journal of Immunology 189: 4981-4988).

In some embodiments, the antibodies disclosed herein and the antigen-binding fragments thereof comprise an Fc polypeptide or fragment thereof for binding to FcγRIIb, in particular an Fc region, such as, for example IgG-type antibodies. Moreover, it is possible to engineer the Fc moiety to enhance FcγRIIB binding by introducing the mutations S267E and L328F as described by Chu, S. Y. et al., 2008: Inhibition of B cell receptor-mediated activation of primary human B cells by coengagement of CD19 and FcgammaRIIb with Fc-engineered antibodies. Molecular Immunology 45, 3926-3933. Thereby, the clearance of immune complexes can be enhanced (Chu, S., et al., 2014: Accelerated Clearance of IgE In Chimpanzees Is Mediated By Xmab7195, An Fc-Engineered Antibody With Enhanced Affinity For Inhibitory Receptor FcγRIIb. Am J Respir Crit, American Thoracic Society International Conference Abstracts). In some embodiments, the antibodies of the present disclosure, or the antigen binding fragments thereof, comprise an engineered Fc moiety with the mutations S267E and L328F, in particular as described by Chu, S. Y. et al., 2008: Inhibition of B cell receptor-mediated activation of primary human B cells by coengagement of CD19 and FcgammaRIIb with Fc-engineered antibodies. Molecular Immunology 45, 3926-3933.

On B cells, FcγRIIB may function to suppress further immunoglobulin production and isotype switching to, for example, the IgE class. On macrophages, FcγRIIB is thought to inhibit phagocytosis as mediated through FcγRIIA. On eosinophils and mast cells, the B form may help to suppress activation of these cells through IgE binding to its separate receptor.

Regarding FcγRI binding, modification in native IgG of at least one of E233-G236, P238, D265, N297, A327 and P329 reduces binding to FcγRI. IgG2 residues at positions 233-236, substituted into corresponding positions IgG1 and IgG4, reduces binding of IgG1 and IgG4 to FcγRI by 10³-fold and eliminated the human monocyte response to antibody-sensitized red blood cells (Armour, K. L., et al. Eur. J. Immunol. 29 (1999) 2613-2624).

Regarding FcγRII binding, reduced binding for FcγRIIA is found, e.g., for IgG mutation of at least one of E233-G236, P238, D265, N297, A327, P329, D270, Q295, A327, R292 and K414.

Two allelic forms of human FcγRIIA are the “H131” variant, which binds to IgG1 Fc with high affinity, and the “R131” variant, which binds to IgG1 Fc with low affinity. See, e.g., Bruhns et al., Blood 113:3716-3725 (2009).

Regarding FcγRIII binding, reduced binding to FcγRIIIA is found, e.g., for mutation of at least one of E233-G236, P238, D265, N297, A327, P329, D270, Q295, A327, S239, E269, E293, Y296, V303, A327, K338 and D376. Mapping of the binding sites on human IgG1 for Fc receptors, the above-mentioned mutation sites, and methods for measuring binding to FcγRI and FcγRIIA, are described in Shields, R. L., et al., J. Biol. Chem. 276 (2001) 6591-6604.

Two allelic forms of human FcγRIIIA are the “F158” variant, which binds to IgG1 Fc with low affinity, and the “V158” variant, which binds to IgG1 Fc with high affinity. See, e.g., Bruhns et al., Blood 113:3716-3725 (2009).

Regarding binding to FcγRII, two regions of native IgG Fc appear to be involved in interactions between FcγRIIs and IgGs, namely (i) the lower hinge site of IgG Fc, in particular amino acid residues L, L, G, G (234-237, EU numbering), and (ii) the adjacent region of the CH2 domain of IgG Fc, in particular a loop and strands in the upper CH2 domain adjacent to the lower hinge region, e.g. in a region of P331 (Wines, B. D., et al., J. Immunol. 2000; 164: 5313-5318). Moreover, FcγRI appears to bind to the same site on IgG Fc, whereas FcRn and Protein A bind to a different site on IgG Fc, which appears to be at the CH2-CH3 interface (Wines, B. D., et al., J. Immunol. 2000; 164: 5313-5318).

Also contemplated are mutations that increase binding affinity of an Fc polypeptide or fragment thereof of the present disclosure to a (i.e., one or more) Fcγ receptor (e.g., as compared to a reference Fc polypeptide or fragment thereof or containing the same that does not comprise the mutation(s)). See, e.g., Delillo and Ravetch, Cell 161(5):1035-1045 (2015) and Ahmed et al., J. Struc. Biol. 194(1):78 (2016), the Fc mutations and techniques of which are incorporated herein by reference.

In any of the herein disclosed embodiments, an antibody or antigen-binding fragment can comprise a Fc polypeptide or fragment thereof comprising a mutation selected from G236A; S239D; A330L; and I332E; or a combination comprising any two or more of the same; e.g., S239D/I332E; S239D/A330L/I332E; G236A/S239D/I332E; G236A/A330L/I332E (also referred to herein as “GAALIE”); or G236A/S239D/A330L/I332E. In some embodiments, the Fc polypeptide or fragment thereof does not comprise S239D.

In certain embodiments, the Fc polypeptide or fragment thereof may comprise or consist of at least a portion of an Fc polypeptide or fragment thereof that is involved in binding to FcRn binding. In certain embodiments, the Fc polypeptide or fragment thereof comprises one or more amino acid modifications that improve binding affinity for (e.g., enhance binding to) FcRn (e.g., at a pH of about 6.0) and, in some embodiments, thereby extend in vivo half-life of a molecule comprising the Fc polypeptide or fragment thereof (e.g., as compared to a reference Fc polypeptide or fragment thereof or antibody that is otherwise the same but does not comprise the modification(s)). In certain embodiments, the Fc polypeptide or fragment thereof comprises or is derived from a IgG Fc and a half-life-extending mutation comprises any one or more of: M428L; N434S; N434H; N434A; N434S; M252Y; S254T; T256E; T250Q; P257I Q311I; D376V; T307A; E380A (EU numbering). In certain embodiments, a half-life-extending mutation comprises M428L/N434S (also referred to herein as “MLNS”). In certain embodiments, a half-life-extending mutation comprises M252Y/S254T/T256E. In certain embodiments, a half-life-extending mutation comprises T250Q/M428L. In certain embodiments, a half-life-extending mutation comprises P257I/Q311I. In certain embodiments, a half-life-extending mutation comprises P257I/N434H. In certain embodiments, a half-life-extending mutation comprises D376V/N434H. In certain embodiments, a half-life-extending mutation comprises T307A/E380A/N434A.

In some embodiments, an antibody or antigen-binding fragment includes a Fc moiety that comprises the substitution mutations M428L/N434S. In some embodiments, an antibody or antigen-binding fragment includes a Fc polypeptide or fragment thereof that comprises the substitution mutations G236A/A330L/I332E. In certain embodiments, an antibody or antigen-binding fragment includes a (e.g., IgG) Fc moiety that comprises a G236A mutation, an A330L mutation, and a 1332E mutation (GAALIE), and does not comprise a S239D mutation (e.g., comprises a native S at position 239). In particular embodiments, an antibody or antigen-binding fragment includes an Fc polypeptide or fragment thereof that comprises the substitution mutations: M428L/N434S and G236A/A330L/I332E, and optionally does not comprise S239D. In certain embodiments, an antibody or antigen-binding fragment includes a Fc polypeptide or fragment thereof that comprises the substitution mutations: M428L/N434S and G236A/S239D/A330L/1332E.

In certain embodiments, the antibody or antigen-binding fragment comprises a mutation that alters glycosylation, wherein the mutation that alters glycosylation comprises N297A, N297Q, or N297G, and/or the antibody or antigen-binding fragment is partially or fully aglycosylated and/or is partially or fully afucosylated. Host cell lines and methods of making partially or fully aglycosylated or partially or fully afucosylated antibodies and antigen-binding fragments are known (see, e.g., PCT Publication No. WO 2016/181357; Suzuki et al. Clin. Cancer Res. 13(6):1875-82 (2007); Huang et al. MAbs 6:1-12 (2018)).

In certain embodiments, the antibody or antigen-binding fragment is capable of eliciting continued protection in vivo in a subject even once no detectable levels of the antibody or antigen-binding fragment can be found in the subject (i.e., when the antibody or antigen-binding fragment has been cleared from the subject following administration). Such protection is referred to herein as a vaccinal effect. Without wishing to be bound by theory, it is believed that dendritic cells can internalize complexes of antibody and antigen and thereafter induce or contribute to an endogenous immune response against antigen. In certain embodiments, an antibody or antigen-binding fragment comprises one or more modifications, such as, for example, mutations in the Fc comprising G236A, A330L, and 1332E, that are capable of activating dendritic cells that may induce, e.g., T cell immunity to the antigen.

In any of the presently disclosed embodiments, the antibody or antigen-binding fragment comprises a Fc polypeptide or a fragment thereof, including a CH2 (or a fragment thereof, a CH3 (or a fragment thereof), or a CH2 and a CH3, wherein the CH2, the CH3, or both can be of any isotype and may contain amino acid substitutions or other modifications as compared to a corresponding wild-type CH2 or CH3, respectively. In certain embodiments, a Fc polypeptide of the present disclosure comprises two CH2-CH3 polypeptides that associate to form a dimer.

In any of the presently disclosed embodiments, the antibody or antigen-binding fragment can be monoclonal. The term “monoclonal antibody” (mAb) as used herein refers to an antibody obtained from a population of substantially homogeneous antibodies, i.e., individual antibodies comprising the population are identical except for possible naturally occurring mutations that may be present, in some cases in minor amounts. Monoclonal antibodies are highly specific, being directed against a single antigenic site. Furthermore, in contrast to polyclonal antibody preparations that include different antibodies directed against different epitopes, each monoclonal antibody is directed against a single epitope of the antigen. In addition to their specificity, the monoclonal antibodies are advantageous in that they may be synthesized uncontaminated by other antibodies. The term “monoclonal” is not to be construed as requiring production of the antibody by any particular method. For example, monoclonal antibodies useful in the present invention may be prepared by the hybridoma methodology first described by Kohler et al., Nature 256:495 (1975), or may be made using recombinant DNA methods in bacterial, eukaryotic animal, or plant cells (see, e.g., U.S. Pat. No. 4,816,567). Monoclonal antibodies may also be isolated from phage antibody libraries using the techniques described in Clackson et al., Nature, 352:624-628 (1991) and Marks et al., J. Mol. Biol., 222:581-597 (1991), for example. Monoclonal antibodies may also be obtained using methods disclosed in PCT Publication No. WO 2004/076677A2.

Antibodies and antigen-binding fragments of the present disclosure include “chimeric antibodies” in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, U.S. Pat. Nos. 4,816,567; 5,530,101 and 7,498,415; and Morrison et al., Proc. Natl. Acad. Sci. USA, 81:6851-6855 (1984)). For example, chimeric antibodies may comprise human and non-human residues. Furthermore, chimeric antibodies may comprise residues that are not found in the recipient antibody or in the donor antibody. These modifications are made to further refine antibody performance. For further details, see Jones et al., Nature 321:522-525 (1986); Riechmann et al., Nature 332:323-329 (1988); and Presta, Curr. Op. Struct. Biol. 2:593-596 (1992). Chimeric antibodies also include primatized and humanized antibodies.

A “humanized antibody” is generally considered to be a human antibody that has one or more amino acid residues introduced into it from a source that is non-human. These non-human amino acid residues are typically taken from a variable domain. Humanization may be performed following the method of Winter and co-workers (Jones et al., Nature, 321:522-525 (1986); Reichmann et al., Nature, 332:323-327 (1988); Verhoeyen et al., Science, 239:1534-1536 (1988)), by substituting non-human variable sequences for the corresponding sequences of a human antibody. Accordingly, such “humanized” antibodies are chimeric antibodies (U.S. Pat. Nos. 4,816,567; 5,530,101 and 7,498,415) wherein substantially less than an intact human variable domain has been substituted by the corresponding sequence from a non-human species. In some instances, a “humanized” antibody is one which is produced by a non-human cell or animal and comprises human sequences, e.g., Hc domains.

A “human antibody” is an antibody containing only sequences that are present in an antibody that is produced by a human. However, as used herein, human antibodies may comprise residues or modifications not found in a naturally occurring human antibody (e.g., an antibody that is isolated from a human), including those modifications and variant sequences described herein. These are typically made to further refine or enhance antibody performance. In some instances, human antibodies are produced by transgenic animals. For example, see U.S. Pat. Nos. 5,770,429; 6,596,541 and 7,049,426. In certain embodiments, an antibody or antigen-binding fragment of the present disclosure is chimeric, humanized, or human.

Exemplary antibodies of the present disclosure include S309, sotrovimab, and VIR-7832. S309 is a human monoclonal antibody obtained from a B cell of a SARS-CoV survivor. S309 comprises the VH amino acid sequence of SEQ ID NO.:105 and the VL amino acid sequence of SEQ ID NO.:168. Sotrovimab (IgG1*01 G1m17; VH of SEQ ID NO.:113, M428L and N434S Fc mutations; VL of SEQ ID NO.:168 (kappa light chain IgKC*01 k1m3)) and VIR-7832 ((IgG1*01 G1m17; VH of SEQ ID NO.: 113, G236A, A330L, 1332E, M428L, and N434S Fc mutations; VL of SEQ ID NO.:168 (kappa light chain IgKC*01 k1m3)) are engineered human monoclonal antibodies derived from S309.

### Polynucleotides, Vectors, and Host Cells

Presently disclosed antibodies and antigen-binding fragments (and portions thereof; e.g., a CDR, a VH, a VL, a heavy chain, or a light chain) can be encoded by a polynucleotide. The polynucleotide can comprise deoxyribonucleic acid (DNA) or ribonucleic acid (RNA). RNA can comprise messenger RNA (mRNA). Polynucleotides can be codon-optimized for expression in a host cell. Once a coding sequence is known or identified, codon optimization can be performed using known techniques and tools, e.g., using the GenScript® OptimiumGene™ tool; see also Scholten et al., Clin. Immunol. 119:135, 2006). Codon-optimized sequences include sequences that are partially codon-optimized (i.e., one or more codon is optimized for expression in the host cell) and those that are fully codon-optimized. It will also be appreciated that polynucleotides encoding antibodies and antigen-binding fragments of the present disclosure may possess different nucleotide sequences while still encoding a same antibody or antigen-binding fragment due to, for example, the degeneracy of the genetic code, splicing, and the like. It will be appreciated that a polynucleotide encoding an antibody or antigen-binding fragment can be comprised in a polynucleotide that includes other sequences and/or features for, e.g., expression of the antibody or antigen-binding fragment in a host cell. Exemplary features include a promoter sequence, a polyadenylation sequence, a sequence that encodes a signal peptide (e.g., located at the N-terminus of a expressed antibody heavy chain or light chain), or the like.

Polynucleotides can be comprised or contained in a vector. A vector can comprise any one or more of the vectors disclosed herein. A vectors can comprise, for example, a DNA plasmid construct encoding the antibody or antigen-binding fragment, or a portion thereof (e.g., so-called “DMAb”; see, e.g., Muthumani et al., J Infect Dis. 214(3):369-378 (2016); Muthumani et al., Hum Vaccin Immunother 9:2253-2262 (2013)); Flingai et al., Sci Rep. 5:12616 (2015); and Elliott et al., NPJ Vaccines 18 (2017), which antibody-coding DNA constructs and related methods of use, including administration of the same, are incorporated herein by reference). A DNA plasmid construct can comprise a single open reading frame encoding a heavy chain and a light chain (or a VH and a VL) of the antibody or antigen-binding fragment, wherein the sequence encoding the heavy chain and the sequence encoding the light chain are optionally separated by polynucleotide encoding a protease cleavage site and/or by a polynucleotide encoding a self-cleaving peptide. Substituent components of the antibody or antigen-binding fragment can be encoded by a polynucleotide comprised in a single plasmid. Alternatively, the substituent components of the antibody or antigen-binding fragment can be encoded by a polynucleotide comprised in two or more plasmids (e.g., a first plasmid comprises a polynucleotide encoding a heavy chain, VH, or VH+CH, and a second plasmid comprises a polynucleotide encoding the cognate light chain, VL, or VL+CL). A single plasmid can comprise a polynucleotide encoding a heavy chain and/or a light chain from two or more antibodies or antigen-binding fragments of the present disclosure. An exemplary expression vector is pVax1, available from Invitrogen®. A DNA plasmid of the present disclosure can be delivered to a subject by, for example, electroporation (e.g., intramuscular electroporation), or with an appropriate formulation (e.g., hyaluronidase). A vector can comprise a nucleotide sequence encoding a signal peptide. The signal peptide may or may not be present (e.g., can be enzymatically cleaved from) on the mature antibody or antigen-binding fragment. Nucleic acid sequence encoding a signal peptide include the nucleotide sequence set forth in SEQ ID NO.: 252 or SEQ ID NO.: 263. A signal peptide can comprise or consist of the amino acid sequence set forth in SEQ ID NO.:256 or SEQ ID NO.: 264. A vector can comprise a polyadenylation signal sequence. An example of a polyadenylation signal sequence comprises or consists of the nucleotide sequence as set forth in SEQ ID NO.: 253.

A vector can comprise a CMV promoter (e.g., comprising or consisting of the nucleotide sequence as set forth in SEQ ID NO.: 251).

Examples of host cells that can be used to express a presently disclosed antigen or antigen-binding fragment cells include but are not limited to, eukaryotic cells, e.g., yeast cells, animal cells, insect cells, plant cells; and prokaryotic cells, including E. coli. In some embodiments, the cells are mammalian cells. Cells include a mammalian cell line such as CHO cells (e.g., DHFR-CHO cells (Urlaub et al., PNAS 77:4216 (1980)), human embryonic kidney cells (e.g., HEK293T cells), PER.C6 cells, Y0 cells, Sp2/0 cells. NS0 cells, human liver cells, e.g. Hepa RG cells, myeloma cells or hybridoma cells. Other examples of mammalian host cell lines include mouse sertoli cells (e.g., TM4 cells); monkey kidney CV1 line transformed by SV40 (COS-7); baby hamster kidney cells (BHK); African green monkey kidney cells (VERO-76); monkey kidney cells (CV1); human cervical carcinoma cells (HELA); human lung cells (W138); human liver cells (Hep G2); canine kidney cells (MDCK; buffalo rat liver cells (BRL 3A); mouse mammary tumor (MMT 060562); TRI cells; MRC 5 cells; and FS4 cells. Mammalian host cell lines suitable for antibody production also include those described in, for example, Yazaki and Wu, Methods in Molecular Biology, Vol. 248 (B. K. C. Lo, ed., Humana Press, Totowa, N.J.), pp. 255-268 (2003).

Host cells also include a prokaryotic cell, such as an E. coli. The expression of peptides in prokaryotic cells such as E. coli is well established (see, e.g., Pluckthun, A. Bio Technology 9:545-551 (1991). For example, antibodies may be produced in bacteria, in particular when glycosylation and Fe effector function are not needed. For expression of antibody fragments and polypeptides in bacteria, see, e.g., U.S. Pat. Nos. 5,648,237; 5,789,199; and 5,840,523. A cell may be transfected with a vector according to the present description with an expression vector. The term “transfection” refers to the introduction of nucleic acid molecules, such as DNA or RNA (e.g. mRNA) molecules, into cells, such as into eukaryotic cells. In the context of the present description, the term “transfection” encompasses any method known to the skilled person for introducing nucleic acid molecules into cells, such as into eukaryotic cells, including into mammalian cells. Such methods encompass, for example, electroporation, lipofection, e.g., based on cationic lipids and/or liposomes, calcium phosphate precipitation, nanoparticle based transfection, virus based transfection, or transfection based on cationic polymers, such as DEAE-dextran or polyethylenimine, etc. In certain embodiments, the introduction is non-viral.

Moreover, host cells may be transfected stably or transiently with a vector, e.g. for expressing an antibody, or an antigen-binding fragment thereof. Cells may be stably transfected with a vector. Alternatively, cells may be transiently transfected with a vector encoding an antibody or antigen-binding fragment.

An antibody or antigen-binding fragment (or polynucleotide encoding the same) can be heterologous to the host cell. For example, the cell may be of a species that is different to the species from which the antibody was fully or partially obtained (e.g., CHO cells expressing a human antibody or an engineered human antibody). The cell type of the host cell may not express the antibody or antigen-binding fragment in nature. Moreover, the host cell may impart a post-translational modification (PTM; e.g., glycosylation or fucosylation) on the antibody or antigen-binding fragment that is not present in a native state of the antibody or antigen-binding fragment (or in a native state of a parent antibody from which the antibody or antigen binding fragment was engineered or derived). Such a PTM may result in a functional difference (e.g., reduced immunogenicity). Accordingly, an antibody or antigen-binding fragment of the present disclosure that is produced by a host cell as disclosed herein may include one or more post-translational modification that is distinct from the antibody (or parent antibody) in its native state (e.g., a human antibody produced by a CHO cell can comprise one or more post-translational modification that is distinct from the antibody when isolated from the human and/or produced by the native human B cell or plasma cell).

Insect cells useful expressing a binding protein of the present disclosure are known in the art and include, for example, Spodoptera frugipera Sf9 cells, Trichoplusia ni BTI-TN5B1-4 cells, and Spodoptera frugipera SfSWT01 “Mimic™” cells. See, e.g., Palmberger et al., J. Biotechnol. 153(3-4):160-166 (2011). Numerous baculoviral strains have been identified which may be used in conjunction with insect cells, particularly for transfection of Spodoptera frugiperda cells.

Eukaryotic microbes such as filamentous fungi or yeast are also suitable hosts for cloning or expressing protein-encoding vectors, and include fungi and yeast strains with “humanized” glycosylation pathways, resulting in the production of an antibody with a partially or fully human glycosylation pattern. See Gerngross, Nat. Biotech. 22:1409-1414 (2004); Li et al., Nat. Biotech. 24:210-215 (2006).

Plant cells can also be utilized as hosts for expressing a binding protein of the present disclosure. For example, PLANTIBODIES™ technology (described in, for example, U.S. Pat. Nos. 5,959,177; 6,040,498; 6,420,548; 7,125,978; and 6,417,429) employs transgenic plants to produce antibodies.

Mammalian host cells include, for example, a CHO cell, a HEK293 cell, a PER.C6 cell, a Y0 cell, a Sp2/0 cell, a NS0 cell, a human liver cell, a myeloma cell, or a hybridoma cell.

Methods for producing an antibody, or antigen-binding fragment can comprise culturing a host cell under conditions and for a time sufficient to produce the antibody, or the antigen-binding fragment. Methods useful for isolating and purifying recombinantly produced antibodies, by way of example, may include obtaining supernatants from suitable host cell/vector systems that secrete the recombinant antibody into culture media and then concentrating the media using a commercially available filter. Following concentration, the concentrate may be applied to a single suitable purification matrix or to a series of suitable matrices, such as an affinity matrix or an ion exchange resin. One or more reverse phase HPLC steps may be employed to further purify a recombinant polypeptide. These purification methods may also be employed when isolating an immunogen from its natural environment. Methods for large scale production of one or more of the isolated/recombinant antibody described herein include batch cell culture, which is monitored and controlled to maintain appropriate culture conditions. Purification of soluble antibodies may be performed according to methods described herein and known in the art and that comport with laws and guidelines of domestic and foreign regulatory agencies.

### Pharmaceutical Compositions and Methods and Uses Thereof

Also provided herein are compositions that comprise any one or more of the presently disclosed antibodies or antigen-binding fragments and can further comprise a pharmaceutically acceptable carrier, excipient, or diluent. Carriers, excipients, and diluents are discussed in further detail herein.

In certain embodiments, a composition comprises two or more different antibodies or antigen-binding fragments according to the present disclosure. In certain embodiments, antibodies or antigen-binding fragments to be used in a combination each independently have one or more of the following characteristics: neutralize naturally occurring SARS-CoV-2 variants; do not compete with one another for Spike protein binding; bind distinct Spike protein epitopes; have a reduced formation of resistance to SARS-CoV-2; when in a combination, have a reduced formation of resistance to SARS-CoV-2; potently neutralize live SARS-CoV-2 virus; exhibit additive or synergistic effects on neutralization of live SARS-CoV-2 virus when used in combination; exhibit effector functions; are protective in relevant animal model(s) of infection; are capable of being produced in sufficient quantities for large-scale production.

In some embodiments, a composition comprises a polynucleotide or vector that encodes an antibody or antigen-binding fragment. In certain embodiments, a composition comprises a first vector comprising a first plasmid, and a second vector comprising a second plasmid, wherein the first plasmid comprises a polynucleotide encoding a heavy chain, VH, or VH+CH, and a second plasmid comprises a polynucleotide encoding the cognate light chain, VL, or VL+CL of the antibody or antigen-binding fragment thereof. In certain embodiments, a composition comprises a polynucleotide (e.g., mRNA) coupled to a suitable delivery vehicle or carrier. Exemplary vehicles or carriers for administration to a human subject include a lipid or lipid-derived delivery vehicle, such as a liposome, solid lipid nanoparticle, oily suspension, submicron lipid emulsion, lipid microbubble, inverse lipid micelle, cochlear liposome, lipid microtubule, lipid microcylinder, or lipid nanoparticle (LNP) or a nanoscale platform (see, e.g., Li et al. Wilery Interdiscip Rev. Nanomed Nanobiotechnol. 11(2):e1530 (2019)). Principles, reagents, and techniques for designing appropriate mRNA and formulating mRNA-LNP and delivering the same are described in, for example, Pardi et al. (J Control Release 217345-351 (2015)); Thess et al. (Mol Ther 23: 1456-1464 (2015)); Thran et al. (EMBO Mol Med 9(10):1434-1448 (2017); Kose et al. (Sci. Immunol. 4 eaaw6647 (2019); and Sabnis et al. (Mol. Ther. 26:1509-1519 (2018)), which techniques, include capping, codon optimization, nucleoside modification, purification of mRNA, incorporation of the mRNA into stable lipid nanoparticles (e.g., ionizable cationic lipid/phosphatidylcholine/cholesterol/PEG-lipid; ionizable lipid:distearoyl PC:cholesterol:polyethylene glycol lipid), and subcutaneous, intramuscular, intradermal, intravenous, intraperitoneal, and intratracheal administration of the same, are incorporated herein by reference.

Also provided herein are methods of treating a pediatric subject using an antibody or antigen-binding fragment of the present disclosure, or a composition comprising the same, wherein the pediatric subject has, is believed to have, or is at risk for having an infection by a SARS-CoV-2, optionally having, believed to have, or at risk for COVID-19.

Accordingly, in certain embodiments, methods are provided for treating a SARS-CoV-2 infection in a pediatric subject (e.g. having or at risk of contracting a SARS-CoV-2 infection or COVID-19), wherein the methods comprise administering to the pediatric subject an effective amount of an antibody, antigen-binding fragment, or composition as disclosed herein.

The pediatric subjects can be male or female and can be any suitable age, including infant, juvenile, and adolescent subjects. In certain embodiments, the pediatric subject may be in utero with administration of the antibody, antigen-binding fragment or composition to the mother, less than 2 years of age, optionally also at least 32 weeks gestational age at birth or less than 32 weeks gestational age at birth, at least 2 years of age, but less than 6 years of age, at least 6 years of age, but less than 12 years of age, or at least 12 years of age, but less than 18 years of age.

Briefly, pharmaceutical compositions according to certain embodiments of the present disclosure are formulated so as to allow the active ingredients contained therein to be bioavailable upon administration of the composition to a patient. Compositions that will be administered to a pediatric subject may take the form of one or more dosage units, where for example, a tablet may be a single dosage unit, and a container of a herein described an antibody or antigen-binding in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain an effective amount of an antibody or antigen-binding fragment, polynucleotide, vector, host cell, or composition of the present disclosure, for treatment of a disease or condition of interest in accordance with teachings herein.

A composition may be in the form of a solid or liquid. In some embodiments, the carrier(s) are particulate, so that the compositions are, for example, in tablet or powder form. The carrier(s) may be liquid, with the compositions being, for example, an oral oil, injectable liquid or an aerosol, which is useful in, for example, inhalatory administration. When intended for oral administration, the pharmaceutical composition is preferably in either solid or liquid form, where semi solid, semi liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent. When the composition is in the form of a capsule, for example, a gelatin capsule, it may contain, in addition to materials of the above type, a liquid carrier such as polyethylene glycol or oil.

The composition may be in the form of a liquid, for example, an elixir, syrup, solution, emulsion or suspension. The liquid may be for oral administration or for delivery by injection, as two examples. When intended for oral administration, preferred compositions contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

Liquid pharmaceutical compositions, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid composition intended for either parenteral or oral administration should contain an amount of an antibody or antigen-binding fragment as herein disclosed such that a suitable dosage will be obtained. Typically, this amount is at least 0.01% of the antibody or antigen-binding fragment in the composition. When intended for oral administration, this amount may be varied to be between 0.1 and about 70% of the weight of the composition. Certain oral pharmaceutical compositions contain between about 4% and about 75% of the antibody or antigen-binding fragment. In certain embodiments, pharmaceutical compositions and preparations according to the present invention are prepared so that a parenteral dosage unit contains between 0.01 to 10% by weight of antibody or antigen-binding fragment prior to dilution.

The composition may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a composition for topical administration. If intended for transdermal administration, the composition may include a transdermal patch or iontophoresis device. The pharmaceutical composition may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter and polyethylene glycol.

A composition may include various materials which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule. The composition in solid or liquid form may include an agent that binds to the antibody or antigen-binding fragment of the disclosure and thereby assists in the delivery of the compound. Suitable agents that may act in this capacity include monoclonal or polyclonal antibodies, one or more proteins or a liposome. The composition may consist essentially of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols may be delivered in single phase, bi phasic, or tri phasic systems in order to deliver the active ingredient(s). Delivery of the aerosol includes the necessary container, activators, valves, subcontainers, and the like, which together may form a kit. One of ordinary skill in the art, without undue experimentation, may determine preferred aerosols.

It will be understood that compositions of the present disclosure also encompass carrier molecules for polynucleotides, as described herein (e.g., lipid nanoparticles, nanoscale delivery platforms, and the like).

The pharmaceutical compositions may be prepared by methodology well known in the pharmaceutical art. For example, a composition intended to be administered by injection can be prepared by combining a composition that comprises an antibody, antigen-binding fragment thereof, or antibody conjugate as described herein and optionally, one or more of salts, buffers and/or stabilizers, with sterile, distilled water so as to form a solution. A surfactant may be added to facilitate the formation of a homogeneous solution or suspension. Surfactants are compounds that non-covalently interact with the peptide composition so as to facilitate dissolution or homogeneous suspension of the antibody or antigen-binding fragment thereof in the aqueous delivery system.

In general, an appropriate dose and treatment regimen provide the composition(s) in an amount sufficient to provide therapeutic and/or prophylactic benefit (such as described herein, including an improved clinical outcome (e.g., a decrease in frequency, duration, or severity of diarrhea or associated dehydration, or inflammation, or longer disease-free and/or overall survival, or a lessening of symptom severity). For prophylactic use, a dose should be sufficient to prevent, delay the onset of, or diminish the severity of a disease associated with disease or disorder. Prophylactic benefit of the compositions administered according to the methods described herein can be determined by performing pre-clinical (including in vitro and in vivo animal studies) and clinical studies and analyzing data obtained therefrom by appropriate statistical, biological, and clinical methods and techniques, all of which can readily be practiced by a person skilled in the art.

Compositions are administered in an effective amount (e.g., to treat a SARS-CoV-2 infection), which will vary depending upon a variety of factors including the activity of the specific compound employed; the metabolic stability and length of action of the compound; the age, body weight, general health, sex, and diet of the pediatric subject; the mode and time of administration; the rate of excretion; the drug combination; the severity of the particular disorder or condition; and the pediatric subject undergoing therapy. In certain embodiments, following administration of therapies according to the formulations and methods of this disclosure, test pediatric subjects will exhibit about a 10% up to about a 99% reduction in one or more symptoms associated with the disease or disorder being treated as compared to placebo-treated or other suitable control pediatric subjects.

Generally, a therapeutically effective daily dose of an antibody or antigen binding fragment is (for a 70 kg mammal) from about 0.001 mg/kg (i.e., 0.07 mg) to about 100 mg/kg (i.e., 7.0 g); preferably a therapeutically effective dose is (for a 70 kg mammal) from about 0.01 mg/kg (i.e., 0.7 mg) to about 50 mg/kg (i.e., 3.5 g); more preferably a therapeutically effective dose is (for a 70 kg mammal) from about 1 mg/kg (i.e., 70 mg) to about 25 mg/kg (i.e., 1.75 g).

For polynucleotides, vectors, host cells, and related compositions of the present disclosure, a therapeutically effective dose may be different than for an antibody or antigen-binding fragment.

In certain embodiments, a method according to the present disclosure comprises administering to a pediatric subject a presently disclosed antibody or antigen-binding fragment, particularly sotrovimab or VIR-7832, at a dose of up to 62.5 mg, up to 100 mg, up to 125 mg, up to 150 mg, up to 187.5 mg, up to 200 mg, up to 250 mg, up to 300 mg, up to 350 mg, up to 375 mg, up to 400 mg, up to 450 mg, or up to 500 mg. In certain embodiments, a method comprises administering to a pediatric subject a presently disclosed antibody or antigen-binding fragment, particularly sotrovimab or VIR-7832 at a dose in a range from about 50 mg to about 500 mg, or in a range from about 50 mg to about 250 mg, or in a range from about 50 mg to 100 mg, or in a range from about 100 mg to about 500 mg, in a range from about 250 mg to about 500 mg, in a range from about 62.5 mg to about 500 mg, or in a range from about 62.5 mg to about 375 mg. In some embodiments, a method comprises administering to a pediatric subject 50, 62.5, 75, 100, 125, 150, 175, 187.5, 200, 225, 250, 275, 300, 325, 350, 375, 400, 425, 450, 475, or 500 mg of the antibody or antigen-binding fragment, particularly sotrovimab or VIR-7832. In some embodiments, a method comprises administering to a pediatric subject 62.5, 125, 187.5, 250, or 375 mg of the antibody or antigen-binding fragment, particularly sotrovimab or VIR-7832. In some embodiments, a method comprises administering to a pediatric subject 100 mg, a dose in a range from 200 mg to 250 mg, or 500 mg of the antibody or antigen-binding fragment, particularly sotrovimab or VIR-7832. In some embodiments, a method comprises administering to a pediatric subject 100 mg, 150 mg, 225 mg, or 350 mg of the antibody or antigen-binding fragment, particularly sotrovimab or VIR-7832. In some embodiments, a method comprises administering to a pediatric subject 500 mg of the antibody or antigen-binding fragment, particularly sotrovimab or VIR-7832, particularly if the pediatric subject is aged 12 years or more and weighs more than 40 kg.

For antibodies or antigen binding fragments according to certain embodiments of the present disclosure, particularly sotrovimab or VIR-7832, the effective dose may vary by age. In some embodiments, pediatric dosing may be scaled from an adult dose of 500 mg for a 77 kg adult.

In some embodiments, in pediatric subjects aged less than 2 years, optionally also at least 32 weeks gestational age at birth or less than 32 weeks gestational age at birth, dosing may be weight based.

In some embodiments, in pediatric subjects aged 2 years or more, dosing may be based on age.

In one embodiment, for a pediatric subject aged less than 2 years, but at least 32 weeks gestational age at birth, and weighing at least 2 kg but less than 4 kg, a dose of 62.5 mg may be administered, and more particularly may be in a volume of 1.0 mL, 0.83 mL, 0.63 mL, 0.5 mL, or 0.42 mL.

In another embodiment, for a pediatric subject aged less than 2 years, but at least 32 weeks gestational age at birth, and weighing at least 4 kg, a dose of 125 mg may be administered, and more particularly may be in a volume of 2.0 mL, 1.67 mL, 1.25 mL, 1.0 mL, or 0.83 mL.

In another embodiment, for a pediatric subject aged at least 2 years to less than 6 years, a dose of 187.5 mg may be administered, and more particularly may be in a volume of 3.0 mL, 2.5 mL 1.88 mL, 1.5 mL, or 1.25 mL. In some embodiments, a pediatric subject aged less than 2 years, but weighing more than 10 kg may also be administered this dose In another embodiment, for a pediatric subject aged at least 6 years to less than 12 years, a dose of 250 mg may be administered, and more particularly may be in a volume of 4.0 mL, 3.33 mL, 2.5 mL, 2.0 mL, or 1.66 mL.

In another embodiment, for a pediatric subject aged at least 12 years to less than 18 years, a dose of 375 mg may be administered, and more particularly may be in a volume of 6.0 mL, 5 mL, 3.75 mL, 3.0 mL, or 2.5 mL.

In another embodiment, for a pediatric subject aged less than 1 year, but at least 32 weeks gestational age at birth, a dose of 100 mg may be administered, and more particularly may be in a volume of 1.6 ml, 1.33 mL, 1 mL, 0.8 mL, or 0.67 mL.

In another embodiment, for a pediatric subject aged at least 1 year to less than 12 years, a dose in a range from 200 and 250 mg may be administered, and more particularly may be in a volume in a range from of 3.2 mL to 4 mL, 2.67 mL to 3.33 mL, 2 mL to 2.5 mL, 1.6 mL to 2 mL, 1.33 mL to 1.67 mL.

In another embodiment, for a pediatric subject aged at least 12 years to less than 18 years, a dose of 500 mg may be administered, and more particularly may be in a volume of 8 mL, 6.67 mL, 5 mL, 4 mL, or 3.33 mL.

In another embodiment, for a pediatric subject aged less than 2 years, but at least 32 weeks gestational age at birth, a dose of 100 mg may be administered, and more particularly may be in a volume of 1.6 ml, 1.33 mL, 1 mL, 0.8 mL, or 0.67 mL. In some embodiments, any pediatric subject weighing less than 10 kg may be administered this dose, regardless of age.

In another embodiment, for a pediatric subject aged at least 2 years to less than 6 years, a dose of 150 mg may be administered, and more particularly may be in a volume of 2.4 mL, 2.0 mL, 1.5 mL, 1.2 mL, or 1 mL.

In another embodiment, for a pediatric subject aged at least 6 years to less than 12 years, a dose of 225 mg may be administered, and more particularly may be in a volume of 3.6 mL, 3 mL, 2.25 mL, 1.8 mL, or 1.5 mL.

In another embodiment, for a pediatric subject aged at least 12 years to less than 18 years, a dose of 350 mg may be administered, and more particularly may be in a volume of 5.6 mL, 4.67 mL, 3.5 mL, 2.8 mL, or 2.33 mL.

In some embodiments, in pediatric subjects (aged less than 18 years old), dosing may be based on a combination of age and weight.

In some embodiments, for a pediatric subject aged less than 2 years and weighing at least 2 kg, but less than 5 kg, a dose of 62.5 mg may be administered, and more particularly may be in volume of 1.0 mL, 0.83 mL, 0.63 mL, 0.5 mL, or 0.42 mL.

In some embodiments, for a pediatric subject aged less than 2 years and weighing at least 5 kg, but less than 15 kg, a dose of 125 mg may be administered, and more particularly may be in a volume of 2.0 mL, 1.67 mL, 1.25 mL, 1.0 mL, or 0.83 mL.

In some embodiments, for a pediatric subject aged less than 2 years and weighing at least 15 kg, but less than 40 kg, a dose of 250 mg may be administered, and more particularly may be in a volume of 4.0 mL, 3.33 mL, 2.5 mL, 2.0 mL, or 1.66 mL.

In some embodiments, for a pediatric subject aged at least 2 years to less than 6 years and weighing at least 5 kg, but less than 15 kg, a dose of 125 mg may be administered, and more particularly may be in a volume of 2.0 mL, 1.67 mL, 1.25 mL, 1.0 mL, or 0.83 mL.

In some embodiments, for a pediatric subject aged at least 2 years to less than 6 years and weighing at least 15 kg, but less than 40 kg, a dose of 250 mg may be administered, and more particularly may be in a volume of 4.0 mL, 3.33 mL, 2.5 mL, 2.0 mL, or 1.66 mL.

In some embodiments, for a pediatric subject aged at least 6 years to less than 12 years and weighing at least 15 kg, but less than 40 kg, a dose of 250 mg may be administered, and more particularly may be in a volume of 4.0 mL, 3.33 mL, 2.5 mL, 2.0 mL, or 1.66 mL.

In some embodiments, for a pediatric subject aged at least 6 years to less than 12 years and weighing at least 40 kg, a dose of 500 mg may be administered, and more particularly may be in a volume of 8 mL, 6.67 mL, 5 mL, 4 mL, or 3.33 mL.

In some embodiments, for a pediatric subject aged at least 12 years to 18 years and weighing at least 15 kg, but less than 40 kg, a dose of 250 mg may be administered, and more particularly may be in a volume of 4.0 mL, 3.33 mL, 2.5 mL, 2.0 mL, or 1.66 mL.

In some embodiments, for a pediatric subject aged at least 12 years to 18 years and weighing at least 40 kg, a dose of 500 mg may be administered, and more particularly may be in a volume of 8 mL, 6.67 mL, 5 mL, 4 mL, or 3.33 mL.

Doses may be administered intravenously or intramuscularly, particularly by deltoid, thigh, or gluteal injection. Intramuscular administration, particularly in pediatric subjects, may be facilitate by administering a higher concentration of antibody or antigen binding fragment in a lower volume, such as any of the four lower volumes in the above embodiments. Doses administered to the deltoid may be 2.5 mL/injection or less. Doses administered to gluteal or thigh muscles may be 5.0 mL/injection or less.

In some embodiments, each subject may be administered a single dose, such as only a single dose, of antibody or antigen-binding fragment.

The single dose may be administered by more than one injection. The use of multiple injections to administer a single dose may facilitate administration to very young or very elderly patients, or those with low muscle mass as well as other patients who also benefit from high concentration formulations. For example, a single dose may be administered by more than one deltoid injections. In particular, it may be administered by an injection to each deltoid, gluteal (e.g. dorsogluteal or ventrogluteal), or thigh (e.g. anterolateral thigh) site. A single dose may be administered by injections to different sites, such dorsogluteal and ventrogluteal injections, dorsogluteal and deltoid injection, dorsogluteal and anterolateral thigh injections, ventrogluteal and deltoid injections, ventrogluteal and anterolateral thigh injections, anterolateral thigh and deltoid injections, dorsogluteal, ventrogluteal, and anterolateral thigh injections, dorsogluteal, ventrogluteal, and deltoid injections, ventrogluteal, anterolateral thigh, and deltoid injections, dorsogluteal, ventrogluteal, anterolateral thigh, and deltoid injections, deltoid and gluteal injections, deltoid and thigh injections, gluteal and thigh injections, or deltoid, gluteal, and thigh injections.

In some embodiments, a “dose” may be a set amount of antibody or antigen-binding fragment (e.g. 250 mg or 500 mg) administered within a set period of time (e.g. less than one day, less than 6 hours, less than 2 hours). In a specific embodiment, a dose is a set amount administered in less than one day.

In some embodiments, the antibody or antigen-binding fragment may be provided at a concentration of 100 mg/mL. This concentration may be used to provide doses with volumes of 5 mL, 2.5 mL, 1.5 mL, 1.25 mL, 0.63 mL, or 0.5 mL. Doses having these volumes may be administered at a single injection site or at two or more injection sites.

In some embodiments, the pediatric subject may be a preterm newborn infant or term newborn infant weighing 2 kg or more.

In some embodiments, the antibody or antigen binding fragment may have a concentration of about 62.5 mg/mL prior to administration, about 75 mg/mL, about 80 mg/mL, about 85 mg/mL, about 90 mg/mL, about 95 mg/mL, about 100 mg/mL, about 105 mg/mL, about 110 mg/mL, about 115 mg/mL, about 120 mg/mL, about 125 mg/mL, about 130 mg/mL, about 135 mg/mL, about 140 mg/mL, about 145 mg/mL, or about 150 mg/mL prior to administration, or any concentration in a range from about 75 mg/mL to about 150 mg/mL, about 95 mg/mL to about 105 mg/mL, or about 120 mg/mL to about 130 mg/mL prior to administration.

In some embodiments, the antibody is proved at a high concentration in a range from 62.5 mg/mL to 150 mg/mL, in a range from 75 mg/mL to 150 mg/mL, in a range from 100 mg/mL to 125 mg/mL, in a range from 95 mg/mL to 105 mg/mL, or in a range from 120 mg/mL to 130 mg/mL.

In some embodiments, the antibody or antigen-binding fragment is provided at a high concentration of at least 62.5 mg/mL, at least 65 mg/mL, at least 70 mg/mL, at least 75 mg/mL, at least 80 mg/mL, at least 85 mg/mL, at least 90 mg/mL, at least 95 mg/mL, at least 100 mg/mL, at least 105 mg/mL, at least 110 mg/mL, at least 115 mg/mL, at least 120 mg/mL, at least 125 mg/mL, at least 130 mg/mL, at least 135 mg/mL, at least 140 mg/mL, at least 145 mg/mL, or at least 150 mg/mL.

In some embodiments, the antibody or antigen-binding fragment is provided at a high concentration of up to 65 mg/mL, 70 mg/mL, 85, mg/mL, 80 mg/mL, at least 85 mg/mL, at least 90 mg/mL, at least 95 mg/mL, at least 100 mg/mL, at least 105 mg/mL, at least 110 mg/mL, at least 115 mg/mL, at least 120 mg/mL, at least 125 mg/mL, at least 130 mg/mL, at least 135 mg/mL, at least 140 mg/mL, at least 145 mg/mL, or at least 150 mg/mL.

In some embodiments, the antibody may be Sotrovimab and it may be formulated for intravenous infusion or intramuscular injection at a concentration of 62.5 mg/mL. In some embodiments, the antibody may be diluted prior to administration to a patient. The formulation may be a clear, liquid solution, more particularly a sterile solution.

The formulation may contain at least 10 mM histidine, at least 15 mM histidine, at least 20 mM histidine, at least 25 mM histidine, a range from 10 mM to 25 mM histidine, a range from 10 mM to 20 mM histidine, a range from 15 mM to 20 mM histidine, a range from 20 mM to 25 mM histidine, or 20 mM histidine.

The formulation may contain at least 5%, at least 6%, at least 7%, at least 8%, or at least 9% sucrose (w/v), a range from 5% to 9%, from 6% to 9%, from 7% too 9%, from 5% to 7%, from 5% to 8%, from 6% to 7%, or from 6% to 8% sucrose (w/v), or 7% sucrose (w/v).

The formulation may contain at least 0.02%, at least 0.03%, at least 0.04%, at least 0.05%, or at least 0.06% PS80 (w/v), a range from 0.02% to 0.06%, from 0.02% to 0.04%, or from 0.04% to 0.06% PS80 (w/v), or 0.04% PS80 (w/v).

The formulation may contain at least 4 mM, at least 4.5 mM, at least 5 mM, at least 5.5 mM, or at least 6 mM L-methionine, a range from 4 mM to 6 mM, from 4 mM to 5 mM, or from 5 mM to 6 mM L-methionine, or 5 mM L-methionine.

The formulation may have a pH in a range from 5.0-7.0, from 5.0-6.0, from 6.0-7.0, or from 5.5 to 6.5, or a pH of 6.0.

The formulation may also contain 20 mM histidine, 7% sucrose (w/v), 0.04% PS80 (w/v), 5 mM L-methionine and may be at pH 6.0.

The formulation may contain no preservatives.

In some embodiments, the sotrovimab may be provided in a single-use 10 mL vial that contains 500 mg of sotrovimab.

In some embodiments, each pediatric subject may be administered only a single dose of antibody or antigen-binding fragment.

In particular embodiments, the antibody or antigen-binding fragment comprises CDRH1, CDRH2, CDRH3, CDRL1, CDRL2, and CDRL3 amino acid sequences of SEQ ID NOs.:106, 121, 108, 169, 170, and 171, respectively. In further embodiments, the antibody or antigen-binding fragment comprises a VH comprising the amino acid sequence of SEQ ID NO.:113 and a VL comprising the amino acid sequence of SEQ ID NO.:168. In certain further embodiments, the antibody or antigen-binding fragment comprises M428L and N434S Fc mutations (sotrovimab aka VIR-7831) and/or M428L, N434S, G236A, A330L, and 1332E Fc mutations (VIR-7832).

A number of criteria are believed to contribute to high risk for contraction, transmission, progression of disease, and/or severe symptoms or death associated with a SARS CoV-2 infection. These include age, occupation, general health, pre-existing health conditions, close contacts with subjects who have or are suspected to or are at risk of having a SARS-CoV-2 infection, and lifestyle habits. In pediatric subjects, particular risk factors include age of less than one year, asthma or chronic lung disease, diabetes, genetic, neurologic, or metabolic conditions, sickle cell disease, heart disease since birth, immunosuppression (weakened immune system due to certain medical conditions or being on medications that weaken the immune system), medical complexity (children with multiple chronic conditions that affect many parts of the body or are dependent on technology and other significant supports for daily life), and obesity (obesity was defined as body mass index (kg/m2) ≥95th percentile for age and sex based on CDC growth charts). In some embodiments, a pediatric subject treated according to the present disclosure has one or more risk factors.

In some embodiments, a close contact comprises a pediatric subject that: (a) has resided with an index case in the 7 days prior to index diagnosis, and (b) is less than 3 days since last exposure (close contact with a person with SARS-CoV-2 infection) to the index case.

In certain embodiments, a pediatric subject treated according to the present disclosure has received a vaccine for SARS-CoV-2. In some embodiments, the vaccine is determined to be ineffective, e.g., by post-vaccine infection or symptoms in the pediatric subject, by clinical diagnosis or scientific or regulatory criteria. In certain embodiments, a pediatric subject treated according to the present disclosure has not received a vaccine for SARS-CoV-2. In certain embodiments, a pediatric subject treated according to the present disclosure is receiving or has received i) convalescent plasma therapy, ii) remdesivir, sofosbuvir, acyclovir, zidovudine, molnupiravir, nirmatrelvir, ritonavir, or favipiravir, or brequinar, dexamethasone, tocilizumab, etesevimab, lopinavir, leronlimab, bebtelovimab, casirivimab, imdevimab, or any combination thereof, or both i) and ii), for SARS-CoV-2.

In certain embodiments, treatment is administered as pre-exposure or peri-exposure prophylaxis. In certain embodiments, treatment is administered to a pediatric subject with mild-to-moderate disease, which may be in an outpatient setting, for example intravenously or by intramuscular injection. In certain embodiments, treatment is administered to a pediatric subject with moderate-to-severe disease, such as requiring hospitalization. Sequelae of severe disease can include: respiratory failure; thromboembolic disease leading to pulmonary embolism and stroke; arrhythmia; shock; or any combination thereof. In certain embodiments, severe COVID-19 comprises (i) hypoxemia (O₂ saturation ≤93% on room air or PaO₂/FiO₂<300) requiring oxygen supplementation for more than 1 day or (ii) the pediatric subject requiring ≥4 L/min oxygen supplementation or equivalent.

In some embodiments, wherein the pediatric subject has, or is at risk for progressing to, critical COVID-19. Critical disease generally includes an increased risk of mortality as compared to severe disease. In some embodiments, critical COVID-19 comprises respiratory failure requiring at least one of the following: invasive mechanical ventilation and extracorporeal membrane oxygenation (ECMO); shock; and multi-organ dysfunction/failure.

In certain embodiments, a pediatric subject is hospitalized with COVID-19, which can include, for example, admission or transfer to an intensive care unit (ICU).

In any of the presently disclosed embodiments, the pediatric subject having a SARS-CoV-2 infection: has mild-to-moderate COVID-19; is experiencing any one or more of: fever; cough; fatigue; shortness of breath or difficulty breathing; muscle aches; chills; sore throat; runny nose; headache; chest pain; loss of taste and/or smell; and pink eye (conjunctivitis); malaise; and abnormal imaging; has evidence of lower respiratory disease by clinical assessment or imaging and a saturation of oxygen (SaO₂) greater than (>)93 percent (%) on room air at sea level, has a positive SARS-CoV-2 viral testing result, and/or is at high risk for progressing to severe COVID-19 and/or hospitalization, e.g., the human pediatric subject is 12-17 years of age and has a BMI ≥85% for their age and gender, or sickle cell disease, congenital or acquired heart disease, neurodevelopmental disorders (e.g., cerebral palsy), a medical-related technological dependence (e.g., tracheostomy, gastrostomy, or positive pressure ventilation not related to COVID-19), or asthma, reactive airway or other chronic respiratory disease that requires daily medication for control; has recently been diagnosed with COVID-19 (e.g., within 1 day, 2 days, 3 days, 4 days, 5 days, 6 days, 7 days, 8 days, 9 days, 10 days, 11 days, 12 days, 13 days, or 14 days) and/or is within 10 days of symptom onset; or has or is experiencing any combination of the foregoing.

In some embodiments, the pediatric subject has a laboratory confirmed COVID-19 infection by positive polymerase chain reaction (PCR; e.g., RT-PCR) test; e.g., on any type of respiratory tract sample). In some embodiments, a pediatric subject has peripheral capillary oxygen saturation (SpO₂) >94% room air (RA), who have experienced one or more symptoms of COVID-19 for ≤120 h (5 days). In some embodiments, a paediatric subject receiving therapy according to the present disclosure is receiving or has received remdesivir, sofosbuvir, acyclovir, zidovudine, molnupiravir, nirmatrelvir, ritonavir, or favipiravir, or brequinar, dexamethasone, tocilizumab, etesevimab, lopinavir, leronlimab, bebtelovimab, casirivimab, imdevimab. or any combination thereof. In any of the presently disclosed embodiments, the method can comprise administering a single dose of the antibody, antigen-binding fragment, or composition to the paediatric subject. Typical routes of administering the presently disclosed compositions thus include, without limitation, oral, topical, transdermal, inhalation, parenteral, sublingual, buccal, rectal, vaginal, and intranasal. The term “parenteral”, as used herein, includes subcutaneous injections, intravenous, intramuscular, intrasternal injection or infusion techniques. In certain embodiments, administering comprises administering by a route that is selected from oral, intravenous, parenteral, intragastric, intrapleural, intrapulmonary, intrarectal, intradermal, intraperitoneal, intratumoral, subcutaneous, topical, transdermal, intracisternal, intrathecal, intranasal, and intramuscular. In particular embodiments, a method comprises orally administering the antibody, antigen-binding fragment, polynucleotide, vector, host cell, or composition to the pediatric subject.

In preferred embodiments, a method comprises administering the antibody, antigen-binding fragment, or composition to the pediatric subject intravenously or intramuscularly. A single dose of the antibody or antigen-binding fragment may be administered to a pediatric subject intravenously over the course of 30 minutes, 60 minutes, or 90 minutes. Intramuscular administration may be by deltoid, thigh, or gluteal injection. In particular intramuscular administration may be administered in the dorsogluteal, ventrogluteal, or anterolateral thigh, or deltoid muscle location (if pediatric subject 2 years of age or older).

In some embodiments, the pediatric subject: has mild to moderate COVID-19; has severe COVID-19; has severe to critical COVID-19; has had fewer than seven days or 5 or fewer days since onset of symptoms; has had seven days or more since onset of symptoms; has had a positive reverse-transcriptase-polymerase-chain-reaction or antigen SARS-CoV-2 test result; has one or more of: diabetes requiring medication, obesity (obesity was defined as body mass index (kg/m2) ≥95^{th} percentile for age and sex based on CDC growth charts), chronic kidney disease (estimated glomerular filtration rate <60 mL/min/1.73 m²), congestive heart failure (New York Heart Association class II or higher), chronic obstructive pulmonary disease (history of chronic bronchitis, chronic obstructive lung disease, or emphysema with dyspnea on physical exertion), and moderate to severe asthma (pediatric subject requires an inhaled steroid to control symptoms or has been prescribed a course of oral steroids in the past year); or any combination of the above.

In some embodiments, the pediatric subject: is aged less than two years, optionally at least 32 weeks gestational age at birth or less than 32 weeks gestational age at birth; is aged two years to less than six years; is aged six years to less than twelve years; is aged twelve years to less than eighteen years; has mild to moderate COVID-19; has severe COVID-19; has severe to critical COVID-19; has had fewer than seven days since onset of symptoms; has had a positive reverse-transcriptase-polymerase-chain-reaction or antigen SARS-CoV-2 test result; has one or more of: age less than one, diabetes mellitus, Pompe Disease, Mucopolysaccharidoses, glycogen storage diseases, fatty acid oxidation disorders, maple syrup urine disease, organic acidemias, obesity, congenital heart disease, hypertension, cardiomyopathy, heart failure, sickle cell disease, moderate to severe asthma, chronic lung disease, obstructive sleep apnea, Cystic Fibrosis, seizure disorder, global developmental delay, cerebral palsy, structural brain defect/malformation, primary immunodeficiency, HIV infection with CD4+ count <200 cells/mm3, solid organ or bone marrow transplant, long-term use of systemic corticosteroids, immunosuppressive biologic agents, or disease-modifying anti-rheumatic drugs, gastrostomy- or jejunostomy-dependence, parenteral nutrition dependence, tracheostomy-dependence, baseline oxygen requirement, use of CPAP/BiPAP/ventilator support, or other baseline medical complexity; weighs at least 2 kg, or any combinations thereof.

In certain embodiments, a method comprises administering the antibody, antigen-binding fragment, polynucleotide, vector, host cell, or composition to the pediatric subject one time.

In certain embodiments, a method comprises administering the antibody, antigen-binding fragment, polynucleotide, vector, host cell, or composition to the pediatric subject at 2, 3, 4, 5, 6, 7, 8, 9, 10 times, or more.

In certain embodiments, a method comprises administering the antibody, antigen-binding fragment, or composition to the pediatric subject a plurality of times, wherein a second or successive administration is performed at about 6, about 7, about 8, about 9, about 10, about 11, about 12, about 24, about 48, about 74, about 96 hours, or more, following a first or prior administration, respectively.

In certain embodiments, a method comprises administering the antibody, antigen-binding fragment, polynucleotide, vector, host cell, or composition at least one time prior to the pediatric subject being infected by the SARS-CoV-2.

In certain embodiments, the pediatric subject receiving treatment is 18 or more years of age with laboratory-confirmed (e.g., by PCR test) SARS-CoV-2 infection.

In some embodiments, the pediatric subject has a clinical status of Grade 4 (hospitalized, oxygen by mask or nasal prongs), 5 (hospitalized, on non-invasive ventilation, or high flow oxygen), 6 (hospitalized, intubation and mechanical ventilation) or 7 (ventilation and additional organ support—pressors, renal replacement therapy (RRT), extracorporeal membrane oxygenation (ECMO)), as defined by the WHO clinical severity score, 9-point ordinal scale.

In some embodiments, the pediatric subject has mild-to-moderate COVID-19. In some embodiments, the pediatric subject is at-risk of progression to severe COVID-19. In some embodiments, following administration of the antibody, antigen-binding fragment, or composition to the pediatric subject, the pediatric subject is at a reduced risk of hospitalization for COVID-19. In certain embodiments, following administration of the antibody, antigen-binding fragment, or composition to the pediatric subject, the risk of hospitalization for COVID-19 is reduced by 10% or more, 20% or more, 30% or more, 40% or more, 50% or more, 60% or more, 70% or more, 80% or more, or 85% or more.

In some embodiments, the pediatric subject has or is at risk for progressing to severe COVID-19, wherein, optionally, severe COVID-19 comprises (i) hypoxemia (O₂ saturation ≤93% on room air or PaO₂/FiO₂<300) requiring oxygen supplementation for more than 1 day or (ii) the pediatric subject requiring ≥4 L/min oxygen supplementation or equivalent.

In some embodiments, the pediatric subject has or is at risk for progressing to critical COVID-19, wherein, optionally, critical COVID-19 comprises respiratory failure requiring at least one of the following: invasive mechanical ventilation and ECMO; shock; and multi-organ dysfunction/failure.

In some embodiments, the pediatric subject is less than seven days since onset of symptoms. In other embodiments, the pediatric subject is seven days or more since onset of symptoms.

In some embodiments, the pediatric subject is any one or more of (i)-(iii): (i) has a positive SARS-CoV-2 test result (by any validated test e.g. RT-PCR on any specimen type); (ii) (1) hospitalized with severe COVID-19 disease defined as requirement for supplemental oxygen or non-invasive ventilation consistent with Grade 4 or Grade 5 disease or (2) hospitalized with critical COVID-19 disease defined as those on mechanical ventilation (Grade 6 or Grade 7 disease)); (iii) is male or female, wherein, optionally, (1) the woman is non-childbearing potential (WONCBP) or (2) is a woman of child-bearing potential (WOCBP) and uses a contraceptive method.

In any of the presently disclosed embodiments, the pediatric subject can have had or can have close contacts to a person with a confirmed SARS-CoV-2 infection.

In any of the presently disclosed embodiments, treating can comprise preventing infection by SARS-CoV-2 and/or COVID-19. In any of the presently disclosed embodiments, treating can comprise preventing progression of COVID-19 in the pediatric subject. In any of the presently disclosed embodiments, treating can comprise preventing contraction and/or transmission of symptomatic COVID-19. In any of the presently disclosed embodiments, treating can comprise preventing contraction and/or transmission of asymptomatic COVID-19. In any of the presently disclosed embodiments, the pediatric subject can be at-risk for contracting or progressing on COVID-19.

In any of the presently disclosed embodiments, treating can comprise preventing or reducing: (1) one or more acute respiratory symptom selected from: cough; sputum production; sore throat; and shortness of breath; or (2) fever of greater than 38° C.; (3) two or more of the following symptoms: fatigue; myalgias/arthralgias; chills; nausea/vomiting; diarrhea; and anosmia/dysgeusia.

In any of the presently disclosed embodiments, treating can comprise preventing or reducing one or more of the following symptoms: fever of greater than 38° C.; chills; cough; sore throat; malaise; headache; myalgia; a change in smell or taste; nasal congestion/rhinorrhea; vomiting; diarrhea; shortness of breath on exertion.

In any of the presently disclosed embodiments, treating can comprise preventing or reducing one or more of the following symptoms: fever of greater than 38° C.; chills, cough, sore throat, malaise, headache, joint or muscle pain, change in smell or taste, vomiting, diarrhea, shortness of breath, poor appetite or poor feeding, nasal congestion/runny nose, lethargy.

In any of the presently disclosed embodiments, administering the antibody, antigen-binding fragment, or composition can comprise intravenous infusion. In any of the presently disclosed embodiments, administering the antibody, antigen-binding fragment, or composition can comprise intramuscular injection.

In any of the presently disclosed embodiments, the pediatric subject can have a mild-to-moderate SARS-2-CoV infection (e.g., has mild-to-moderate COVID-19) and, optionally, be at risk for progression to severe disease.

In any of the presently disclosed embodiments, the pediatric subject: (i) can be 12 years old or older; and (ii) have last had contact with a person with a confirmed SARS-CoV-2 infection less than three days prior to administration of the composition.

In any of the presently disclosed embodiments, the pediatric subject has mild-to-moderate COVID-19 and the method comprises administering a single dose of the antibody, antigen-binding fragment, or composition to the pediatric subject intramuscularly.

In some embodiments: (i) the pediatric subject is 12 years of age or older and is at high risk of progression of COVID-19 and/or (ii) the pediatric subject has a positive SARS-CoV-2 test result (e.g., by PCR test), has oxygen saturation >94% on room air, has COVID-19 symptoms, and is less than or equal to 7 days from onset of symptoms.

In any of the presently disclosed embodiments: (i) the pediatric subject can be 12 years of age or older and is at high risk of progression of COVID-19 and (ii) the pediatric subject can have a positive SARS-CoV-2 test result (e.g., by PCR test), has oxygen saturation ≥94% on room air, has COVID-19 symptoms, and is less than or equal to 7 days from onset of symptoms.

In any of the presently disclosed embodiments, the pediatric subject is not hospitalized and is at high-risk for (i) hospitalization and/or (ii) progression of COVID-19.

In any of the presently disclosed embodiments, the pediatric subject can be: 12 or more years of age and, optionally, at high risk of progression of COVID-19. In any of the presently disclosed embodiments, the pediatric subject can have had a positive SARS-CoV-2 test result, has oxygen saturation ≥94% on room air, has COVID-19 symptoms, and is less than or equal to 7 days from onset of symptoms.

In any of the presently disclosed embodiments, the antibody or antigen-binding fragment was obtained from a non-clonal pool of cells stably transfected with a polynucleotide encoding the antibody or antigen-binding fragment. In any of the presently disclosed embodiments, the antibody or antigen-binding fragment was obtained from a clonal master cell bank. A Master Cell Bank (MCB) is produced from an original antibody/antigen-binding fragment-producing cell line. A MCB is generally cryopreserved in multiple vials to prevent genetic variation and potential contamination by eliminating the total number of times a cell line is passaged or handled during the manufacturing process. A MCB is preferably tested for contaminants such as bacteria, fungi, and mycoplasmas; these should not be present in the MCB.

In any of the presently disclosed embodiments, the pediatric subject: is a family member or other close contact of a subject diagnosed with or suspected of having a SARS-CoV-2 infection, is overweight or clinically obese; is or has been a smoker; has or had chronic obstructive pulmonary disease (COPD); is asthmatic (e.g., having moderate to severe asthma); has an autoimmune disease or condition (e.g., diabetes); has a compromised or depleted immune system (e.g., due to AIDS/HIV infection, a cancer such as a blood cancer, a lymphodepleting therapy such as a chemotherapy, a bone marrow or organ transplantation, or a genetic immune condition); has chronic liver disease; has cardiovascular disease; and/or has a pulmonary or heart defect; and/or works or otherwise spends time in close proximity with others, such as in a factory, shipping center, hospital setting, or the like. In any of the presently disclosed embodiments, the pediatric subject has received a vaccine for SARS-CoV-2 and the vaccine is determined to be ineffective, e.g., by post-vaccine infection or symptoms in the pediatric subject, by clinical diagnosis or scientific or regulatory criteria, or by infection with as SARS-CoV-2 variant against which the vaccine is less effective than variants for which the vaccine was originally tested or not effective. In any of the presently disclosed embodiments, the pediatric subject has not received a vaccine for SARS-CoV-2.

In any of the presently disclosed embodiments, the pediatric subject is receiving or has received i) convalescent plasma therapy, ii) remdesivir, sofosbuvir, acyclovir, zidovudine, molnupiravir, nirmatrelvir, ritonavir, or favipiravir, or brequinar, dexamethasone, tocilizumab, etesevimab, lopinavir, leronlimab, bebtelovimab, casirivimab, imdevimab, or any combination thereof, or both i) and ii), for SARS-CoV-2. In any of the presently disclosed embodiments, treatment comprises pre-exposure or peri-exposure prophylaxis. In any of the presently disclosed embodiments, treatment is administered to the pediatric subject having mild-to-moderate disease, optionally in an outpatient setting. In any of the presently disclosed embodiments, treatment is administered to a pediatric subject with moderate-to-severe disease, such as requiring hospitalization. In any of the presently disclosed embodiments, the pediatric subject is hospitalized with COVID-19.

In any of the presently disclosed embodiments, the pediatric subject having a SARS-CoV-2 infection: has mild-to-moderate COVID-19; is experiencing any one or more of: fever; cough; fatigue; shortness of breath or difficulty breathing; muscle aches; chills; sore throat; runny nose; headache; chest pain; loss of taste and/or smell; and pink eye (conjunctivitis); malaise; and abnormal imaging; has evidence of lower respiratory disease by clinical assessment or imaging and a saturation of oxygen (SaO₂) greater than (>)93 percent (%) on room air at sea level, has a positive SARS-CoV-2 viral testing result, and/or is at high risk for progressing to severe COVID-19 and/or hospitalization, e.g., the human has chronic kidney disease; has diabetes; has immunosuppressive disease, is receiving immunosuppressive treatment; and/or is 12-17 years of age and has a BMI ≥85% for their age and gender, or sickle cell disease, congenital or acquired heart disease, neurodevelopmental disorders (e.g., cerebral palsy), a medical-related technological dependence (e.g., tracheostomy, gastrostomy, or positive pressure ventilation not related to COVID-19), or asthma, reactive airway or other chronic respiratory disease that requires daily medication for control; has recently been diagnosed with COVID-19 (e.g., within 1 day, 2 days, 3 days, 4 days, 5 days, 6 days, 7 days, 8 days, 9 days, 10 days, 11 days, 12 days, 13 days, or 14 days) and/or is within 10 days of symptom onset; or has or is experiencing any combination of the foregoing.

In any of the presently disclosed embodiments, the pediatric subject has a laboratory confirmed COVID-19 infection by positive polymerase chain reaction (PCR; e.g., RT-PCR) test; e.g., on any type of respiratory tract sample). In any of the presently disclosed embodiments, the pediatric subject has peripheral capillary oxygen saturation (SpO₂) >94% room air (RA), and has experienced one or more symptoms of COVID-19 for ≤120 h (5 days). In any of the presently disclosed embodiments, the paediatric subject is further receiving or has received is receiving or has received i) convalescent plasma therapy, ii) remdesivir, sofosbuvir, acyclovir, zidovudine, molnupiravir, nirmatrelvir, ritonavir, or favipiravir, or brequinar, dexamethasone, tocilizumab, etesevimab, lopinavir, leronlimab, bebtelovimab, casirivimab, imdevimab, or any combination thereof, iii) supplemental oxygen or respiration therapy, or two or more of i), ii), and iii) for SARS-CoV-2.

In some embodiments, one or more of the following does not apply to a paediatric subject receiving therapy according to the present disclosure: any condition that would prohibit receipt of intramuscular injections such as coagulation disorder, bleeding diathesis, or thrombocytopenia; known allergy or hypersensitivity to any constituent present in an antibody composition; previous anaphylaxis or hypersensitivity to a monoclonal antibody; has previously received a COVID-19 vaccine; has previously received SARS-CoV-2 hyperimmune intravenous immunoglobulin (hIVIG) from COVID-19 survivors; has previously received convalescent plasma from a recovered COVID-19 patient or an anti-SARS-CoV-2 mAb; is a pregnant or breast-feeding female; Alanine aminotransferase (ALT) and/or aspartate aminotransferase (AST) ≥5 times the upper limit of normal (ULN); Stage 4 severe chronic kidney disease or requiring dialysis (i.e., estimated glomerular filtration rate <30 mL/min/1.73 m²); has symptoms consistent with severe COVID-19 as defined by shortness of breath at rest or respiratory distress or requiring supplemental oxygen; severely immunocompromised participants including but not limited to cancer patients receiving immunosuppressive chemotherapy or immunotherapy, those with a solid organ transplant or allogeneic stem cell transplant within the last 3 months, any history of heart or lung transplant or high dose long-term systemic corticosteroids (equivalent to ≥20 mg a day of prednisone or the systemic equivalent for over 2 weeks); had diabetes (requiring medication), chronic kidney disease (i.e., eGFR <60 as determined by the Modification of Diet in Renal Disease (MDRD) study), chronic liver disease (e.g., cirrhosis), congestive heart failure (New York Heart Association (NYHA) class II or more), chronic obstructive pulmonary disease (history of chronic bronchitis, chronic obstructive lung disease, or emphysema with dyspnoea on physical exertion), and moderate to severe asthma (participant requires an inhaled steroid to control symptoms or has been prescribed a course of oral steroids in the past year); previous anaphylaxis or hypersensitivity to a monoclonal antibody; end-organ dysfunction such as—a. stroke b. meningitis c. encephalitis d. myelitis e. myocardial infarction f. myocarditis g. pericarditis h. symptomatic congestive heart failure (New York Heart Association [NYHA] class III-IV) i. arterial or deep venous thrombosis or pulmonary embolism; end-organ failure category such as—a. requirement for high-flow oxygen, non-invasive ventilation, or invasive mechanical ventilation b. extracorporeal membrane oxygenation (ECMO) c. mechanical circulatory support (e.g., intra-aortic balloon pump, ventricular assist device) d. vasopressor therapy e. commencement of renal replacement therapy (RRT) during this admission (i.e. not patients on chronic RRT); stroke; meningitis; encephalitis; myelitis; myocardial ischemia; myocarditis; pericarditis; symptomatic congestive heart failure; arterial or deep venous thrombosis or pulmonary embolism; and current or imminent requirement for invasive mechanical ventilation, ECMO (extracorporeal membrane oxygenation), Mechanical circulatory support, vasopressor therapy, or commencement of renal replacement therapy at this admission (i.e. not patients on chronic renal replacement therapy).

Compositions comprising an antibody, antigen-binding fragment, polynucleotide, vector, host cell, or composition of the present disclosure may also be administered simultaneously with, prior to, or after administration of one or more other therapeutic agents. Such combination therapy may include administration of a single pharmaceutical dosage formulation which contains a compound of the invention and one or more additional active agents, as well as administration of compositions comprising an antibody or antigen-binding fragment of the disclosure and each active agent in its own separate dosage formulation. For example, an antibody or antigen-binding fragment thereof as described herein and the other active agent can be administered to the patient together in a single oral dosage composition such as a tablet or capsule, or each agent administered in separate oral dosage formulations. Similarly, an antibody or antigen-binding fragment as described herein and the other active agent can be administered to the pediatric subject together in a single parenteral dosage composition such as in a saline solution or other physiologically acceptable solution, or each agent administered in separate parenteral dosage formulations. Where separate dosage formulations are used, the compositions comprising an antibody or antigen-binding fragment and one or more additional active agents can be administered at essentially the same time, i.e., concurrently, or at separately staggered times, i.e., sequentially and in any order; combination therapy is understood to include all these regimens.

In certain embodiments, a combination therapy is provided that comprises one or more anti-SARS-CoV-2 antibody (or one or more nucleic acid, host cell, vector, or composition) of the present disclosure and one or more anti-inflammatory agent and/or one or more anti-viral agent. In particular embodiments, the one or more anti-inflammatory agent comprises a corticosteroid such as, for example, dexamethasone, prednisone, or the like. In some embodiments, the one or more anti-inflammatory agents comprise a cytokine antagonist such as, for example, an antibody that binds to IL6 (such as siltuximab), or to IL-6R (such as tocilizumab), or to IL-1β, IL-7, IL-8, IL-9, IL-10, FGF, G-CSF, GM-CSF, IFN-γ, IP-10, MCP-1, MIP-1A, MIP1-B, PDGR, TNF-α, or VEGF. In some embodiments, anti-inflammatory agents such as ruxolitinib and/or anakinra are used. In some embodiments, the one or more anti-viral agents comprise nucleotide analogs or nucleotide analog prodrugs such as, for example, remdesivir, sofosbuvir, acyclovir, zidovudine, molnupiravir, nirmatrelvir, ritonavir, or favipiravir, and brequinar. In particular embodiments, an anti-viral agent comprises lopinavir, ritonavir, favipiravir, leronlimab or any combination thereof. Other anti-inflammatory agents for use in a combination therapy of the present disclosure include non-steroidal anti-inflammatory drugs (NSAIDS). It will be appreciated that in such a combination therapy, the one or more antibody (or one or more nucleic acid, host cell, vector, or composition) and the one or more anti-inflammatory agent and/or one or the more antiviral agent can be administered in any order and any sequence, or together.

In some embodiments, the present disclosure provides a composition comprising an anti-SARS-CoV-2 antibody or antigen-binding fragment and one or more of the above combination therapy agents in any combinations (e.g. the anti-SARS-CoV antibody or antigen-binding fragment and a nucleotide analog or nucleotide analog prodrug).

In some embodiments, an antibody (or one or more nucleic acid, host cell, vector, or composition) is administered to a pediatric subject who has previously received one or more anti-inflammatory agent and/or one or more antiviral agent. In some embodiments, one or more anti-inflammatory agent and/or one or more antiviral agent is administered to a pediatric subject who has previously received an antibody (or one or more nucleic acid, host cell, vector, or composition).

In certain embodiments, a combination therapy is provided that comprises two or more anti-SARS-CoV-2 antibodies of the present disclosure. A method can comprise administering a first antibody to a pediatric subject who has received a second antibody, or can comprise administering two or more antibodies together. For example, in particular embodiments, a method is provided that comprises administering to the pediatric subject (a) a first antibody or antigen-binding fragment, when the pediatric subject has received a second antibody or antigen-binding fragment; (b) the second antibody or antigen-binding fragment, when the pediatric subject has received the first antibody or antigen-binding fragment; or (c) the first antibody or antigen-binding fragment, and the second antibody or antigen-binding fragment.

In a related aspect, uses of the presently disclosed antibodies, antigen-binding fragments, vectors, host cells, and compositions are provided.

In certain embodiments, any of the presently disclosed antibodies, antigen-binding fragments, polynucleotides, vectors, host cells, or compositions is provided for use in a method (e.g., any of the presently disclosed methods) of treating a SARS-CoV-2 infection and/or COVID-19 in a pediatric subject.

In certain embodiments, the disclosure further provides a kit that includes any anti-SARS-CoV-2 antibody or antigen-binding fragment or any composition (including or one or more nucleic acid, host cell, or vector) described herein. The kit can further include instructions for administering the anti-SARS-CoV-2 antibody or antigen-binding fragment or compostions to a pediatric subject. A kit can further include a device (e.g. a syringe or an inhaler) for administering the anti-SARS-CoV-2 antibody or antigen-binding fragment or composition to a pediatric subject. In some embodiments, the antibody or antigen-binding fragment or composition is in the form of an aerosol, and the kit may include at least one of a container, an activator, a valve, a subcontainer, and the like, used in aerosol delivery, which together may form a kit. In certain embodiments, any of the presently disclosed antibodies, antigen-binding fragments, or compositions is provided for use in a method of manufacturing or preparing a medicament for treating a SARS-CoV-2 infection and/or COVID-19 in a pediatric subject.

The present disclosure also provides the following Embodiments.

Embodiment 1. A method of treating a SARS-CoV-2 infection in a pediatric subject, the method comprising administering to the pediatric subject a single dose of a composition comprising a SARS-CoV-2 neutralizing antibody or antigen-binding fragment that comprises: (A)(i) a heavy chain variable domain (VH) that comprises: the complementarity determining region (CDR)H1 amino acid sequence set forth in SEQ ID NO.:106, the CDRH2 amino acid sequence set forth in SEQ ID NO.:121, and the CDRH3 amino acid sequence set forth in SEQ ID NO.:108; and (ii) a light chain variable domain (VL) that comprises: the CDRL1 amino acid sequence set forth in SEQ ID NO.:169, the CDRL2 amino acid sequence set forth in SEQ ID NO.:170, and the CDRL3 amino acid sequence set forth in SEQ ID NO.:171; (B)(i) a heavy chain variable domain (VH) that comprises the CDRH1, CDRH2, and CDRH3, of the amino acid sequence set forth in SEQ ID NO: 113 or 105, optionally as determined by IMGT; and (ii) and a light chain variable region that comprises the CDRL1, CDRL2, and CDRL3 of the amino acid sequence set forth in SEQ ID NO: 168, optionally as determined by IMGT; or (C)(i) a heavy chain variable domain (VH) that comprises or consists of the amino acid sequence set forth in SEQ ID NO: 113 or 105; and (ii) a light chain variable region that comprises or consists of the amino acid sequence set forth in SEQ ID NO: 168.

Embodiment 2. A method of treating a SARS-CoV-2 infection in a pediatric subject, the method comprising administering to the pediatric subject an effective amount of (i) a SARS-CoV-2 neutralizing antibody or antigen-binding fragment, or (ii) a composition comprising (ii)(a) the antibody or antigen-binding fragment and (ii)(b) a pharmaceutically acceptable excipient, carrier, or diluent, wherein the antibody or antigen-binding fragment comprises: (A)(i) a heavy chain variable domain (VH) that comprises: the complementarity determining region (CDR)H1 amino acid sequence set forth in SEQ ID NO.:106, the CDRH2 amino acid sequence set forth in SEQ ID NO.:121, and the CDRH3 amino acid sequence set forth in SEQ ID NO.:108; and (ii) a light chain variable domain (VL) that comprises: the CDRL1 amino acid sequence set forth in SEQ ID NO.:169, the CDRL2 amino acid sequence set forth in SEQ ID NO.:170, and the CDRL3 amino acid sequence set forth in SEQ ID NO.:171; (B)(i) a heavy chain variable domain (VH) that comprises the CDRH1, CDRH2, and CDRH3, of the amino acid sequence set forth in SEQ ID NO: 113 or 105, optionally as determined by IMGT; and (ii) and a light chain variable region that comprises the CDRL1, CDRL2, and CDRL3 of the amino acid sequence set forth in SEQ ID NO: 168, optionally as determined by IMGT; or (C)(i) a heavy chain variable domain (VH) that comprises or consists of the amino acid sequence set forth in SEQ ID NO: 113 or 105; and (ii) a light chain variable region that comprises or consists of the amino acid sequence set forth in SEQ ID NO: 168.

Embodiment 3: The method of Embodiment 1 or Embodiment 2, wherein the method comprises administering to the pediatric subject only a single dose of the antibody or antigen-binding fragment of Embodiment 1 or the composition of Embodiment 2.

Embodiment 4: The method of any one of Embodiments 1-3, wherein the VH comprises the amino acid sequence set forth in SEQ ID NO.:113 and the VL comprises the amino acid sequence set forth in SEQ ID NO.:168.

Embodiment 5: The method of any one of Embodiments 1-3, wherein the VH comprises the amino acid sequence set forth in SEQ ID NO.:105 and the VL comprises the amino acid sequence set forth in SEQ ID NO.:168.

Embodiment 6: The method of any one of Embodiments 1-5, wherein the antibody or antigen-binding fragment further comprises an Fc polypeptide or a fragment thereof.

Embodiment 7: The method of any one of Embodiments 1-6, wherein the antibody or antigen-binding fragment is an IgG, IgA, IgM, IgE, or IgD isotype.

Embodiment 8: The method of any one of Embodiments 1-7, wherein the antibody or antigen-binding fragment is an IgG isotype selected from IgG1, IgG2, IgG3, and IgG4.

Embodiment 9: The method of any one of Embodiments 1-8, wherein the antibody or antigen-binding fragment is an IgG1 isotype.

Embodiment 10: The method of any one of Embodiments 6-9, wherein the Fc polypeptide or fragment thereof comprises the following mutations, wherein the numbering of amino acid residues is according to the EU numbering system: (i) M428L/N434S; or (ii)M428L/N434S/G236A/A330L/I332E.

Embodiment 11: The method of any one of Embodiments 1-10, wherein the antibody or antigen-binding fragment comprises the CH1-CH3 amino acid sequence of SEQ ID NO.:173 or 265 and the CL amino acid sequence of SEQ ID NO.:174.

Embodiment 12: The method of any one of Embodiments 1-10, wherein the antibody or antigen-binding fragment comprises the CH1-CH3 amino acid sequence of SEQ ID NO.:175 or 266 and the CL amino acid sequence of SEQ ID NO.:174.

Embodiment 13: The method of any one of Embodiments 1-12, wherein the antibody or antigen-binding fragment comprises a heavy chain polypeptide and a light chain polypeptide, wherein: i) the heavy chain polypeptide comprises the VH amino acid sequence set forth in SEQ ID NO.:113 and the CH1-CH3 amino acid sequence set forth in SEQ ID NO.:173 or 265; and (ii) the light chain comprises the VL amino acid sequence set forth in SEQ ID NO.:168 and the CL amino acid sequence set forth in SEQ ID NO.:174.

Embodiment 14: The method of any one of Embodiments 1-12, wherein the antibody or antigen-binding fragment comprises a heavy chain polypeptide and a light chain polypeptide, wherein: (i) the heavy chain polypeptide comprises the VH amino acid sequence set forth in SEQ ID NO.:113 and the CH1-CH3 amino acid sequence set forth in SEQ ID NO.:175 or 266; and (ii) the light chain comprises the VL amino acid sequence set forth in SEQ ID NO.:168 and the CL amino acid sequence set forth in SEQ ID NO.:174.

Embodiment 15: The method of any one of Embodiments 1-14, wherein the pediatric subject: (i) is aged less than two years, but at least 32 weeks gestational age at birth; (ii) is aged two years to less than eighteen years, (iii) is aged two years to less than six years; (iv) is aged six years to less than twelve years; (v) is aged twelve years to less than eighteen years; (vi) has mild to moderate COVID-19; (vii) has had fewer than seven days since onset of symptoms; (viii) has had a positive reverse-transcriptase-polymerase-chain-reaction or antigen SARS-CoV-2 test result; (ix) has any one or more of: age less than one year, diabetes mellitus, Pompe Disease, Mucopolysaccharidoses, glycogen storage diseases, fatty acid oxidation disorders, maple syrup urine disease, organic acidemias, obesity (e.g. as defined as body mass index (kg/m2) ≥95th percentile for age and sex based on local growth charts for children ≥2 years of age [or if not available based on the CDC or WHO growth charts respectively]), congenital heart disease, hypertension, cardiomyopathy, heart failure, sickle cell disease, moderate to severe asthma, chronic lung disease, obstructive sleep apnea, Cystic Fibrosis, seizure disorder, global developmental delay, cerebral palsy, structural brain defect/malformation, primary immunodeficiency, HIV infection with CD4+ count <200 cells/mm3, solid organ or bone marrow transplant, long-term use of systemic corticosteroids (defined by either ≥0.5 mg/kg/day by body weight or ≥20 mg/day prednisone equivalents [whichever is the lower dose of the two] taken for ≥2 weeks), immunosuppressive biologic agents (e.g. rituximab), disease-modifying anti-rheumatic drugs (e.g., azathioprine, methotrexate, leflunomide), gastrostomy- or jejunostomy-dependence, parenteral nutrition dependence, tracheostomy-dependence, baseline oxygen requirement, use of CPAP/BiPAP/ventilator support, or other baseline medical complexity; (x) is a preterm newborn infant or term newborn infant weighing 2 kg or more; or (xi) any combination of (i)-(x).

Embodiment 16: The method of any one of Embodiments 1-15, comprising administering the antibody, antigen-binding fragment, or composition to the pediatric subject intravenously.

Embodiment 17: The method of Embodiment 16, comprising administering the antibody, antigen-binding fragment, or composition to the pediatric subject intravenously over the course of 30 minutes, 60 minutes, or 90 minutes.

Embodiment 18: The method of any one of Embodiments 1-15, comprising administering the antibody, antigen-binding fragment, or composition to the pediatric subject intramuscularly.

Embodiment 19: The method of Embodiment 18, comprising administering the antibody, antigen-binding fragment, or composition to the pediatric subject by deltoid or gluteal (e.g. dorsogluteal, ventrogluteal), or thigh (e.g. anterolateral thigh) injection, optionally, the antibody, antigen-binding fragment, or composition is administered in the dorsogluteal, ventgrogluteal, or anterolateral thigh and the prediatric subject is less than 2 years of age.

Embodiment 20: The method of any one of Embodiments 1-19, wherein the method comprises administering the antibody or antigen-binding fragment to the pediatric subject at a dose of up to 62.5 mg, up to 100 mg, up to 125 mg, up to 150 mg, up to 187.5 mg, up to 200 mg, up to 225 mg, up to 250 mg, up to 300 mg, up to 350 mg, up to 375 mg, up to 400 mg, up to 450 mg, or up to 500 mg.

Embodiment 21: The method of any one of Embodiments 1-20, wherein the method comprises administering the antibody or antigen-binding fragment to the pediatric subject at a dose in a range from about 50 mg to about 500 mg, or in a range from about 50 mg to about 250 mg, or in a range from about 50 mg to 100 mg, or in a range from about 100 mg to about 500 mg, or in a range from about 250 mg to about 500 mg, or in a range from about 62.5 mg to about 500 mg, or in a range from about 62.5 mg to about 375 mg, or in a range from about 100 mg to about 350 mg.

Embodiment 22: The method of any one of Embodiments 1-21, wherein the method comprises administering 50, 67.5, 75, 100, 125, 150, 175, 187.5, 200, 225, 250, 275, 300, 325, 350, 375, 400, 425, 450, 475, or 500 mg of the antibody or antigen-binding fragment to the pediatric subject.

Embodiment 23: The method of any one of Embodiments 1-22, wherein the method comprises administering 62.5, 125, 187.5, 250, or 375 mg of the antibody or antigen-binding fragment to the pediatric subject.

Embodiment 24: The method of any one of Embodiments 1-22, wherein the method comprises administering 100 mg, a range from 200 mg to 250 mg, or 500 mg of the antibody or antigen-binding fragment to the pediatric subject.

Embodiment 25: The method of any one of Embodiments 1-22, wherein the method comprises administering 100 mg, 150 mg, 225 mg, or 350 mg of the antibody or antigen-binding fragment to the pediatric subject.

Embodiment 26: The method of any one of Embodiments 1-22, wherein the method comprises: (i) administering 62.5 mg of the antibody or antigen-binding fragment to the pediatric subject, wherein the pediatric subject is aged less than two years, but at least 32 weeks gestational age at birth, and weighs at least 2 kg but less than 4 kg; (ii) administering 125 mg of the antibody or antigen-binding fragment to the pediatric subject, wherein the pediatric subject is aged less than two years, but at least 32 weeks gestational age at birth, and weighs at least 4 kg; (iii) administering 187.5 mg of the antibody or antigen-binding fragment to the pediatric subject, wherein the pediatric subject is aged two years to less than six years; (iv) administering 250 mg of the antibody or antigen-binding fragment to the pediatric subject, wherein the pediatric subject is aged six years to less than twelve years; and/or (v) administering 375 mg of the antibody or antigen-binding fragment to the pediatric subject, wherein the pediatric subject is aged twelve years to less than eighteen years.

Embodiment 27: The method of Embodiment 26, further comprising: (i) administering 62.5 mg of the antibody or antigen-binding fragment in a volume of 1.0 mL, 0.83 mL, 0.63 mL, 0.5 mL, or 0.42 mL; (ii) administering 125 mg of the antibody or antigen-binding fragment in a volume of 2.0 mL, 1.67 mL, 1.25 mL, 1.0 mL, or 0.83 mL; (iii) administering 187.5 mg of the antibody or antigen-binding fragment in a volume of 3.0 mL, 2.5 mL 1.88 mL, 1.5 mL, or 1.25 mL; (iv) administering 250 mg of the antibody or antigen-binding fragment in a volume of 4.0 mL, 3.33 mL, 2.5 mL, 2.0 mL, or 1.66 mL; and/or (v) administering 375 mg of the antibody or antigen-binding in a volume of 6.0 mL, 5 mL, 3.75 mL, 3.0 mL, or 2.5 mL.

Embodiment 28: The method of any one of Embodiment 1-22, wherein the method comprises: (i) administering 100 mg of the antibody or antigen-binding fragment to the pediatric subject, wherein the pediatric subject is aged less than one year, but at least 32 weeks gestational age at birth; (ii) administering a range from 200 and 250 mg of the antibody or antigen-binding fragment to the pediatric subject, wherein the pediatric subject is aged at least one year to less than twelve years; and/or (iii) administering 500 mg of the antibody or antigen-binding fragment to the pediatric subject, wherein the pediatric subject is aged at least twelve years to less than eighteen years.

Embodiment 29: The method of Embodiment 28, further comprising: (i) administering 100 mg of the antibody or antigen-binding fragment in a volume of 1.6 ml, 1.33 mL, 1 mL, 0.8 mL, or 0.67 mL; (ii) administering a range from 200 and 250 mg of the antibody or antigen-binding fragment in a volume in a range from 3.2 mL to 4 mL, 2.67 mL to 3.33 mL, 2 mL to 2.5 mL, 1.6 mL to 2 mL, 1.33 mL to 1.67 mL; and/or (iii) administering 500 mg of the antibody or antigen-binding fragment in a volume of 8 mL, 6.67 mL, 5 mL, 4 mL, or 3.33 mL.

Embodiment 30: The method of any one of Embodiment 1-22, wherein the method comprises: (i) administering 100 mg of the antibody or antigen-binding fragment to the pediatric subject, wherein the pediatric subject is aged less than two years, but at least 32 weeks gestational age at birth; (ii) administering 150 mg of the antibody of antigen-binding fragment to the pediatric subject, wherein the pediatric subject is aged at least two years to less than six years; (iii) administering 225 mg of the antibody or antigen-binding fragment to the pediatric subject, wherein the pediatric subject is aged at least six years to less than twelve years; and/or (iv) administering 350 mg of the antibody or antigen-binding fragment to the pediatric subject, wherein the pediatric subject is aged at least twelve years to less than eighteen years.

Embodiment 31: The method of Embodiment 30, further comprising: (i) administering 100 mg of the antibody or antigen-binding fragment in a volume of 1.6 ml, 1.33 mL, 1 mL, 0.8 mL, or 0.67 mL; (ii) administering 150 mg of the antibody of antigen-binding fragment in a volume of 2.4 mL, 2.0 mL, 1.5 mL, 1.2 mL, or 1 mL; (iii) administering 225 mg of the antibody or antigen-binding fragment in a volume of 3.6 mL, 3 mL, 2.25 mL, 1.8 mL, or 1.5 mL; and/or (iv) administering 350 mg of the antibody or antigen-binding fragment in a volume of 5.6 mL, 4.67 mL, 3.5 mL, 2.8 mL, or 2.33 mL.

Embodiment 32: The method of any one of Embodiments 1-31, wherein the pediatric subject weighs 40 kg or less.

Embodiment 33: The method of any one of Embodiments 1-31, comprising administering 500 mg of the antibody or antigen-binding fragment to the pediatric subject, wherein the pediatric subject is aged at least twelve years and weights more than 40 kg.

Embodiment 34: The method of any one of Embodiments 1-33, comprising administering the antibody, antigen-binding fragment, or composition to the pediatric subject one time.

Embodiment 35. The method of any one of Embodiments 1-33, comprising administering the antibody, antigen-binding fragment, or composition to the pediatric subject a plurality of times, wherein a second or successive administration is performed at about 6, about 7, about 8, about 9, about 10, about 11, about 12, about 24, about 48, about 74, or about 96 hours following the first or preceding administration.

Embodiment 36. The method of any one of Embodiments 1-35, wherein the pediatric subject has a PCR or other nucleic acid amplification test-confirmed SARS-CoV-2 infection.

Embodiment 37. The method of any one of Embodiments 1-36, wherein the pediatric subject has mild-to-moderate COVID-19.

Embodiment 38. The method of Embodiment 37, wherein the pediatric subject has SpO₂ ≥94% in room air and one or more of: fever, chills, cough, sore throat, malaise, headache, joint or muscle pain, change in smell or taste, vomiting, diarrhea, shortness of breath, poor appetite or poor feeding, nasal congestion/runny nose, and lethargy, but does not have hypoxemia (02 saturation ≤93% on room air or PaO₂/FiO₂<300) requiring oxygen supplementation for more than 1 day, require ≥4 L/min oxygen supplementation or equivalent, respiratory failure requiring at least one of invasive mechanical ventilation or ECMO, shock, or multi-organ dysfunction/failure.

Embodiment 39: The method of Embodiment 37, wherein the pediatric subject is at-risk of progression to severe COVID-19.

Embodiment 40: The method of Embodiment 39, wherein severe COVID comprises (i) hypoxemia (O₂ saturation ≤93% on room air or PaO₂/FiO₂<300) requiring oxygen supplementation for more than 1 day or (ii) the pediatric subject requiring ≥4 L/min oxygen supplementation or equivalent.

Embodiment 41: The method of Embodiment 37, wherein following administration of the antibody, antigen-binding fragment, or composition to the pediatric subject, the pediatric subject is at a reduced risk of hospitalization for COVID-19.

Embodiment 42: The method of Embodiment 41, wherein following administration of the antibody, antigen-binding fragment, or composition to the pediatric subject, the pediatric subject's risk of hospitalization for COVID-19 is reduced by 10%, 20%, 30%, 40%, 50%, 60%, 70%, 80%, or 85%.

Embodiment 43: The method of any one of Embodiments 1-42, wherein the pediatric subject is less than seven days since onset of SARS-CoV-2 infection symptoms or COVID-19 symptoms.

Embodiment 44: The method of any one of Embodiments 1-43, wherein the pediatric subject had, or has close contacts to a person with a confirmed SARS-CoV-2 infection.

Embodiment 45: The method of any one of Embodiments 1-35 or 44, wherein treating comprises preventing infection by SARS-CoV-2 and/or preventing COVID-19.

Embodiment 46: The method of any one of Embodiments 1-44, wherein treating comprises preventing progression of COVID-19 in the pediatric subject.

Embodiment 47: The method of any one of Embodiments 1-46, wherein treating comprises preventing contraction and/or transmission of symptomatic COVID-19.

Embodiment 48: The method of any one of Embodiments 1-46, wherein treating comprises preventing contraction and/or transmission of asymptomatic COVID-19.

Embodiment 49: The method of any one of Embodiments 1-48, wherein the pediatric subject is at-risk for contracting or progressing in COVID-19.

Embodiment 50: The method of any one of Embodiments 1-49, wherein treating comprises preventing or reducing: (1) one or more acute respiratory symptom selected from: cough; sputum production; sore throat; and shortness of breath; or (2) fever of greater than 38° C.; (3) two or more of the following symptoms: fatigue; myalgias/arthralgias; chills; nausea/vomiting; diarrhea; and anosmia/dysgeusia.

Embodiment 51: The method of any one of Embodiments 1-50, wherein treating comprises preventing or reducing one or more of the following symptoms: fever of greater than 38° C.; chills, cough, sore throat, malaise, headache, joint or muscle pain, change in smell or taste, vomiting, diarrhea, shortness of breath, poor appetite or poor feeding, nasal congestion/runny nose, lethargy.

Embodiment 52: The method of any one of Embodiments 1-44 and 46-51, wherein the pediatric subject has had a positive SARS-CoV-2 test result, has oxygen saturation ≥94% on room air, has COVID-19 symptoms, and is less than or equal to 7 days from onset of symptoms.

Embodiment 53: The method of Embodiment 52, wherein the pediatric subject has one or more of: age less than one, diabetes mellitus, Pompe Disease, Mucopolysaccharidoses, glycogen storage diseases, fatty acid oxidation disorders, maple syrup urine disease, organic acidemias, obesity, congenital heart disease, hypertension, cardiomyopathy, heart failure, sickle cell disease, moderate to severe asthma, chronic lung disease, obstructive sleep apnea, Cystic Fibrosis, seizure disorder, global developmental delay, cerebral palsy, structural brain defect/malformation, primary immunodeficiency, HIV infection with CD4+ count <200 cells/mm3, solid organ or bone marrow transplant, long-term use of systemic corticosteroids, immunosuppressive biologic agents, or disease-modifying anti-rheumatic drugs, gastrostomy- or jejunostomy-dependence, parenteral nutrition dependence, tracheostomy-dependence, baseline oxygen requirement, use of CPAP/BiPAP/ventilator support, or other baseline medical complexity

Embodiment 54: The method of any one of Embodiments 1-53, wherein the antibody or antigen-binding fragment was obtained from a non-clonal pool of cells stably transfected with a polynucleotide encoding the antibody or antigen-binding fragment.

Embodiment 55: The method of any one of Embodiments 1-53, wherein the antibody or antigen-binding fragment was obtained from a clonal master cell bank.

Embodiment 56: The method of any one of Embodiments 1-55, wherein the pediatric subject has received a vaccine for SARS-CoV-2 and the vaccine is determined to be ineffective against the SARS-CoV-2 infection, e.g., by post-vaccine infection or symptoms in the pediatric subject, by clinical diagnosis or scientific or regulatory criteria or by infection with as SARS-CoV-2 variant against which the vaccine is less effective than variants for which the vaccine was originally tested or not effective.

Embodiment 57: The method of any one of Embodiments 1-55, wherein the pediatric subject has not received a vaccine for SARS-CoV-2.

Embodiment 58: The method of any one of Embodiments 1-57, wherein the pediatric subject s receiving or has received i) convalescent plasma therapy, ii) remdesivir, sofosbuvir, acyclovir, zidovudine, molnupiravir, nirmatrelvir, ritonavir, or favipiravir, or brequinar, dexamethasone, tocilizumab, etesevimab, lopinavir, leronlimab, bebtelovimab, casirivimab, imdevimab, or any combination thereof, iii) supplemental oxygen or respiration therapy, or two or more of i), ii), and iii) for SARS-CoV-2.

Embodiment 59: The method of any one of Embodiments 1-58, wherein treatment comprises pre-exposure or peri-exposure prophylaxis.

Embodiment 60: The method of any one of Embodiments 1-44 and 46-60, wherein treatment is administered to the pediatric subject having mild-to-moderate disease, optionally in an outpatient setting.

Embodiment 61: The method of any one of Embodiments 1-44 and 46-61, wherein the pediatric subject is further receiving or has received remdesivir, sofosbuvir, acyclovir, zidovudine, molnupiravir, nirmatrelvir, ritonavir, or favipiravir, or brequinar, dexamethasone, tocilizumab, etesevimab, lopinavir, leronlimab, bebtelovimab, casirivimab, imdevimab, or any combination thereof for SARS-CoV-2.

Embodiment 62. An anti-SARS-CoV-2 antibody or antigen-binding fragment, for use in the preparation of a medicament for treating a SARS-CoV-2 infection in a subject according to any one of Embodiments 1-61.

Embodiment 63. An anti-SARS-CoV-2 antibody or antigen-binding fragment, for use in the preparation of a medicament for treating a SARS-CoV-2 infection in a pediatric subject, wherein the medicament is a liquid composition comprising anti-SARS-CoV-2 antibody or antigen-binding fragment, and wherein the anti-SARS-CoV-2 antibody or antigen-binding fragment comprises (A)(i) a heavy chain variable domain (VH) that comprises: the complementarity determining region (CDR)H1 amino acid sequence set forth in SEQ ID NO.:106, the CDRH2 amino acid sequence set forth in SEQ ID NO.:121, and the CDRH3 amino acid sequence set forth in SEQ ID NO.:108; and (ii) a light chain variable domain (VL) that comprises: the CDRL1 amino acid sequence set forth in SEQ ID NO.:169, the CDRL2 amino acid sequence set forth in SEQ ID NO.:170, and the CDRL3 amino acid sequence set forth in SEQ ID NO.:171; (B)(i) a heavy chain variable domain (VH) that comprises the CDRH1, CDRH2, and CDRH3, of the amino acid sequence set forth in SEQ ID NO: 113 or 105, optionally as determined by IMGT; and (ii) and a light chain variable region that comprises the CDRL1, CDRL2, and CDRL3 of the amino acid sequence set forth in SEQ ID NO: 168, optionally as determined by IMGT; or (C)(i) a heavy chain variable domain (VH) that comprises or consists of the amino acid sequence set forth in SEQ ID NO: 113 or 105; and (ii) a light chain variable region that comprises or consists of the amino acid sequence set forth in SEQ ID NO: 168.

Embodiment 64. The use of Embodiment 63, wherein the medicament is formulated to administer a single dose of the anti-SARS-CoV-2 antibody or antigen-binding fragment.

Embodiment 65. The use of any one of Embodiments 62-64, wherein the liquid composition comprises at least 67.5 mg/mL anti-SARS-CoV-2 antibody or antigen-binding fragment.

Embodiment 66. A kit comprising: a liquid composition comprising an anti-SARS-CoV-2 antibody or antigen binding fragment comprising: (A)(i) a heavy chain variable domain (VH) that comprises: the complementarity determining region (CDR)H1 amino acid sequence set forth in SEQ ID NO.:106, the CDRH2 amino acid sequence set forth in SEQ ID NO.:121, and the CDRH3 amino acid sequence set forth in SEQ ID NO.:108; and (ii) a light chain variable domain (VL) that comprises: the CDRL1 amino acid sequence set forth in SEQ ID NO.:169, the CDRL2 amino acid sequence set forth in SEQ ID NO.:170, and the CDRL3 amino acid sequence set forth in SEQ ID NO.:171; (B)(i) a heavy chain variable domain (VH) that comprises the CDRH1, CDRH2, and CDRH3, of the amino acid sequence set forth in SEQ ID NO: 113 or 105, optionally as determined by IMGT; and (ii) and a light chain variable region that comprises the CDRL1, CDRL2, and CDRL3 of the amino acid sequence set forth in SEQ ID NO: 168, optionally as determined by IMGT; or (C)(i) a heavy chain variable domain (VH) that comprises or consists of the amino acid sequence set forth in SEQ ID NO: 113 or 105; and (ii) a light chain variable region that comprises or consists of the amino acid sequence set forth in SEQ ID NO: 168.

Embodiment 67. The kit of Embodiment 66, for use in any method of Embodiments 1-61, or for any use according to Embodiments 62-64.

Embodiment 68. The kit of Embodiment 65 or Embodiment 66, wherein the liquid composition comprises at least 67.5 mg/mL of the anti-SARS-CoV-2 antibody.

Embodiment 69. The kit of any one of Embodiments 66-68, wherein the liquid composition is formulated for administration by intramuscular injection.

TABLE 1
Sequences

| | SEQ | |
|---|---|---|
| Sequence | ID | |
| Description | NO. | Sequence |
| Reserved | 1-104 | |
| SARS-COV-2 | 105 | QVQLVQSGAEVKKPGASVKVSCKASGYPFTSYGISWVRQAPGQGL |
| S309-v1 mAb | | EWMGWISTYNGNTNYAQKFQGRVTMTTDTSTTTGYMELRRLRSD |
| VH (aa) | | DTAVYYCARDYTRGAWFGESLIGGFDNWGQGTLVTVSS |
| SARS-COV-2 | 106 | GYPFTSYG |
| S309-v1 | | |
| (Sotrovimab | | |
| and VIR- | | |
| 7832) mAb | | |
| CDRH1 (aa) | | |
| SARS-COV-2 | 107 | ISTYNGNT |
| S309-v1 mAb | | |
| CDRH2 (aa) | | |
| SARS-COV-2 | 108 | ARDYTRGAWFGESLIGGFDN |
| S309-v1 | | |
| (Sotrovimab | | |
| and VIR- | | |
| 7832 mAb | | |
| CDRH3 (aa) | | |
| Reserved | 109- | |
| | 112 | |
| SARS-COV-2 | 113 | QVQLVQSGAEVKKPGASVKVSCKASGYPFTSYGISWVRQAPGQGL |
| S309-v1.1 | | EWMGWISTYQGNTNYAQKFQGRVTMTTDTSTTTGYMELRRLRSD |
| mAb | | DTAVYYCARDYTRGAWFGESLIGGFDNWGQGTLVTVSS |
| (Sotrovimab | | |
| and VIR- | | |
| 7832)VH (aa) | | |
| Reserved | 114- | |
| | 118 | |
| SARS-COV-2 | 119 | QVQLVQSGAEVKKPGASVKVSCKASGYPFTSYGISWVRQAPGQGL |
| S309-v1.7 | | EWMGWISTYNGNTNYAQKFQGRVTMTTDTSTTTGYMELRRLRSD |
| mAb VH (aa) | | DTAVYYCARDYTRGAFFGESLIGGFDNWGQGTLVTVSS |
| SARS-COV-2 | 120 | QVQLVQSGAEVKKPGASVKVSCKASGYPFTSYGISWVRQAPGQGL |
| S309-v1.8 | | EWMGWISTYNGNTNYAQKFQGRVTMTTDTSTTTGYMELRRLRSD |
| mAb VH (aa) | | DTAVYYCARDYTRGAYFGESLIGGFDNWGQGTLVTVSS |
| SARS-COV-2 | 121 | ISTYQGNT |
| S309-v1.1 | | |
| mAb | | |
| (Sotrovimab | | |
| and VIR- | | |
| 7832) | | |
| CDRH2 (aa) | | |
| Reserved | 122- | |
| | 164 | |
| surface | 165 | mfvflvllpl vssqcvnltt rtqlppaytn sftrgvyypd kvfrssvlhs tqdlflpffs 61 |
| glycoprotein | | nvtwfhaihv sgtngtkrfd npvlpfndgv yfasteksni irgwifgttl dsktqslliv 121 |
| [Wuhan | | nnatnvvikv cefqfondpf lgvyyhknnk swmesefrvy ssannctfey vsqpflmdle 181 |
| seafood | | gkqgnfknlr efvfknidgy fkiyskhtpi nlvrdlpqgf saleplvdlp iginitrfqt 241 |
| market | | llalhrsylt pgdsssgwta gaaayyvgyl qprtfllkyn engtitdavd caldplsetk 301 |
| pneumonia | | ctlksftvek giyqtsnfrv qptesivrfp nitnlcpfge vfnatrfasv yawnrkrisn 361 |
| virus]; | | cvadysvlyn sasfstfkcy gvsptklndl cftnvyadsf virgdevrqi apgqtgkiad 421 |
| GenBank: | | ynyklpddft gcviawnsnn ldskvggnyn ylyrlfrksn lkpferdist eiyqagstpc 481 |
| QHD43416.1; | | ngvegfncyf plqsygfqpt ngvgyqpyrv vvlsfellha patvcgpkks tnlvknkcvn 541 |
| Jan 23, | | fnfngltgtg vltesnkkfl pfqqfgrdia dttdavrdpq tleilditpc sfggvsvitp 601 |
| 2020 | | gtntsnqvav lyqdvnctev pvaihadqlt ptwrvystgs nvfqtragcl igaehvnnsy 661 |
| | | ecdipigagi casyqtqtns prrarsvasq siiaytmslg aensvaysnn siaiptnfti 721 |
| | | svtteilpvs mtktsvdctm yicgdstecs nlllqygsfc tqlnraltgi aveqdkntqe 781 |
| | | vfaqvkqiyk tppikdfggf nfsqilpdps kpskrsfied llfnkvtlad agfikqygdc 841 |
| | | lgdiaardli caqkfngltv lpplltdemi aqytsallag titsgwtfga gaalqipfam 901 |
| | | qmayrfngig vtqnvlyenq klianqfnsa igkiqdslss tasalgklqd vvnqnaqaln 961 |
| | | tlvkqlssnf gaissvlndi lsrldkveae vqidrlitgr lqslqtyvtq qliraaeira 1021 |
| | | sanlaatkms ecvlgqskrv dfcgkgyhlm sfpqsaphgv vflhvtyvpa qeknfttapa 1081 |
| | | ichdgkahfp regvfvsngt hwfvtqrnfy epqiittdnt fvsgncdvvi givnntvydp 1141 |
| | | lqpeldsfke eldkyfknht spdvdlgdis ginasvvniq keidrlneva knlneslidl 1201 |
| | | qelgkyegyi kwpwyiwlgf iagliaivmv timlccmtsc csclkgccsc gscckfdedd 1261 |
| | | sepvlkgvkl hyt |
| surface | 166 | nitnlcpfgevfnatrfasvyawnrkrisncvadysvlynsasfstfkcygvsptklndlcftnvyadsfvi |
| glycoprotein | | rgdevrqiapgqtgkiadynyklpddftgcviawnsnnldskvggnynylyrlfrksnlkpferdistei |
| RBD [Wuhan | | yqagstpcngvegfncyfplqsygfqptngvgyqpyrvvvlsfellhapatvcgpkkstnlvknkcvn |
| seafood | | fnfngltgtg |
| market | | |
| pneumonia | | |
| virus]; | | |
| GenBank: | | |
| QHD43416.1; | | |
| Jan. 23, | | |
| 2020 | | |
| Receptor | 167 | Nsnnldskvggnynylyrlfrksnlkpferdisteiyqagstpcngvegfncyfplqsygfqptngvgy |
| Binding | | qpy |
| Motif (RBM) | | |
| in surface | | |
| glycoprotein | | |
| RBD [Wuhan | | |
| seafood | | |
| market | | |
| pneumonia | | |
| virus]; | | |
| GenBank: | | |
| QHD43416.1; | | |
| Jan. 23, | | |
| 2020 | | |
| SARS-COV-2 | 168 | EIVLTQSPGTLSLSPGERATLSCRASQTVSSTSLAWYQQKPGQAPRL |
| S309-v13 | | LIYGASSRATGIPDRFSGSGSGTDFTLTISRLEPEDFAVYYCQQHDTS |
| (Sotrovimab | | LTFGGGTKVEIK |
| and VIR- | | |
| 7832) mAb | | |
| VL (VK) (aa) | | |
| SARS-COV-2 | 169 | QTVSSTS |
| S309-v13 | | |
| (Sotrovimab | | |
| and VIR- | | |
| 7832) mAb | | |
| CDRL1 (aa) | | |
| SARS-COV-2 | 170 | GAS |
| S309-v13 | | |
| (Sotrovimab | | |
| and VIR- | | |
| 7832) mAb | | |
| CDRL2 (aa) | | |
| SARS-COV-2 | 171 | QQHDTSLT |
| S309-v13 | | |
| (Sotrovimab | | |
| and VIR- | | |
| 7832) mAb | | |
| CDRL3 (aa) | | |
| Reserved | 172 | |
| SARS-COV-2 | 173 | ASTKGPSVFPLAPSSKSTSGGTAALGCLVKDYFPEPVTVSWNSGAL |
| CH1-CH3 | | TSGVHTFPAVLQSSGLYSLSSVVTVPSSSLGTQTYICNVNHKPSNTK |
| G1m17; | | VDKKVEPKSCDKTHTCPPCPAPELLGGPSVFLFPPKPKDTLMISRTP |
| IgG1*01 LS | | EVTCVVVDVSHEDPEVKFNWYVDGVEVHNAKTKPREEQYNSTYR |
| (aa) (present | | VVSVLTVLHQDWLNGKEYKCKVSNKALPAPIEKTISKAKGQPREPQ |
| in | | VYTLPPSRDELTKNQVSLTCLVKGFYPSDIAVEWESNGQPENNYKT |
| Sotrovimab) | | TPPVLDSDGSFFLYSKLTVDKSRWQQGNVFSCSVLHEALHSHYTQK |
| | | SLSLSPGK |
| SARS-COV-2 | 174 | RTVAAPSVFIFPPSDEQLKSGTASVVCLLNNFYPREAKVQWKVDN |
| mAb CL (Ck) | | ALQSGNSQESVTEQDSKDSTYSLSSTLTLSKADYEKHKVYACEVT |
| IgKC*01 | | HQGLSSPVTKSFNRGEC |
| klm3 (aa) | | |
| (present in | | |
| Sotrovimab | | |
| and VIR- | | |
| 7832) | | |
| SARS-COV-2 | 175 | ASTKGPSVFPLAPSSKSTSGGTAALGCLVKDYFPEPVTVSWNSGAL |
| CH1-CH3 | | TSGVHTFPAVLQSSGLYSLSSVVTVPSSSLGTQTYICNVNHKPSNTK |
| G1m17; | | VDKKVEPKSCDKTHTCPPCPAPELLAGPSVFLFPPKPKDTLMISRTP |
| IgG1*01 LS | | EVTCVVVDVSHEDPEVKFNWYVDGVEVHNAKTKPREEQYNSTYR |
| GAALIE (aa) | | VVSVLTVLHQDWLNGKEYKCKVSNKALPLPEEKTISKAKGQPREP |
| (present in | | QVYTLPPSRDELTKNQVSLTCLVKGFYPSDIAVEWESNGQPENNYK |
| VIR-7832) | | TTPPVLDSDGSFFLYSKLTVDKSRWQQGNVFSCSVLHEALHSHYTQ |
| | | KSLSLSPGK |
| Reserved | 176- | |
| | 192 | |
| SARS-COV-2 | 193 | GQPKAAPSVTLFPPSSEELQANKATLVCLISDFYPGAVTVAWKADS |
| mAb CL | | SPVKAGVETTTPSKQSNNKYAASSYLSLTPEQWKSHRSYSCQVTHE |
| IgLC*01 | | GSTVEKTVAPTECS |
| Reserved | 194- | |
| | 205 | |
| Linker (aa) | 206 | GSTSGSGKPGSGEGSTKG |
| Linker (aa) | 207 | GSGKPGSGEG |
| Linker (aa) | 208 | GKPGSGEG |
| Linker (aa) | 209 | SGKPGSGE |
| Linker (aa) | 210 | BPXXXZ, wherein each X is independently a glycine (G) |
| | | or serine (S), B is a positively charged amino acid and |
| | | Z is glycine (G) or a negatively charged amino acid |
| Linker (aa) | 211 | (GxS)y, wherein x is 1-10 and y is 1-10 |
| Linker (aa) | 212 | GGGGSGGGGSGGGGS |
| Linker (aa) | 213 | GGGGSGGGGSGGGGSGGGGSGGGGS |
| | | GGGGSGGGGSGGGGSGGGGSGGGGS |
| Linker (aa) | 214 | GSTSGGGSGGGSGGGGSS |
| Linker (aa) | 215 | EGKSSGSGSESKVD |
| Linker (aa) | 216 | KESGSVSSEQLAQFRSLD |
| Linker (aa) | 217 | GGGGS |
| SARS-COV-2 | 218 | QVQLVQSGAEVKKPGASVKVSCKASGYPFTSYGISWVRQAPGQGL |
| S309-scFab | | EWMGWISTYNGNTNYAQKFQGRVTMTTDTSTTTGYMELRRLRSD |
| (H-L) (aa) | | DTAVYYCARDYTRGAWFGESLIGGFDNWGQGTLVTVSSASTKGP |
| | | SVFPLAPSSKSTSGGTAALGCLVKDYFPEPVTVSWNSGALTSGVHT |
| | | FPAVLQSSGLYSLSSVVTVPSSSLGTQTYICNVNHKPSNTKVDKRVE |
| | | PKSCGGGGSGGGGSGGGGSGGGGSGGGGSGGGGSGGGGSGGGGS |
| | | GGGGSGGGGSEIVLTQSPGTLSLSPGERATLSCRASQTVSSTSLAW |
| | | YQQKPGQAPRLLIYGASSRATGIPDRFSGSGSGTDFTLTISRLEPEDF |
| | | AVYYCQQHDTSLTFGGGTKVEIKRTVAAPSVFIFPPSDEQLKSGTA |
| | | SVVCLLNNFYPREAKVQWKVDNALQSGNSQESVTEQDSKDSTYSL |
| | | SSTLTLSKADYEKHKVYACEVTHQGLSSPVTKSFNRGEC |
| SARS-COV-2 | 219 | EIVLTQSPGTLSLSPGERATLSCRASQTVSSTSLAWYQQKPGQAPRL |
| S309-scFab | | LIYGASSRATGIPDRFSGSGSGTDFTLTISRLEPEDFAVYYCQQHDT |
| (L-H) (aa) | | SLTFGGGTKVEIKRTVAAPSVFIFPPSDEQLKSGTASVVCLLNNFYP |
| | | REAKVQWKVDNALQSGNSQESVTEQDSKDSTYSLSSTLTLSKADY |
| | | EKHKVYACEVTHQGLSSPVTKSFNRGECGGGGSGGGGSGGGGSGG |
| | | GGSGGGGSGGGGSGGGGSGGGGSGGGGSGGGGSQVQLVQSGAEV |
| | | KKPGASVKVSCKASGYPFTSYGISWVRQAPGQGLEWMGWISTYN |
| | | GNTNYAQKFQGRVTMTTDTSTTTGYMELRRLRSDDTAVYYCARD |
| | | YTRGAWFGESLIGGFDNWGQGTLVTVSSASTKGPSVFPLAPSSKS |
| | | TSGGTAALGCLVKDYFPEPVTVSWNSGALTSGVHTFPAVLQSSGLY |
| | | SLSSVVTVPSSSLGTQTYICNVNHKPSNTKVDKRVEPKSC |
| SARS-COV-2 | 220 | QVQLVQSGAEVKKPGASVKVSCKASGYPFTSYGISWVRQAPGQGL |
| S309-scFv | | EWMGWISTYNGNTNYAQKFQGRVTMTTDTSTTTGYMELRRLRSD |
| (VH-VL) (aa) | | DTAVYYCARDYTRGAWFGESLIGGFDNWGQGTLVTVSSGGGGS |
| | | GGGGSGGGGSEIVLTQSPGTLSLSPGERATLSCRASQTVSSTSLAW |
| | | YQQKPGQAPRLLIYGASSRATGIPDRFSGSGSGTDFTLTISRLEPEDF |
| | | AVYYCQQHDTSLTFGGGTKVEIK |
| SARS-COV-2 | 221 | EIVLTQSPGTLSLSPGERATLSCRASQTVSSTSLAWYQQKPGQAPRL |
| S309-scFv | | LIYGASSRATGIPDRFSGSGSGTDFTLTISRLEPEDFAVYYCQQHDT |
| (VL-VH) (aa) | | SLTFGGGTKVEIKGGGGSGGGGSGGGGSQVQLVQSGAEVKKPGAS |
| | | VKVSCKASGYPFTSYGISWVRQAPGQGLEWMGWISTYNGNTNYA |
| | | QKFQGRVTMTTDTSTTTGYMELRRLRSDDTAVYYCARDYTRGAW |
| | | FGESLIGGFDNWGQGTLVTVSS |
| SARS-COV-2 | 222 | QVQLVQSGAEVKKPGASVKVSCKASGYPFTSYGISWVRQAPGQGL |
| S309-scFv | | EWMGWISTYNGNTNYAQKFQGRVTMTTDTSTTTGYMELRRLRSD |
| (VH-VL)- | | DTAVYYCARDYTRGAWFGESLIGGFDNWGQGTLVTVSSGGGGS |
| (VH-VL) (aa) | | GGGGSGGGGSEIVLTQSPGTLSLSPGERATLSCRASQTVSSTSLAW |
| | | YQQKPGQAPRLLIYGASSRATGIPDRFSGSGSGTDFTLTISRLEPEDF |
| | | AVYYCQQHDTSLTFGGGTKVEIKGGGGSGGGGSGGGGSGGGGSQ |
| | | VQLVQSGAEVKKPGASVKVSCKASGYPFTSYGISWVRQAPGQGLE |
| | | WMGWISTYNGNTNYAQKFQGRVTMTTDTSTTTGYMELRRLRSDD |
| | | TAVYYCARDYTRGAWFGESLIGGFDNWGQGTLVTVSSGGGGSG |
| | | GGGSGGGGSEIVLTQSPGTLSLSPGERATLSCRASQTVSSTSLAWY |
| | | QQKPGQAPRLLIYGASSRATGIPDRFSGSGSGTDFTLTISRLEPEDFA |
| | | VYYCQQHDTSLTFGGGTKVEIK |
| SARS-COV-2 | 223 | QVQLVQSGAEVKKPGASVKVSCKASGYPFTSYGISWVRQAPGQGL |
| S309-scFv- | | EWMGWISTYNGNTNYAQKFQGRVTMTTDTSTTTGYMELRRLRSD |
| (VH-VL)- | | DTAVYYCARDYTRGAWFGESLIGGFDNWGQGTLVTVSSGGGGS |
| (VL-VH) (aa) | | GGGGSGGGGSEIVLTQSPGTLSLSPGERATLSCRASQTVSSTSLAW |
| | | YQQKPGQAPRLLIYGASSRATGIPDRFSGSGSGTDFTLTISRLEPEDF |
| | | AVYYCQQHDTSLTFGGGTKVEIKGGGGSGGGGSGGGGSGGGGSEI |
| | | VLTQSPGTLSLSPGERATLSCRASQTVSSTSLAWYQQKPGQAPRLLI |
| | | YGASSRATGIPDRFSGSGSGTDFTLTISRLEPEDFAVYYCQQHDTSL |
| | | TFGGGTKVEIKGGGGSGGGGSGGGGSQVQLVQSGAEVKKPGASV |
| | | KVSCKASGYPFTSYGISWVRQAPGQGLEWMGWISTYNGNTNYAQ |
| | | KFQGRVTMTTDTSTTTGYMELRRLRSDDTAVYYCARDYTRGAWF |
| | | GESLIGGFDNWGQGTLVTVSS |
| SARS-COV-2 | 224 | EIVLTQSPGTLSLSPGERATLSCRASQTVSSTSLAWYQQKPGQAPRL |
| S309-scFv- | | LIYGASSRATGIPDRFSGSGSGTDFTLTISRLEPEDFAVYYCQQHDT |
| (VL-VH)- | | SLTFGGGTKVEIKGGGGSGGGGSGGGGSQVQLVQSGAEVKKPGAS |
| (VH-VL) (aa) | | VKVSCKASGYPFTSYGISWVRQAPGQGLEWMGWISTYNGNTNYA |
| | | QKFQGRVTMTTDTSTTTGYMELRRLRSDDTAVYYCARDYTRGAW |
| | | FGESLIGGFDNWGQGTLVTVSSGGGGSGGGGSGGGGSGGGGSQV |
| | | QLVQSGAEVKKPGASVKVSCKASGYPFTSYGISWVRQAPGQGLE |
| | | WMGWISTYNGNTNYAQKFQGRVTMTTDTSTTTGYMELRRLRSDD |
| | | TAVYYCARDYTRGAWFGESLIGGFDNWGQGTLVTVSSGGGGSG |
| | | GGGSGGGGSEIVLTQSPGTLSLSPGERATLSCRASQTVSSTSLAWY |
| | | QQKPGQAPRLLIYGASSRATGIPDRFSGSGSGTDFTLTISRLEPEDFA |
| | | VYYCQQHDTSLTFGGGTKVEIK |
| SARS-COV-2 | 225 | EIVLTQSPGTLSLSPGERATLSCRASQTVSSTSLAWYQQKPGQAPRL |
| S309-scFv- | | LIYGASSRATGIPDRFSGSGSGTDFTLTISRLEPEDFAVYYCQQHDT |
| (VL-VH)- | | SLTFGGGTKVEIKGGGGSGGGGSGGGGSQVQLVQSGAEVKKPGAS |
| (VL-VH) (aa) | | VKVSCKASGYPFTSYGISWVRQAPGQGLEWMGWISTYNGNTNYA |
| | | QKFQGRVTMTTDTSTTTGYMELRRLRSDDTAVYYCARDYTRGAW |
| | | FGESLIGGFDNWGQGTLVTVSSGGGGSGGGGSGGGGSGGGGSEIV |
| | | LTQSPGTLSLSPGERATLSCRASQTVSSTSLAWYQQKPGQAPRLLIY |
| | | GASSRATGIPDRFSGSGSGTDFTLTISRLEPEDFAVYYCQQHDTSLT |
| | | FGGGTKVEIKGGGGSGGGGSGGGGSQVQLVQSGAEVKKPGASVK |
| | | VSCKASGYPFTSYGISWVRQAPGQGLEWMGWISTYNGNTNYAQK |
| | | FQGRVTMTTDTSTTTGYMELRRLRSDDTAVYYCARDYTRGAWFG |
| | | ESLIGGFDNWGQGTLVTVSS |
| SARS-COV-2 | 226 | QVQLVQSGAEVKKPGASVKVSCKASGYPFTSYGISWVRQAPGQGL |
| S309-scFab- | | EWMGWISTYQGNTNYAQKFQGRVTMTTDTSTTTGYMELRRLRSD |
| (H-L) v1.1 | | DTAVYYCARDYTRGAWFGESLIGGFDNWGQGTLVTVSSASTKGP |
| (aa) | | SVFPLAPSSKSTSGGTAALGCLVKDYFPEPVTVSWNSGALTSGVHT |
| | | FPAVLQSSGLYSLSSVVTVPSSSLGTQTYICNVNHKPSNTKVDKRVE |
| | | PKSCGGGGSGGGGSGGGGSGGGGSGGGGSGGGGSGGGGSGGGGS |
| | | GGGGSGGGGSEIVLTQSPGTLSLSPGERATLSCRASQTVSSTSLAW |
| | | YQQKPGQAPRLLIYGASSRATGIPDRFSGSGSGTDFTLTISRLEPEDF |
| | | AVYYCQQHDTSLTFGGGTKVEIKRTVAAPSVFIFPPSDEQLKSGTA |
| | | SVVCLLNNFYPREAKVQWKVDNALQSGNSQESVTEQDSKDSTYSL |
| | | SSTLTLSKADYEKHKVYACEVTHQGLSSPVTKSFNRGEC |
| SARS-COV-2 | 227 | EIVLTQSPGTLSLSPGERATLSCRASQTVSSTSLAWYQQKPGQAPRL |
| S309-scFab- | | LIYGASSRATGIPDRFSGSGSGTDFTLTISRLEPEDFAVYYCQQHDT |
| (L-H) v1.1 | | SLTFGGGTKVEIKRTVAAPSVFIFPPSDEQLKSGTASVVCLLNNFYP |
| (aa) | | REAKVQWKVDNALQSGNSQESVTEQDSKDSTYSLSSTLTLSKADY |
| | | EKHKVYACEVTHQGLSSPVTKSFNRGECGGGGSGGGGSGGGGSGG |
| | | GGSGGGGSGGGGSGGGGSGGGGSGGGGSGGGGSQVQLVQSGAEV |
| | | KKPGASVKVSCKASGYPFTSYGISWVRQAPGQGLEWMGWISTYQ |
| | | GNTNYAQKFQGRVTMTTDTSTTTGYMELRRLRSDDTAVYYCARD |
| | | YTRGAWFGESLIGGFDNWGQGTLVTVSSASTKGPSVFPLAPSSKS |
| | | TSGGTAALGCLVKDYFPEPVTVSWNSGALTSGVHTFPAVLQSSGLY |
| | | SLSSVVTVPSSSLGTQTYICNVNHKPSNTKVDKRVEPKSC |
| SARS-COV-2 | 228 | QVQLVQSGAEVKKPGASVKVSCKASGYPFTSYGISWVRQAPGQGL |
| S309-scFv- | | EWMGWISTYQGNTNYAQKFQGRVTMTTDTSTTTGYMELRRLRSD |
| (VH-VL) | | DTAVYYCARDYTRGAWFGESLIGGFDNWGQGTLVTVSSGGGGS |
| v1.1 (aa) | | GGGGSGGGGSEIVLTQSPGTLSLSPGERATLSCRASQTVSSTSLAW |
| | | YQQKPGQAPRLLIYGASSRATGIPDRFSGSGSGTDFTLTISRLEPEDF |
| | | AVYYCQQHDTSLTFGGGTKVEIK |
| SARS-COV-2 | 229 | EIVLTQSPGTLSLSPGERATLSCRASQTVSSTSLAWYQQKPGQAPRL |
| S309-scFv- | | LIYGASSRATGIPDRFSGSGSGTDFTLTISRLEPEDFAVYYCQQHDT |
| (VL-VH) | | SLTFGGGTKVEIKGGGGSGGGGGGGGSQVQLVQSGAEVKKPGAS |
| v1.1 (aa) | | VKVSCKASGYPFTSYGISWVRQAPGQGLEWMGWISTYQGNTNYA |
| | | QKFQGRVTMTTDTSTTTGYMELRRLRSDDTAVYYCARDYTRGAW |
| | | FGESLIGGFDNWGQGTLVTVSS |
| SARS-COV-2 | 230 | QVQLVQSGAEVKKPGASVKVSCKASGYPFTSYGISWVRQAPGQGL |
| S309-scFv- | | EWMGWISTYQGNTNYAQKFQGRVTMTTDTSTTTGYMELRRLRSD |
| (VH-VL)- | | DTAVYYCARDYTRGAWFGESLIGGFDNWGQGTLVTVSSGGGGS |
| (VH-VL) | | GGGGSGGGGSEIVLTQSPGTLSLSPGERATLSCRASQTVSSTSLAW |
| v1.1 (aa) | | YQQKPGQAPRLLIYGASSRATGIPDRFSGSGSGTDFTLTISRLEPEDF |
| | | AVYYCQQHDTSLTFGGGTKVEIKGGGGSGGGGSGGGGSGGGGSQ |
| | | VQLVQSGAEVKKPGASVKVSCKASGYPFTSYGISWVRQAPGQGLE |
| | | WMGWISTYQGNTNYAQKFQGRVTMTTDTSTTTGYMELRRLRSDD |
| | | TAVYYCARDYTRGAWFGESLIGGFDNWGQGTLVTVSSGGGGSG |
| | | GGGSGGGGSEIVLTQSPGTLSLSPGERATLSCRASQTVSSTSLAWY |
| | | QQKPGQAPRLLIYGASSRATGIPDRFSGSGSGTDFTLTISRLEPEDFA |
| | | VYYCQQHDTSLTFGGGTKVEIK |
| SARS-COV-2 | 231 | QVQLVQSGAEVKKPGASVKVSCKASGYPFTSYGISWVRQAPGQGL |
| S309-scFv- | | EWMGWISTYQGNTNYAQKFQGRVTMTTDTSTTTGYMELRRLRSD |
| (VH-VL)- | | DTAVYYCARDYTRGAWFGESLIGGFDNWGQGTLVTVSSGGGGS |
| (VL-VH) | | GGGGSGGGGSEIVLTQSPGTLSLSPGERATLSCRASQTVSSTSLAW |
| v1.1 (aa) | | YQQKPGQAPRLLIYGASSRATGIPDRFSGSGSGTDFTLTISRLEPEDF |
| | | AVYYCQQHDTSLTFGGGTKVEIKGGGGSGGGGSGGGGSGGGGSEI |
| | | VLTQSPGTLSLSPGERATLSCRASQTVSSTSLAWYQQKPGQAPRLLI |
| | | YGASSRATGIPDRFSGSGSGTDFTLTISRLEPEDFAVYYCQQHDTSL |
| | | TFGGGTKVEIKGGGGSGGGGSGGGGSQVQLVQSGAEVKKPGASV |
| | | KVSCKASGYPFTSYGISWVRQAPGQGLEWMGWISTYQGNTNYAQ |
| | | KFQGRVTMTTDTSTTTGYMELRRLRSDDTAVYYCARDYTRGAWF |
| | | GESLIGGFDNWGQGTLVTVSS |
| SARS-COV-2 | 232 | EIVLTQSPGTLSLSPGERATLSCRASQTVSSTSLAWYQQKPGQAPRL |
| S309-scFv- | | LIYGASSRATGIPDRFSGSGSGTDFTLTISRLEPEDFAVYYCQQHDT |
| (VL-VH)- | | SLTFGGGTKVEIKGGGGSGGGGSGGGGSQVQLVQSGAEVKKPGAS |
| (VH-VL) | | VKVSCKASGYPFTSYGISWVRQAPGQGLEWMGWISTYQGNTNYA |
| v1.1 (aa) | | QKFQGRVTMTTDTSTTTGYMELRRLRSDDTAVYYCARDYTRGAW |
| | | FGESLIGGFDNWGQGTLVTVSSGGGGSGGGGSGGGGSGGGGSQV |
| | | QLVQSGAEVKKPGASVKVSCKASGYPFTSYGISWVRQAPGQGLE |
| | | WMGWISTYQGNTNYAQKFQGRVTMTTDTSTTTGYMELRRLRSDD |
| | | TAVYYCARDYTRGAWFGESLIGGFDNWGQGTLVTVSSGGGGSG |
| | | GGGSGGGGSEIVLTQSPGTLSLSPGERATLSCRASQTVSSTSLAWY |
| | | QQKPGQAPRLLIYGASSRATGIPDRFSGSGSGTDFTLTISRLEPEDFA |
| | | VYYCQQHDTSLTFGGGTKVEIK |
| SARS-COV-2 | 233 | EIVLTQSPGTLSLSPGERATLSCRASQTVSSTSLAWYQQKPGQAPRL |
| S309-scFv- | | LIYGASSRATGIPDRFSGSGSGTDFTLTISRLEPEDFAVYYCQQHDT |
| (VL-VH)- | | SLTFGGGTKVEIKGGGGSGGGGSGGGGSQVQLVQSGAEVKKPGAS |
| (VL-VH) | | VKVSCKASGYPFTSYGISWVRQAPGQGLEWMGWISTYQGNTNYA |
| v1.1 (aa) | | QKFQGRVTMTTDTSTTTGYMELRRLRSDDTAVYYCARDYTRGAW |
| | | FGESLIGGFDNWGQGTLVTVSSGGGGSGGGGSGGGGSGGGGSEIV |
| | | LTQSPGTLSLSPGERATLSCRASQTVSSTSLAWYQQKPGQAPRLLIY |
| | | GASSRATGIPDRFSGSGSGTDFTLTISRLEPEDFAVYYCQQHDTSLT |
| | | FGGGTKVEIKGGGGSGGGGSGGGGSQVQLVQSGAEVKKPGASVK |
| | | VSCKASGYPFTSYGISWVRQAPGQGLEWMGWISTYQGNTNYAQK |
| | | FQGRVTMTTDTSTTTGYMELRRLRSDDTAVYYCARDYTRGAWFG |
| | | ESLIGGFDNWGQGTLVTVSS |
| Reserved | 234- | |
| | 250 | |
| CMV | 251 | GACATTGATTATTGACTAGTTATTAATAGTAATCAATTACGGGGT |
| promoter (nt) | | CATTAGTTCATAGCCCATATATGGAGTTCCGCGTTACATAACTTA |
| | | CGGTAAATGGCCCGCCTGGCTGACCGCCCAACGACCCCCGCCCA |
| | | TGACGTCAATAATGACGTATGTTCCCATAGTAACGCCAATAGGG |
| | | ACTTTCCATTGACGTCAATGGGTGGAGTATTTACGGTAAACTGC |
| | | CCACTTGGCAGTACATCAAGTGTATCATATGCCAAGTACGCCCC |
| | | CTATTGACGTCAATGACGGTAAATGGCCCGCCTGGCATTATGCC |
| | | CAGTACATGACCTTATGGGACTTTCCTACTTGGCAGTACATCTAC |
| | | GTATTAGTCATCGCTATTACCATGGTGATGCGGTTTTGGCAGTAC |
| | | ATCAATGGGCGTGGATAGCGGTTTGACTCACGGGGATTTCCAAG |
| | | TCTCCACCCCATTGACGTCAATGGGAGTTTGTTTTGGCACCAAAA |
| | | TCAACGGGACTTTCCAAAATGTCGTAACAACTCCGCCCCATTGA |
| | | CGCAAATGGGCGGTAGGCGTGTACGGTGGGAGGTCTATATAAGC |
| | | AGAGCTCGTTTAGTGAACCGTCAGATCGCCTGGAGACGCCATCC |
| | | ACGCTGTTTTGACCTCCATAGAAGACACCGGGACCGATCCAGCC |
| | | TCCGCGGCCGGGAACGGTGCATTGGAACGCGGATTCCCCGTGCC |
| | | AAGAGTGACGTAAGTACCGCCTATAGAGTCTATAGGCCCACCCC |
| | | CTTGGCTTCGTTAG |
| Signal | 252 | ATGGGATGGTCATGTATCATCCTTTTTCTAGTAGCAACTGCAACC |
| peptide (nt) | | GGTGT |
| Poly- | 253 | AACTTGTTTATTGCAGCTTATAATGGTTACAAATAAAGCAATAG |
| adenylation | | CATCACAAATTTCACAAATAAAGCATTTTTTTCACTGCATTCTAG |
| signal | | TTGTGGTTTGTCCAAACTCATCAATGTATCTTATCATGTCTGGAT |
| sequence (nt) | | C |
| Reserved | 254- | |
| | 255 | |
| Signal | 256 | MGWSCIILFLVATATG |
| peptide (aa) | | |
| Reserved | 257- | |
| | 262 | |
| Signal | 263 | atgggctggtcctgcatcatcctgttcctggtggccacagccaccggcgtgcacagc |
| peptide (nt- | | |
| CO) | | |
| Signal | 264 | MGWSCIILFLVATATGVHS |
| peptide (aa) | | |
| SARS-COV-2 | 265 | ASTKGPSVFPLAPSSKSTSGGTAALGCLVKDYFPEPVTVSWNSGAL |
| CH1—CH3 | | TSGVHTFPAVLQSSGLYSLSSVVTVPSSSLGTQTYICNVNHKPSNTK |
| G1m17; | | VDKKVEPKSCDKTHTCPPCPAPELLGGPSVFLFPPKPKDTLMISRTP |
| IgG1*01 LS | | EVTCVVVDVSHEDPEVKFNWYVDGVEVHNAKTKPREEQYNSTYR |
| no C-term | | VVSVLTVLHQDWLNGKEYKCKVSNKALPAPIEKTISKAKGQPREPQ |
| Lys (aa) (may | | VYTLPPSRDELTKNQVSLTCLVKGFYPSDIA VEWESNGQPENNYKT |
| be used in | | TPPVLDSDGSFFLYSKLTVDKSRWQQGNVFSCSVLHEALHSHYTQK |
| Sotrovimab | | SLSLSPG |
| or VIR-7832) | | |
| SARS-COV-2 | 266 | ASTKGPSVFPLAPSSKSTSGGTAALGCLVKDYFPEPVTVSWNSGAL |
| CH1—CH3 | | TSGVHTFPAVLQSSGLYSLSSVVTVPSSSLGTQTYICNVNHKPSNTK |
| G1m17; | | VDKKVEPKSCDKTHTCPPCPAPELLAGPSVFLFPPKPKDTLMISRTP |
| IgG1*01 LS | | EVTCVVVDVSHEDPEVKFNWYVDGVEVHNAKTKPREEQYNSTYR |
| GAALIE no | | VVSVLTVLHQDWLNGKEYKCKVSNKALPLPEEKTISKAKGQPREP |
| C-term Lys | | QVYTLPPSRDELTKNQVSLTCLVKGFYPSDIAVEWESNGQPENNYK |
| (aa) (may be | | TTPPVLDSDGSFFLYSKLTVDKSRWQQGNVFSCSVLHEALHSHYTQ |
| used in | | KSLSLSPG |
| Sotrovimab | | |
| or VIR-7832) | | |

### EXAMPLES

### Example 1

### Human Monoclonal Antibodies that Bind Spike Protein of Sars-Cov-2

B cells from a donor with previous SARS-CoV infection were sorted and immortalized with EBV and screened in 384-well plates, as described in European patent EP1597280B1.

Two weeks after immortalization, supernatants of immortalized B cells were tested for antibodies binding to SARS-CoV-2 Spike protein using a flow cytometry-based method. ExpiCHO™ (Thermo Fisher Scientific) cells were transfected with S protein of SARS-Cov-2 (strain BetaCoV/Wuhan-Hu-1/2019), or with an empty plasmid as a negative control. Fourteen monoclonal antibodies were identified that bind SARS-CoV-2 S, and were termed SARS-CoV-2 S300 through SARS-CoV-2 S312 and SARS-CoV-2 S315.

### Example 2

### Binding of Human Monoclonal Antibodies to RBD of SARS-COV-2 Using Octet

Strepavidin biosensors (Pall ForteBio) were used to immobilize anti-Strep Tag II antibody at 3 ug/ml (clone 5A9F9, Biotin, LabForce AG, Muttenz CH), after a hydration step for 10 min with Kinetics Buffer (KB; 0.01% endotoxin-free BSA, 0.002% Tween-20, 0.005% NaN3 in PBS). SARS-CoV-2 RBD with a Strep Tag II (produced in-house) was then loaded for 6 min at a concentration of 4 μg/ml in KB. Antibodies from B cell supernatant were allowed to associate for a period of time. To observe dissociation, sensors were moved from the antibody solution into KB and antibody dissociation was monitored.

The S309 mAb comprises the S309-v1 VH and S309-v13 VL amino acid sequences provided in Table 1 (SEQ ID Nos.: 105 and 168, respectively). Comparison of the binding curves for S303 and S309 indicates that S303 has both a faster on-rate and a faster off-rate than S309, suggesting that S309 may bind with higher affinity.

### Example 3

### Binding of Human Monoclonal Antibodies to RBD of SARS-COV-2 and SARS-COV-1 Using Octet

The affinity of three cross-reactive recombinant antibodies (S303 rIgG1, S304 rIgG1, S309 rIgG1) and two SARS-CoV-1 specific antibodies was tested using Octet. The affinity was measured by immobilizing the antibody on sensors and dipping the sensors into wells with different concentrations of RBD.

The kinetics of antibody binding to RBD was recorded during the association phase, after which the sensors were dipped into buffer without antibody to observe the kinetics of antibody detaching from the RBD during the dissociation phase. Briefly, protein A biosensors (Pall ForteBio) were used to immobilize recombinant antibodies at 2.7 ug/ml for 1 minute, after a hydration step for 10 minutes with Kinetics Buffer (KB; 0.01% endotoxin-free BSA, 0.002% Tween-20, 0.005% NaN3 in PBS). Association curves were recorded for 5 minutes by incubating the antibody-coated sensors with different concentration of SARS-CoV-1 RBD (Sino Biological) or SARS-CoV-2 RBD (produced in house in Expi-CHO cells; residues 331-550 of spike from BetaCoV/Wuhan-Hu-1/2019, accession number MN908947). The highest RBD concentration tested was 10 ug/ml, then 1:2.5 serially diluted. Dissociation was recorded for 9 minutes by moving the sensors to wells containing KB. Affinities, represented by KD values, were calculated using a global fit model (Octet). Octet Red96 (ForteBio) equipment was used.

Three cross-reactive antibodies (5303 rIgG1, S304 rIgG1, S309 rIgG1) and two SARS-CoV-1 specific antibodies (5230 and S109) were tested. All antibodies showed strong binding to SARS-CoV-1 RBD. S230 and S109 did not bind to SARS-CoV-2 RBD. Binding of S303 rIgG1, S304 rIgG1, and S309 rIGg1 to SARS-CoV-2 RBD was in the nanomolar range, with S309 rIgG1 showing the strongest affinity. KD values are indicated below the graphs. KD values are only estimates (KD=<1.0×10⁻¹²M) if the antibody binding is very strong and dissociation is slow. An exact KD for S309 rIgG1 could not be measured by this assay since the dissociation was too slow.

### Example 4

### Neutralization of Sars-Cov-2 by Human Monoclonal Antibodies

Replication-incompetent viruses pseudotyped with the SARS-CoV-2 S gene (isolate BetaCoV/Wuhan-Hu-1/2019; accession number MN908947) were produced using methods as previously described (Temperton N J, et al. (2005) Longitudinally profiling neutralizing antibody response to SARS coronavirus with pseudotypes. Emerg Infect Dis 11(3):411-416.). Briefly, HEK293T/17 was cotransfected with a SARS-CoV-2 S-expressing plasmid (phCMV1, Genlantis) and with a complementing viral-genome reporter gene vector, pNL4-3. Luc+.E-R+. A single-cycle infectivity assay was used to measure the neutralization of luciferase-encoding virions pseudotyped with the SARS-CoV-2 S protein, as previously described (Temperton N J, et al. (2007) A sensitive retroviral pseudotype assay for influenza H5N1-neutralizing antibodies. Influenza Other Respi Viruses 1(3):105-112.). Briefly, appropriate dilutions of the virion-containing culture supernatants were preincubated at 37° C. for 1 h with antibodies at various concentrations and the virus-mAb mixtures was then added to Vero E6 cells that were seeded the day before infection. The cells were then lysed with Steady-Glo® reagent (Promega, E2520), and the relative luminescence units (RLU) in the cell lysates was determined on a luminometer microplate reader (Synergy H1 Hybrid Multi-Mode Reader; Biotek). The reduction of infectivity was determined by comparing the RLU in the presence and absence of antibody and expressed as percentage of neutralization.

Antibodies SARS-CoV-2 S300-v1, S301, S302, S303-v1, S304, S306, S307, S308-v1, S309 (comprising the S309-v1 VH sequence and the S309-v13 VL (VK) sequence), and S310 were tested for neutralization capacity. Antibodies SARS-CoV-2 S300-v1 and SARS-CoV-2 S309 were shown to neutralize SARS-CoV-2.

Further neutralization assays were carried out using SARS donor plasma and antibodies SARS-CoV-2 S309, S311, S312, S303-v1 (rIgG1), S304 (rIgG1), S306 (rIgG1), S310 (rIgG1), and S315. Using this assay, antibodies S309, S311, S312, and S315 were shown to neutralize SARS-CoV-2.

Additional neutralization assays were carried out using monoclonal antibodies S303, S304, S306, S309, S310, and S315.

### Example 5

### Neutralization of SARS-CoV-2 by Recombinant Human Monoclonal Antibodies

The neutralizing activity of two recombinant SARS-CoV-1 and SARS-Cov-2 cross-neutralizing antibodies, S304 rIgG1 and S309 rIgG1, against SARS-CoV-2 pseudotyped viruses (SARS-CoV-2pp) was determined.

Murine leukemia virus (MLV) pseudotyped with SARS-CoV-2 Spike protein (SARS-CoV-2pp) were used. DBT cells stably transfected with ACE2 (DBT-ACE2) were used as target cells. SARS-CoV-2pp was activated with trypsin TPCK at 10 ug/ml. Activated SARS-CoV-2pp was added to a dilution series of antibodies (starting with 50 ug/ml final concentration per antibody, 3-fold dilution). Antibodies were tested at concentrations between 50 ug/ml and 0.02 ug/ml. for the combination of S304 rIgG1 and S309 rIgG1, starting concentrations were 50 ug/ml for each antibody, i.e. the total starting antibody amount was 100 ug/ml. DBT-ACE2 cells were added to the antibody-virus mixtures and incubated for 48 hours. Luminescence was measured after aspirating cell culture supernatant and adding Steady-GLO® substrate (Promega). S309 rIgG1 showed an IC50 of 0.37 ug/ml, and S304 rIgG1 showed an IC50 of approximately 17 ug/ml. A combination of both antibodies was strongly neutralizing, with an IC50 of 0.077 μg/ml. Further neutralization assays were carried out using the same procedure for recombinant monoclonal antibodies S309 and S315, singly and in combination. S309 showed an IC50 of 1.091 ug/ml, and S315 showed an IC50 of 25.1 ug/ml. The combination of both antibodies showed an IC50 of 0.3047 ug/ml.

### Example 6

### Reactivity of Human Monoclonal Antibodies to SARS-CoV and SARS-CoV-2

Reactivity of additional human mAbs S311 and S312 with the spike Si subunit protein and the RBD of SARS-CoV and SARS-CoV-2 protein was determined by enzyme-linked immunosorbent assays (ELISA).

96-well plates were coated with recombinant SARS-CoV-2 Spike S1 Subunit Protein (Sino Biological), SARS-CoV-2 RBD (Sino Biological or produced in house; residues 331-550 of spike from BetaCoV/Wuhan-Hu-1/2019, accession number MN908947), recombinant SARS-CoV Spike S1 Subunit Protein (Sino Biological), or SARS-CoV RBD (Sino Biological).

Wells were washed and blocked with PBS+1% BSA for 1 hour at room temperature and were then incubated with serially diluted mAbs for 1 hour at room temperature. Bound mAbs were detected by incubating alkaline phosphatase-conjugated goat anti-human IgG (Southern Biotechnology: 2040-04) for 1 hour at room temperature and were developed by 1 mg/ml p-nitrophenylphosphate substrate in 0.1 M glycine buffer (pH 10.4) for 30 min at room temperature. The optical density (OD) values were measured at a wavelength of 405 nm in an ELISA reader (Powerwave 340/96 spectrophotometer, BioTek).

Further assays were carried out to determine reactivity of human monoclonal antibodies S300, S307, and S309 to RBD of SARS-CoV-2 and SARS-CoV-1 using the same procedure.

### Example 7

### Neutralization of SARS-CoV-2 by Recombinant Human Monoclonal Antibodies S309 and S315

Neutralizing activity of recombinant antibodies S309 rIgG1-LS and S315 rIgG1-LS against SARS-CoV-2 pseudotyped viruses (SARS-CoV-2pp) was determined.

Murine leukemia virus (MLV) pseudotyped with SARS-CoV-2 Spike protein (SARS-CoV-2pp) were used. DBT cells stably transfected with ACE2 (DBT-ACE2) were used as target cells. SARS-CoV-2pp was activated with trypsin TPCK at 10 ug/ml. Activated SARS-CoV-2pp was added to a dilution series of antibodies. DBT-ACE2 cells were added to the antibody-virus mixtures and incubated for 48 hours. Luminescence was measured after aspirating cell culture supernatant and adding Steady-GLO® substrate (Promega). Luciferase signal of infected cells was used to calculate the percentage of neutralization relative to a no-antibody control.

S309 rIgG1-LS showed an IC50 of approximately 3.9 nM, and S315 rIgG1-LS showed an IC50 of approximately 111.7 mM.

The neutralizing activity of S309-rFab was compared to that of full length S309 rIgG1-LS. Full length S309 rIgG-LS showed an IC50 of 3.821 nM, while S309-rFab showed in IC50 of 3.532 nM.

### Example 8

### Reactivity of Human Monoclonal Antibodies to RBD of SARS-CoV-1, RBD of SARS-CoV-2, and Ectodomains of Various Coronaviruses

The reactivity of monoclonal antibodies with the RBD of SARS-CoV-1 and SARS-CoV-2 and the Spike protein of SARS-CoV-1, SARS-CoV-2, OC43, and MERS was determined by enzyme-linked immunosorbent assays (ELISA).

384-well shallow ELISA plates were coated with stabilized prefusion Spike protein trimer of SARS-CoV-1, SARS-CoV-2, OC43, or MERS at 1 μg/ml, or with SARS-CoV-2 RBD (produced in house; residues 331-550 of spike from BetaCoV/Wuhan-Hu-1/2019, accession number MN908947) at 10 μg/ml, or SARS-CoV-1 RBD (Sino Biological) at 1 μg/ml.

Wells were washed and blocked with PBS+1% BSA for 1 hour at room temperature and were then incubated with serially diluted antibodies for 1-2 hours at room temperature. Antibodies were tested at a concentration range of 5 to 0.00028 g/ml. Plates were washed and bound antibodies were detected by incubating alkaline phosphatase-conjugated goat anti-human IgG (Southern Biotechnology: 2040-04) for 1 hour at room temperature followed by color development using 1 mg/ml p-nitrophenylphosphate substrate (Sigma-Aldrich 71768) in 0.1 M glycine buffer (pH 10.4) for 30 min at room temperature. The optical density (OD) values were measured at a wavelength of 405 nm in an ELISA reader (Powerwave 340/96 spectrophotometer, BioTek).

### Example 9

### Binding of Monoclonal Antibodies to SARS-CoV-1 and SARS-CoV-2 Spike Protein

ExpiCHO™ cells were transfected with phCMV1-SARS-CoV-2-S, SARS-spike_pcDNA.3 (strain SARS), or empty phCMV1 using Expifectamine™ CHO Enhancer (Thermo Fisher Scientific). Two days after transfection, cells were collected for immunostaining with antibody. An Alexa647-labelled secondary antibody anti-human IgG Fc was used for detection. Binding of monoclonal antibody to transfected cells was analyzed by flow cytometry using a ZE5 Cell Analyzer (Biorad) and FlowJo™ software (TreeStar). Positive binding was defined by differential staining of CoV-S transfectants versus mock transfectants. Monoclonal antibody S309 was tested by flow-cytometry at 10 μg/ml for the ability to stain ExpiCHO™ cells expressing the S protein of SARS-CoV-1 or SARS-CoV-2.

Binding to SARS-CoV-1 S protein or SARS-CoV-2 S protein was measured by flow cytometry for monoclonal antibodies S303, S304, S306, S309, S310, S315, S110, S124, S230, and S109, and the EC50 values were calculated. Eight of these antibodies was calculated to have EC50 values ranging between 1.4 ng/ml and 6,100 ng/ml for SARS-CoV-2 S protein binding and between 0.8 ng/ml and 254 ng/ml for SARS-CoV-1 S protein binding.

Further binding assays using the same procedure were carried out on recombinant monoclonal antibody S309 and four S309 variants. The EC50 values for each antibody are shown in Table 2. The VHs and VLs are summarized in Example 17.

TABLE 2

| Antibody | EC50 (ng/ml) - Exp. 1 | EC50 (ng/ml) - Exp. 2 |
|---|---|---|
| S309-11 (“11”) | 23.1 | 11.5 |
| S309-12 (“12”) | 22.3 | 9.6 |
| S309-13 (“13”) | 21.8 | 8.9 |
| S309-14 (“14”) | 21.4 | 8.4 |
| S309-15 (“15”) | 18.8 | 7.8 |

Additional assays using the same procedure were carried out using monoclonal antibodies S303, S304, S306, S309, S310, S315, and comparator antibodies S109, S110, S124, and S230.

Assays using the same procedure were also carried out using recombinant monoclonal antibodies S300 and S307.

### Example 10

### Binding of Human Monoclonal Antibodies S309, S303, S304, and S315 to RBD of SARS-CoV-2 and SARS-CoV-1 Using Octet

Affinity of recombinant monoclonal antibodies S309, S303, S304, and S315 was tested using Octet.

His-tagged RBD of SARS-CoV-1 or SARS-CoV-2 were loaded at 3 g/ml in kinetics buffer (KB) for 15 minutes onto anti-HIS (HIS2) biosensors (Molecular Devices, ForteBio). Association of full-length antibodies was performed in KB at 15 μg/ml for 5 minutes. Association of Fab fragments was performed in KB at 5 μg/mL for 5 minutes. Dissociation in KB was measured for 10 minutes.

Affinities, represented by KD values, were calculated using a global fit model (Octet). Octet Red96 (ForteBio) equipment was used.

Each of these antibodies bound SARS-CoV-2 and SARS-CoV-1 RBD with nanomolar to sub-picomolar affinity.

### Example 11

### Binding of Human Monoclonal Antibody S309 IgG and S309 Fab to SARS-CoV-2 S Protein Ectodomain Trimer and RBD

Affinity and avidity determination of IgG1 compared to Fab fragment: biotinylated RBD of SARS-CoV-2 (produced in house; residues 331-550 of spike protein from BetaCoV/Wuhan-Hu-1/2019, accession number MN908947, biotinylated with EZ-Link NHS-PEG4-Biotin from ThermoFisher) and biotinylated SARS-CoV-2 2P S avi-tagged were loaded at 7.5 μg/ml in Kinetics Buffer (KB; 0.01% endotoxin-free BSA, 0.002% Tween-20, 0.005% NaN3 in PBS) for 8 minutes onto Streptavidin biosensors (Molecular Devices, ForteBio). Association of IgG1 and Fab was performed in KB at 100, 33, 11, 3.6, 1.2 nM for 5 minutes. Dissociation in KB was measured for 10 minutes. KD values were calculated using a 1:1 global fit model (Octet). S309 IgG bound to the SARS-CoV-2 RBD and to the S ectodomain trimer with sub-picomolar and picomolar avidities, respectively. S309 Fab bound to both the SARS-CoV-2 RBD and the S ectodomain trimer with nanomolar to sub-nanomolar affinities.

### Example 12

### Competitive Binding of Human Monoclonal Antibodies to RBD of SARS-CoV-1 or SARS-CoV-2

Competitive binding of pairs of monoclonal antibodies to SARS-CoV-1 RBD or SARS-CoV-2 RBD was measured to define the binding sites of the antibodies.

Strepavidin biosensors (Pall ForteBio) were used to immobilize anti-Strep Tag II antibody at 3 ug/ml (clone 5A9F9, Biotin, LabForce AG, Muttenz CH), after a hydration step for 10 min with Kinetics Buffer (KB; 0.01% endotoxin-free BSA, 0.002% Tween-20, 0.005% NaN3 in PBS). Either SARS-CoV-1 or SARS-CoV-2 RBD with a Strep Tag II (produced in-house) was then loaded for 6 min at a concentration of 4 μg/ml in KB. The first antibody was allowed to associate for a period of time, then the second antibody was allowed to associate for a period of time.

### Example 13

### Competitive Binding of Human Monoclonal Antibody S309 and RBD to Human ACE2

Competitive binding of monoclonal antibodies and RBD to human ACE2 was measured. ACE2-His (Bio-Techne AG) was loaded for 30 minutes at 5 μg/ml in kinetics buffer (KB) onto anti-HIS (HIS2) biosensors (molecular Devices-ForteBio)SARS-CoV-1 RBD-rabbit Fc or SARS-CoV-2 RBD-mouse Fc (Sino Biological Europe GmbH) at 1 μg/ml was associated for 15 minutes, after a preincubation with or without antibody at 30 μg/ml for 30 minutes. Dissociation was monitored for 5 minutes.

### Example 14

### Antibody-Dependent Cytotoxicity and Antibody-Dependent Cellular Phagocytosis of Human Monoclonal Antibodies

Natural killer (NK)-mediated antibody-dependent cell cytotoxicity (ADCC) can contribute to viral control by killing infected cells displaying viral protein on their surface. To investigate the ability of the Abs isolated to leverage this function, ADCC was investigated in vitro using freshly isolated human NK cells as effector cells and SARS-CoV-2 S-transfected ExpiCHO™ cells as target cells. Target cells were incubated with different amounts of antibody and after 10 minutes were incubated with primary human NK cells as effector cells at a target:effector ratio of 9:1. NK cells were isolated from fresh blood of healthy donors using the MACSxpress® NK Isolation Kit (Miltenyi Biotec, Cat. Nr.: 130-098-185). Antibody-dependent cell killing was measured using an LDH release assay (Cytotoxicity Detection Kit (LDH) (Roche; Cat. Nr.: 11644793001) after 4 hours of incubation at 37° C.

Macrophage or dendritic cell-mediated antibody-dependent cellular phagocytosis (ADCP) can also contribute to viral control by clearing infected cells and by potentially stimulating T cell response with viral antigen presentation. ADCP was tested with peripheral blood mononuclear cells as phagocytes and ExpiCHO™ transfected with SARS-CoV-2 S fluorescently labeled with PKH67 Fluorescent Cell Linker Kits (Sigma Aldrich, Cat. Nr.: MINI67) as target cells. Target cells were incubated with different amounts of antibody for 10 minutes, followed by incubation with human PBMCs isolated from healthy donors that were fluorescently labeled with Cell Trace Violet (Invitrogen, Cat. Nr.: C34557) at an effector:target ratio of 20:1. After an overnight incubation at 37° C., cells were stained with anti-human CD14-APC antibody (BD Pharmingen, Cat. Nr.: 561708, Clone M5E2) to stain phagocytic cells. Antibody-mediated phagocytosis was determined by flow cytometry, measuring the % of monocytes that were positive for PKH67 fluorescence.

Human monoclonal antibodies S309, S304, S306, S315, S230, and the combination of S309 and S304 were assayed for antibody-dependent cell-mediated cytotoxicity (ADCC) and antibody-dependent cellular phagocytosis (ADCP).

Fc variants of monoclonal antibody S309 were tested for ADCC. S309-LS includes the MLNS Fc mutation. S309-GRLR includes the G236R/L328R Fc mutation, which exhibits minimal binding to FcγRs. S309-LS-GAALIE includes both the MLNS and GAALIE Fc mutations.

Human monoclonal antibodies S303, S304, S306, S309, S315, and the combination of S309 and S315 were assayed for ADCC and ADCP. At least the non-GRLR S309 antibodies (alone or in combination with S304) demonstrated ADCC and ADCP.

### Example 15

### Reactivity of Monoclonal Antibodies to Cell Lysate of SARS-CoV-2-Infected Cells

Reactivity of monoclonal antibodies S304, S306, S309, and S310 to cell lysate of SARS-CoV-2-infected VeroE6 cells was measured.

### Example 16

### Neutralization of SARS-CoV-2 Infection by Monoclonal Antibodies S304 and S309, Alone and in Combination

Neutralization of SARS-CoV-2 infection by monoclonal antibodies S304 and S309 was assessed using a SARS-CoV-2 live virus assay. The live virus neutralization assay quantifies the number of infected cells by staining for viral nucleoprotein (NP) with an NP-specific polyclonal rabbit serum. Inhibition was assessed by measuring NP expression at 24 and 45 hours post infection. Enzyme immunoassay (EIA) was used to quantify the level of infection for each antibody dilution tested.

Neutralization was carried out for one hour at room temperature at the indicated antibody concentrations using Vero E6 cells in monolayer in 96-well plates. Wells were infected with 100 TCID50 of virus. After 24 or 45 hours, monolayers were fixed and stained for inhibition of NP expression. Monoclonal antibodies S304 and S309 show a synergistic enhancement of neutralization.

### Example 17

### Production of S309 RIgG Variants

Recombinant IgG1 antibodies were produced using the VH and VL sequences of monoclonal antibody S309. S309-11 comprises the wild-type VH sequence (SEQ ID NO: 105) and wild-type VL sequence (SEQ ID NO: 168) of S309. S309-12 comprises an N55Q VH variant sequence (SEQ ID NO: 113) and the wild-type VL sequence (SEQ ID NO: 168) of S309. S309-13 comprises a W50F variant sequence and the wild-type VL sequence (SEQ ID NO: 168) of S309. S309-14 comprises a W105F variant sequence (SEQ ID NO: 119) and the wild-type VL sequence (SEQ ID NO: 168) of S309. S309-15 comprises a W50F/G56A/W105F VH variant sequence and the wild-type VL sequence of S309. S309 recombinant antibody and each of the four variants were produced by transient transfection and expression of a plasmid vector encoding the recombinant antibody in HD 293F cells (GenScript). Cells were harvested on day 4 and IgG expression was validated by Western blot and protein A titer analysis.

### Example 18

### Binding of S309 RIgG and Variants to SARS-CoV-2 RBD Measured by SPR

Binding of recombinant monoclonal antibody S309 and four variants (see Example 17) to RBD was measured using surface plasmon resonance (SPR). SPR experiments were carried out with a Biacore™ T200 instrument using a single-cycle kinetics approach. S309 IgG was captured on the surface and increasing concentrations of purified SARS-CoV-2 RBD, either glycosylated or deglycosylated, were injected. SPR was conducted using a sensor chip with anti-human Fc covalently immobilized (GE). Buffer used was 10 mM HEPES pH 7.4, 150 mM NaCl, 3 mM EDTA, and 0.05% P20 detergent. Assays were conducted at 25° C. Recombinant antibodies were diluted from supernatant to approximately 2 μg/ml. RBD concentrations were 0.8 nM, 3.1 nM, 12.5 nM, 50 nM, and 200 nM. Glycosylated RBD was obtained by expression in HEK293 cells and purified using one-step Ni affinity purification. Deglycosylated RBD was obtained by expression in-house in Expi293 cells grown in the presence of kifunensine, purification using one-step Ni affinity purification, and treatment with endoglycosidase H. Single-cycle kinetics assays were carried out with 3 minute injections and 20 minute dissociation periods. Association and dissociation kinetics were monitored and fit to a binding model to determine affinity. The results are summarized in Table 3.

TABLE 3

| | Glycosylated RBD | | | | Deglycosylated RBD | | | |
|---|---|---|---|---|---|---|---|---|
| S309 variant | K_{D} | | Kₐ (1/Ms) | K_{d} (1/s) | K_{D} | | Kₐ (1/Ms) | K_{d} (1/s) |
| S309-11 (WT) | 0.50 | nM | 10.0e4 | 5.0e−5 | 0.91 | nM | 3.0e5 | 2.8e−4 |
| S309-11 (WT) | 0.68 | nM | 9.5e4 | 6.5e−5 | 0.98 | nM | 2.9e5 | 2.9e−4 |
| replicate | | | | | | | | |
| S309-12 (N55Q) | 0.46 | nM | 9.2e4 | 4.2e−5 | 1.3 | nM | 2.7e5 | 3.6e−4 |
| S309-13 (W50F) | 0.51 | nM | 9.9e4 | 5.0e−5 | 1.8 | nM | 3.0e5 | 5.3e−4 |
| S309-14 (W105F) | 0.38 | nM | 1.0e5 | 3.9e−5 | 7.9 | nM | 9.8e5 | 7.7e−3 |

| | | | | | | | |
|---|---|---|---|---|---|---|---|
| S309-15 | 1.7 | nM | 9.9e4 | 1.6e−4 | >10 | nM | estimate Kd with |
| (W50F/G56A/W105F) | | | | | | | steady-state fit |

Binding to deglycosylated RBD was measured in two different SPR assays using different parameters. Experiment 1 used 10 minute injections and an RBD concentration series of 4-fold dilutions from 100 nM. Experiment 2 used 3 minute injections and a concentration series of 4-fold dilutions from 200 nM, as described above. Results are shown in Table 4.

TABLE 4

| | Experiment 1 | | | | Experiment 2 | | | |
|---|---|---|---|---|---|---|---|---|
| S309 variant | K_{D} | | Kₐ (1/Ms) | K_{d} (1/s) | K_{D} | | Kₐ (1/Ms) | K_{d} (1/s) |
| S309-11 (WT) | 0.83 | nM | 3.0e5 | 2.5e−4 | 0.91 | nM | 3.0e5 | 2.8e−4 |
| S309-11 (WT) | 0.91 | nM | 3.0e5 | 2.7e−4 | 0.98 | nM | 2.9e5 | 2.9e−4 |
| replicate | | | | | | | | |
| S309-12 (N55Q) | 1.2 | nM | 2.7e5 | 3.2e−4 | 1.3 | nM | 2.7e5 | 3.6e−4 |
| S309-13 (W50F) | 1.7 | nM | 2.8e5 | 4.6e−4 | 1.8 | nM | 3.0e5 | 5.3e−4 |

| | | | | | | | |
|---|---|---|---|---|---|---|---|
| S309-14 (W105F) | 14 | nM | Fit to steady state | 7.9 | nM | 9.8e5 | 7.7e−3 |

| | | | | |
|---|---|---|---|---|
| S309-15 | 37 | nM | Fit to steady state | Steady-state fit not possible |
| (W50F/G56A/W105F) | | | | |

Binding of recombinant monoclonal antibody 5309 and five variants to RBD was measured by surface plasmon resonance (SPR) using the same procedure described above, except using purified recombinant antibodies rather than cell culture supernatant. Results are shown in Table 5.

TABLE 5

| | Glycosylated RBD | | | | Deglycosylated RBD | | | |
|---|---|---|---|---|---|---|---|---|
| S309 variant | K_{D} | | Kₐ (1/Ms) | K_{d} (1/s) | K_{D} | | Kₐ (1/Ms) | K_{d} (1/s) |
| S309 WT | 0.26 | nM | 9.3e4 | 2.4e−5 | 0.67 | nM | 3.4e5 | 2.3e−4 |
| S309 N55Q | 0.39 | nM | 8.5e4 | 3.3e−5 | 1.1 | nM | 3.1e5 | 3.2e−4 |
| S309 W50F | 0.39 | nM | 9.2e4 | 3.6e−5 | 1.4 | nM | 3.5e5 | 4.9e−4 |
| S309 W105F | 0.35 | nM | 9.6e5 | 3.4e−5 | 5.1 | nM | 1.5e6 | 7.9e−3 |
| S309 | 1.6 | nM | 9.4e4 | 1.5e−4 | >10 | nM | estimate Kd with | |
| W50F/G56A/W105F | | | | | | | steady-state fit | |
| S306 G56A | 0.54 | nM | 9.3e4 | 5.1e−5 | 0.70 | nM | 3.4e5 | 2.4e−4 |

### Example 19

### Neutralization of SARS-CoV-2 by Recombinant Monoclonal Antibody S309 and Four Variants

Neutralizing activity of recombinant monoclonal antibody 5309 and four variants (Example 17) was determined using a VSV-based luciferase reporter pseudotyping system (Kerafast). VSV pseudoparticles and antibody are mixed in DMEM and allowed to incubate for 30 minutes at 37° C. The infection mixture is then allowed to incubate with Vero E6 cells for 1h at 37° C., followed by the addition of DMEM with Pen-Strep and 10% FBS (infection mixture is not removed). The cells are incubated at 37 C for 18-24 hours. Luciferase is measured using an Ensight Plate Reader® (Perkin Elmer) after the addition of Bio-Glo™ reagent (Promega). Calculated EC50 values based on this experiment are shown in Table 6.

TABLE 6

| Recombinant Antibody | EC50 |
|---|---|
| S309-11 (WT) | 109 |
| S309-12 (N55Q) | 103 |
| S309-13 (W50F) | 97 |
| S309-14 (W105F) | 65 |
| S309-15 (W50F/G56A/W105F) | 53 |

### Example 20

### Determination of Antibody-Dependent Activation of Human FcγRIIa or FcγRIIa

Determination of antibody-dependent activation of human FcgRIIIa or FcgRIIa was performed using ExpiCHO™ cells transiently transfected with SARS-CoV-2 S (BetaCoV/Wuhan-Hu-1/2019), incubated with titrated concentrations of antibody for 10 minutes. ExpiCHO™ cells then were incubated with Jurkat cells expressing FcγRIIIa receptor or FcgRIIa on their surface and stably transfected with NFAT-driven luciferase gene (Promega, Cat. Nr.: G9798 and G7018) at an effector to target ratio of 6:1 for FcγRIIIa and 5:1 for FcγRIIa. Activation of human FcγRs in this bioassay results in the NFAT-mediated expression of the luciferase reporter gene. Luminescence was measured after 21 hours of incubation at 37° C. with 5% C02, using the Bio-Glo™ Luciferase Assay Reagent according to the manufacturer's instructions. Monoclonal antibodies S303, S304, S306, S309, S315, and a combination of S309 and S315 were assayed, along with comparator antibody S320. S309, alone or in combination with S315, activated FcγRIIIa (V158) and FcγRIIa (H131).

### Example 21

### Analysis of SARS-CoV-2 S Glycoprotein Sequences

Analysis of the S glycoprotein sequences of 2,229 SARS-CoV-2 isolates was carried out, and indicates that several mutations have occurred with variable frequency on the SARS-CoV-2 S ectodomain.

Further analysis of the S glycoprotein sequences was carried out using 11,839 SARS-CoV-2 isolates. The epitope bound by S309 is conserved in all but four isolates, and those isolates contained N354D or S359N substitutions that are not expected to affect S309 recognition.

### Example 22

### Competition of Antibody S309 with Antibodies Isolated from SARS-CoV-2 Patients

Human monoclonal antibodies isolated from patients who recovered from SARS-CoV-2 infection were tested for overlapping RBD binding sites with monoclonal antibody S309. Competition assays were performed using Octet (instrument: Octet Red96, ForteBio). Anti-His sensors (BIOSENSOR ANTI-PENTA-HIS (HIS1K)1*1ST) were used to immobilize in house produced HIS-tagged RBD of SARS-CoV-2 (residues 331-550 of Spike protein from BetaCoV/Wuhan-Hu-1/2019, accession number MN908947) at a concentration of 3 μg/ml. Antibodies were associated for 6 min at 15 μg/ml. All proteins were diluted in kinetics buffer (KB). Competing antibodies were then associated at the same concentration for additional 6 mins. Two antibodies were shown to compete with S309 for binding to RBD but, unlike S309, they were not neutralizing for SARS-CoV-2. Data not shown.

### Example 23

### Resistance Selection of SARS-CoV-2 Against Monoclonal Antibody S309-12-MLNS

To examine resistance selection, SARS CoV-2 was passaged for over one month in the presence of Vero E6 cells and fixed concentrations of monoclonal antibody S309-12-MLNS (i.e. S309 N55Q (VH) with MLNS mutations in Fc). Cytopathogenic effect (CPE) was evaluated by visual inspection of plates. Even when no CPE was observed, viral titres were evaluated by focus-forming assay with a methylcellulose overlay. No evidence of viral breakthrough in antibody-treated wells was observed, even at the minimum antibody concentration tested. Data are representative of wells in triplicate.

### Example 24

### Neutralization of SARS-CoV-2 Infection of Calu-3 Human Lung Cells by Monoclonal Antibody S309

Monoclonal antibody S309 was tested for its ability to neutralize live SARS-CoV-2 virus infection of Calu-3 human lung cells (which are positive for the transmembrane protease TMPRSS2) and VeroE6 cells using a nano luciferase assay. In this assay, S309 had an IC50 in Calu-3 cells of 97.70 μg/mL and in VeroE6 cells of 158.5 μg/mL.

### Example 25

### Neutralization of SARS-CoV-2 Infection by Monoclonal Antibody S309

Monoclonal antibody S309 was tested neutralization of live SARS-CoV-2 virus infection using both a nano luciferase assay and IFA assay. Briefly, model cells were infected with live SARS-CoV-2 luciferase virus for six hours. Data were collected using three different antibody concentrations: 1, 0.1, and 0.01 MOI.

Results from the nano-luciferase assay were as follows: at 1 MOI, S309 IC50 was 240.6 μg/mL; at 0.1 MOI, S309 IC50 was 235.3 μg/mL, and at 0.01 MOI, S309 IC50 was 206.6 μg/mL.

Results from the IFA assay were as follows: at 1 MOI, S309 IC50 was 233.0 g/mL; at 0.1 MOI, S309 IC50 was 156.5 μg/mL, and at 0.01 MOI, S309 IC50 was 142.8 μg/mL. Notably, no clusters of infection (or foci) were observed in this infection format.

### Example 26

### Neutralization of SARS-CoV-2 Infection by Monoclonal Antibodies S309 N55Q Ls and S309 N55Q LS GAALIE

Monoclonal antibodies S309 N55Q LS (also referred to herein as S309 N55Q MLNS, comprising M428L/N434S Fc mutations) and S309 N55Q LS GAALIE (also referred to herein as S309 N55Q MLNS GAALIE, comprising G236A, A330L, 1332E, M428L, and N434S Fc mutations) were assayed for ability to neutralize live SARS-CoV-2 virus infection. Each of S309 N55Q LS and S309 N55Q LS GAALIE comprises a VH having the sequence set forth in SEQ ID NO.: 113 and a VL having the sequence set forth in SEQ ID NO.: 168. The calculated EC50 for S309 N55Q LS was 100.1 ng/ml. The calculated EC50 for S309 N55Q LS GAALIE was 78.3 ng/ml.

### Example 27

### Neutralization of SARS-CoV-2 Pseudotyped Virus by Monoclonal Antibodies S309 N55Q LS and S309 N55Q LS GAALIE

Neutralization of SARS-CoV-2 pseudotyped virus by monoclonal antibodies S309 N55Q LS and S309 N55Q LS GAALIE was tested. The pseudotyped virus was VSV pseudotyped with SARS-CoV-2 Spike protein. The calculated EC50 value for S309 N55Q LS was 24.06 ng/ml. The calculated EC50 value for S309 N55Q LS GAALIE was 22.09 ng/ml.

### Example 28

### Binding of Monoclonal Antibodies S309 N55Q LS and S309 N55Q LS GAALIE to SARS-CoV-2 RBD

Binding of monoclonal antibodies S309 N55Q LS and S309 N55Q LS GAALIE to SARS-CoV-2 RBD was measured by surface plasmon resonance (SPR). Both antibodies exhibited strong binding.

### Example 29

### Binding of Monoclonal Antibodies S309 N55Q LS and S309 N55Q LS GAALIE to SARS-CoV-2 Spike Protein

Binding of monoclonal antibodies S309 N55Q LS and S309 N55Q LS GAALIE to SARS-CoV-2 to SARS-CoV-2 Spike protein was measured by flow cytometry. Both antibodies bound to Spike protein at concentrations as low as approximately 10 ng/mL, and binding to more than 60% of cells at 10,000 ng/mL antibody.

### Example 30

### Binding of Monoclonal Antibodies S309 N55Q LS and S309 N55Q LS GAALIE to Human Fcγ Receptors

Binding of monoclonal antibodies S309 N55Q LS and S309 N55Q LS GAALIE to human Fcγ receptors was assayed using SPR. Binding to FcγRIIa (both low affinity R131 and high affinity H131 alleles), FcγRIIIa (both low affinity F158 and high affinity V158 alleles), and FCγRIIb was measured. Biotin CAPture Reagent (modified streptavidin) was injected across all flow cells of a CAP sensor chip docked in a Biacore™ T200 (Cytiva). Biotinylated Fc receptors at 1 μg/mL were injected across a single flow cell at 10 μL/min for 60 seconds (one receptor per flow cell), with one flow cell reserved as a reference surface. Antibody at 100 μg/mL (diluted in HBS-EP+) was injected across all flow cells for 200 seconds using a flow rate of 30 μL/min and association was monitored. Dissociation was monitored for another 200 seconds after injection. Data was collected at 10 Hz. After each binding measurement, CAP Regeneration reagent was injected to prepare the surface for a new cycle. Experiments were performed at 25° C., with the samples held at 15° C. in the instrument prior to injection. Both antibodies had at least measurable binding to all Fcγ receptors tested. S309 N55Q LS GAALIE had increased binding (as compared to S309 N55Q LS) to all Fcγ receptors except for FcγRIIb.

### Example 31

### Binding of Monoclonal Antibodies S309 LS, S309 N55Q LS, and S309 N55Q LS GAALIE to Complement Component C1Q

Binding of monoclonal antibodies S309 LS, S309 N55Q LS, and S309 N55Q LS GAALIE to complement component C1q was measured by biolayer interferometry (BLI) on an Octet instrument. Anti-human Fab (CH1-specific) sensors were used to capture antibody at 10 μg/ml for 10 minutes. The IgG-loaded sensors were then exposed to kinetics buffer containing 3 μg/ml of purified human C1q for 4 minutes, followed by a dissociation step in the same buffer for additional 4 minutes. Association and dissociation profiles were measured in real time as changes in the interference pattern. S309 LS and S309 N55Q LS, but not S309 N55Q LS GAALIE, bound strongly to C1q.

### Example 32

### In Vitro Activation of Human Fc Gamma Receptors by Monoclonal Antibodies S309 LS, S309 N55Q LS, and S309 N55Q LS GAALIE

The ability of monoclonal antibodies S309 LS, S309 N55Q LS, and S309 N55Q LS GAALIE to elicit antibody-dependent activation of human Fcγ receptors was assayed in vitro. Each of S309 LS, S309 N55Q LS, S309 N55Q LS GAALIE, and negative control antibody S309-GRLR was serially diluted 6-fold in assay buffer from 10,000 ng/ml to 0.006 ng/ml. Nine point serial dilutions of antibody were incubated with 12,500 (for FcγRIIIa and FcγRIIb) or 10,000 (for FcγRIIa) CHO-CoV-2-Spike cells per 96-plate well in a white, flat-bottom plate for 15 minutes at room temperature. Jurkat effector cells expressing the indicated FcγRs and stably transfected with an NFAT-driven luciferase gene were thawed, diluted in assay buffer, and added to the plate at an effector to target cell ratio of 6:1 for FcRγIIIa and FcγRIIb or 5:1 for FcγRIIa. Control wells were included to measure antibody-independent activation (containing target cells and effector cells but no antibody) and background luminescence of the plate (wells containing assay buffer only). Plates were incubated for 18 hours at 37° C. with 5% C02. Activation of human FcγRs in this bioassay results in the NFAT-mediated expression of the luciferase reporter gene. Luminescence was measured with a luminometer after adding the Bio-Glo™ Luciferase Assay Reagent according to the manufacturer's instructions. The negative control showed low-level activation of FcγRIIb and did not activate any of the other FcγRs. S309 N55Q LS GAALIE was the only antibody to strongly activate FcγRIIIa (F158). All non-GRLR S309 antibodies activated FcγRIIa, FcγRIIb, and FcγRIIIa (V158).

### Example 33

### Effector Function of Human Monoclonal Antibodies S309 LS, S309 N55Q LS, and S309 N55Q LS GAALIE

Human monoclonal antibodies S309 LS, S309 N55Q LS, and S309 N55Q LS GAALIE were assayed for their ability to promote NK-cell mediated antibody-dependent cell-mediated cytotoxicity (ADCC) and monocyte-mediated antibody-dependent cellular phagocytosis (ADCP) against cells expressing CoV2-spike protein.

ADCC was measured in vitro by exposing freshly isolated human NK cells from two genotyped donors expressing homozygous low-affinity (F/F158) or high-affinity (V/V158) FcγRIIIa to antibody pre-incubated with CHO-CoV-2-Spike cells and measuring LDH release as a readout according to the manufacturer's instructions (Cytotoxicity Detection Kit (LDH), Roche) after 4 hours of incubation at 37° C. In brief, plates were centrifuged for 4 minutes at 400×g, and 35 μl of supernatant was transferred to a flat 384-well plate. LDH reagent was prepared and 35 μl were added to each well. Using a kinetic protocol, the absorbance at 490 nm and 650 nm was measured once every 2 minutes for 8 minutes, and the slope of the kinetics curve was used as result. The percent specific lysis was determined by applying the following formula: (specific release −spontaneous release)/(maximum release −spontaneous release)×100. S309 GRLR was used as a negative control. All non-GRLR S309 antibodies elicited ADCC.

The ability of monoclonal antibodies S309 LS, S309 N55Q LS, S309 N55Q LS GAALIE, and control antibody S309-GRLR to promote ADCP by primary CD14+ monocytes was measured in vitro by exposing freshly isolated human PBMCs (labeled with cell trace violet) to CHO-CoV-2-Spike expressing cells (labeled with PKH67 Fluorescent Cell Linker Kit (Sigma Aldrich)) that were pre-incubated with antibody. Serial dilutions of mAbs (serially diluted 5-fold from 5′,000 ng/ml to 0.32 ng/ml in RPMI-1640+L-glutamine supplemented with 10% Hyclone™ (Cytiva) FBS+2× anti-anti (antibiotic-antimycotic)) were incubated with 10,000 CHO-CoV-2-Spike cells per well of a 96 well polypropylene plate for 10 minutes. Primary PBMCs were fluorescently labeled with Cell Trace Violet according to the manufacturer's instructions. Target cell and antibody mixtures were then incubated with labeled PBMCs at an effector-to-target ratio of 16:1. ADCP activity was measured after overnight incubation by labeling the monocyte population for CD14, and measuring the percentage of cell trace violet+PKH67+ cells amongst CD14+ monocytes by flow cytometry.

### Example 34

### Effect of Monoclonal Antibody S309 ON SARS-CoV-2 Spike Protein-Mediated Cell Fusion

The effect of monoclonal antibody S309 on SARS-CoV-2 Spike protein-mediated fusion was tested using cells engineered to over-express Spike protein on the cell surface. The addition of S309 these cell cultures inhibited S protein-mediated cell-cell fusion.

### Example 35

### Effect of Monoclonal Antibodies S309 N55Q LS and S309 N55Q LS GAALIE on SARS-CoV-2 Replication

The effect of monoclonal antibodies S309 N55Q LS and S309 N55Q LS GAALIE on SARS-CoV-2 replication was tested in VeroE6 cells, PBMCs, and dendritic cells. SARS-CoV-2 virus was incubated for one hour with antibody S309 N55Q LS or S309 N55Q LS GAALIE. The virus/antibody mixture was then added to plated VeroE6, PBMC, or monocyte-derived dendritic (MoDC) cells. After incubating the cells with the virus/antibody mixture for one hour at 37° C., the cells were washed and incubated for a further 72 hours in fresh medium. The supernatant from the cultured cells then assayed for focus-forming units (FFU). The supernatant was diluted 1:5 and added to VeroE6 cells. After one hour at 37° C., the VeroE6 cells were overlaid with methylcellulose. After 24 hours' further incubation, the VeroE6 cell cultures were stained for SARS-CoV-2 nucleoprotein. The results showed that S309 variant antibodies do not cause antibody-mediated enhancement of SARS-CoV-2 replication in human donor-derived PBMCs or dendritic cells.

### Example 36

### RBD Epitope-Specific Antibodies in Sera of Individuals Who Recovered From SARS-CoV-2 Infection

Sera from hospitalized, symptomatic, and asymptomatic individuals diagnosed with COVID-19 were tested for competition for binding with monoclonal antibodies that bind identified antigenic sites of SARS-CoV-2 Spike protein RBD.

Six antibodies (S2H13, S2H14, S2A4, S2X35, S304 and S309), whose antigenic binding sites were identified on RBD molecule by X-crystallography or Cryo-EM, were produced, purified and biotinylated using EZ-Link™ NHS-PEO solid phase biotinylation kit (Thermo Scientific #21450) according to manufacturer's instructions. Binding to RBD was measured by ELISA. Briefly, 96-well half-area plates (corning, cat.3690) were coated with 1 μg/ml RBD (mouse Fc Tag, Sino Biological Europa GmbH, cat.no 40592-V05H) and blocked with Blocker buffer (Casein 1%, Thermo Fisher Scientific, cat.no 37528, +0.05% Tween 20). Biotinylated monoclonal antibodies were titrated and added to the plate, followed by incubation with streptavidin-AP and pNPP substrate (Sigma N2765-100TAB). Plates were read by spectrophotometer at 405 nm to determine optical densities (OD). Concentration of biotinylated antibody that determines 80% of binding to RBD (EC80) was calculated by non-linear regression analysis using Graph Prism software. Most sera (58-62%) contained antibodies to RBD Site Ia, which is one of the main portions of the receptor binding motif that interacts with ACE2 receptor. This portion in accessible only in the open conformation of RBD. A smaller fraction of sera (25-41%) contained antibodies recognizing the other RBD sites.

To determine the amount of RBD antigenic site-specific antibodies, titrated sera were tested by ELISA as described above with the additional step whereby a 2×BC80 concentration of biotinylated antibody was added on top of the sera after 20 min of incubation. ODs reflecting residual binding of biotinylated antibodies were measured and percentage of inhibition of binding was measured compared to control wells (100% in wells without sera and biotinylated antibodies and 0% in wells without sera). Dilution of sera that determine 80% of blocking of binding (BD80) was calculated by non-linear regression analysis using Graph Prism software. Similarly, 80% of self-blocking of binding of the 6 monoclonal antibodies was determined (BC80) by testing titrations of purified monoclonal antibodies instead of sera. The estimated amount of RBD antigenic site-specific antibodies was determined for each serum as concentration equivalents (ug/ml) sample with the following formula: BD80×BC80. The antibody response to the six RBD sites varied among individuals, with some individuals showing a poor antibody response.

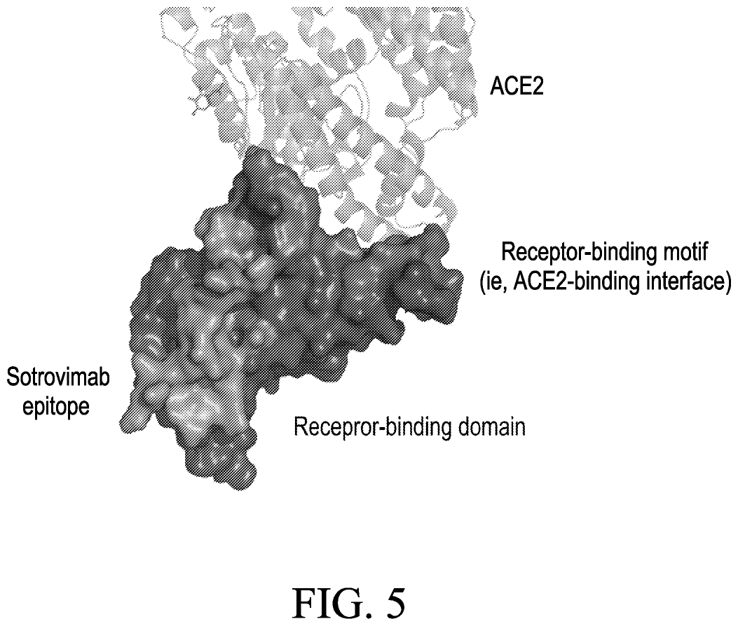

Further analysis was conducted to determine the binding site of sotrovimab, which is illustrated in FIG. 5.

### Example 37

### Materials and Methods

### Flow-Cytometry Based Screening for Binding to CoV S Protein Expressed on Mammalian Cells

ExpiCHO™ cells were transfected with S protein of SARS-CoV-2, SARS-CoV and MERS-CoV, or with an empty plasmid as a negative control. The monoclonal antibodies were then tested by flow-cytometry at 10 μg/ml for their ability to stain ExpiCHO™ cells expressing the S protein of 2019-nCoV, SARS-CoV, MERS-CoV or Mock cell transfectants.

### Transient Expression of Recombinant SARS-CoV-2 Protein

The full-length S gene of SARS-CoV-2 strain (2019-nCoV-S) isolate BetaCoV/Wuhan-Hu-1/2019 (accession number MN908947) was codon optimized for human cell expression and cloned into the phCMV1 expression vector (Genlantis). Expi-CHO cells were transiently transfected with phCMV1-SARS-CoV-2-S, phCMV1-MERS-CoV-S (London1/2012), SARS-spike_pcDNA.3 (strain SARS) or the empty phCMV1 (Mock) using Expifectamine™ CHO Enhancer. Two days after transfection, cells were collected, fixed, or fixed and permeabilized with saponin for immunostaining with a panel of monoclonal antibodies reactive to SARS-CoV Receptor Binding Domain (RBD). An Alexa647-labelled secondary antibody anti-human IgG Fc was used for detection. Binding of antibodies to transfected cells was analyzed by flow-cytometry using a ZE5 Cell Analyzer (Biorad) and FlowJo™ software (TreeStar). Positive binding was defined by differential staining of CoV-S-transfectants versus mock-transfectants.

### Competition Experiments Using Octet (BLI, Biolayer Interferometry)

Unless otherwise indicated herein, anti-His sensors (BIOSENSOR ANTI-PENTA-HIS (HIS1K)) were used to immobilize the Si subunit protein of SARS-CoV (Sino Biological Europe GmbH). Sensors were hydrated for 10 min with Kinetics Buffer (KB; 0.01% endotoxin-free BSA, 0.002% Tween-20, 0.005% NaN3 in PBS). SARS-CoV S1 subunit protein was then loaded for 8 min at a concentration of 10 μg/ml in KB. Antibodies were associated for 6 min at 15 μg/ml for full length mAbs nCoV-10 and nCov-6 mAbs or 5 μg/ml for Fab nCoV-4, and in a subsequent experiment comprising nCoV-1 all at 10 μg/ml. Competing antibodies were then associated at the same concentration for additional 6 mins.

### Competition Experiments Using Octet (BLI, Biolayer Interferometry)

For ACE2 competition experiments, ACE2-His (Bio-Techne AG) was loaded for 30 minutes at 5 μg/ml in KB onto anti-HIS (HIS2) biosensors (Molecular Devices-ForteBio). SARS-CoV-1 RBD-rabbit Fc or SARS-CoV-2 RBD-mouse Fc (Sino Biological Europe GmbH) at 1 μg/ml was associated for 15 minutes, after a preincubation with or without antibody (30 μg/ml, 30 minutes). Dissociation was monitored for 5 minutes.

### Affinity Determination Using Octet (BLI, Biolayer Interferometry)

For K_{D} determination of full-length antibodies, protein A biosensors (Pall ForteBio) were used to immobilize recombinant antibodies at 2.7 μg/ml for 1 minute, after a hydration step for 10 minutes with Kinetics Buffer. Association curves were recorded for 5 min by incubating the antibody-coated sensors with different concentration of SARS-CoV-1 RBD (Sino Biological) or SARS-CoV-2 RBD (produced in house; residues 331-550 of spike from BetaCoV/Wuhan-Hu-1/2019, accession number MN908947). Highest RBD concentration tested was 10 ug/ml, then 1:2.5 serially diluted. Dissociation was recorded for 9 min by moving the sensors to wells containing KB. K_{D} values were calculated using a global fit model (Octet). Octet Red96 (ForteBio) equipment was used.

For K_{D} determination of full-length antibodies compared to Fab fragments, His-tagged RBD of SARS-CoV-1 or SARS-CoV-2 were loaded at 3 μg/ml in KB for 15 minutes onto anti-HIS (HIS2) biosensors (Molecular Devices, ForteBio). Association of full-length antibody and Fab was performed in KB at 15 μg/ml and 5 μg/ml respectively for 5 minutes. Dissociation in KB was measured for 10 min.

### ELISA Binding

The reactivities of mAbs with SARS-CoV Spike S1 Subunit Protein (strain WH20) protein were determined by enzyme-linked immunosorbent assays (ELISA). Briefly, 96-well plates were coated with 3 μg/ml of recombinant SARS-CoV Spike S1 Subunit Protein (Sino. Biological). Wells were washed and blocked with PBS+1% BSA for 1 h at room temperature and were then incubated with serially diluted mAbs for 1 h at room temperature. Bound mAbs were detected by incubating alkaline phosphatase-conjugated goat anti-human IgG (Southern Biotechnology: 2040-04) for 1 h at room temperature and were developed by 1 mg/ml p-nitrophenylphosphate substrate in 0.1 M glycine buffer (pH 10.4) for 30 min at room temperature. The optical density (OD) values were measured at a wavelength of 405 nm in an ELISA reader (Powerwave 340/96 spectrophotometer, BioTek).

### Neutralization Assay

Unless otherwise indicated, Murine leukemia virus (MLV) pseudotyped with SARS-CoV-2 Spike protein (SARS-CoV-2pp) or SARS-CoV-1 Spike protein (SARS-CoV-1pp) were used. DBT cells stably transfected with ACE2 (DBT-ACE2) were used as target cells. SARS-CoV-2pp or SARS-CoV-1pp was activated with trypsin TPCK at 10 ug/ml. Activated SARS-CoV-2pp or SARS-CoV-1pp was added to a dilution series of antibodies (starting 50 ug/ml final concentration per antibody, 3-fold dilution). DBT-ACE2 cells were added to the antibody-virus mixtures and incubated for 48h. Luminescence was measured after aspirating cell culture supernatant and adding Steady-GLO® substrate (Promega).

Unless otherwise indicated, pseudoparticle neutralization assays use a VSV-based luciferase reporter pseudotyping system (Kerafast). VSV pseudoparticles and antibody are mixed in DMEM and allowed to incubate for 30 minutes at 37 C. The infection mixture is then allowed to incubate with Vero E6 cells for 1 h at 37 C, followed by the addition of DMEM with Pen-Strep and 10% FBS (infection mixture is not removed). The cells are incubated at 37 C for 18-24 hours. Luciferase is measured using an Ensight Plate Reader® (Perkin Elmer) after the addition of Bio-Glo™ reagent (Promega).

### SPR Single-Cycle Kinetics

SPR experiments were carried out with a Biacore™ T200 instrument using a single-cycle kinetics approach. S309 IgG was captured on the surface and increasing concentrations of purified SARS-CoV-2 RBD, either glycosylated or deglycosylated, were injected. Association and dissociation kinetics were monitored and fit to a binding model to determine affinity.

### Expression of Recombinant Antibodies

Recombinant antibodies were expressed in ExpiCHO™ cells transiently cotransfected with plasmids expressing the heavy and light chain as previously described. (Stettler et al. (2016) Specificity, cross-reactivity, and function of antibodies elicited by Zika virus infection. Science, 353(6301), 823-826) Monoclonal antibodies S303, S304, S306, S309, S310, and S315 were expressed as rIgG-LS antibodies. The LS mutation confers a longer half-life in vivo. (Zalevsky et al. (2010) Enhanced antibody half-life improves in vivo activity. Nature Biotechnology, 28(2), 157-159)

### Sequence Alignment

SARS-CoV-2 genomics sequences were downloaded from GISAID on March 29th 2020, using the “complete (>29,000 bp)” and “low coverage exclusion” filters. Bat and pangolin sequences were removed to yield human-only sequences. The spike ORF was localized by performing reference protein (YP_009724390.1)-genome alignments with GeneWise2. Incomplete matches and indel-containing ORFs were rescued and included in downstream analysis. Nucleotide sequences were translated in silico using seqkit. Sequences with more than 10% undetermined amino acids (due to N basecalls) were removed. Multiple sequence alignment was performed using MAFFT. Variants were determined by comparison of aligned sequences (n=2,229) to the reference sequence using the R/Bioconductor package Biostrings. A similar strategy was used to extract and translate spike protein sequences from SARS-CoV genomes sourced from ViPR (search criteria: SARS-related coronavirus, full-length genomes, human host, deposited before December 2019 to exclude SARS-CoV-2, n=53). Sourced SARS-CoV genome sequences comprised all the major published strains, such as Urbani, Tor2, TW1, P2, Frankfurt1, among others. Pangolin sequences as shown by Tsan-Yuk Lam et al were sourced from GISAID. Bat sequences from the three clades of Sarbecoviruses as shown by Lu et al (Lancet 2020) were sourced from Genbank. Civet and racoon dog sequences were similarly sourced from Genbank.

### Example 38

### Phase 1/2 Clinical Study of Sotrovimab (S309 N55Q LS) and VIR-7832 (S309 N55Q LS GAALIE) for Treatment of Mild to Moderate Covid-19 Disease in Adults

In one arm of a multi-center, multi-arm, multi-dose, multi-stage open-label, adaptive, seamless phase I/II Bayesian randomised platform trial (600 patients) to determine the optimal dose, activity and safety of multiple candidate agents for the treatment of COVID-19, sotrovimab (S309 N55Q LS; in more detail, sotrovimab is an engineered monoclonal antibody (IgG1*01 G1m17; VH of SEQ ID NO.:113, M428L and N434S Fc mutations; VL of SEQ ID NO.:168 (kappa light chain IgKC*01 k1m3)) and VIR-7832 ((IgG1*01 G1m17; VH of SEQ ID NO.:113, G236A, A330L, 1332E, M428L, and N434S Fc mutations; VL of SEQ ID NO.:168 (kappa light chain IgKC*01 k1m3)) are evaluated, randomizing between candidate and control with 2:1 allocation in favor of the candidate. Each dose is assessed for safety sequentially in cohorts of 6 patients. This arm comprises 3:1 randomised, blinded, placebo-controlled phase I of VIR-7831 (sotrovimab), followed by a 2:2:1 blinded, parallel group Phase II trial of VIR-7832 versus sotrovimab versus placebo. Single doses of VR-7832 are administered by intravenous (IV) infusion. The starting dose is 50 mg, and dose escalations of 150 and 500 mg may be used. The active comparator is sotrovimab (500 mg by i.v. infusion over 1 hour). Placebo is given by i.v. infusion over 1 hour.

### Primary Outcome Measures:

1. Dose-finding/Phase I [Time Frame: 29 days from randomisation]
Determination of a dose(s) for efficacy evaluation. Dose limiting toxicities (Safety and Tolerability of drug under study—CTCAE v5 Grade >3 adverse events)
2. Efficacy evaluation/Phase II—Severe patients (Group A) [Time Frame: 29 days from randomisation]
Determination of activity and safety.
In severe patients (Group A): time to clinical improvement. Improvement will be determined according to the WHO Clinical Progression Scale; improvement is defined as a minimum 2-step change from randomisation in the scale up to day 29 post-randomisation.
3. Efficacy evaluation/Phase II—Mild to moderate patients (Group B) [Time Frame: 15 days from randomisation]
Determination of activity and safety.
In mild to moderate patients (Group B): pharmacodynamics of drug under study, defined as time to negative viral titers in nose and/or throat swab, measured up to 15 days post-randomisation.

### Secondary Outcome Measures:

1. Safety assessed by rate of adverse events [Time Frame: Up to 29 days from randomisation]
Adverse event rate according to CTCAE v5
2. To evaluate clinical improvement [Time Frame: From randomisation to day 29]
Proportion of patients with clinical improvement (as defined above) at day 8, 15 and day 29.
3. To evaluate clinical improvement using WHO clinical progression scale [Time Frame: From randomisation to day 15]
Change at day 8 and 15 from randomisation in the WHO Clinical Progression Scale
4. To evaluate clinical improvement using WHO clinical progression scale [Time Frame: From randomisation to day 29]
Time to a one point change on the WHO Clinical Progression Scale
5. To evaluate clinical improvement using SpO₂/FiO₂ [Time Frame: From randomisation to day 29]
The ratio of the oxygen saturation to fractional inspired oxygen concentration (SpO₂/FiO₂)
6. To evaluate discharge [Time Frame: From randomisation to day 29]
Proportion of patient discharged at days 8, 15 and 29
7. To evaluate admission to ICU [Time Frame: From randomisation to day 29]
Admission rate to ICU
8. To evaluate safety further (WCC) [Time Frame: From randomisation to day 29]
White cell count on day 1, 3, 5, 8, 11 (while hospitalised); and Day 15 and 29
9. To evaluate safety further (Hg) [Time Frame: From randomisation to day 29]
Hemoglobin on day 1, 3, 5, 8, 11 (while hospitalised); and Day 15 and 29
10. To evaluate safety further (platelets) [Time Frame: From randomisation to day 29]
Platelets on day 1, 3, 5, 8, 11 (while hospitalised); and Day 15 and 29
11. To evaluate safety further (creatinine) [Time Frame: From randomisation to day 29]
Creatinine on day 1, 3, 5, 8, 11 (while hospitalised); and Day 15 and 29
12. To evaluate safety further (ALT) [Time Frame: From randomisation to day 29]
ALT on day 1, 3, 5, 8, 11 (while hospitalised); and Day 15 and 29
13. To evaluate overall mortality [Time Frame: From randomisation to day 29]
Mortality at Days 8, 15 and 29. Time to death from randomisation
14. To evaluate the number of oxygen-free days [Time Frame: From randomisation to day 29]
Duration (days) of oxygen use and oxygen-free days
15. To evaluate ventilator-free days [Time Frame: From randomisation to day 29]
Duration (days) of mechanical ventilation and mechanical ventilation-free days
16. To evaluate incidence of new mechanical ventilation use [Time Frame: From randomisation to day 29]
Incidence of new mechanical ventilation use
17. To evaluate National Early Warning Score (NEWS)2/qSOFA [Time Frame: From randomisation to day 29]
NEWS2/qSOFA assessed daily while hospitalised
18. To evaluate translational outcomes (Viral Load) [Time Frame: From randomisation to day 29]
Change in viral load over time
19. To evaluate translational outcomes (Baseline SARS-COV-2) [Time Frame: From randomisation to day 29]
Change in viral load over time

### Eligibility

Ages Eligible for Study: 18 Years and older (Adult, Older Adult)
Sexes Eligible for Study: All
Accepts Healthy Volunteers: No

### Inclusion Criteria

1. Adults (≥18 years) with laboratory-confirmed SARS-CoV-2 infection (PCR)
2. Ability to provide informed consent signed by study patient or legally acceptable representative
3. Women of childbearing potential (WOCBP) and male patients who are sexually active with WOCBP agree to use a highly effective method of contraception (as outlined in the protocol) from the first administration of trial treatment, throughout trial treatment and for the duration outlined in the candidate-specific trial protocol after the last dose of trial treatment
Additional criteria may be applied:
Group A (severe disease) 4a. Patients with clinical status of Grades 4 (hospitalised, oxygen by mask or nasal prongs), 5 (hospitalised, on non-invasive ventilation, or high flow oxygen), 6 (hospitalised, intubation and mechanical ventilation) or 7 (ventilation and additional organ support—pressors, renal replacement therapy (RRT), extracorporeal membrane oxygenation (ECMO)), as defined by the WHO clinical severity score, 9-point ordinal scale.
   Group B (mild-moderate disease) 4b. Ambulant or hospitalised patients with the following characteristics peripheral capillary oxygen saturation (SpO₂) >94% RA N.B.

The main trial exclusion criteria are outlined in the master protocol as:
1. *
2. *
3. Pregnant or breast feeding
4. *
5. Allergy to any study medication
6. Patients taking other prohibited drugs (as outline in CST protocol) within 30 days or
5 times the half-life (whichever is longer) of enrolment
7. Patients participating in another clinical trial of an investigational medicinal product (CTIMP)
*‘The master protocol stipulates the following for ‘1’, ‘2’ and ‘4’;
2. Stage 4 severe chronic kidney disease or requiring dialysis (i.e., estimated glomerular filtration rate <30 mL min 1.73 m²)
4. ‘anticipated transfer to another hospital which is not a study site within 72 hours’Those criteria are not applicable to this protocol as the IMP is a monoclonal antibody, which is not cleared from the body by the liver or kidneys (l and 2) and patients who are hospitalised are excluded from the study.
For the purpose of the VIR-7832 and sotrovimab candidate-specific trial the following exclusion criteria also apply:
8. Currently hospitalised or judged by the investigator as likely to require hospitalisation for acute medical care in the next 24 hours
9. Symptoms consistent with severe COVID-19 as defined by shortness of breath at rest or respiratory distress or requiring supplemental oxygen.
10. Participants who, in the judgement of the investigator are likely to die in the next 7 days
11. Severely immunocompromised participants including but not limited to cancer patients receiving immunosuppressive chemotherapy or immunotherapy, those with a solid organ transplant or allogeneic stem cell transplant within the last 3 months, any history of heart or lung transplant or high dose long-term systemic corticosteroids (equivalent to ≥20 mg a day of prednisone or the systemic equivalent for over 2 weeks)
12. Phase I only: Diabetes (requiring medication), chronic kidney disease (i.e., eGFR <60 as determined by the Modification of Diet in Renal Disease (MDRD) study), chronic liver disease (e.g., cirrhosis), congestive heart failure (New York Heart Association (NYHA) class II or more), chronic obstructive pulmonary disease (history of chronic bronchitis, chronic obstructive lung disease, or emphysema with dyspnoea on physical exertion), and moderate to severe asthma (participant requires an inhaled steroid to control symptoms or has been prescribed a course of oral steroids in the past year).
13. Any current medical condition that, in the Investigator's judgment, precludes the participant's safe participation in and completion of the study.
14. Known hypersensitivity to any constituent present in the investigational product
15. Previous anaphylaxis or hypersensitivity to a monoclonal antibody
16. Have ever received a vaccine for SARS-CoV-2, as part of clinical trial or otherwise.
17. Receipt of any vaccine within 48 hours prior to enrolment. Vaccination will not be allowed for 4 weeks after dosing.
18. Receipt of convalescent plasma from a recovered COVID-19 patient or anti SARS-CoV-2 mAb within the last 3 months.
19. Participants who, in the judgment of the investigator, will be unlikely or unable to comply with the requirements of the protocol

### Example 39

### Phase 2/3 Clinical Study of Sotrovimab for Treatment of Mild to Moderate COVID-19 Disease

A randomized, multi-center, double-blind, placebo-controlled study is performed to assess safety and efficacy of sotrovimab (5309 N55Q LS) for the early treatment of COVID-19 in outpatients. Sotrovimab is an engineered monoclonal antibody (IgG1*01 G1m17; VH of SEQ ID NO.:113, M428L and N434S Fc mutations; VL of SEQ ID NO.:168 (kappa light chain IgKC*01 k1m3)).

A primary objective is to evaluate efficacy of sotrovimab versus placebo in preventing the progression of mild/moderate to severe or critical COVID-19 disease. Primary endpoints are the proportion of participants to develop severe disease, critical disease, or death, with a primary analysis up to Day 29.

Key secondary objectives are to determine safety and tolerability of sotrovimab compared to placebo, evaluate efficacy of sotrovimab versus placebo in preventing COVID-19 disease progression by days 8, 15, and 22, evaluate efficacy of sotrovimab versus placebo in preventing mortality, evaluate efficacy of sotrovimab versus placebo in shortening ventilator days, ICU length of stay (LOS), and total hospital LOS, evaluate efficacy of sotrovimab versus placebo in shortening the duration and decreasing the severity of COVID-19 clinical signs and symptoms, evaluate efficacy of sotrovimab versus placebo in preventing non-respiratory complications of COVID-19, evaluate efficacy of sotrovimab versus placebo in reducing duration of SARS-CoV-2 viral shedding, evaluate efficacy of sotrovimab versus placebo in inducing an anti-viral response, evaluate efficacy of sotrovimab versus placebo in facilitating a return to normal living, assess pharmacokinetics (PK) of sotrovimab in serum, and assess immunogenicity of sotrovimab.

Exploratory objectives are to evaluate efficacy of sotrovimab against versus placebo in preventing COVID-19 disease progression by days 8, 15, and 22, monitor on-treatment emergence of SARS-CoV-2 resistant mutants against sotrovimab, evaluate efficacy of sotrovimab versus placebo in reducing SARS-CoV-2 viral load, evaluate effect of sotrovimab versus placebo on potential biomarkers of host response to SARS-CoV-2, evaluate potential relationships between subject genetic polymorphisms and sotrovimab mechanisms of action and/or PK, and measure impact of sotrovimab treatment on time away from work and work productivity due to COVID-19 illness.

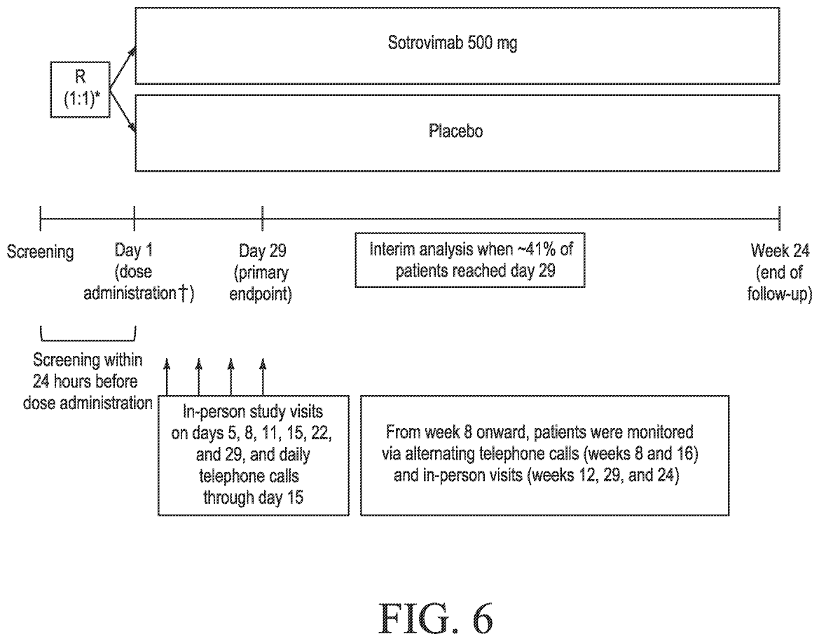

Further study details are shown in FIG. 6.

### Details of Criteria for Evaluation

Primary endpoints of this study are as follows:
Proportion of subjects who progress to severe or critical COVID-19 or die through Day 29 as defined by:
1. Severe disease: hypoxemia (O₂ saturation ≤93% on room air or PaO₂/FiO₂<300) requiring oxygen supplementation >1 day OR
subject requires ≥4 L/min oxygen supplementation or equivalent AND the investigator or designee determine that the measurement of 02 saturation on room air is medically unsafe. OR
2. Critical disease: Respiratory failure requiring at least one of the following: invasive mechanical ventilation, ECMO; OR shock; OR multi-organ dysfunction/failure OR
3. Death

Secondary endpoints of this study are as follows:
1. Occurrence of adverse events (AEs)
2. Occurrence of serious adverse events (SAEs)
3. Occurrence of adverse events of special interest (AESI)
4. Proportion of subjects who progress to develop severe or critical COVID-19 at Day 8, Day 15, or Day 22
5. 29-day, 60-day, and 90-day all-cause mortality
6. Total number of ventilator days from randomization through 29 days
7. Total intensive care length of stay (LOS)
8. Total hospital LOS
9. Severity and duration of subject reported signs and symptoms of COVID-19 related illness using the Flu-PRO patient-reported outcome measurement instrument
10. Occurrence of cardiac, thromboembolic, renal, neurologic events
11. Detection of SARS-CoV-2 in nasal secretions by PCR at baseline and during follow-up period through Day 29
12. Sotrovimab pharmacokinetics (PK) in serum
13. Incidence and titers (if applicable) of serum ADA to Sotrovimab

Exploratory endpoints of this study are as follows:
1. Proportion of subjects at Day 8, Day 15, Day 22, and Day 29 in each category of the ordinal scale for clinical improvement (see below)
2. Emergence of viral resistance mutants to mAb by SARS-CoV-2
3. Viral load in nasal secretions and blood by qRT-PCR
4. Host transcriptome and immunophenotyping analysis
5. FcTR polymorphisms as determined by genotyping
6. IgG1 allotypes as determined by genotyping
7. Change from baseline in Work Productivity and Activity Impairment (WPAI)
8. Change from baseline in health-related quality of life according to EQ-5D-5L

The ordinal scale for use in categorizing clinical improvement is as follows:
1. Uninfected: No clinical or virologic evidence of infection
2. Non-hospitalized, no limitation of activity (if using oxygen at home, report as category 4)
3. Non-hospitalized, limitation of activity
4. Hospitalized, no oxygen therapy
5. Hospitalized, low-flow oxygen by mask or nasal prongs
6. Hospitalized, high-flow oxygen, Continuous positive airway pressure (CPAP), bilevel positive airway pressure (BiPAP), non-invasive ventilation
7. Hospitalized, intubation and mechanical ventilation
8. Hospitalized, mechanical ventilation plus organ support (vasopressors, RRT, ECMO)
9. Death

### Number of Subjects

This study is comprised of two parts. The lead-in phase enrolls approximately 20 subjects with early, mild to moderate COVID-19 who are at high risk for progression of disease. The expansion phase enrolls approximately 850 subjects with early, mild to moderate COVID-19 who are at high risk for progression of disease.

### Diagnosis and Main Criteria for Inclusion

Inclusion criteria include:

### Age:

1. Subjects aged 18 years and older at high risk of complications from COVID-19 disease including diabetes, obesity (BMI>30), chronic kidney disease, congestive heart failure, chronic obstructive pulmonary disease, and moderate to severe asthma; AND
2. Subjects ≥55 years old, irrespective of comorbidities

### Disease Characteristics:

1. Subjects who have a positive SARS-CoV-2 test result (by any validated test e.g. RT-PCR on any specimen type); AND
2. Have an oxygen saturation ≥94% on room air; AND
1. Have mild to moderate or moderate uncomplicated COVID-19 illness defined by one or more of the following symptoms:
fever, chills, cough, sore throat, malaise, headache, joint or muscle pain, change in smell or taste, vomiting, diarrhea, shortness of breath on exertion; AND
3. Are less than or equal to 4 days from onset of symptoms

### Sex:

1. No gender restriction
2. Female subjects must meet and agree to abide by the following contraceptive criteria. Contraception use by women should be consistent with local regulations regarding the methods of contraception for those participating in clinical studies.
3. A female subject is eligible to participate if she is not pregnant or breastfeeding, and one of the following conditions applies:
Is a woman of non-childbearing potential (WONCBP); OR
Is a woman of child-bearing potential (WOCBP) and using a contraceptive method that is highly effective, with a failure rate of <1% during the study intervention period and for up to one year after the last dose of study intervention. The investigator should evaluate potential for contraceptive method failure (e.g., noncompliance, recently initiated) in relationship to the first dose of study intervention.
4. A WOCBP must have a negative highly sensitive pregnancy test (urine or serum as required by local regulations) at hospital admission or before the first dose of study intervention. If a urine test cannot be confirmed as negative (e.g., an ambiguous result), a serum pregnancy test is required. In such cases, the subject must be excluded from participation if the serum pregnancy result is positive.

### Informed Consent:

5. Willing to comply with the study requirements and capable of giving signed informed consent or, if not able to give signed informed consent, if alternative consent procedures are able to be followed.
   Exclusion criteria:

### Medical Conditions

Currently hospitalized or judged by the investigator as likely to require hospitalization in the next 24 hours
Symptoms consistent with severe COVID-19 as defined by shortness of breath at rest or respiratory distress or requiring supplemental oxygen
Patients who, in the judgment of the investigator, are likely to die in the next 7 days
Severely immunocompromised patients, including but not limited to cancer patients actively receiving immunosuppressive chemotherapy or immunotherapy, those with a solid organ transplant or allogeneic stem cell transplant within the last 3 months, or those having conditions requiring the use of systemic corticosteroids equivalent to ≥0.5 mg/kg of body weight per day of prednisone within 6 weeks of randomization
Known hypersensitivity to any constituent present in the investigational product
Previous anaphylaxis or hypersensitivity to a monoclonal antibody

### Prior/Concurrent Clinical Study Experience

Enrollment in any investigational vaccine study within the last 180 days or any other investigational drug study within 30 days prior to day 1 or within five half-lives of the investigational compound, whichever is longer
Enrollment in any trial of an investigational vaccine for SARS-CoV-2

### Other Exclusions

Receipt of any vaccine within 48 hours prior to enrollment. Receipt of a SARS-CoV-2 vaccine prior to randomization at any time point. Vaccination (including vaccination for SARS-CoV-2) will not be allowed for 4 weeks after dosing
Receipt of convalescent plasma from a recovered COVID-19 patient or anti-SARS-CoV-2 monoclonal antibody within the last 3 months
Patients who, in the judgment of the investigator, will be unlikely or unable to comply with the requirements of the protocol through day 29

### Duration of Study Participation and Follow Up

The duration of study drug treatment is a single dose. The estimated total time on study, inclusive of screening and follow-up, for each subject is approximately nine months. All subjects are monitored through at least two hours post-dose prior to discharge from the study unit. Subjects enrolled in the lead-in portion of the study are monitored in an in-patient setting for a minimum of seven days. Subjects are subsequently actively monitored on an outpatient basis with in-person study visits at Weeks 1, 2, 3, and 4 and daily telephone calls on non-study visit days through Day 14. After Day 29, patients are monitored monthly via remote telehealth or phone call for a total of nine months from dosing.

### Study Design

An Independent Data Monitoring Committee (IDMC) actively monitors interim unblinded safety data (Lead-in Phase) and interim unblinded safety and efficacy data (Expansion Phase) to make recommendations regarding ongoing study conduct. The IDMC members include physicians with relevant medical specialist training and one statistician. The IDMC reviews unblinded safety data from the Lead-in Phase of the study prior to initiation of the Expansion Phase. In addition, the IDMC performs regular safety reviews during the Expansion Phase. The first safety review includes available safety and tolerability data though Day 14 from a total of 60 patients (30 per arm). If there are no safety or tolerability concerns according to pre-specified criteria, the IDMC recommends initiation of a separate study in hospitalized patients with severe to critical COVID-19. If necessary, additional safety reviews are conducted after each additional approximately 100 patients are enrolled (50 per arm). Finally, two interim analyses are conducted at approximately 33% of subjects enrolled (145 per arm) and approximately 67% of subjects enrolled (290 per arm) to evaluate safety, futility, and efficacy. A Joint Safety Review Team (JSRT) determines if a safety concern identified during the instream blinded data review needs to be escalated to the IDMC.

This study is a randomized, double-blind, multi-center, placebo-controlled trial of sotrovimab, a monoclonal antibody (mAb) against SARS-CoV-2 for the prevention of progression of mild to moderate COVID-19 disease in high-risk subjects, with interim monitoring to allow early stopping for futility, efficacy, or safety. Subjects with early, mild to moderate COVID-19 who are at high-risk for progression of disease are randomized 1:1 to receive a single, intravenous infusion of either sotrovimab or equal volume saline placebo. Comparisons of safety and efficacy are based on data from concurrently randomized participants.

The study is comprised of 2 parts. The lead-in phase enrolls 20 subjects who have early, mild to moderate COVID-19 and are at high risk of disease progression. Following a safety assessment of unblinded data by an independent data monitoring committee (IDMC), the expansion phase progresses, where additional subjects with early, mild to moderate COVID-19 and who are at high risk of disease progression are enrolled.

### Lead-In Phase:

The Lead-In Phase of the study evaluates the safety and tolerability of sotrovimab in subjects with early, mild to moderate COVID-19 who are at high-risk of progression to severe disease. Subjects are monitored in an in-patient setting for seven days including assessments of respiratory status, oxygenation and other vital signs and laboratory evaluations. A single dose level is studied and is delivered via intravenous infusion. Subjects are admitted to a study unit and monitored closely for adverse events, changes in laboratory parameters and progression or improvement in disease signs and symptoms.

The first two eligible subjects enrolled are randomized 1:1 to sotrovimab or placebo. These sentinel subjects are dosed and monitored for at least 48 hours in an in-patient setting. During dose administration, vital signs are monitored every 15 minutes over the one hour IV infusion. Vital signs are also monitored every one hour after infusion for two hours. Vital signs, ECG, symptom-directed physical examinations(s), and adverse events (AEs) are reviewed by the investigator. If the investigator has no immediate safety concerns, the remainder of the subjects in the Lead-In phase is dosed (total of ten per arm inclusive of the sentinel subjects).

All 20 subjects enrolled in the Lead-In (N=10 per arm; sotrovimab or placebo) are assigned to an intensive PK and immunogenicity substudy. Sparse samples are collected from all participants not enrolled in the substudy. Serum PK and anti-drug antibodies (ADA) samples are collected.

### Expansion Phase:

The Expansion Phase of the study progress following assessment of available unblinded safety data from the Lead-in Phase (N=10 per arm through 14 days of follow-up) by an IDMC. The purpose of the Expansion Phase is to evaluate the safety and efficacy of sotrovimab in comparison to the placebo control arm. Subjects with early mild to moderate COVID-19 who are at risk for progression to severe disease are randomized in a 1:1 ratio (435 per arm) to receive a single, IV infusion of sotrovimab or placebo.

Subjects are monitored through at least 2 hours post-dose prior to discharge from the study unit. Subjects are subsequently actively monitored on an outpatient basis with in-person study visits at Weeks 1, 2, 3, and 4 and daily telephone calls on non-study visit days through Day 14 for AEs and worsening of illness. In addition, the subjects are provided a device to monitor for hypoxemia.

After Day 29, patients are monitored monthly via remote telehealth or phone call to assess for the incidence and of severity of subsequent COVID-19 illness, if any, for a total of 9 months from dosing.

Two interim analyses at approximately 33% of subjects enrolled (145 per arm) and approximately 67% of subjects enrolled (290 per arm) are conducted to evaluate futility, efficacy, and safety, as well as allow for sample size reestimation if the overall disease progression rate is lower than expected.

A placebo control distinguishes safety and tolerability of sotrovimab from the background signs and symptoms of COVID-19 and evaluates its potential benefit on clinical outcomes. The use of a placebo arm allows for a valid evaluation of any changes in efficacy and safety attributable to sotrovimab versus those attributable to background supportive care given during the study.

A primary endpoint to assess efficacy of treatment is progression to severe disease defined as hypoxia requiring oxygen supplementation, or the development of critical disease (respiratory failure requiring at least one of the following: invasive mechanical ventilation, ECMO; OR shock; OR multi-organ dysfunction/failure), or death within 29 days of randomization. The transition from outpatient status without hypoxia or oxygen requirement to hypoxia with a need for oxygen therapy or higher levels of respiratory/organ support is a clinically significant milestone.

A significant secondary endpoints is all-cause mortality at Day 29. Other secondary efficacy endpoints related to hospital stay—length of hospital stay, ICU stay, time ventilated and the severity and duration of subject-reported signs and symptoms of COVID-19 are also clinically relevant.

### Immunology Assessment Substudy:

To explore the host immune response and potential biomarkers of infection, subjects may consent to an optional sub-study in which peripheral blood mononuclear cells (PBMCs) are collected at specified timepoints. This optional substudy is performed at selected sites. Approximately 20-50 participants per arm are enrolled in the optional sub-study. Subjects from both the Lead-In and Expansion Phases of the study may be included in the Immunology Assessment Substudy.

### Study Intervention Groups and Duration

Screening assessments are performed within 24 hours before the first dose. Eligible subjects are treated in a blinded manner with a single IV dose on Day 1 and followed up to 9 months (36 weeks).

In the Lead-In Phase, 20 subjects with mild to moderate COVID-19 are randomized 1:1 to receive a single IV dose of sotrovimab or placebo. Subjects enrolled in the Lead-in Phase of the study are closely monitored in an in-patient setting for a minimum of 7 days. At the end of 7 days they are either discharged to home, remain in the inpatient unit or are formally hospitalized based on investigator assessment of clinical status.

In the Expansion Phase, subjects with mild to moderate COVID-19 are randomized 1:1 to receive a single IV dose of sotrovimab or placebo.

Participants in the Expansion phase are stratified by the following criteria:
1. Duration of symptoms: ≤2 days vs. 3-4 days
2. Age: ≤70 vs. >70 years old

### Study Interventions

Study interventions and dosing instructions are as follows in Table 7:

TABLE 7

| | | |
|---|---|---|
| Dose Formulation | Solution in single-use vial (25 mg/mL) | Sterile 0.9% (w/v) sodium |
| | | chloride solution |
| Unit Dose Strength(s) | 250 mg/vial (250 mg/10 mL) | Not applicable |
| Dosage Level(s) | nn mg once | Once |
| Route of Administration | IV infusion | IV infusion |
| Dosing instructions | Withdraw 10.0 mL from the vial into a 10-20 mL | Dose per IV infusion |
| | small syringe and transfer to a 150 mL or 250 mL | instructions |
| | IV empty saline bag by an unblinded pharmacist. | |
| | Repeat this procedure until the required dose is | |
| | reached and administer over 1 hour. | |
| Special instructions | Gently mix sotrovimab and saline prior to IV | None |
| | infusion. | |
| Packaging and Labelling | Study intervention is provided in a single-use | Labelled as required per |
| | vial in an individual carton and labelled as | country requirement |
| | required per country requirement. | |

Dose selection is guided by live virus neutralization data, resistance data, human PK projections informed by non-human primate PK and the no observed adverse effect level (NOAEL) in a cynomolgus monkey.

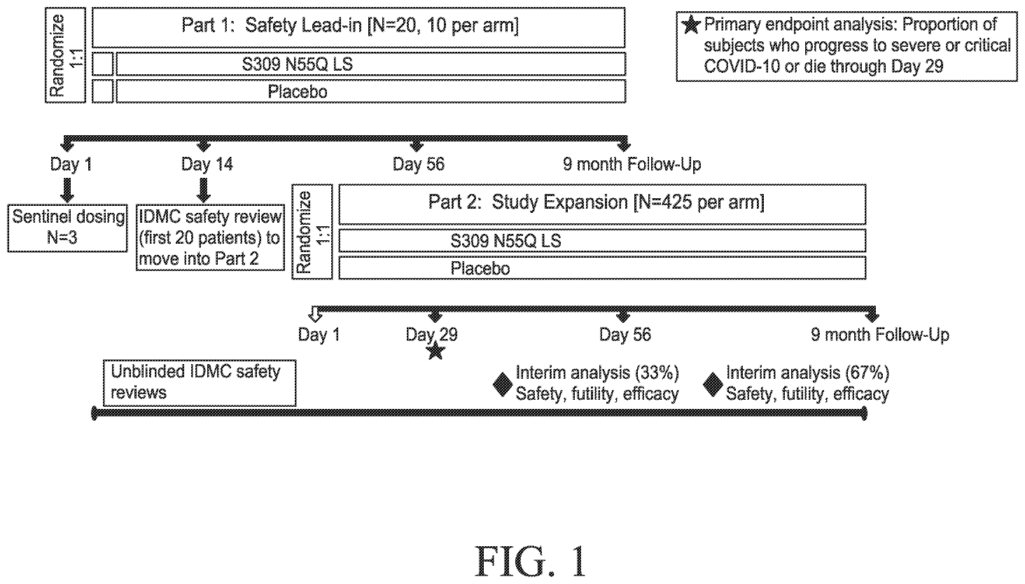

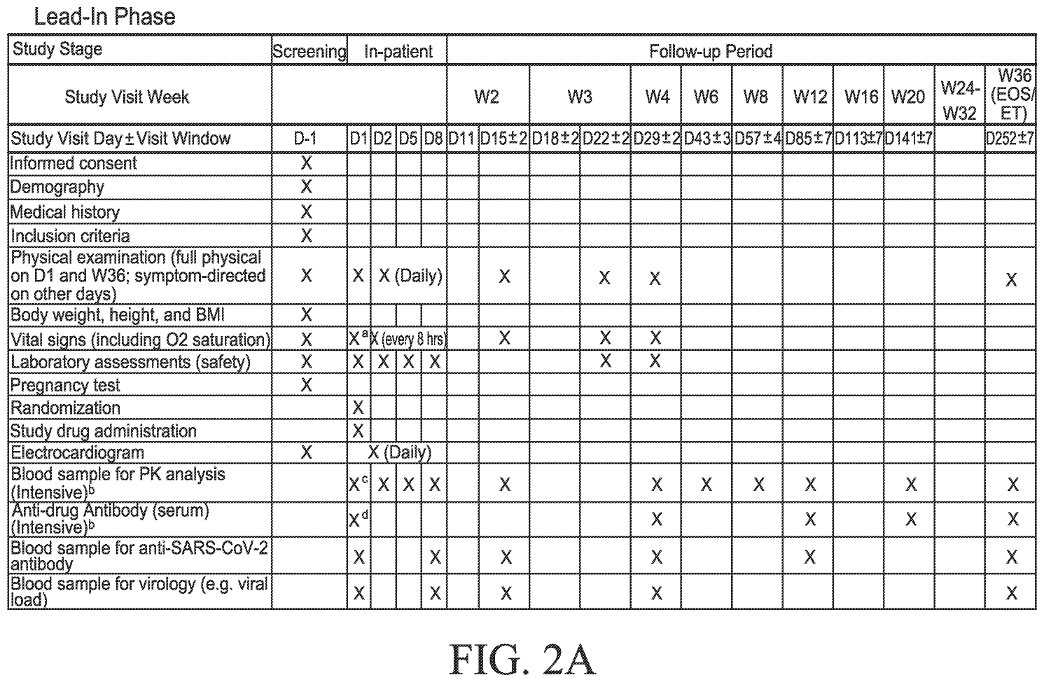

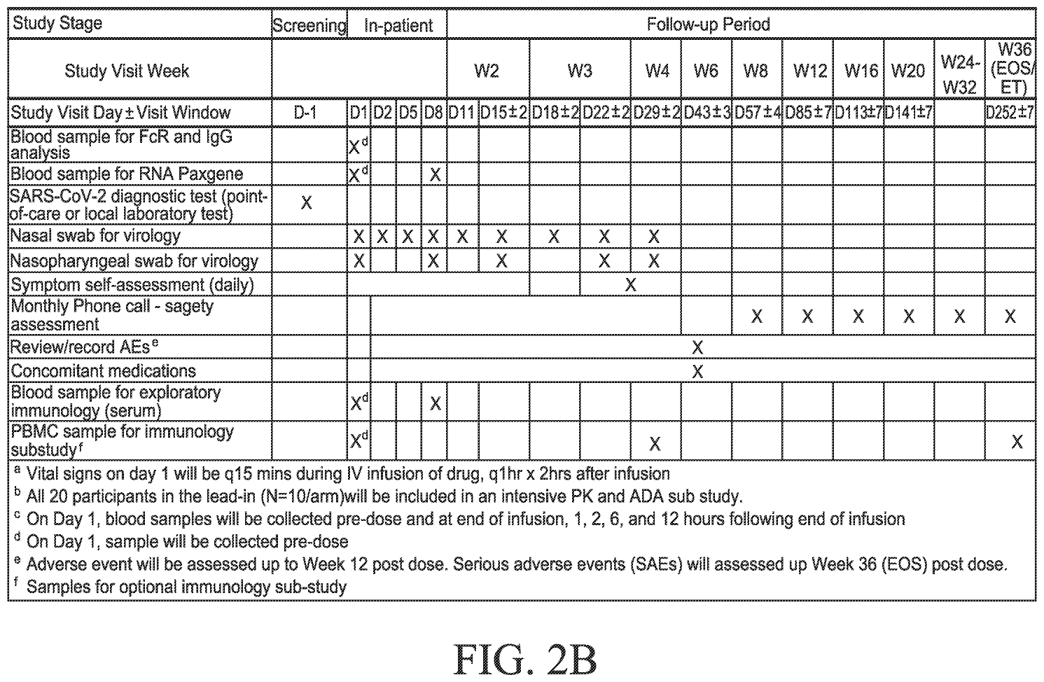

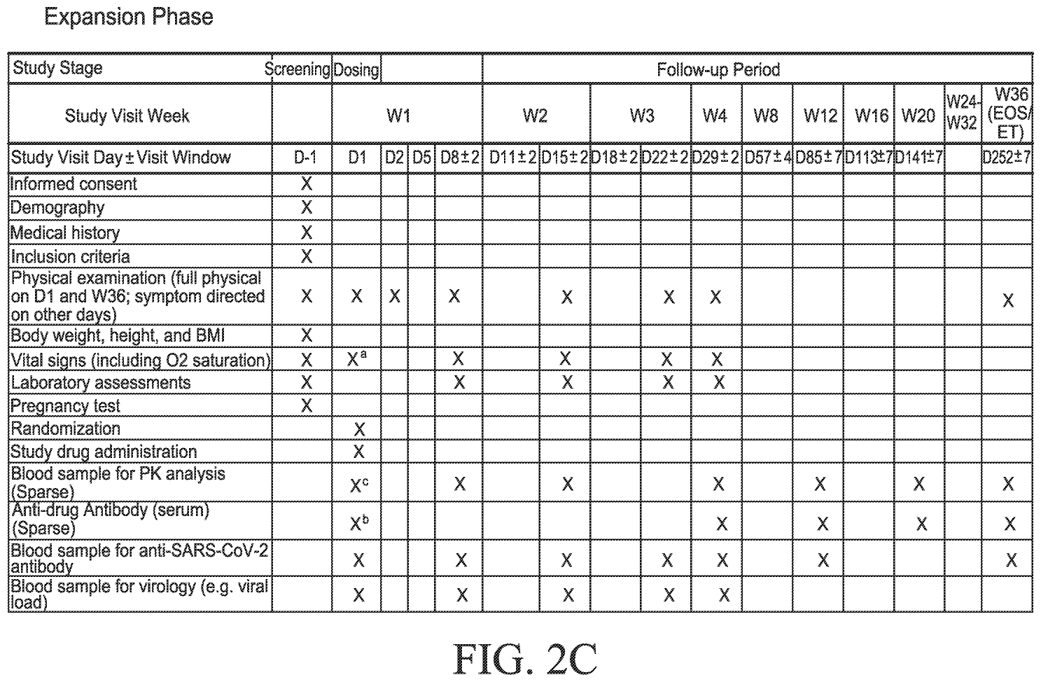

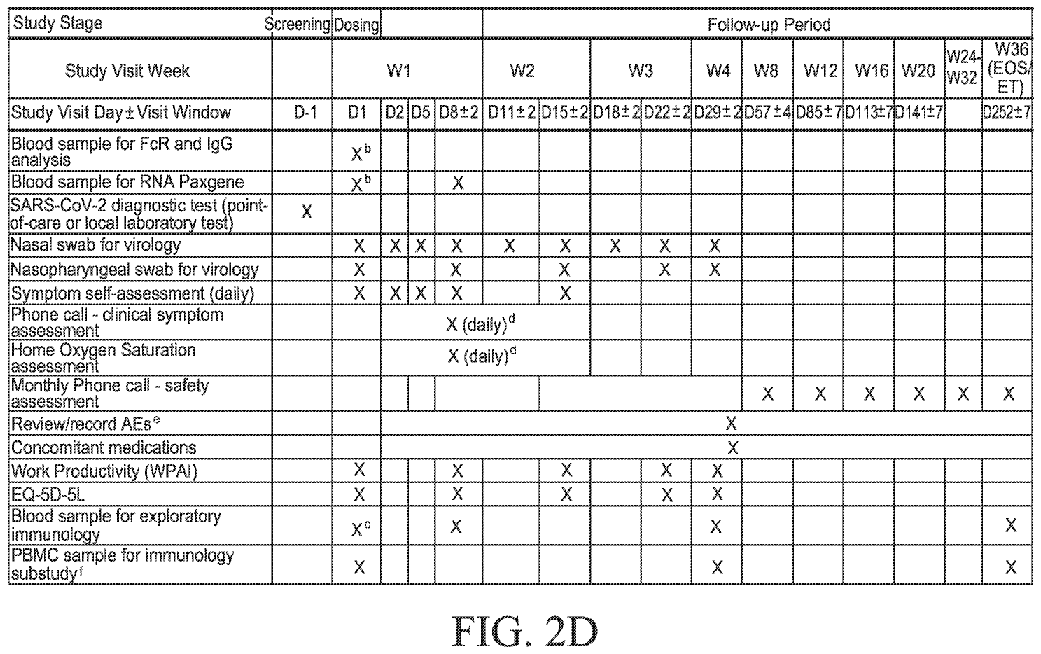

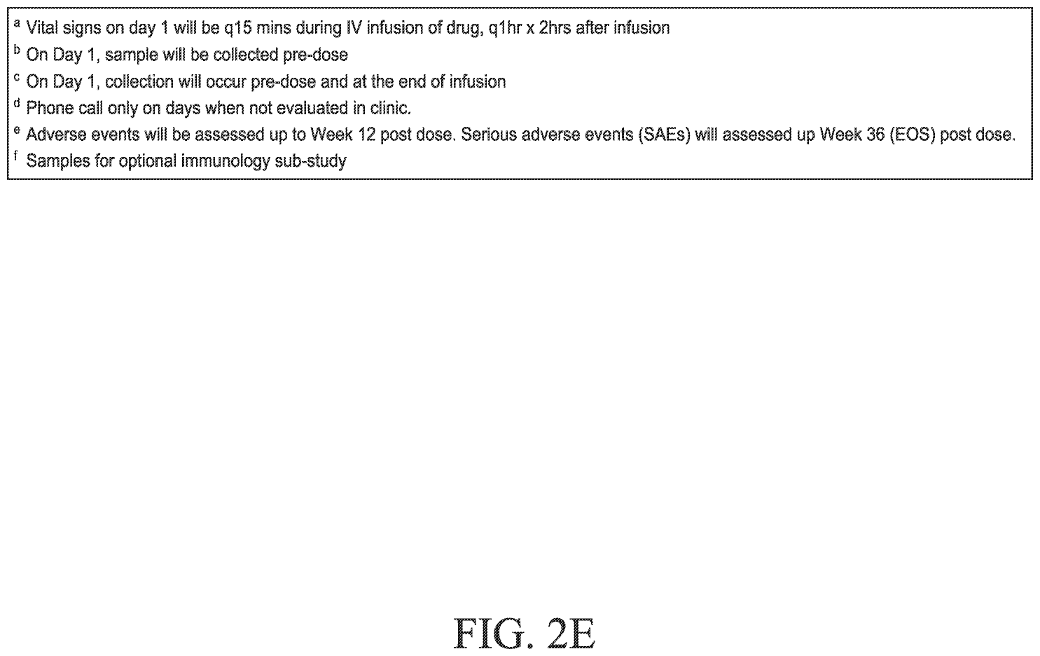

Further study details are shown in FIGS. 1 and 2A-E.

### Example 40

### Sotrovimab Monotherapy Reduces Hospitalization and Risk of Death in Early Treatment of Adults with Covid-19

In the Phase 3 portion of the clinical trial described in Example 39, safety and efficacy of a single intravenous infusion of sotrovimab (500 mg) or placebo was assessed in 583 non-hospitalized participants globally (291 patients in the treatment arm and 292 patients in the placebo arm). The primary efficacy endpoint is the proportion of patients who have progression of COVID-19 as defined by the need for hospitalization for at least 24 hours or death within 29 days of randomization. Among those studied, 63% were Hispanic or Latinx and 7% were Black or African-American. According to the Centers for Disease Control and Prevention, these populations are approximately three times more likely to be hospitalized and approximately two times more likely to die of COVID-19 (Data source: COVID-NET (www.cdc.gov/coronavirus/2019-ncov/covid-data/covid-net/purpose-methods.html, accessed Mar. 1, 2020, through Jan. 30, 2021); Numbers are ratios of age-adjusted rates standardized to the 2019 US standard COVID-NET catchment population; Data source: NCHS provisional death counts (data.cdc.gov/NCHS/Deaths-involving-coronavirus-disease-2019-COVID-19/ks3g-spdg, data through Jan. 30, 2021). Numbers are ratios of age-adjusted rates standardized to the 2019 US intercensal population estimate).

Interim analysis of data from the 583 patients enrolled in the trial demonstrated an 85% (p=0.002) reduction in hospitalization or death in patients sotrovimab as monotherapy compared to placebo, the primary endpoint of the trial. Sotrovimab was well tolerated. The trial remains ongoing and blinded with patients continuing to be followed for 24 weeks; additional results, including epidemiology and virology data, are collected.

This study and results are described in further detail:
Methods: In a multicenter, double-blind, phase 3 trial, nonhospitalized patients with symptomatic COVID-19 and at least one risk factor for disease progression were randomized (1:1) to an intravenous infusion of sotrovimab 500 mg or placebo. The primary efficacy endpoint was the proportion of patients with COVID-19 progression, defined as hospitalization longer than 24 hours or death, through day 29.
Results: In this preplanned interim analysis, which included an intent-to-treat population of 583 patients (sotrovimab, 291; placebo, 292), the primary efficacy endpoint was met. The risk of COVID-19 progression was significantly reduced by 85% (97.24% confidence interval, 44% to 96%; P=0.002) with a total of three (1%) patients progressing to the primary endpoint in the sotrovimab group versus 21 (7%) patients in the placebo group. All five patients admitted to intensive care, including one who died by day 29, received placebo. Safety was assessed in 868 patients (sotrovimab, 430; placebo, 438). Adverse events were reported by 17% and 19% of patients receiving sotrovimab and placebo, respectively; serious adverse events were less common with sotrovimab (2%) versus placebo (6%).
In the intention-to-treat analysis (N=1057, full dataset), the adjusted relative risk reduction of hospitalization (all causes) was 79% (95% CI, 9% to 50%; p<0.001) through Day 29 in recipients of sotrovimab (n=523) vs. placebo (n=526). Treatment with sotrovimab (ITT) resulted in a numerical reduction in the need for ER visits for illness management, hospitalisation for acute illness management (any duration) or death (any cause) compared to placebo. No participants on sotrovimab required ICU admission, compared to 9 participants on placebo, of whom 4 participants required mechanical ventilation. No participants who received sotrovimab died, compared to 4 participants on placebo. The incidence of adverse events was similar between treatment arms and SAEs were numerically more common in the placebo arm.
Conclusion: Sotrovimab reduced progression of COVID-19 in patients with mild/moderate disease, was well tolerated, and no safety signals were identified.

### Methods

### Trial Objectives and Oversight

This phase 3, randomized, double-blind, multicenter, placebo-controlled study evaluates a single intravenous infusion of sotrovimab 500 mg for the prevention of progression of mild/moderate COVID-19 in high-risk, nonhospitalized patients. For this preplanned interim analysis, patients were recruited beginning on Aug. 27, 2020 and followed through Mar. 4, 2021 at 37 study sites in four countries (United States, Canada, Brazil, and Spain). Any changes made to the protocol and statistical analysis plan after the trial started are summarized in the Supplementary Appendix of this Example.

This study is conducted in accordance with the principles of the Declaration of Helsinki and Council for International Organizations of Medical Sciences International Ethical Guidelines, applicable International Council for Harmonisation Good Clinical Practice guidelines, and applicable laws and regulations. All patients provided written informed consent.

### Patients and Procedures

Adult patients 18 years of age or older with a positive reverse-transcriptase-polymerase-chain-reaction or antigen SARS-CoV-2 test result and onset of symptoms within the prior 5 days were screened for eligibility; screening was performed within 24 hours before study drug administration. Patients were required to be at high risk for COVID-19 progression, defined as older adults (age >55 years) or adults with at least one of the following risk factors: diabetes requiring medication, obesity (body-mass index >30 kg/m²), chronic kidney disease (estimated glomerular filtration rate <60 mL/min/1.73 m²),²³ congestive heart failure (New York Heart Association class II or higher), chronic obstructive pulmonary disease, and moderate to severe asthma.²⁴

Patients with already severe COVID-19, defined by shortness of breath at rest, respiratory distress, or requiring supplemental oxygen, were excluded. Inclusion criteria are described in the Supplementary Appendix of this Example.

Eligible patients were randomized 1:1 using an interactive web response system to receive either a single 500-mg, 1-hour infusion of sotrovimab or equal volume saline placebo on day.

### Efficacy Assessments

The primary endpoint was the proportion of patients with hospitalization for more than 24 hours or death, due to any cause, through day 29. Secondary efficacy endpoints included the proportion of patients with an emergency room visit, hospitalization, or death; mortality; patient-reported outcomes; changes in viral load; and the proportion of patients who progressed to require supplemental oxygen.

### Safety Assessments

Safety endpoints included adverse events, serious adverse events, and adverse events of special interest, defined as infusion-related reactions (including hypersensitivity reactions), immunogenicity testing for anti-drug antibodies, and evaluation of antibody-dependent enhancement. All hospitalizations, including those due to COVID-19, were counted as serious adverse events.

### Statistical Analyses

A preplanned interim analysis for safety, futility, and profound efficacy was triggered when approximately 41% of the required number of study patients reached day 29. Sample size calculations were based on a group sequential design with two interim analyses to assess both futility due to lack of efficacy or profound efficacy. A Lan-DeMets alpha spending function to control type I error was used, employing a Pocock analog rule for futility and a Hwang-Shih-DeCani (with parameter γ=1) analog for efficacy.²⁵ The overall sample size of 1360 would have provided approximately 90% power to detect a 37.5% relative efficacy in reducing progression of COVID-19 through day 29 at the overall two-sided 5% significance level, with an assumed progression rate of 16% in the placebo group.

The interim analysis intent-to-treat (ITT) population included all randomized patients through the prespecified interim analysis cutoff date of Jan. 19, 2021, irrespective of whether they received study drug. The interim analysis safety analysis population included all patients who received study medication and were randomized through Feb. 17, 2021; patients were grouped according to the actual treatment received. The primary endpoint was analyzed in the ITT population using a Poisson regression model with robust sandwich estimators adjusting for treatment, duration of symptoms, age, and gender. Missing progression status was imputed under a missing at random assumption, using multiple imputation. Based on this analysis model, the statistical significance testing, the relative risk of progression, and its appropriate confidence interval (CI) are provided using the adjusted significance level for this interim analysis.

As a result of the observed efficacy, an independent data monitoring committee recommended that enrollment in the study be stopped on Mar. 10, 2021, at which time 1057 patients had been randomized. Analyses of all secondary and exploratory endpoints is conducted when all patients have completed day 29.

### Results

### Patients

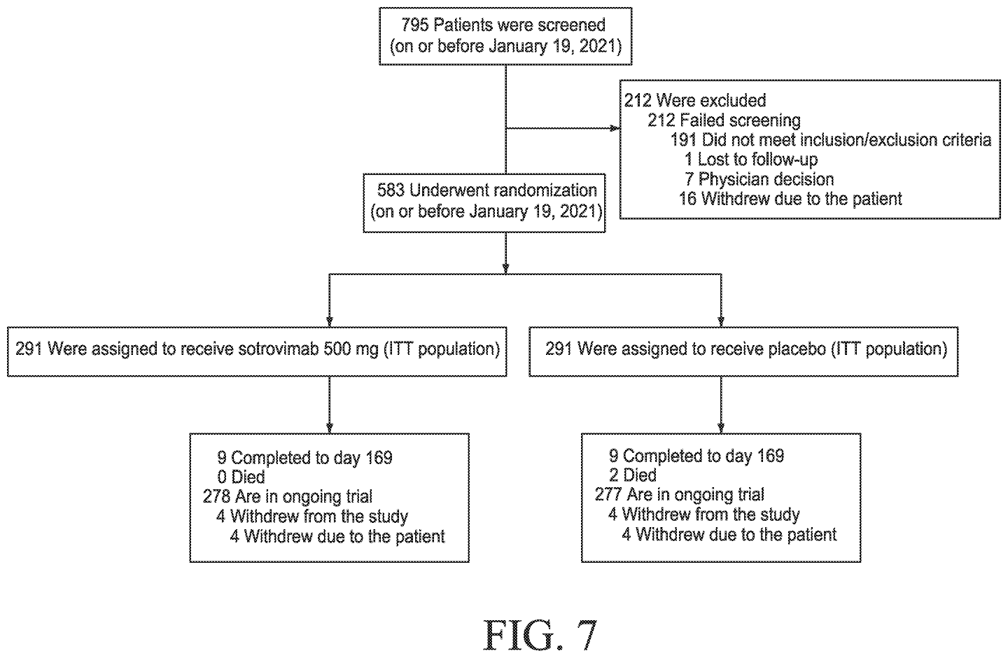

Of 795 patients screened, 583 were randomized through Jan. 19, 2021 to sotrovimab (291 patients) or placebo (292 patients); these patients comprise the interim analysis ITT population (FIG. 7). In this ITT population, similar disposition was observed across treatment groups. Overall, four patients each in the sotrovimab and placebo groups withdrew from the study. Three patients in the sotrovimab group withdrew prior to dosing, with a fourth patient withdrawing consent on day 5 for personal reasons. One patient in the placebo group withdrew consent prior to dosing, and three patients withdrew after dosing for personal reasons (days 16, 25, and 85). The median duration of follow-up in the ITT population was 72 days (range, 5 to 190) for the sotrovimab group and 72 days (range, 16 to 190) for the placebo group.

Overall, 868 patients (sotrovimab, 430 patients; placebo, 438 patients) were randomized and received study drug through Feb. 17, 2021; these patients comprise the interim analysis safety analysis population. The median duration of follow-up in this population was 56 days (range, 5 to 190) for the sotrovimab group and 55 days (range, 2 to 190) for the placebo group.

Treatment groups in the ITT population were well balanced for baseline demographic and disease characteristics (Table 8). Overall, 22% of patients were greater than 65 years of age, 7% were Black or African American, 63% were Hispanic or Latino, and 42% had two or more conditions considered to be risk factors for COVID-19 progression. The most common risk factors were obesity, age 55 years or older, and diabetes requiring medication. The most common presenting symptoms (>60% of all patients) were cough, muscle aches/myalgia, headache, and fatigue (Table 11, in the Supplementary Appendix of this Example). Baseline demographic and disease characteristics in the safety analysis population were similar across treatment groups and are reported in Table 12, in the Supplementary Appendix of this Example.

### Efficacy Outcomes

Treatment with sotrovimab resulted in an 85% reduction in the need for hospitalization over 24 hours or death, due to any cause, compared with placebo (relative risk, 0.15 [97.24% CI, 0.04 to 0.56]). Three (1%) patients progressed to the primary endpoint in the sotrovimab group versus 21 (7%) patients in the placebo group (P=0.002) (Table 9). The primary reasons for the 24 hospitalizations of more than 24 hours were consistent with progressive COVID-19 (Table 13, in the Supplementary Appendix of this Example), with one likely exception: a patient in the sotrovimab group, with a notable past medical history of small intestinal obstruction, presented 22 days after infusion with a small intestinal obstruction. Regarding the severity of these hospitalizations, all five patients who required admission to intensive care were in the placebo group; two of these five patients required invasive mechanical ventilation and a third declined intubation and subsequently died by day 29. Emergency room visits or hospitalizations for less than 24 hours were observed in fewer patients in the sotrovimab group compared with the placebo group (Table 9).

### Safety

The proportion of patients in the safety analysis population who reported an adverse event was 17% (73 of 430 patients) in the sotrovimab group and 19% (85 of 438 patients) in the placebo group (Table 10). A lower proportion of patients reported grade 3 or 4 adverse events in the sotrovimab group (2%) compared with the placebo group (6%). Overall, the only adverse event occurring in at least 1% of patients receiving sotrovimab was diarrhea, which occurred infrequently-six (1%) patients in the sotrovimab group versus three (<1%) patients in the placebo group. Among patients in the sotrovimab group, all cases of diarrhea were mild (five patients) or moderate (one patient) in severity.

Infusion-related reactions were observed in a similar proportion of patients receiving sotrovimab (1%) compared with placebo (1%). One patient receiving sotrovimab had an infusion-related reaction that was considered related to study treatment: moderate (grade 2) dyspnea.

Serious adverse events occurred in 2% of patients receiving sotrovimab and 6% of patients receiving placebo. Most of these events were hospitalizations due to COVID-19-related causes. No serious adverse events were considered related to sotrovimab.

One patient in the placebo group died after day 29; this patient died due to COVID-19 pneumonia on day 37.

No trends were observed in hematologic, liver, or chemistry laboratory data. Overall, laboratory results were similar in the sotrovimab and placebo groups.

### Discussion

In this preplanned interim analysis of the COMET-ICE study, a single 500-mg dose of sotrovimab reduced the risk of hospitalization (>24 hours) or death in high-risk adults with symptomatic COVID-19 by 85% compared with placebo (P=0.002). For every 17 high-risk patients with symptomatic COVID-19, sotrovimab prevented one hospitalization. Among those who were hospitalized, no patient who received sotrovimab required admission to intensive care compared with five patients who received placebo, suggesting that sotrovimab may also prevent more severe complications of COVID-19 in addition to preventing the need for hospitalization itself. The full analysis provided similar results. Furthermore, as a result of investigator site selection, over 60% of the study population consisted of patients self-identifying as Hispanic or Latino; thus, this trial is one of the first to demonstrate efficacy in a population that has been largely underrepresented in COVID-19 clinical trials, despite the disproportionately negative impact the pandemic has had in this ethnic group. Overall, sotrovimab was well tolerated, and no safety signals were identified in this study. There was also no evidence of antibody-dependent enhancement with sotrovimab, which could have manifested as worsening of disease compared with placebo.

Treatments for COVID-19 will need to retain activity in the face of an evolving virus. To that end, sotrovimab may have an intrinsically high barrier to resistance as a result of targeting a pan-sarbecovirus epitope. In one analysis, among more than 584,000 sequences in the Global Influenza Surveillance and Response System database (Global Initiative on Sharing All Influenza Data), amino acid positions comprising the sotrovimab epitope were at least 99.96% conserved in naturally occurring viruses. Moreover, when necessary to further enhance breadth and barrier to resistance, sotrovimab can likely be combined with currently authorized receptor-binding motif-targeted antibodies due to its nonoverlapping resistance profile. These results also indicate that a non-receptor-binding motif binding antibody, which does not directly block the ACE2 receptor interaction, can be clinically therapeutic, and suggests a role for other receptors. Moreover, as sotrovimab has potent effector function, the absence of safety signals and observed efficacy strongly suggest that effector function is neither detrimental nor associated with antibody-dependent enhancement. In fact, preclinical models of COVID-19 suggest that its potent effector function may be beneficial.

Results from this interim analysis (as well as from the full intent-to-treat analysis) of the COMET-ICE trial support that sotrovimab can be an important therapeutic for the outpatient treatment of COVID-19. Notably, a 500-mg dose may be administered intramuscularly, increasing the convenience of and access to antibody therapeutics for patients with COVID-19. Studies are currently underway to evaluate this route of administration.

TABLE 8
Baseline Demographic and Disease Characteristics (ITT Population)

| | Sotrovimab | | Placebo | | Total | |
|---|---|---|---|---|---|---|
| Characteristic | (N = 291) | | (N = 292) | | (N = 583) | |
| Age - yr, median (range) | 53.0 | (18-96) | 52.5 | (18-88) | 53.0 | (18-96) |
| ≥65 yr - no. (%) | 63 | (22) | 65 | (22) | 128 | (22) |
| >70 yr - no. (%) | 33 | (11) | 32 | (11) | 65 | (11) |
| Male gender - no. (%) | 135 | (46) | 131 | (45) | 266 | (46) |
| Race* - no. (%) | | | | | | |
| White | 254 | (88) | 252 | (87) | 506 | (87) |
| Black or African American | 16 | (6) | 22 | (8) | 38 | (7) |
| Asian | 17 | (6) | 17 | (6) | 34 | (6) |
| Mixed race | 2 | (<1) | 0 | | 2 | (<1) |
| American Indian or Alaska Native | 1 | (<1) | 0 | | 1 | (<1) |
| Hispanic or Latino ethnic group - no. (%) | 190 | (65) | 178 | (61) | 368 | (63) |
| Body-mass index† - mean (SD) | 32.0 | (6.4) | 32.1 | (6.3) | 32.1 | (6.3) |
| Duration of symptoms‡ - no. (%) | | | | | | |
| ≤3 days | 167 | (57) | 171 | (59) | 338 | (58) |
| 4-5 days | 123 | (42) | 121 | (41) | 244 | (42) |
| Any risk factor for COVID-19 progression - no. (%) | 291 | (100) | 290 | (>99) | 581 | (>99) |
| Age ≥55 yr | 135 | (46) | 141 | (48) | 276 | (47) |
| Diabetes requiring medication | 66 | (23) | 66 | (23) | 132 | (23) |
| Obesity (body-mass index >30†) | 182 | (63) | 187 | (64) | 369 | (63) |
| Chronic kidney disease (eGFR <60 by MDRD) | 1 | (<1) | 4 | (1) | 5 | (<1) |
| Congestive heart failure (NYHA class II or more) | 1 | (<1) | 3 | (1) | 4 | (<1) |
| Chronic obstructive pulmonary disease | 14 | (5) | 10 | (3) | 24 | (4) |
| Moderate to severe asthma | 46 | (16) | 46 | (16) | 92 | (16) |
| Number of concurrent risk factors | | | | | | |
| for COVID-19 progression - no. (%) | | | | | | |
| 0 | 0 | | 2 | (<1) | 2 | (<1) |
| 1 | 170 | (58) | 168 | (58) | 338 | (58) |
| 2 | 91 | (31) | 86 | (29) | 177 | (30) |
| ≥3 | 30 | (10) | 36 | (12) | 66 | (11) |

ITT denotes intent-to-treat, SD standard deviation, eGFR estimated glomerular filtration rate, MDRD Modification of Diet in Renal Disease, NYHA New York Heart Association.
*Race data were not available for one patient in the sotrovimab group and one patient in the placebo group.
†Body-mass index is the weight in kilograms divided by the square of the height in meters.
‡One patient in the sotrovimab group had a symptom duration of 6 days.

TABLE 9
Summary of Efficacy Outcomes Through Day 29 (ITT Population)

| | Sotrovimab | | Placebo | |
|---|---|---|---|---|
| | (N = 291) | | (N = 292) | |
| Primary outcome | | | | |
| Hospitalized >24 hours or death for any cause - no. (%) | 3 | (1) | 21 | (7) |
| Hospitalized >24 hours for any cause | 3 | (1) | 21 | (7) |
| Death by any cause | 0 | | 1 | (<1) |
| Alive and not hospitalized - no. (%) | 284 | (98) | 270 | (92) |
| Missing - no. (%) | 4 | (1) | 1 | (<1) |
| Withdrew consent prior to dosing - no. (%) | 3 | (1) | 1 | (<1) |
| Percent reduction (97.24% CI), P value | 85% (44% to 96%); P = 0.002 | | | |
| Other clinical outcomes* | | | | |
| Emergency room visit, hospitalization, or death for any | 6 | (2) | 28 | (10) |
| cause - no. (%) | | | | |
| Emergency room visit for any cause | 2 | (<1) | 8 | (3) |
| Hospitalized for any cause | 4 | (1)† | 21 | (7) |
| Death by any cause | 0 | | 1 | (<1) |
| Emergency room visit without hospitalization or | 3 | (1) | 7 | (2) |
| hospitalized for <24 hours for any cause - no. (%) | | | | |
| Severe or critical progression‡ - no. (%) | 2 | (<1) | 19 | (7) |
| Low-flow nasal cannula or face mask | 2 | (<1) | 11 | (4) |
| Non-rebreather mask, high-flow nasal cannula, or | 0 | | 5 | (2) |
| noninvasive ventilation | | | | |

| | | | |
|---|---|---|---|
| Invasive mechanical ventilation | 0 | 2 | (<1) |
| Death by any cause | 0 | 1 | (<1) |
| Admission to intensive care for any cause - no. (%) | 0 | 5 | (2) |

ITT denotes intent-to-treat, CI confidence interval.
*Inferential testing of secondary endpoints has not been performed at this interim analysis.
†One patient was hospitalized for less than 24 hours for diabetes management.
‡Severe or critical progression as manifested by supplemental oxygen use.

TABLE 10
Summary of Adverse Events (Safety Analysis Population)

| | Sotrovimab | | Placebo | |
|---|---|---|---|---|
| | (N = 430) | | (N = 438) | |
| Any adverse event - no. (%) | 73 | (17) | 85 | (19) |
| Related to study treatment* | 8 | (2) | 8 | (2) |
| Leading to permanent discontinuation of study treatment† | 0 | | 0 | |
| Leading to dose interruption/delay | 2 | (<1)‡ | 0 | |
| Occurring in ≥1% of patients receiving sotrovimab by preferred term | | | 3 | (<1) |
| Diarrhea | 6 | (1) | | |
| Any infusion-related reaction§ - no. (%) | 6 | (1) | 5 | (1) |
| Related to study treatment* | 1 | (<1) | 2 | (<1) |
| Leading to permanent discontinuation of study treatment† | 0 | | 0 | |
| Leading to dose interruption/delay | 0 | | 0 | |
| Any grade 3 or 4 adverse event - no. (%) | 7 | (2) | 27 | (6) |
| Any serious adverse event - no. (%) | 7 | (2) | 26 | (6) |

| | | | |
|---|---|---|---|
| Related to study treatment* | 0 | 1 | (<1) |
| Fatal | 0 | 2 | (<1)¶ |

| | | |
|---|---|---|
| Related to study treatment* | 0 | 0 |

*Relatedness was determined by individual study investigators while blinded to study treatment.
†A patient was permanently discontinued from the completion of drug infusion if they experienced life-threatening infusion-related reactions, including severe allergic or hypersensitivity reactions during the intravenous infusion.
‡For both patients, the adverse event was infusion extravasation; both infusions were completed.
§Infusion-related reactions were defined as adverse events with preferred terms of pyrexia, chills, dizziness, dyspnea, pruritus, rash, and infusion-related reaction within 24 hours of study drug administration.

In addition to the patient in the placebo group who died by day 29, another patient in the placebo group died due to COVID-19 pneumonia on day 37; this patient was admitted to the hospital prior to day 29 and was thus considered to have met the primary endpoint definition of COVID-19 progression.

### Supplementary Methods

### Inclusion Criteria

Patients are eligible to be included in the study only if all of the following criteria apply:

### Age and Risk Factors

Patient must be 18 years of age or older AND at high risk of progression of COVID-19 based on presence of one or more of the following risk factors:
1. Diabetes (requiring medication)
2. Obesity (body-mass index >35 kg/m²)
Change: body-mass index threshold was >30 kg/m² in the original protocol. See “Changes to Protocol/SAP” section for more information
1. Chronic kidney disease (i.e., estimated glomerular filtration rate <60 mL/min/1.73 m² according to the Modification of Diet in Renal Disease study equation)
2. Congestive heart failure (New York Heart Association class II or more)
3. Chronic obstructive pulmonary disease (history of chronic bronchitis, chronic obstructive lung disease, or emphysema with dyspnea on physical exertion) and moderate to severe asthma (patient requires an inhaled steroid to control symptoms or has been prescribed a course of oral steroids in the past year); or
Patient 55 years of age or older, irrespective of comorbidities
Note: target enrollment of ˜15% of patients over 70 years of age
Change: target enrollment criterion was not in the original protocol. See “Changes to Protocol/SAP” section for more information

### Type of Patient and Disease Characteristics

Patients who have a positive SARS-CoV-2 test result (by any validated diagnostic test [e.g. RT-PCR, antigen-based testing on any specimen type]); and
Oxygen saturation ≥94% on room air; and
Have COVID-19 defined by one or more of the following symptoms: fever, chills, cough, sore throat, malaise, headache, joint or muscle pain, change in smell or taste, vomiting, diarrhea, shortness of breath on exertion; and
Less than or equal to 5 days from onset of symptoms

### Sex and Contraceptive Barrier Requirements

### No Gender Restrictions

Female patients must meet and agree to abide by the following contraceptive criteria:
1. Contraception use by women should be consistent with local regulations regarding the methods of contraception for those participating in clinical studies
2. A female patient is eligible to participate if she is not pregnant or breastfeeding and one of the following conditions applies:
Is a woman of nonchildbearing potential
Is a woman of childbearing potential and is using a contraceptive method that is highly effective, with a failure rate of <1%, during the study intervention period and for up to 24 weeks after the last dose of study intervention. The investigator should evaluate potential for contraceptive method failure (e.g., noncompliance, recently initiated) in relationship to the first dose of study intervention
A woman of childbearing potential must have a negative highly sensitive pregnancy test (urine or serum as required by local regulations) before the first dose of study intervention. If a urine test cannot be confirmed as negative (e.g., an ambiguous result), a serum pregnancy test is required. In such cases, the patient must be excluded from participation if the serum pregnancy result is positive
The investigator is responsible for the review of medical history, menstrual history, and recent sexual activity to decrease the risk for inclusion of a woman with an early undetected pregnancy

### Informed Consent

Capable of giving signed informed consent, which includes compliance with the requirements and restrictions listed in the informed consent form and in this protocol; or
If patients are not capable of giving written informed consent, alternative consent procedures will be followed

### Prior Concurrent Clinical Study Experience

Enrollment in any investigational vaccine study within the last 180 days or any other investigational drug study within 30 days prior to day 1 or within five half-lives of the investigational compound, whichever is longer
Enrollment in any trial of an investigational vaccine for SARS-CoV-2

### Procedures

In-person study visits occurred on days 5, 8, 11, 15, 22, and 29, as well as daily telephone calls through day 15 (except on in-person visit days) to assess for adverse events and worsening of COVID-19. Starting at week 8, patients were monitored monthly via alternating telephone calls (weeks 8 and 16) and in-person visits (weeks 12, 20, and 24) for COVID-19 illness for a total of 24 weeks from dosing. Blood samples were collected for laboratory assessments at days 1, 15, 22, and 29 and for anti-drug antibodies on days 1 and 29. Patient-reported outcome assessments are administered through week 24.

### Changes to Protocol/SAP

Local safety laboratory assessments at screening were modified to specify that only ABO typing was required per protocol

### Rationale:

While certain screening assessments to confirm study patient eligibility must be performed or documented locally (e.g., pregnancy test for women of childbearing potential and SARS-CoV-2 infection via a validated diagnostic test), ABO typing is the only required local laboratory assessment at screening for safety purposes All other local safety laboratory assessments during the screening visit (hematology, clinical chemistry, and coagulation parameters) may or may not be performed according to the clinical discretion of the study investigator or as required by local regulations Considering the time to randomization and requirement for central laboratory testing on day 1 (randomization), the amount of sample collection was overly cumbersome for sites
Study objectives and endpoints (secondary and exploratory endpoints) were modified, including the introduction of an additional secondary endpoint (i.e., evaluation of the proportion of patients who have progression of COVID-19 through day 29 as defined by a visit to a hospital emergency room for management of illness, hospitalization for acute management of illness, or death), amendment to existing patient-reported outcome and virology endpoints, and introduction of additional exploratory endpoints

### Rationale:

Preliminary clinical trial data generated for other monoclonal antibodies targeting COVID-19 suggest that the rate of progression of COVID-19 leading to the requirement for hospitalization may be lower than originally estimated and the efficacy of antispike protein monoclonal antibodies in the treatment of COVID-19 in the outpatient population is greater than a 50% decrease in the proportion of medically attended visits through day 29
In light of new data from recent clinical trials of anti-SARS-CoV-2 mAbs, the secondary and exploratory endpoints were modified to reflect the clinical outcomes that are most relevant in the outpatient populations who receive COVID-19 monoclonal antibodies
Modified the “Age and Risk Factors” eligibility criteria (inclusion criteria #3 and #4) to enrich high-risk populations with the highest unmet medical need

### Rationale:

The “Age and Risk Factors” eligibility criteria has been modified to increase the body-mass index requirement defining obesity and introduce a targeted minimum number of patients (approximately 15%) >70 years of age
In light of emerging clinical data on the progression of COVID-19 in the outpatient setting, eligibility criteria were amended to enrich for populations at high risk for progression to severe COVID-19
Modified the “Medical Conditions” eligibility criteria (exclusion criteria #10) to enrich high-risk populations with the highest unmet medical need

### Rationale:

The “Medical Conditions” eligibility criteria have been modified to clarify the definition of “severely immunocompromised”
In light of emerging clinical data on the progression of COVID-19 in the outpatient setting, eligibility criteria were modified to enrich for immunosuppressed populations at high risk for progression to severe COVID-19
Modified the “Other Exclusions” eligibility criteria and “Medication Not Permitted During the Study” to clarify restrictions around dosing with a SARS-CoV-2 vaccine

### Rationale:

Considering the recent data published on SARS-CoV-2 vaccines, the protocol was amended to clarify restrictions surrounding administration of an experimental or approved SARS-CoV-2 vaccine
Clarified guidance for infusion-related reactions

### Rationale:

Protocol language was amended to clarify that sites should follow local or institutional guidelines for the treatment of infusion-related reactions
Statistical analysis plan (interim analysis #1, interim analysis #2, and statistical approach) were modified

### Rationale:

Preliminary clinical trial data generated for other monoclonal antibodies targeting COVID-19 suggest that the rate of progression of COVID-19 to requirement for hospitalization may be lower than originally estimated and the efficacy of antispike protein monoclonal antibodies in the treatment of COVID-19 in the outpatient population is greater than a 50% decrease in the proportion of medically attended visits through day 29
In light of this emerging data, the interim analyses were modified to more accurately reflect the most recent data on progression of COVID-19 and the potential efficacy of monoclonal antibodies against SARS-CoV-2 Specified an intention to conduct a “non-COVID-19” safety analysis

### Rationale:

Since it is not be possible to delineate in a single patient whether the hospitalization is directly related to COVID-19 complications or could be related to sotrovimab causing more severe disease due to antibody-dependent enhancement, all hospitalizations regardless of cause are included in the primary endpoint and are counted as serious adverse events
To inform on the number and nature of non-COVID-19 adverse events and serious adverse events, additional safety analyses are performed in which select, prespecified terms consistent with known progression of COVID-19 disease are excluded.

TABLE 11
Presenting Symptoms (ITT Population)

| | Sotrovimab | | Placebo | | Total | |
|---|---|---|---|---|---|---|
| Symptom - no. (%) | (N = 291) | | (N = 292) | | (N = 538) | |
| Cough | 240 | (82) | 247 | (85) | 487 | (84) |
| Muscle aches/myalgia | 215 | (74) | 215 | (74) | 430 | (74) |
| Headache | 202 | (69) | 216 | (74) | 418 | (72) |
| Fatigue | 180 | (62) | 183 | (63) | 363 | (62) |
| Malaise | 172 | (59) | 172 | (59) | 344 | (59) |
| Sore throat | 171 | (59) | 172 | (59) | 343 | (59) |
| Fever | 164 | (56) | 168 | (58) | 332 | (57) |
| Loss of taste | 171 | (59) | 159 | (54) | 330 | (57) |
| Loss of smell | 175 | (60) | 152 | (52) | 327 | (56) |
| Chills | 164 | (56) | 158 | (54) | 322 | (55) |
| Joint pain/arthralgia | 153 | (53) | 153 | (52) | 306 | (52) |
| Shortness of breath | 131 | (45) | 131 | (45) | 262 | (45) |
| Diarrhea | 87 | (30) | 101 | (35) | 188 | (32) |
| Nausea | 85 | (29) | 91 | (31) | 176 | (30) |
| Vomiting | 34 | (12) | 37 | (13) | 71 | (12) |

ITT denotes intent-to-treat.

TABLE 12
Baseline Demographic and Disease Characteristics (Safety Analysis Population)

| | Sotrovimab | | Placebo | | Total | |
|---|---|---|---|---|---|---|
| Characteristic | (N = 430) | | (N = 438) | | (N = 868) | |
| Age - yr, median (range) | 53.0 | (18-96) | 52.0 | (17-88) | 53.0 | (17-96) |
| ≥65 yr - no. (%) | 84 | (20) | 88 | (20) | 172 | (20) |
| >70 yr - no. (%) | 42 | (10) | 42 | (10) | 84 | (10) |
| Male gender - no. (%) | 194 | (45) | 212 | (48) | 406 | (47) |
| Race* - no. (%) | | | | | | |
| White | 374 | (87) | 384 | (88) | 758 | (88) |
| Black or African American | 27 | (6) | 33 | (8) | 60 | (7) |
| Asian | 21 | (5) | 19 | (4) | 40 | (5) |
| Mixed race | 6 | (1) | 0 | | 6 | (<1) |
| American Indian or Alaska Native | 1 | (<1) | 1 | (<1) | 2 | (<1) |
| Hispanic or Latino ethnic group - no. (%) | 280 | (65) | 280 | (64) | 560 | (65) |
| Body-mass index† - mean (SD) | 32.1 | (6.4) | 32.5 | (6.7) | 32.3 | (6.5) |
| Duration of symptoms‡ - no. (%) | | | | | | |
| ≤3 days | 254 | (59) | 260 | (59) | 514 | (59) |
| 4-5 days | 173 | (40) | 178 | (41) | 351 | (40) |
| Any risk factor for COVID-19 progression - | 427 | (>99) | 434 | (>99) | 861 | (>99) |
| no. (%) | | | | | | |
| Age ≥55 yr | 195 | (45) | 205 | (47) | 400 | (46) |
| Diabetes requiring medication | 93 | (22) | 88 | (20) | 181 | (21) |
| Obesity (body-mass index >30†) | 267 | (62) | 292 | (67) | 559 | (64) |
| Chronic kidney disease (eGFR <60 by MDRD) | 2 | (<1) | 5 | (1) | 7 | (<1) |
| Congestive heart failure (NYHA class II or | 4 | (<1) | 3 | (<1) | 7 | (<1) |
| more) | | | | | | |
| Chronic obstructive pulmonary disease | 24 | (6) | 18 | (4) | 42 | (5) |
| Moderate to severe asthma | 69 | (16) | 72 | (16) | 141 | (16) |
| Number of concurrent risk factors for | | | | | | |
| COVID-19 progression - no. (%) | | | | | | |
| 0 | 3 | (<1) | 4 | (<1) | 7 | (<1) |
| 1 | 251 | (58) | 250 | (57) | 501 | (58) |
| 2 | 132 | (31) | 130 | (30) | 262 | (30) |
| ≥3 | 44 | (10) | 54 | (13) | 98 | (11) |

SD denotes standard deviation, eGFR estimated glomerular filtration rate, MDRD Modification of Diet in Renal Disease, NYHA New York Heart Association.
*Race data were not available for one patient in the sotrovimab group and one patient in the placebo group.
†Body-mass index is the weight in kilograms divided by the square of the height in meters.
‡One patient in the sotrovimab group had a symptom duration of 6 days. For two other patients in the sotrovimab group, symptom duration data were not available at the time of this interim analysis.

TABLE 13
Primary Reasons for Hospitalizations of More Than 24 Hours (ITT Population)

| | | | | | | | |
|---|---|---|---|---|---|---|---|
| | | | | | Intensive | Invasive | |
| | Age | | Hospitalization | | care unit | mechanical | |
| Patient | (yr) | Sex | day | Primary reason | admission | ventilation | Fatal |

Sotrovimab-treated patients

| | | | | | | | |
|---|---|---|---|---|---|---|---|
| A | 96 | M | 19 | COVID-19 | N | N | N |
| B | 65 | F | 22 | Small | N | N | N |
| | | | | intestinal | | | |
| | | | | obstruction | | | |
| C | 71 | F | 2 | COVID- | N | N | N |
| | | | | pneumonia | | | |

Placebo-treated patients

| | | | | | | | |
|---|---|---|---|---|---|---|---|
| D | 52 | F | 4 | COVID- | N | N | N |
| | | | | pneumonia | | | |
| E | 50 | F | 4* | COVID- | N | N | N |
| | | | | pneumonia | | | |
| F | 66 | F | 6 | COVID- | N | N | N |
| | | | | pneumonia | | | |
| G | 38 | M | 9* | COVID- | N | N | N |
| | | | | pneumonia | | | |
| H | 50 | F | 4* | COVID- | Y | N | N |
| | | | | pneumonia | | | |
| I | 82 | F | 7 | Acute | N | N | N |
| | | | | respiratory | | | |
| | | | | failure | | | |
| J | 70 | M | 12 | Respiratory | Y | Y | N |
| | | | | distress | | | |
| K | 70 | M | 5 | Pneumonia | Y | N | Y |
| L | 65 | M | 6 | Dehydration | N | N | N |
| M | 52 | F | 5 | COVID- | N | N | N |
| | | | | pneumonia | | | |
| N | 62 | M | 7 | COVID- | N | N | N |
| | | | | pneumonia | | | |
| O | 57 | M | 6 | Pneumonia | N | N | N |
| P | 65 | M | 12 | Pneumonia | N | N | N |
| Q | 68 | M | 7 | COVID- | N | N | N |
| | | | | pneumonia | | | |
| R | 55 | F | 7 | Pulmonary | N | N | N |
| | | | | embolism | | | |
| S | 60 | M | 10 | COVID- | N | N | N |
| | | | | pneumonia | | | |
| T | 71 | F | 10 | COVID- | Y | Y | Y |
| | | | | pneumonia | | | |
| U | 37 | F | 6* | COVID- | N | N | N |
| | | | | pneumonia | | | |
| V | 83 | M | 8 | Dyspnea | N | N | N |
| W | 56 | M | 2 | COVID- | Y | N | N |
| | | | | pneumonia | | | |
| X | 55 | M | 7 | COVID- | N | N | N |
| | | | | pneumonia | | | |

ITT denotes intent-to-treat, M male, F female, N no, Y yes.
*The adverse event associated with the hospitalization started the day before the patient was admitted to the hospital.

### Example 41

### Phase 2/3 Clinical Study of Sotrovimab for Treatment of Hospitalized Subjects with Severe to Critical Covid-19 Disease

A randomized, multi-center, double-blind, placebo-controlled study is performed to assess safety and efficacy of monoclonal antibody sotrovimab in patients with severe or critical COVID-19 disease.

A primary objective is to compare efficacy of sotrovimab versus placebo. Secondary objectives are to compare efficacy of sotrovimab versus placebo on viral shedding in upper and lower respiratory samples, to compare safety and tolerability of sotrovimab versus placebo, to determine efficacy of sotrovimab versus placebo for induction of an anti-viral response, to determine efficacy of sotrovimab versus placebo for increasing ICU- and hospital-free days, and to determine serum pharmacokinetics (PK) of sotrovimab.

Exploratory objectives are to compare efficacy of sotrovimab against versus placebo in preventing non-respiratory complications of COVID-19 disease, to compare effect of sotrovimab versus placebo on viral shedding in upper and lower respiratory samples and blood samples, to assess emergence of resistance to sotrovimab, to evaluate effect of sotrovimab on potential biomarkers of host response, and to assess correlations between efficacy, safety, PK, viral shedding, and immunogenicity.

### Details of Criteria for Evaluation

Primary endpoints of this study are as follows:
Proportion of subjects who progress to severe or critical COVID-19 or die through Day 29 as defined by:
1. Severe disease: hypoxemia (O₂ saturation ≤93% on room air or PaO₂/FiO₂<300) requiring oxygen supplementation >1 day OR subject requires ≥4 L/min oxygen supplementation or equivalent AND the investigator or designee determine that the measurement of O₂ saturation on room air is medically unsafe; OR
2. Critical disease: Respiratory failure requiring at least one of the following: invasive mechanical ventilation, ECMO; OR shock; OR multi-organ dysfunction/failure; OR
3. Death
Time to clinical response, defined as being alive and independent of oxygen supplementation through Day 29, as compared to standard of care (SOC).
Secondary endpoints of this study are as follows:
1. Occurrence of adverse events (AEs)
2. Occurrence of serious adverse events (SAEs)
3. Occurrence of adverse events of special interest (AESI)
4. Proportion of subjects who progress to develop severe or critical COVID-19 at Day
8, Day 15, or Day 22
5. 29-day, 60-day, and 90-day all-cause mortality
6. Total number of ventilator days from randomization through 29 days
7. Total number of days requiring supplemental oxygen through 29 days
8. Total intensive care length of stay (LOS)
9. Total hospital LOS
10. Severity and duration of subject reported signs and symptoms of COVID-19 related illness using the Flu-PRO patient-reported outcome measurement instrument
11. Occurrence of cardiac, thromboembolic, renal, neurologic events
12. Detection of SARS-CoV-2 in nasal secretions by PCR at baseline and during follow-up period through Day 29
13. Time to no detectable viral RNA (<LLOQ) by quantitative RT-PCR from nasopharyngeal samples
14. Sotrovimab pharmacokinetics (PK) in serum
15. Incidence and titers (if applicable) of serum ADA to Sotrovimab
Exploratory endpoints of this study are as follows:
1. Proportion of subjects at Day 8, Day 15, Day 22, and Day 29 in each category of the ordinal scale for clinical improvement (see below)
2. Emergence of viral resistance mutants to mAb by SARS-CoV-2
3. Viral load in nasal secretions and blood by qRT-PCR
4. Host transcriptome and immunophenotyping analysis
5. FcTR polymorphisms as determined by genotyping
6. IgG1 allotypes as determined by genotyping
7. Change from baseline in Work Productivity and Activity Impairment (WPAI)
8. Change from baseline in health-related quality of life according to EQ-5D-5L.
The ordinal scale to be used for categorization of clinical improvement is as follows:
1. Uninfected: No clinical or virologic evidence of infection
2. Non-hospitalized, no limitation of activity (if using oxygen at home, report as category 4)
3. Non-hospitalized, limitation of activity
4. Hospitalized, no oxygen therapy
5. Hospitalized, low-flow oxygen by mask or nasal prongs
6. Hospitalized, high-flow oxygen, Continuous positive airway pressure (CPAP), bilevel positive airway pressure (BiPAP), non-invasive ventilation
7. Hospitalized, intubation and mechanical ventilation
8. Hospitalized, mechanical ventilation plus organ support (vasopressors, RRT, ECMO)
9. Death

### Number of Subjects Planned

This study is comprised of two parts. Part 1 enrolls approximately 20 subjects with severe COVID-19 disease. Part 2 enrolls approximately 500 subjects with severe to critical COVID-19 disease; approximately 250 subjects per treatment arm.

### Randomization and Stratification

Subjects are randomized in a 1:1 ratio to receive a single IV dose of sotrovimab plus standard of care (SoC) or equal volume saline placebo plus SoC.

For Part 1, the first 20 subjects enrolled in the study (ten per arm) are in the severe COVID-19 disease population (Grade 4 or 5). These subjects are stratified by time from onset of symptoms, either 1) less than seven days or 2) seven days or more. All subjects enrolled in Part 1 are assigned to intensive PK sampling. Serum PK samples are collected from these subjects.

For Part 2, the clinical status of the subjects are stratified by disease status (severe or critical) and time from onset of symptoms, as follows:
1. Severe (Grade 4 or 5) and less than seven days since onset of symptoms
2. Severe (Grade 4 or 5) and seven days or more since onset of symptoms
3. Critical (Grade 6 or 7) and less than seven days since onset of symptoms
4. Critical (Grade 6 or 7) and seven days or more since onset of symptoms.
Sparse PK samples are collected from subjects enrolled in Part 2.

### Diagnosis and Main Criteria for Inclusion

Inclusion criteria include:
Age: Subjects aged 18 years and older
Disease characteristics:
Subjects who have a positive SARS-CoV-2 test result (by any validated test e.g. RT-PCR on any specimen type); AND
Hospitalized with severe COVID-19 disease defined as requirement for supplemental oxygen or non-invasive ventilation consistent with Grade 4 or Grade 5 disease; OR
Hospitalized with critical COVID-19 disease defined as those on mechanical ventilation (Grade 6 or Grade 7 disease); AND
Onset of COVID-19 symptoms within 14 days of Day 14; AND
Randomized within 48 hours of admission to hospital
Sex:
No gender restriction
Female subjects must meet and agree to abide by the following contraceptive criteria.
Contraception use by women should be consistent with local regulations regarding the methods of contraception for those participating in clinical studies.
A female subject is eligible to participate if she is not pregnant or breastfeeding, and one of the following conditions applies:
Is a woman of non-childbearing potential (WONCBP); OR
Is a woman of child-bearing potential (WOCBP) and using a contraceptive method that is highly effective, with a failure rate of <1% during the study intervention period and for up to one year after the last dose of study intervention. The investigator should evaluate potential for contraceptive method failure (e.g., noncompliance, recently initiated) in relationship to the first dose of study intervention.
A WOCBP must have a negative highly sensitive pregnancy test (urine or serum as required by local regulations) at hospital admission or before the first dose of study intervention. If a urine test cannot be confirmed as negative (e.g., an ambiguous result), a serum pregnancy test is required. In such cases, the subject is excluded from participation if the serum pregnancy result is positive.

### Informed Consent:

Willing to comply with the study requirements and capable of giving signed informed consent or legally acceptable representative is willing and able to give written informed consent on behalf of the subject to participate in the study for unconscious adults and those incapable of consenting themselves due to their medical condition.
Exclusion Criteria:
   in the opinion of the responsible investigator, any condition for which participation would not be in the best interest of the participant, or that could limit protocol-specified assessments; expected inability to participate in study procedures; participants who, in the opinion of the investigator, are not likely to survive beyond 48 hours at the time of randomization; planned or anticipated discharge or transfer to another hospital within 72 hours at the time of randomization; history of hypersensitivity to other mAbs or any of the excipients present in the investigational product;
   For the lead-in phase: participants with end organ failure or dysfunction are not eligible for Lead-in phase but are eligible for Expansion-Phase.
   End-organ dysfunction categories are: a. stroke b. meningitis c. encephalitis d. myelitis e. myocardial infarction f. myocarditis g. pericarditis h. symptomatic congestive heart failure (New York Heart Association [NYHA] class III-IV) i. arterial or deep venous thrombosis or pulmonary embolism
   End-organ failure categories are: a. requirement for high-flow oxygen, non-invasive ventilation, or invasive mechanical ventilation b. extracorporeal membrane oxygenation (ECMO) c. mechanical circulatory support (e.g., intra-aortic balloon pump, ventricular assist device) d. vasopressor therapy e. commencement of renal replacement therapy (RRT) during this admission (i.e. not patients on chronic RRT).
   Prior receipt of
   any SARS-CoV-2 hyperimmune intravenous immunoglobulin (hIVIG) from COVID-19 survivors
   convalescent plasma from a person who recovered from COVID-19 or
   SARS-CoV-2 neutralizing mAbs at any time.
   Participating in other clinical trials involving an investigational study intervention, including for COVID-19
   Pregnant or breast-feeding females

### Duration of Study Participation and Follow Up

The duration of study drug treatment is a single dose. The estimated total time of study, inclusive of screening and follow-up, for each subject is approximately 36 weeks. Screening assessments are performed up to 48 hours before the first dose. After completion of screening assessments, eligible subjects are treated with a single IC dose of sotrovimab or placebo. Subjects are assessed daily up to the day of hospital discharge. Following discharge from hospital, assessments occur on Day 15 and Day 29 and follow up continues to Week 36 after randomization.

### Study Design

An Independent Data Monitoring Committee (IDMC) actively monitors interim unblinded safety data (Part 1) and interim unblinded safety and efficacy data (Part 2) to make recommendations regarding ongoing study conduct. The IDMC members include 3-4 physicians with relevant medical specialist training and one statistician. A Joint Safety Review Team (JSRT) determines if a safety concern based on instream blinded data review needs to be escalated to the IDMC.

This study is a randomized, double-blind, multi-center, placebo-controlled trial of sotrovimab, a monoclonal antibody (mAb) against SARS-CoV-2, to assess efficacy and safety of a single IV dose of sotrovimab versus placebo on top of investigator-defined standard of care (SoC) as treatment in adult participants hospitalized with COVID-19 disease. The study population consists of hospitalized subjects with COVID-19 disease with a positive SARS-CoV-2 test result. These participants can have either critical (Grade 6 or 7) or severe (Grade 4 or 5) disease. All subjects receive SoC as per institutional protocols, in addition to study treatment.

The study comprises 2 parts. Part 1 enrolls 20 participants who have severe COVID-19 disease. Following an initial safety assessment of unblinded data at Day 14 by the IDMC, Part 2 (the main cohort) progresses, where participants with both severe and critical COVID-19 disease are enrolled. Only severe participants are enrolled in Part 2 without pausing the study until the IDMC recommend recruiting critical participants as well.

To explore the host immune response and potential biomarkers of infection, subjects may consent to an optional sub-study in which peripheral blood mononuclear cells (PBMCs) are collected at specified timepoints. This optional substudy is performed at selected sites. Approximately 50-100 participants per arm are enrolled into the optional sub-study.

During this study, an unblinded safety data review conducted by an IDMC determines whether it is safe to expand from Part 1, which includes subjects with severe (Grade 4 or 5) COVID-19 disease to Part 2, which additionally includes subjects with critical (Grade 6 or 7) COVID-19 disease.

A placebo control distinguishes the safety and tolerability of sotrovimab from the background signs and symptoms of COVID-19 and evaluates its potential benefit on clinical outcomes. The use of a placebo arm allows for a valid evaluation of any changes in efficacy and safety attributable to sotrovimab versus those attributable to SoC and other treatments given during the study.

The primary endpoint to assess efficacy of treatment is time to clinical response defined as subjects being alive and independent of supplementary oxygen or return to pre-morbid oxygen requirement (for subjects with chronic oxygen use) for at least 24 hours within 29 days of randomization. The transition from requiring oxygen therapy or higher levels of respiratory/organ support is a clinically significant milestone.

A significant secondary endpoints is all-cause mortality at Day 29. Other secondary efficacy endpoints related to hospital stay—length of hospital stay, ICU stay, time ventilated and the severity and duration of subject-reported signs and symptoms of COVID-19 are also clinically relevant.

### Study Intervention Groups and Duration

Screening assessments are performed up to 48 hours before the first dose. Screening procedures include taking a medical and disease history; testing for SARS-CoV-2 infection by nasal, nasopharyngeal, or endotracheal swab for subjects without a prior confirmed positive test; a full physical examination including measurement of height and weight; measurement of vital signs including blood pressure, pulse, respiratory rate, and temperature; oxygen saturation (SpO₂) for subjects not on invasive mechanical ventilation; oxygen delivery and ventilation status; 12-lead ECG (triplicate); urine or serum pregnancy test for all female subjects of childbearing potential; laboratory assessments including hematology, clinical chemistry profile, coagulation parameters, CRP, D-dimer, urinalysis, and cardiac troponin; chest radiography (chest x-ray or computed tomography scan taken after hospitalization per 30 SoC); and review of prior or concomitant medication.

Eligible subjects are treated with a single IV dose (sotrovimab or placebo) plus SoC on Day 1. They are assessed daily up to the day of hospital discharge. Following discharge from hospital, assessments occur on Day 15 and Day 29 and follow up continues to Week 36 after randomization.

In Part 1, subjects with severe COVID-19 disease (Grade 4 or 5) are randomized 1:1 by interactive response technology (IRT) in a blinded manner to receive a single IV dose of sotrovimab or placebo. In Part 2 (the main cohort), subjects with severe (Grade 4 or 5) or critical (Grade 6 or 7) COVID-19 disease are randomized 1:1 to receive a single IV dose of sotrovimab or placebo. All participants receive SoC as per institutional protocols, in addition to study treatment.

The randomization is stratified based on the disease severity (severe or critical) and time from onset of symptoms (less than seven days or seven days or more) at randomization.

### Study Interventions

Study interventions and dosing instructions are as follows in Table 14:

TABLE 14

| | | |
|---|---|---|
| Dose Formulation | Solution in single-use vial (25 mg/mL) | Sterile 0.9% (w/v) sodium |
| | | chloride solution |
| Unit Dose Strength(s) | 250 mg/vial (250 mg/10 mL) | Not applicable |
| Dosage Level(s) | (tbc) mg once | Once |
| Route of Administration | IV infusion | IV infusion |
| Dosing instructions | Withdraw 10.0 mL from the vial into a 10-20 mL | Dose per IV infusion |
| | small syringe and transfer to a 150 mL or 250 mL | instructions |
| | IV empty saline bag by an unblinded pharmacist. | |
| | Repeat this procedure until the required dose is | |
| | reached and administer over 1 hour. | |
| Special instructions | Gently mix sotrovimab and saline prior to IV | None |
| | infusion. | |
| Packaging and Labelling | Study intervention are provided in a single-use | Labelled as required per |
| | vial in an individual carton and labelled as | country requirement |
| | required per country requirement. | |

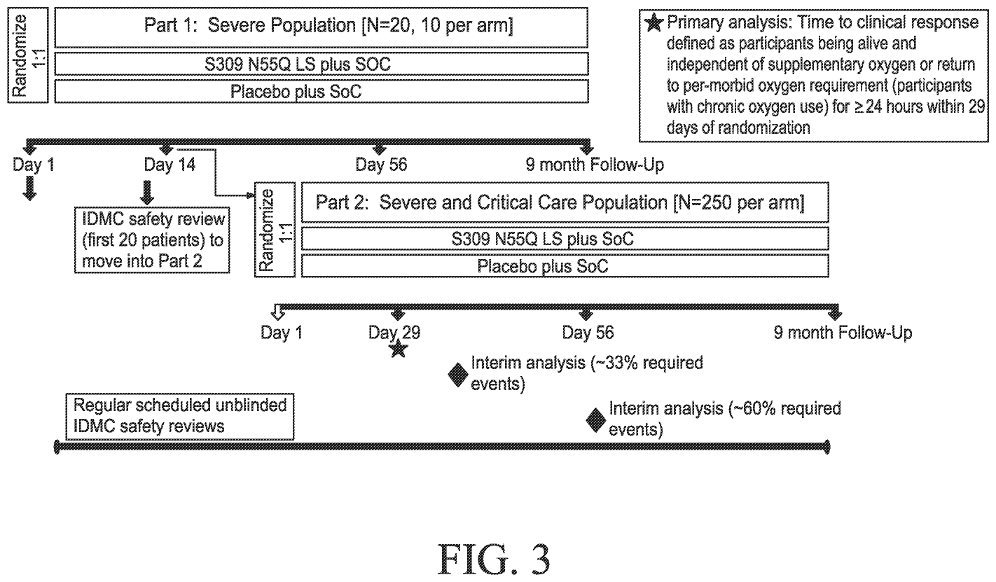

Further study details are shown in FIG. 3.

### Example 42

### Phase 3 Clinical Study of Sotrovimab in Patients Who have been Hospitalized with Covid-19

A Phase 3 clinical study is performed evaluate safety and effectiveness of different drugs in treating COVID-19 in people who have been hospitalized with the infection. Participants in the study are treated with either a study drug (e.g., sotrovimab) plus current standard of care (SOC), or with placebo plus current SOC.

The protocol is for a randomized, blinded, controlled platform study that allows investigational drugs to be added and dropped during the course of the study. This allows for efficient testing of drugs against placebo and standard of care (SOC) treatment within the same study. When more than one drug is tested at the same time, participants are randomly allocated to treatments or placebo.

Randomization are stratified by study site pharmacy and disease severity. There are 2 disease severity strata: Participants without organ failure (severity stratum 1); and participants with organ failure (severity stratum 2).

An independent Data and Safety Monitoring Board (DSMB) regularly reviews interim analyses and summarizes safety and efficacy outcomes. For investigational drugs with minimal pre-existing safety knowledge, the pace of enrollment is initially restricted, and there is an early review of safety data by the DSMB. For the study of each agent, at the outset of the trial, only participants in disease severity stratum 1 are enrolled. This continues until approximately 300 participants are enrolled and followed for 5 days. The exact number can vary according to the speed of enrollment and the timing of DSMB meetings. Prior to expanding enrollment to also include patients in disease severity stratum 2, safety is evaluated and a pre-specified futility assessment by the DSMB is carried out using 2 ordinal outcomes assessed at Day 5.

Both ordinal outcomes are used to assess futility because it is unclear whether the investigational agents under study will primarily influence non-pulmonary outcomes, for which risk is increased with SARS-CoV-2 infection, in part, through mechanisms that may be different from those that influence pulmonary outcomes.

For investigational agents passing this futility assessment, enrollment of participants is expanded, seamlessly and without any data unblinding, to include participants in disease severity stratum 2 as well as those in disease severity stratum 1. Future interim analyses are based on the primary endpoint of sustained recovery and use pre-specified guidelines to determine early evidence of benefit, harm or futility for the investigational agent. Participants are followed for 18 months following randomization.

The international trials within this protocol are conducted in several hundred clinical sites. Participating sites are affiliated with networks funded by the United States National Institutes of Health (NIH) and the US Department of Veterans Affairs.

Approximately 10,000 participants are enrolled. The allocation is randomized. The intervention model is parallel assignment. Masking is triple (Participant, Care Provider, Investigator). The primary purpose is treatment. In one arm, participants are randomized to receive drug (e.g. sotrovimab) plus SOC or placebo plus SOC. Subsequently, participants are no longer randomized to sotrovimab. In a placebo arm, placebo (commercially available 0.9% sodium chloride solution administered by IV infusion) plus remdesivir is provided absent contraindication.

### Primary Outcome Measures

Time from randomization to sustained recovery [Time Frame: Up to Day 90]. Sustained recovery defined as being discharged from the index hospitalization, followed by being alive and home for 14 consecutive days prior to Day 90.

### Secondary Outcome Measures

1. All-cause mortality [Time Frame: Thru Day 90]
2. Composite of time to sustained recovery and mortality [Time Frame: Thru Day 90]
3. Days alive outside short-term acute care hospital [Time Frame: Up to Day 90]
4. Pulmonary ordinal outcome [Time Frame: Days 1-7, 14 and 28]
Oxygen requirements measured by 7 categories (1=least severe, 7=most severe). The participant's highest (i.e. most severe) observed score is used.
5. Pulmonary+ ordinal outcome [Time Frame: Days 1-7]
Extrapulmonary complications and respiratory dysfunction measured by 7 categories (1=least severe, 7=most severe). The participant's highest (i.e. most severe) observed score is used.
6. Incidence of clinical organ failure [Time Frame: Thru Day 28]
7. Composite of death or serious clinical COVID-19 related events [Time Frame: Thru Day 90]
8. Composite of cardiovascular events and thromboembolic events [Time Frame: Thru Day 90]
9. Composite of grade 3 and 4 clinical adverse events, serious adverse events (SAEs) or death [Time Frame: Thru Days 5 and 28]
10. Incidence of infusion reactions [Time Frame: Thru Day 0]
11. Composite of SAEs or death [Time Frame: Thru 18 months]
12. Change in SARS-CoV-2 neutralizing antibody levels [Time Frame: Baseline to Days 1, 3, 5, 28 and 90]
13. Change in overall titers of antibodies [Time Frame: Baseline to Days 1, 3, 5, 28 and 90]
14. Change in neutralizing antibody levels [Time Frame: Baseline to Days 1, 3, 5, 28 and 90]
15. Incidence of home use of supplemental oxygen above pre-morbid oxygen use [Time Frame: 18 months]
Measured as: Alive at home and no use of continuous supplemental oxygen for an uninterrupted 14 day period
16. Incidence of no home use of supplemental oxygen above pre-morbid oxygen use [Time Frame: 14 days]
Measured as: alive at home for an uninterrupted 14 day period and no use of continuous supplemental oxygen at end of 14 day time period.
Adults aged 18 years and older (adults and older adults) of all sexes are eligible.
Inclusion criteria are: signing of informed consent; positive test for COVID-19 and progressive disease suggestive of ongoing COVID-19 infection; symptoms of COVID-19 for ≤12 days; and require admission to hospital for acute medical care (not for purely public health or quarantine purposes). Exclusion criteria are:
Patients who have received plasma from a person who recovered from COVID-19 or who have received neutralizing monoclonal antibodies at any time prior to hospitalization.
Patients not willing to abstain from participation in other COVID-19 treatment trials until after Day 5 of the study. Co-enrollment in certain trials that compare recommended Standard of Care treatments may be allowed, based on the opinion of the study leadership team.
Any condition which, in the opinion of the responsible investigator, participation would not be in the best interest of the participant or that could prevent, limit, or confound the protocol-specified assessments.
Patients considered unable to participate in study procedures.
Women of child-bearing potential who are not already pregnant at study entry and who are unwilling to acknowledge strong advice to abstain from sexual intercourse with men or practice appropriate contraception through 18 months of the study.
Men who are unwilling to acknowledge the strong advice to abstain from sexual intercourse with women of child-bearing potential or to use barrier contraception through 18 months of the study.
Pregnant women
Nursing mothers
Presence at study enrollment of any of the following:
1. stroke;
2. meningitis
3. encephalitis
4. myelitis
5. myocardial ischemia
6. myocarditis
7. pericarditis
8. symptomatic congestive heart failure
9. arterial or deep venous thrombosis or pulmonary embolism
Current or imminent requirement for any of the following:
1. invasive mechanical ventilation
2. ECMO (extracorporeal membrane oxygenation)
3. Mechanical circulatory support
4. vasopressor therapy
5. commencement of renal replacement therapy at this admission (i.e. not patients on chronic renal replacement therapy).

In addition, prior to the initial futility assessment which is performed when approximately 150 participants have been enrolled on sotrovimab and 150 on placebo, patients on high-flow oxygen or non-invasive ventilation (category 5 of the pulmonary ordinal outcome) will be excluded. These patients may be eligible for the trial if the initial futility assessment is passed by this agent.

A single dose of 500 mg sotrovimab is administered. The dose is selected based on in vitro neutralization data, in vitro resistance data, expected human PK extrapolated from a study in cynomolgus monkeys, and results of a GLP monkey toxicology study. Sotrovimab neutralized SARS-CoV-2 live virus with an average EC90 value of 186.3 ng/mL (range: 125.8-329.5 ng/mL). In resistance analyses, no viral breakthrough was observed through 10 passages at fixed concentrations of antibody even at the lowest dose tested (˜10×EC50), indicating the potential for sotrovimab to have a high barrier to resistance.

Using an increasing concentration selection method to force resistance emergence, modest fold changes in EC50 were observed during viral selection (5- to 6-fold change in EC50) for some passages. Sequencing and testing of spike variants from these passages using a pseudotyped virus system did not identify causal variants for this modest shift in potency. One passage of virus did demonstrate a >10-fold shift in EC50 which correlated with an E340A mutation. Further assessment has identified E340A to be a monoclonal antibody-resistant mutant (MARM) that confers a >100-fold reduction in susceptibility to sotrovimab. Notably, E340 is 100% conserved among available SARS-CoV-2 sequences. Due to the binary nature of the resistance selection results, a specific inhibitory quotient (IQ) was not informed by the resistance profiling. However, as very few strains are available to directly assess breadth of coverage, a conservative IQ (>10) is appropriate in this case. The cynomolgus monkey PK study (single IV dose, 5 mg/kg) was fit to a 2 compartment PK model. Human PK parameters were scaled from the cynomolgus monkey using an allometric scaling approach for fully human IgGs (allometric coefficient of 0.85 and 1 for CL and V, respectively; 10). The predicted serum clearance of sotrovimab in humans is estimated to be 141 mL/day and estimated volume of distribution is 6500 mL (˜93 mL/kg) assuming human weight of 70 kg. The projected human terminal elimination half-life is approximately 32 days.

In order to reduce risk to patients (treatment failure, emergence of viral resistance), a single therapeutic dose was selected that ensures sotrovimab concentrations in the lung are maintained far above levels anticipated to be protective for SARS-CoV-2 infection for the duration of the 28-day treatment window and beyond. A dose of 500 mg is expected to maintain serum levels at or above 38.5 μg/mL for the duration of the 28-day treatment period. Based on a conservative EC90 (0.33 g/mL) from the highest end of the EC90 range, and accounting for the lung:serum ratio for IgG (assumed conservative value of 0.25; reported range 0.25-0.68 for whole lung and interstitial fluid, respectively; the serum trough concentration following a 500 mg dose is expected to result in lung concentrations associated with maximal (>99%) antiviral activity; >29×tissue-adjusted EC90 for the duration of the 28 day treatment period. This conservative inhibitory quotient (29-fold) in lung is believed to be appropriate to increase potential for treatment success and reduce risk for resistance.

Additionally, a 500 mg dose is anticipated to result in protective levels of sotrovimab in nasopharyngeal secretions (>5×tissue adjusted EC90 assuming NPS:serum ratio of 0.05, 12) which could potentially reduce transmission.

The NOAEL for sotrovimab was 500 mg/kg, the highest dose tested, when sotrovimab was administered via IV infusion once a week for 2 weeks in cynomolgus monkeys. At this NOAEL, preliminary Cmax and area-under-the-curve (AUC)0-t (AUC from time 0 to 168 hr post-end of infusion following the 2nd dose) were 13500 μg/mL and 48200 μg·day/mL, respectively. The human equivalent dose (HED) (HED calculated via direct mg/kg conversion according to FDA guidance on proteins administered intravascularly with Mr >100,000 daltons; is 500 mg/kg or a 30,000 mg fixed dose (using human body weight of 60 kg). Using a safety factor of 10, the maximum recommended starting dose in humans is approximately 50 mg/kg or a 3,000 mg fixed dose. Based on the proposed 500 mg human dose, the margins based on the HED, Cmax, and AUC (conservative AUC margin based on partial AUC0-t from TX-7831-0102 and expected AUCinf in humans) are 60-, 87-, and 13.6-fold, respectively, supporting the proposed clinical dose of 500 mg.

In addition to the inclusion and exclusion criteria outlined in the master protocol, the following patients will be excluded: 1) pregnant women; and 2) nursing mothers. In addition, prior to the initial futility assessment which is performed when approximately 150 participants have been enrolled on sotrovimab and 150 on placebo, patients on high-flow oxygen or non-invasive ventilation (category 5 of the pulmonary ordinal outcome) are excluded. These patients may be eligible for the trial if the initial futility assessment is passed by this agent.

A single infusion is administered over a one-hour period. Study participants are monitored closely, and, per discretion of the physician supervising the infusion, adjustments in the infusion rate is made and/or the infusion paused or stopped.

Physician supervising the infusion may use supportive measures per local practice, if indicated. Sotrovimab is provided in vials of 10 ml solution containing 250 mg antibody each. Sotrovimab must be stored between 2° C. and 8° C. A total of 2 vials are required for dosing of the agent at 500 mg. The agent is administered at a concentration of 1 mg/ml to 10 mg/ml. Placebo is normal saline from local supply. If an infusion reaction occurs during administration or if the participant has a medical history suggesting a potential benefit from premedication, the study investigator(s) should determine the appropriate premedication. If the frequency of infusion reactions among participants warrants it, premedication may be recommended. If minor infusion reactions are observed, administration of acetaminophen, 500 mg to 1000 mg, antihistamines and/or other appropriately indicated medications may be given prior to the start of infusions for subsequent participants. The decision to implement premedication for infusions in subsequent participants is made by the investigator and sponsor and recorded in the study documentation. Any pre-medications given are documented as a concomitant therapy.

At Days 0, 28, and 90, venous blood samples are collected to determine antibody production against sotrovimab. Immunogenicity may be assessed by a validated assay designed to detect ADAs in the presence of sotrovimab. Antibodies may be further characterized for their ability to neutralize the activity of sotrovimab. Remaining volume from the PK sample may also be used for immunogenicity assessments as needed.

At Days 0, 1, 5, 28, and 90, venous blood samples are collected to determine sotrovimab serum concentration for pharmacokinetic assessment. The PK/Immunogenicity assessment requires 2 mL of the serum collected. PK samples may be assessed by a validated assay at a bioanalytical lab. The PK assessment uses the same 2 ml serum. Analysis of samples from placebo-treated subjects is not planned. Remaining sample used for PK may be pooled and used for exploratory metabolism or bioanalytical method experiments as deemed appropriate. Samples may be shipped to the lab for analyses in batches as the substudy is unfolding. Results from such analyses are returned to the trial central database. This data and all other data on the participants remain in the trial central database until the trial is unblinded. All participants should be monitored closely for 2 hours after the infusion, as there is a risk of infusion reaction and hypersensitivity (including anaphylaxis) with any biological agent.

Symptoms and signs that may occur as part of an infusion reaction, include, but are not limited to, fever, chills, nausea, headache, bronchospasm, hypotension, angioedema, throat irritation, rash including urticaria, pruritus, myalgia, and dizziness.

The clinical site should have necessary equipment, medications, adequately qualified and experienced staff with appropriate medical cover for the management of any infusion reaction, which may include, but is not limited to, oxygen, IV fluid, epinephrine (/adrenaline), acetaminophen (/paracetamol) and antihistamine.

If the participant experiences an infusion reaction, then supportive care should be used in accordance with the signs and symptoms. If a severe and potentially life-threatening infusion reaction occurs with sotrovimab/placebo, its use should be permanently discontinued. The following are AESIs for the agent sotrovimab or placebo for sotrovimab: infusion-related reactions; allergic/hypersensitivity reactions.

### Example 43

### Phase 3 Clinical Study of Sotrovimab for Prevention of COVID-19 Among Adult and Adolescent Subjects Exposed to SARS-CoV-2

A randomized, multi-center, double-blind, placebo-controlled Phase 3 study is performed to assess safety and efficacy of monoclonal antibody sotrovimab in preventing COVID-19 in adult and adolescent subject exposed to SARS-CoV-2.

A primary objective is to evaluate efficacy of sotrovimab versus placebo for prevention of symptomatic COVID-19 among close contacts of persons with SARS-CoV-2 infection and to determine safety and tolerability of sotrovimab compared to placebo.

Secondary objectives are:
Evaluate efficacy of sotrovimab in the prevention of asymptomatic SARS-CoV-2 infection compared to placebo among close contacts of persons with SARS-CoV-2 infection
Evaluate efficacy of sotrovimab in reducing duration of viral shedding among those with SARS-CoV-2 infection
Evaluate efficacy of sotrovimab in reducing the incidence, severity, and duration of clinical signs and symptoms of COVID-19 among those with SARS-CoV-2 infection
Evaluate efficacy of sotrovimab on the time to alleviation of clinical symptoms among those with SARS-CoV-2 infection
Evaluate efficacy of sotrovimab in reducing progression to severe disease among those with SARS-CoV-2 infection as assessed by rate of hospitalization, ICU admission, or death due to COVID-19
Evaluate the serum pharmacokinetics (PK) of sotrovimab
Evaluate potential immunogenicity (induction of ADA) response to sotrovimab

The exploratory objectives include:
Evaluate efficacy of sotrovimab in reducing viral load among those with SARS-CoV-2 infection
Evaluate association of SARS-CoV-2 viral load with treatment and with severity of disease
Monitor emergence of viral resistance to sotrovimab in subjects with confirmed SARS-CoV-2
Evaluate potential relationships between subject genetic polymorphisms and sotrovimab mechanisms of action and/or PK
Measure impact of sotrovimab on time away from work and work productivity due to COVID-19
Evaluate effects of sotrovimab on potential biomarkers of host response

### Criteria for Evaluation

A primary efficacy endpoint of this study is as follows:
Proportion of subjects with laboratory confirmed (RT-PCR) COVID-19 defined as one of or more the following through Week 2 (Day 15±1):
1. Acute respiratory symptoms (cough, sputum production, sore throat or shortness of breath); OR 2. Fever >38° C.; OR
3. 2 or more of the following symptoms (fatigue, myalgias/arthralgias, chills, nausea/vomiting, diarrhea, anosmia/dysgeusia)

The primary safety endpoints of this study are as follows:
The proportion of subjects with treatment emergent adverse events (AEs) and serious AEs (SAEs) following administration of sotrovimab
Clinical assessments including but not limited to laboratory test results Secondary endpoints of this study are as follows:
Incidence of laboratory confirmed (RT-PCR) asymptomatic SARS-CoV-2 infection through Week 2 (Day 15±1) in subjects with a negative baseline test
The difference in duration of viral shedding between sotrovimab and placebo treatment groups as assessed by RT-PCR from nasal swabs
The incidence, severity, and duration of clinical signs and symptoms of COVID-19
Percentage of participants requiring hospitalization due COVID-19
Percentage of participants requiring ICU care
Percentage of participants requiring mechanical ventilation or ECMO
Percentage of participants with COVID-19 related death
Concentrations of sotrovimab in serum
Incidence and titers (if applicable) of serum ADA to sotrovimab

The exploratory endpoints of this study include:
The difference in viral load at the time of illness onset between sotrovimab and placebo treatment groups as assessed by qRT-PCR from nasal swabs
Relationship between viral load (qRT-PCR) and severity of COVID-19
Incidence of laboratory confirmed SARS-CoV-2 infection by serology at Week 4 in subjects with a negative baseline test
FcR polymorphisms as determined by genotyping
Immunoglobulin G1 (IgG1) allotypes as determined by genotyping
Change from baseline in Work Productivity and Activity Impairment (WPAI)
Change from baseline in health-related quality of life according to EQ-5D-5L
Impact of sotrovimab administration on biomarkers of host response as assessed by host transcriptome and immunophenotyping analysis

### Number of Subjects

Approximately 1350 subjects (450 per treatment arm) are enrolled.

### Diagnosis and Main Eligibility Criteria

1. Male and female subjects aged 12 years or older
2. Close contact of a person (index) with known PCR-confirmed SARS-CoV-2 infection

Close contact defined as:
a. Residing with the index case in the 7 days prior to index diagnosis, including residence or staff in a congregate setting such as long-term care facility or nursing home
b. Medical staff, first responders, or other care persons
c. Less than 3 days since last exposure (close contact with a person with SARS-CoV-2 infection) to the index case
4. Female subjects must have a negative pregnancy test or confirmation of post-menopausal status
5. Negative rapid test for SARS-CoV-2 infection within 4 hours prior to dosing

### Duration of Study Participation

The estimated total time on study for each subject, inclusive of screening, is up to 36 weeks. Subjects are screened within 24 hours prior to randomization. Following study drug administration, subjects are evaluated for up to Week 6 (D43±3) for endpoint assessments and up to 36 weeks for safety follow-up.

### Study Design

This is a randomized, double-blind, placebo-controlled study of 2 dose levels of sotrovimab for the prevention of symptomatic SARS-CoV-2 infection in adults exposed to the virus. The study is designed to evaluate efficacy of a low and a high dose sotrovimab compared to placebo in prevention of symptomatic SARS-CoV-2 infection.

This study enrolls approximately 1350 asymptomatic male and female subjects 12 years of age or older, including those with comorbidities, who are close contacts (i.e. household members or healthcare providers) of persons with reverse transcriptase-polymerase chain reaction (RT-PCR)-confirmed SARS-CoV-2 infection. A rapid diagnostic test is used at screening to enrich for subjects without established infection. Eligible subjects are randomized to receive either sotrovimab low dose, sotrovimab high dose, or placebo in a 1:1:1 ratio as a single administration on Day 1.

An independent data monitoring committee (DMC) consisting of members with relevant expertise convenes to review study data. The purpose of the DMC is to monitor safety, efficacy, and futility and determine the need for sample size readjustment based on observed infection rate in the placebo arm. Two interim analyses are conducted. The first interim analysis is conducted after approximately 20% of subjects have been enrolled (˜90 per arm) to assess safety and tolerability of each dose level of S309 N55Q LS.

The second interim analysis is conducted after approximately 50% of subjects have been enrolled (˜225 per arm) to assess safety, futility, and efficacy. A dose level may be discontinued due to safety concerns or lack of efficacy.

If data emerges on effective agents for SARS-CoV-2 prophylaxis, the protocol may be modified to include new standard of care agents in the control arm.

The study includes a PK sub-study in approximately 75 subjects (25 per arm) for intensive PK and ADA sample collections. All other subjects have sparse PK and ADA sample collections. If a new lot of sotrovimab is introduced into the study, additional subjects may be included into an intensive PK and immunogenicity sub-study.

### Study Procedures

### Screening:

Screening is performed within 24 hours prior to randomization and includes written informed consent, determination of eligibility, collection of demographics, past and current medical conditions (including known pregnancy and/or lactation status), concomitant medications and information regarding exposure to the Index Case.

### Dosing (Day 1):

Following collection of a baseline nasal swab for RT-PCR on Day 1, eligible subjects are randomized to receive either sotrovimab or placebo. Subjects remain in the clinic for a minimum of 4 hours post-dose to assess safety and local tolerability of sotrovimab and complete assessments.

### Follow-Up Period:

Subjects complete a daily diary from Day 1 through Week 2 visit. This survey includes documentation of clinical signs and symptoms of infection and concomitant medication review. In addition, all subjects collect a nasal swab at home on Days 4, 7, and 10 to monitor for asymptomatic SARS-CoV-2 infection. A study team member contacts subjects via telephone to evaluate adverse events and provide support for completion of study procedures. Subjects have an in-clinic visit at Week 2 (Day 15±1) and Week 6 (Day 42±3) and complete assessments.

Subjects are followed per the COVID-19 Monitoring Schedule if they experience symptom onset defined as acute onset of respiratory symptoms (dry cough, shortness of breath, sore throat, or sputum production); OR fever of >38.0° C.; OR >2 of the following symptoms: myalgias/arthralgias, chills, nausea/vomiting, diarrhea, anosmia/dysgeusia.

Subjects meeting criteria for potential symptomatic COVID-19 complete an in-clinic evaluation for SARS-CoV-2 infection, including nasal swab for confirmation of infection. Subjects continue to complete a daily diary for 21 days following symptom onset. Additional nasal swabs are collected to assess viral load and duration of viral shedding. In these subjects, nasal swabs are collected every other day for the first week following a positive test and then every 3 days during the second- and third-week post infection.

Subjects are followed for up to 36 weeks following dosing for assessment of safety, PK and immunogenicity. The duration of follow up is informed by the predicted human t1/2 of sotrovimab based on allometric scaling of NHP PK. The final follow up period is specified in the protocol. Between Week 6 visit and the last follow-up visit, subjects are contacted by phone for adverse event monitoring.

### Product, Dosage, and Mode of Administration

Sotrovimab is an engineered human IgG monoclonal antibody directed against SARS-CoV-2 spike protein with modifications to increase half-life. Sotrovimab is administered in a single dose IV or IM at either a low or high dose level. Subjects randomized to placebo are administered sterile, preservative-free normal saline 0.9% solution by IM injection.

### Statistical Methods

Not all individuals exposed to SARS-CoV-2 will become infected or develop symptoms, and early estimates place family members at a 3% to 15% risk of infection after likely exposure. For the purpose of sample size calculation an estimated 10% attack rate was used. Sample size was chosen to achieve 80% power with 0.05 two-sided Type 1 error for the endpoint of laboratory confirmed symptomatic COVID-19, an attack rate of 10% and 50% efficacy of sotrovimab in reducing symptomatic COVID-19. Based on these assumptions, approximately 450 subjects are enrolled per treatment arm (total of 1350).

Two interim analysis are conducted for this study. The first interim analysis is conducted after approximately 20% of subjects have been enrolled (90 per arm) to assess safety and tolerability of each dose level of sotrovimab. A dose level may be discontinued due to safety concerns. The second interim analysis is conducted after approximately 50% of subjects have been enrolled (225 per arm) to assess safety, futility, and efficacy. A dose level may be discontinued due to safety concerns or lack of efficacy. Based on the attack rate observed at the interim analysis, sample size may be adjusted in order to increase the likelihood of achieving the overall expected number of cases to meet the primary endpoint. In order to properly control the overall type 1 error rate (the ‘alpha’) at the 0.05 level, O'Brien-Fleming method is used to allocate alpha between the second interim analysis (0.0052) and the final analysis (0.048).

All efficacy analyses are performed using the ITT population (all randomized subjects). Incidence of symptomatic and asymptomatic SARS-CoV-2 infection is also evaluated in the mITT population (subjects who are SARS-CoV-2 negative at the baseline visit). Safety is assessed in all subjects who received any amount of study drug.

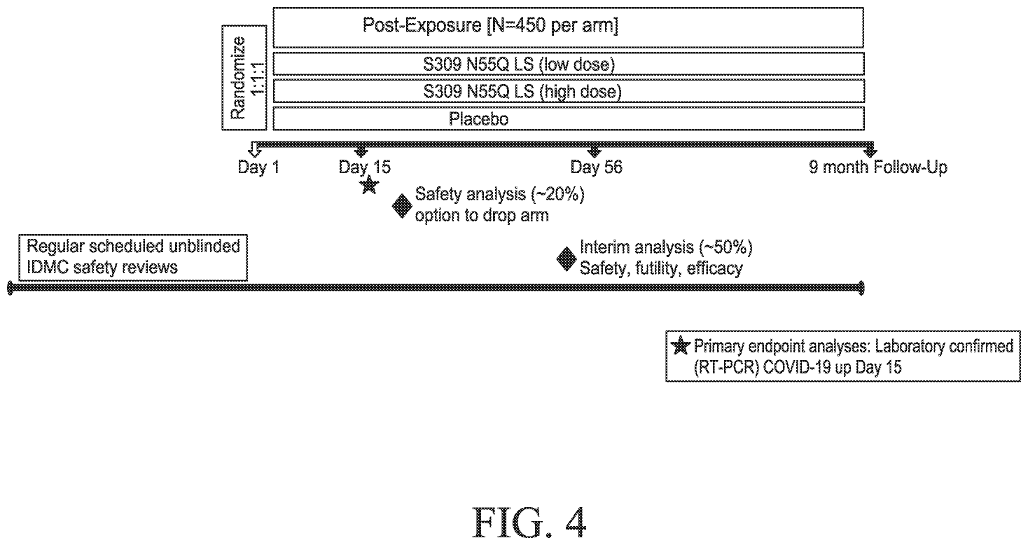

Further details of the study are shown in FIG. 4.

### Example 44

### Phase 3 Clinical Study of Sotrovimab for Prevention of COVID-19 Among Adult and Adolescent Subjects Exposed to SARS-CoV-2

A randomized, double-blind, placebo-controlled Phase 3 study is conducted to evaluate safety and efficacy of sotrovimab for prophylaxis of COVID-19. Approximately 3150 participants are randomized in a 2:1 ratio to receive either sotrovimab or placebo respectively to evaluate the safety and efficacy of sotrovimab for the prevention of COVID-19.

The primary objective is to evaluate safety and efficacy versus placebo in preventing COVID-19. The primary endpoint is incidence rate of protocol-defined COVID-19 through Week 13 (3 months) as defined by:
A positive SARS-CoV-2 reverse transcriptase polymerase chain reaction (RT-PCR) from a nasopharyngeal swab; AND
New onset (or exacerbation) of at least one or more of the following symptoms that persist or reoccur after a period of at least 24 hours:
Fever >38° C., chills, cough, sore throat, malaise, headache, myalgia, change in smell or taste, nasal congestion/rhinorrhea, vomiting, diarrhea, shortness of breath on exertion

This is a clinically meaningful endpoint to both clinicians and participants as prevention of symptomatic COVID-19 may lead to decreased morbidity and mortality from this disease as well as potentially lead to decreased transmission. In addition, a statistical testing hierarchy approach is employed to control the overall type I error to determine the efficacy of sotrovimab in the prevention of protocol-defined symptomatic COVID-19 at additional timepoints (up to Weeks 17, 21 and 26).

Other secondary efficacy endpoints including prevention of CDC-defined symptomatic COVID-19, and prevention of asymptomatic SARS-CoV-2 infection (as assessed by seroconversion at Weeks 13 and 26) are also clinically relevant.

A participant is considered to have completed the study if he/she has completed the study through to Week 26.

Participants 18 years or older at the time of giving informed consent (except for in South Korea, where participants are 19 years or older) and of any gender are eligible.

Female participants must meet and agree to abide by the contraceptive criteria listed below. Contraception use by women should be consistent with local regulations regarding the methods of contraception for those participating in clinical studies.

A female participant is eligible to participate if she is not pregnant or breastfeeding, and one of the following conditions applies:
a. Is a woman of non-childbearing potential (WONCBP), or
b. Is a woman of childbearing potential (WOCBP) and using a contraceptive method that is highly effective, with a failure rate of <1%, during the study intervention period and for up to 26 weeks after the last dose of study intervention.

The investigator should evaluate potential for contraceptive method failure (e.g., noncompliance, recently initiated) in relationship to the first dose of study intervention.

A WOCBP must have a negative highly sensitive pregnancy test (urine or serum as required by local regulations) before the first dose of study intervention.
a. If a urine test cannot be confirmed as negative (e.g., an ambiguous result), a serum pregnancy test is required. In such cases, the participant must be excluded from participation if the serum pregnancy result is positive.
b. Additional requirements for pregnancy testing during and after the study intervention may be in-place.

The investigator reviews medical history, menstrual history, and recent sexual activity to decrease the risk for inclusion of a woman with an early undetected pregnancy.

Participants are capable of giving informed consent. Participants agree to study procedures including the collection of nasopharyngeal swabs, self-collected nasal mid-turbinate swabs and venous blood as specified in the schedule of activities. Participants are able and willing to complete study surveillance questionnaires and activities.

Participants have an anti-spike SARS-CoV-2 antibody test during screening for enrollment. Participants with a positive anti-spike SARS-CoV-2 antibody test are not enrolled. Participants have a nasopharyngeal SARS-CoV-2 RT-PCR and an anti-N SARS-CoV-2 antibody test at Day 1 prior to dosing. Participants found to have a positive SARS-CoV-2 RT-PCR test or a positive anti-N SARS-CoV-2 antibody test at Day 1 are excluded from the primary analysis (mITT) population.

### Exclusion Criteria

Participants are excluded from the study if any of the following criteria apply:

### Medical Conditions

1. History of prior positive SARS-CoV-2 RT-PCR or antigen test or history of positive SARS-CoV-2 serology test including during screening.
2. Febrile illness with or without respiratory symptoms (e.g., cough, nasal congestion) within 7 days prior to randomization
3. Unstable medical condition and not expected to survive for the duration of study participation as judged by the investigator
4. Participant has any condition that would prohibit receipt of intramuscular injections in the investigator's opinion such as coagulation disorder, bleeding diathesis, or thrombocytopenia
5. Known hypersensitivity to any constituent present in the investigational product 6. Previous anaphylaxis or hypersensitivity to a monoclonal antibody
7. Participants who, in the judgment of the investigator, will be unlikely or unable to comply with the requirements of the protocol through Week 26

### Prior Medications

8. Receipt of a COVID-19 vaccine at any time prior to enrollment, or planned receipt of a COVID-19 vaccine during the first 13 weeks of the study
9. Receipt of any vaccine within 48 hours prior to enrollment. Vaccination will not be allowed for 2 weeks after dosing
10. Receipt of any SARS-CoV-2 hyperimmune intravenous immunoglobulin (hIVIG) from COVID-19 survivors
11. Receipt of convalescent plasma from a recovered COVID-19 patient or an anti-SARS-CoV-2 mAb within 3 months or 5 half-lives, whichever is longer, prior to screening and/or during the study

### Prior/Concurrent Clinical Study Experience

12. Enrolment in any investigational drug or device study for SARS-CoV-2/COVID-19 within 90 days prior to Day 1 or within 5 half-lives of the investigational compound, whichever is longer.

### Other Exclusions

13. Pregnant or breast-feeding females.
(all female participants regardless of childbearing potential status must have a negative pregnancy test at screening/pre-dosing)

Sotrovimab is formulated in solution in a single use vial (62.5 mg/mL). the unit dose strength is 500 mg/vial (500 mg/8 mL). Sotrovimab is administered in a single dose (500 mg, once) by intramuscular injection. Placebo is locally sourced sterile 0.9% (w/v) sodium chloride solution, administered in a single dose by intramuscular injection.

### Brief Summary

### Lead-In Phase

The Lead-in phase of the study is intended to evaluate the safety, tolerability, and pharmacokinetics (PK) of sotrovimab with frequent assessments of injection site reactions (ISRs) and intensive blood sampling for PK analysis. These participants are pooled with the Expansion phase participants for efficacy evaluations. Approximately 21 participants are enrolled and randomized 2:1 to receive VIR-7831 (N=14) or equal volume saline placebo (N=7). Participants are monitored for 6 hours after dosing for safety monitoring and local injection site tolerability assessments. At the end of 6 hours, they are discharged. Randomization is paused upon enrollment of the Lead-in phase, until Day 8 safety assessment by the Joint Safety Review Team (JSRT) recommends continuing. If the JSRT requires escalation to the IDMC, then randomization pauses pending a recommendation to proceed by the IDMC.

### Intensive PK Sampling and Immunogenicity

All participants enrolled in the Lead-In phase have additional timepoints for PK blood sampling. Serum PK and anti-drug antibodies (ADA) samples are collected.

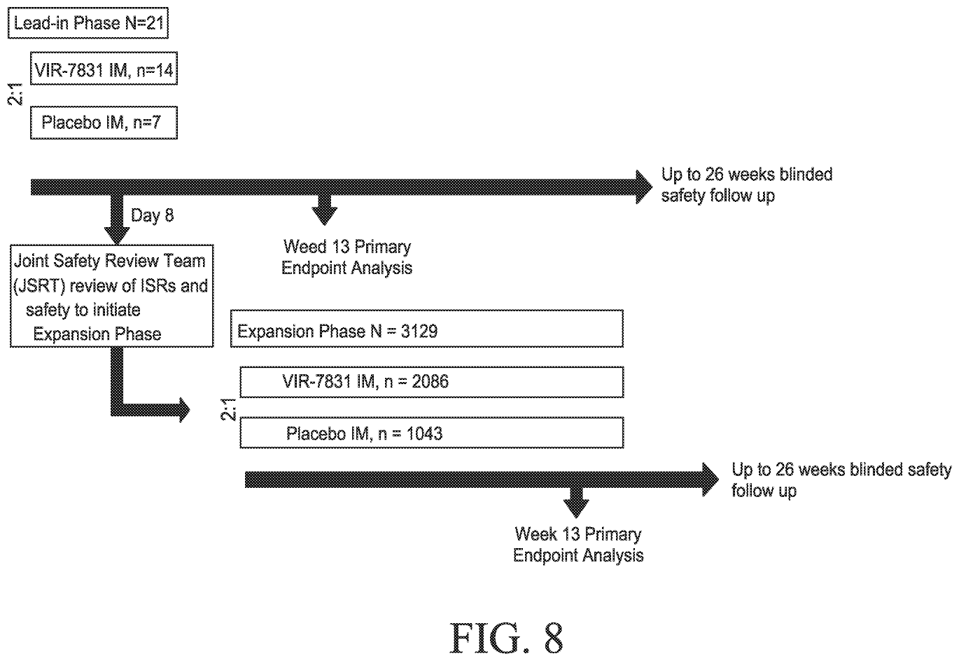

The lead-in phase of the study is summarized in FIG. 8, wherein sotrovimab is identified as VIR-7831.

### Expansion Phase

The Expansion phase of the study is gated to a safe to proceed recommendation from the JSRT. Specifically, randomization into the Expansion phase starts following blinded assessment of local injection site tolerability assessment data (ISRs) and other safety data from the Lead-in phase through Day 8 of follow-up by the JSRT.

Approximately 3129 additional participants are enrolled and randomized in a 2:1 ratio to receive sotrovimab or equal volume saline placebo via IM administration respectively.

Participants in the Expansion phase are monitored through 1 or 2 hours post-dose prior to discharge from the clinic/study unit. The duration of monitoring (1 or 2 hours) is based on assessment of ISRs from the Lead-in phase. The decision regarding the duration of monitoring is communicated as part of the JSRT recommendations to sites, and as a protocol file note.

Participants from both the Lead-in and Expansion phases are monitored for COVID-19 throughout the study via two mechanisms, as follows:
1. Participants are provided with a printed guide of COVID-19 signs and symptoms to look out for, and a study site phone number to call within 24 hours of experiencing any of those symptoms, or if they have any other reason to suspect they may be infected with SARS-CoV-2.
2. In addition, the site makes a regular once-weekly phone call to the participant, from Day 1 through Week 13 (every two weeks from Week 13 to Week 26), to capture adverse events (AEs), serious adverse events (SAEs) and to review the COVID-19 symptoms checklist with the participant.

All participants are provided with a nasal (mid-turbinate) swab self-test kit and return envelope for central lab testing, at the start of the study. Any participant who experiences COVID-19 symptoms during the 26-weeks study follow-up period, are instructed to use the self-test kit immediately, and to follow any additional local guidance for managing suspected COVID-19. Those that test positive complete weekly self-reported COVID-19 symptom surveillance and weekly Work Productivity and Activity Impairment (WPAI) questionnaires and are followed per a COVID-19 Illness Monitoring Schedule for 4 weeks after symptom onset. If a participant tests negative and is still experiencing symptoms, a second self-collection kit is sent to the participant for repeat testing.

All participants have regular follow-up phone calls and attend specific follow-up visits for assessments of efficacy, safety, and PK (sparse).

### Number of Participants:

Approximately 3150 participants are randomly assigned to the study intervention in a 2:1 ratio to sotrovimab or placebo. Any participant who receives the study intervention are considered evaluable.

### Study Screening, Randomization, and Duration

Screening assessments are performed during the 14 days prior to Day 1. Eligible participants are treated in a blinded manner with an IM dose on Day 1 and followed up to End of Study (EOS)—26 weeks (6 months). Participants remain blinded through EOS. Participants who develop COVID-19 (hospitalized or not hospitalized) are followed through 4 weeks post-symptomatic infection or EOS, whichever is earlier.

Randomization of participants in both the Lead-in and Expansion phases are stratified based on the following:
Age strata (<65 years of age, ≥65 years of age) Increased risk exposure group
Presence of an immunocompromising condition

Block randomization occurs by country.

### Independent Data Monitoring Committee

An unblinded Independent Data Monitoring Committee (IDMC) evaluates the overall safety profile of sotrovimab including evaluation of the incidence and severity of COVID-19 cases in participants randomized to sotrovimab compared to those randomized to placebo throughout the conduct of the study. The roles and responsibilities of the IDMC, including membership, scope, frequency of meetings and communication plan are defined in the IDMC charter. IDMC meetings occur regularly throughout the study as outlined by the IDMC charter.

### Joint Safety Review Team

A Joint Safety Review Team (JSRT) comprised of team members from clinical research, pharmacovigilance, and statistics from study sponsors determines if a safety concern identified during instream blinded data review needs to be escalated to the IDMC. The JSRT is responsible for blinded safety review of the Lead-in phase as described above, with escalation to the IDMC if needed. The IDMC acts in accordance with the process defined in the IDMC Charter. Initiation of the Expansion phase is contingent on a safe to proceed recommendation from the JSRT. The responsibilities of the JSRT and frequency of assessments are available in relevant safety review team (SRT) documents.

The incidence rate of protocol-defined COVID-19 through Week 13 (3 months) and is analyzed using a Poisson regression model to estimate the relative risk. The model includes terms for treatment group, age strata, exposure risk group, immunocompromised group and country. However, for countries with no events, countries may be collapsed into regions for the model. The relative risk, 95% confidence interval and corresponding p-value are presented for the comparison of sotrovimab with placebo.

### Example 45

### Intramuscular Sotrovimab for Mild/Moderate COVID-19

A Phase 3 randomized, multi-center, open label study is conducted to assess the efficacy, safety, and tolerability of sotrovimab given intramuscularly versus intravenously for the treatment of mild/moderate COVID-19 in high-risk non-hospitalized patients. Arms and interventions are as in Table 15.

TABLE 15
Arms and Interventions

| | Assigned |
|---|---|
| Arms | Interventions |
| Active Comparator: Sotrovimab 500 mg IV | Sotrovimab |
| Sotrovimab given by intravenous infusion | 500 mg IV |
| Experimental: Sotrovimab 500 mg IM Sotrovimab | Sotrovimab |
| given by intramuscular injection | 500 mg IM |
| Experimental: Sotrovimab 250 mg IM Sotrovimab | Sotrovimab |
| given by intramuscular injection | 250 mg IM |

### Outcome Measures

### Primary Outcome Measure:

1. Progression of COVID-19 [Time Frame: Up to Day 29]

### Secondary Outcome Measure:

2. Occurrence of adverse events (AEs) [Time Frame: Up to 24 weeks]
3. Occurrence of serious adverse events (SAEs) [Time Frame: Up to 24 weeks]
4. Occurrence of adverse events of special interest (AESI) [Time Frame: Up to 24 weeks]
5. Incidence and titers (if applicable) of serum anti-drug antibody (ADA) to sotrovimab [Time Frame: Up to 24 weeks]
6. Mean area under the curve of SARS-CoV-2 viral load in nasal secretions as measured by qRT-PCR [Time Frame: Up to Day 8]
7. Change from baseline in viral load by qRT-PCR [Time Frame: Up to Day 8]
8. Proportion of participants with a persistently high SARS-CoV-2 viral load at Day 8 by qRT-PCR [Time Frame: Up to Day 8]
9. Development of severe and/or critical respiratory COVID-19 [Time Frame: Up to Day 29]
10. IV and IM sotrovimab pharmacokinetics (PK) in serum [Time Frame: Up to Week 24]

### Eligibility

Minimum Age: 12 Years
Maximum Age:
Sex: All
Gender Based: No
Accepts Healthy Volunteers: No
Criteria: Inclusion Criteria:
   Participant must be aged 12 years or older at time of consent AND at high risk of progression of COVID-19 or ≥65 years old
   Participants must have a positive SARS-CoV-2 test result and oxygen saturation ≥94% on room air and have COVID-19 symptoms and be less than or equal to 7 days from onset of symptoms
Exclusion Criteria:
   Currently hospitalized or judged by the investigator as likely to require hospitalization in the next 24 hours
   Symptoms consistent with severe COVID-19
   Participants who, in the judgement of the investigator are likely to die in the next 7 days
Known hypersensitivity to any constituent present in the investigational product

### Example 46

### Phase II Study of a Second Generation Sotrovimab Material in Non-Hospitalized Participants with Mild-to-Moderate Coronavirus Disease 2019 (Covid-19)

Sotrovimab drug material used in the studies described in Examples 39-40 was produced using a non-clonal pool of stably transfected cells. This is referred to as “Gen1” drug material. “Gen2” drug material is made using a clonal master cell bank derived from the original pool of transfectants. A multicenter, randomized, double-blind, parallel group phase II study is performed to evaluate safety, tolerability and pharmacokinetics of Gen2 drug materials in non-hospitalized participants with mild to moderate COVID-19.

The primary objective is to evaluate safety and tolerability of Gen2 and Gen1 sotrovimab drug material. Endpoints are: occurrence of adverse events (AEs) through Day 29; occurrence of serious adverse events (SAEs) through Day 29; occurrence of adverse events of special interest (AESIs) through Day 29; occurrence of clinically significant abnormalities on 12-lead electrocardiogram (ECG) readings through Day 29; and occurrence of disease progression events (not classified as AEs) through Day 29.

Secondary objectives are:
(1) To assess the pharmacokinetics (PK) of sotrovimab Gen2 and Gen1 in serum
   Endpoints: Cₘₐₓ, Cₗₐₛₜ, Tₘₐₓ, Tₗₐₛₜ, AUC_{D0-28}, AUC_{inf}, AUCₗₐₛₜ, % AUCₑₓₚ, t_{1/2}, λ_{z}, V_{z}, Vₛₛ, CL;
(2) To evaluate safety and tolerability profile of sotrovimab Gen2 and Gen1
   Endpoints: occurrence of SAEs through Week 24; occurrence of AESIs through Week 24; occurrence of clinically significant abnormalities on 12-lead ECG readings through Week 24; occurrence of disease progression events (not classified as AEs) through Week 24.

Exploratory endpoints are:
(1) To assess potential immunogenicity of sotrovimab Gen2 and Gen1
   Endpoint: Incidence and titers (if applicable) of serum antidrug antibodies (ADA) to sotrovimab
(2) To characterize the effect of sotrovimab Gen2 and Gen1 on the viral shedding profile in upper respiratory samples.
   Endpoint: Change from baseline in viral load at all visits through Day 29 as measured by quantitative reverse transcriptase polymerase chain reaction (qRT-PCR) from saliva samples.

This study is a randomized, double-blind, multi-center, parallel group phase II trial of sotrovimab, a monoclonal antibody (mAb) against SARS-CoV-2 for the prevention of progression of mild to moderate COVID-19 in non-hospitalized patients. Participants with early mild to moderate COVID-19 are randomized 3:1 to receive a single, 500 mg intravenous infusion of either Gen2 or Gen1 study material. Safety, tolerability and PK are evaluated.

Screening assessments are performed within 24 hours before the start of infusion. Eligible participants are treated in a blinded manner with a single IV dose on Day 1 and followed up to 24 weeks. Participants re monitored for approximately 9 hours on Day 1 for safety assessments and intensive PK sampling.

The study includes nonhospitalized patients with mild to moderate SARS-CoV-2 infection as confirmed by local laboratory tests and/or point of care tests. A single dose level of Gen1 or equal volume Gen2 material is studied and delivered via IV infusion. Participants receive the infusion in a clinic/study unit where they are monitored closely for adverse events in the post-infusion period. Subsequent visits for study activities and clinical monitoring are conducted via clinic or home nursing visits (except for Week 16). A double-blind design is a standard methodology for randomized, controlled studies to minimize bias. A Gen1 study arm is included to maintain blinding of Gen1 versus Gen2 sotrovimab study material receipt during the conduct of the study. This blinding is critical for minimizing bias that may occur during study assessments, including during evaluation of AEs and disease-related safety outcomes.

Blinded safety data are reviewed regularly by the JSRT through Week 24. There is no placebo in this study because the primary aim is to evaluate safety and tolerability of Gen2 material. All participants receive standard of care for COVID-19 disease during this study, including admission to a hospital if deemed necessary by the responsible investigator.

Analytical comparability studies support the use of Gen2 in clinical studies.

### Example 47

### ACE2-Independent Mechanism of SARS-CoV2 Neutralization by S309 Antibody

In the following experiments, S309 antibody (VH of SEQ ID NO.:105, VL of SEQ ID NO.:168) was expressed as recombinant IgG1 with M428L and N434S Fc mutations. The effect of ACE2 overexpression on S309 antibody neutralization of infection was investigated. Vero E6 or Vero E6-TMPRSS2 cells were infected with SARS-CoV-2 (isolate USA-WA1/2020) at MOI 0.01 in the presence of S309 (10 μg/ml). Cells were fixed 24h post infection, viral nucleocapsid protein was immunostained and quantified. Nucleocapsid staining was effectively absent in antibody-treated cells. S309 had an IC50 (ng/mL) in Vero E6 cells of 65 and in Vero E6-TMPRSS2 of 91 (data not shown).

A panel of 7 cell lines (HeLa, 293T (wt), Vero E6, Huh7, 293T ACE2, MRC 5-ACE2-TMPRSS2, A549-ACE2-TMPRSS2 clone 5, A549-ACE2-TMPRSS2 clone 10) were infected with SARS-CoV-2-Nluc or VSV pseudotyped with the SARS-CoV-2 spike protein in the presence of 5309. Luciferase signal was quantified 24h post infection. S309 maximum neutralization values were as shown in Table 16.

TABLE 16
Maximum Neutralization Values of S309

Virus/Pseudotype

| | SARS-CoV- | VSV |
|---|---|---|
| Cell Type | 2-Nluc | Pseudotype |
| Vero E6 | >99% | >99% |
| Vero E6-TMPRSS2 | >99% | 96% |
| Huh7 | 98% | 78% |
| 293T ACE2 | 26% | 34% |
| MRC5-ACE2-TMPRSS2 | 87% | 45% |
| A549-ACE2-TMPRSS2 clone 5 | 89% | 65% |
| A549-ACE2-TMPRSS2 clone 10 | 81% | 42% |

Binding of purified, fluorescently-labelled SARS-CoV-2 spike protein binding to these cell lines was quantified by flow cytometry. HeLa and 239T WT cells had the lowest MFIs, followed by Huh7 and VeroE6 cells. 293T ACE2 cells (highest), MRC 5-ACE2-TMPRSS2 (third-highest), A549-ACE2-TMPRSS2 clone 5 (fourth-highest), and A549-ACE2-TMPRSS2 clone 10 (second-highest) had higher MFIs. Correlation analysis between spike binding maximum neutralization potential of S309 was determined; S309 Spearman correlation values were: r=−0.94 for both viral models. p=0.017.

To further characterize SARS-CoV-2-susceptible cell lines, the seven cell lines described above were incubated with purified, fluorescently-labeled SARS-CoV-2 spike protein or RBD protein and protein binding was quantified by flow cytometry. In descending order of MFI, the cell lines were: A549-ACE2-TMPRSS2 clone 10; 293T ACE2; MRC 5-ACE2-TMPRSS2; A549-ACE2-TMPRSS2 clone 5; Vero E6; Huh7; 293T (wt); and HeLa.

Selected lectins and published receptor candidates were screened using HEK293T cells infected with SARS-CoV-2 VSV pseudoviruses. ACE2, DC-SIGN, L-SIGN, and SIGLEC-1 gave the highest signals. ACE2 provided a signal of approximately 10⁵ relative luminescence units (RLUs), and DC-SIGN, SIGLEC-1, and L-SIGN had signals of approximately 104 RLUs. All other lectins/candidates tested gave signals of approximately 10²-10³ RLUs.

HEK 293T, HeLa and MRC5 cells were transiently transduced to overexpress DC-SIGN, L-SIGN, SIGLECI or ACE2 and infected with SARS-CoV-2 VSV pseudoviruses. Uninfected cells and untransduced cells were included as controls. In HEK293T cells, ACE2, DC-SIGN, SIGLEC-1, and L-SIGN all provided substantial increases in infection. In HeLa and MRC5 cells, only ACE2 increased infection.

Stable HEK293T cell lines overexpressing DC-SIGN, L-SIGN, SIGLEC-1 or ACE2 were infected with authentic SARS-CoV-2 (MOI 0.1), fixed and immunostained at 24 hours for the SARS-CoV-2 nucleoprotein. Wild-type cells (infected and uninfected) were used as controls. Increased staining was observed in cells overexpressing DC-SIGN, L-SIGN, or SIGLEC-1, and staining was significantly increased in cells overexpressing ACE2.

Stable cell lines were infected with SARS-CoV-2-Nluc and luciferase levels were quantified at 24 hours. In ascending order of RLUs: uninfected (approx. 10²-10³ RLUs); parental 293T (approx. 104 RLUs); DC-SIGN (approx. 105 RLUs); L-SIGN (approx. 105 RLUs); SIGLEC-1 (approx. 10⁵-10⁶ RLUs); ACE2 (>107 RLUs).

Stable cell lines were incubated with different concentration of anti-SIGLEC1 mAb (clone 7-239) and infected with SARS-CoV-2-Nluc. Infection as a percentage of untreated cells remained near to exceeded 100% in 293T cells expressing DC-SIGN, L-SIGN, or ACE2, but dropped to below 50% (0.2 μg/mL anti-SIGLEC) to close to 0 (1 g/mL or 5 μg/mL anti-SIGLEC) in 293T cells expressing SIGLEC-1.

Single cell expression levels of selected potential SARS-CoV-2 (co)receptor candidates were determined in different lung cell types derived from the Human Lung Cell Atlas (nature.com/articles/s41586-020-2922-4). DC-SIGN, L-SIGN and SIGLEC-1 are expressed in a variety of cell types in the lung at levels similar to or even higher than ACE2.

Binding of antibodies targeting DC-/L-SIGN, DC-SIGN, SIGLEC1 or ACE2 on HEK293T cells stably over-expressing the respective attachment receptor was analyzed by flow cytometry and immunofluorescence analysis. HEK 293T cells over-expressing the respective attachment receptors were infected with VSV pseudotyped with SARS-COV-2 wild type spike or spike bearing mutations of the B1.1.7 lineage. Luminescence was analyzed one day post infection. Infection was increased in cells expressing the attachment receptors. Infection by VSV pseudotyped with either spike was similar for each test group. Cells expressing ACE2 gave the highest luminescence signal.

Vero E6 cells, in vitro differentiated moDCs or PBMCs were infected with SARS-CoV-2 at MOI 0.01. At 24h post infection, cells were fixed, immunostained for viral nucleocapsid protein and infected cells were quantified. Only VeroE6 cells showed infection (approximately 7% of cells). Supernatant of the infected cells was taken at 24, 48 and 72h and infectious viral titer was quantified by FFU assay on Vero E6 cells.

Major cell types with detectable SARS-CoV-2 genome in bronchoalveolar lavage fluid (BALF) and sputum of severe COVID-19 patients were assessed. A t-SNE plot was generated, and the count of each SARS-CoV-2+ cell type was determined (total n=3,085 cells from 8 subjects in Ren et al. Cell 2021). Cell types were T, NK, plasma, neutrophil, macrophage, ciliated, squamous, and secretory. Expression of ACE2, DC-SIGN, L-SIGN, SIGLEC-1, and combinations of these was assessed for each cell type.

ACE2, DC-SIGN (CD209), L-SIGN (CLEC4M), SIGLEC1 transcript counts were correlated with SARS-CoV-2 RNA counts in macrophages and in secretory cells. Correlation was based on counts (before log transformation), from Ren et al. Cell 2021.

Cell lines were generated to overexpress DC-SIGN, L-SIGN or ACE2 by transducing HEK293T cells with lentivirus encoding the transgene, and immunofluorescence assays were performed to assess transgene expression.

Expression of DC-SIGN or L-SIGN increased pseudovirus infection levels by over 10-fold compared to infection of WT HEK293T cells, and expression of ACE2 increased pseudovirus infection levels by over 100-fold compared to infection of WT HEK293T cells.

Neutralizing activity of exemplary mAb S309 against the VSV pseudovirus was assessed in the engineered HEK293T cells. S309 fully neutralized infection via DC-SIGN and L-SIGN, and to a lesser extent, ACE2.

The ability of live SARS-CoV-2 with luciferase reporter to infect the HEK293T cells was examined using a luminescence assay. Expression of DC-SIGN or L-SIGN increased live virus infection levels by over 3-fold compared to infection of WT HEK293T cells, and expression of ACE2 increased live virus infection levels by over 100-fold compared to infection of WT HEK293T cells.

Neutralizing activity of mAb S309 against the VSV pseudovirus was assessed in the engineered HEK293T cells. S309 fully neutralized infection via DC-SIGN and L-SIGN, and neutralized infection via ACE2 to a lesser extent.

Experiments were performed to investigate whether S309 antibody can neutralize entry of SARS-CoV-2 via SIGLEC-1. Briefly, stable cell HEK293T lines were generated as described above to overexpress DC-SIGN/L-SIGN, DC-SIGN, SIGLEC-1, or ACE2. Expression of DC-SIGN, L-SIGN, or SIGLEC increased live virus infection levels by over 10-fold compared to infection of WT HEK293T cells, and expression of ACE2 increased pseudovirus infection levels by over 100-fold compared to infection of WT HEK293T cells. S309 fully neutralized infection via DC-SIGN, L-SIGN, and SIGLEC-1.

Expression of DC-SIGN (CD209) and other cell surface receptor proteins including SIGLEC-1 and other SIGLECs was determined on a variety of cell types.

Further experiments were performed to investigate the function(s) of DC-SIGN, L-SIGN, and SIGLEC-1 in SARS-CoV-2 infection. In one set of experiments, HEK293T cells stably expressing DC-SIGN, L-SIGN, SIGLEC-1 or ACE2 were infected with live SARS-CoV-2 Nluc at three different multiplicities of infection (MOI): 0.01, 0.1, and 1). Infection was determined using relative luminescence units and compared to infection in HEK293T cells (parental). At the lowest MOI tested, an increase of infection in cells expressing DC-SIGN, L-SIGN, or SIGLEC was observed. At the highest MOI tested, infection was not further increased versus parental by expression of DC-SIGN, L-SIGN, or SIGLEC. These data indicate that the parental 293T cells are susceptible to infection by SARS-CoV-2 and L-SIGN, DC-SIGN, and SIGLEC-1 enhance infection levels but do not function as primary receptors for infection.

In another set of experiments, 293T cells, HeLa cells, and MRC5 cells were transiently transduced with lentivirus encoding DC-SIGN, L-SIGN, SIGLEC-1 or ACE2 and infected with VSV pseudovirus three days after transduction. While the 293T cells showed a low level of susceptibility (compare uninfected with untransduced), HeLa and MRC5 cells were completely refractory to the virus. The low level of infection in 293T cells can be increased by expression of L-SIGN, DC-SIGN, or SIGLEC-1, consistent with a role for these proteins as attachment factors. The HeLa and MRC5 cells remained refractory to infection even after expression of L-SIGN, DC-SIGN, or SIGLEC-1, and only become susceptible after expression of ACE2. These data indicate that L-SIGN, DC-SIGN, and SIGLEC-1 are not primary receptors for SARS-CoV-2.

### Example 48

### In Vivo Efficacy of S309 Antibody in an Animal Model of Infection

The efficacy of S309 was investigated in Syrian hamsters. This animal model represents to-date the most relevant model of SARS-CoV-2 infection that did not require in vivo over-expression of ACE2 to support productive infection and disease. Prophylactic administration of S309 induced dose-dependent protection against SARS-CoV-2 infection and tissue damage in hamsters, as demonstrated by the viral RNA levels, the viral load as well as the histopathological score in the lungs. These data indicate that poor and incomplete neutralization of entry by S309 in vitro when using ACE2 over-expressing cells did not compromise in vivo efficacy of non-RBM mAbs.

S309 carrying the N297A mutation has a reduced capacity to trigger effector functions as a consequence of diminished engagement to Fcγ receptors. This was further confirmed by the reduced binding of S309-N297A variant to hamster monocytes in the spleen. The in vivo efficacy measured with the N297A mAb is similar or just slightly inferior to the wt S309, suggesting that neutralizing capacity of the mAb is prevailing upon its effector function capacity in these conditions. The serum concentration of S309 required to reduce the viral RNA in the lung by 90% was 9 μg/ml.

Sotrovimab demonstrated activity in a hamster model of SARS-CoV-2. IP administration of sotrovimab at >5 mg/kg prior to intranasal inoculation resulted in a dose-dependent improvement in all measured outcomes (body weight, lung weight, total viral RNA in the lungs, and infectious virus levels based on TCID50 measurements).

In addition, sotrovimab activity was assessed in vivo in a Golden Syrian Hamster model of SARS-CoV-2 infection using the UK B.1.1.7 variant. Sotrovimab neutralized the SA and Brazil variants. A slight shift in activity was observed with the UK variant with a geometric mean EC50 3.0-fold shift versus wild-type and EC90 4.1-fold shift versus wild-type. Protection was observed in B.1.1.7-infected hamsters receiving sotrovimab based on significant differences in body weight at both days three and four post-infection in animals receiving 30 mg/kg and 5 mg/kg sotrovimab compared to isotype control antibody-treated animals.

### Example 49

### Antibody Activity Against SARS-CoV-2 Variants

A number of SARS-CoV-2 variants have emerged, with increasing numbers of infection by variants reported in late 2020. The Receptor Binding Motif (RBM) appears to be particularly variable to mutation. Notable emerging variants have been observed in Scotland, the UK, South Africa, California, Columbus, and in minks in Denmark, and some mutations have been reported to confer escape from antibodies or serum neutralization. Experiments were performed to assess the ability of S309 antibodies to neutralize variants. S309 N55Q MLNS (VH: SEQ ID NO.:113; VL: SEQ ID NO.:168; with M428L and N434S Fc mutations) was tested against SARS-CoV-2 bearing a panel of the 20 most-frequent SARS-CoV-2 RBD variant mutations, as determined by sequence reads. Antibodies REGN10933 and REGN10987 (Hansen et al., Science 369 (6506):1010-1014; eabd0827-0810 (2020) and PDB 6XDG (rcsb.org/structure/6XDG)) were included for comparison. Results are summarized in Table 17.

TABLE 17
Summary of Neutralization by Antibodies Against SARS CoV-2 Variants

| | S309 N55Q MLNS | | |
|---|---|---|---|
| Variant Mutation | (sotrovimab) | REGN10933 | REGN10987 |
| N501Y (UK, South African, | Y | Y | Y |
| and Brazilian mutant) | | | |
| S477N | Y | Y | Y |
| N439K (Scottish mutant) | Y | Y | N |
| L452R (Californian mutant) | Y | P | P |
| E484K (South African and | Y | N | Y |
| Brazilian mutant) | | | |
| Y453F (mink mutant) | Y | N | N |
| | | | (4x decrease) |
| A520S | Y | Y | Y |
| K417N (South African mutant) | Y | N | Y |
| | (K417N/V) | | (K417N/E/V) |
| S494P | Y | N | P |
| S477R | P | ? | P |
| V367F | Y | Y | Y |
| P384L | Y | P | P |
| A522S | Y | P | P |
| A522V | Y | P | P |
| V382L | Y | P | P |
| N501T | Y | P | P |
| P330S | Y | P | P |
| T478I | Y | ? | P |
| S477I | Y | ? | P |
| P479S | Y | P | P |

Y = less than three-fold decrease in neutralizing of live virus or pseudovirus
N = greater than three-fold decrease in neutralizing of live virus or pseudovirus
P = neutralization by antibody is predicted due to variant amino acid being outside of epitope
? = unknown.

Total counts of SARS-CoV-2 sequenced mutants known to escape the antibodies (as of Jan. 29, 2021) were: S309 N55Q MLNS=29; REGN10987=10,425; REGN10933=3,621.

Binding of S309 antibodies to SARS-CoV-2 variant RBDs was assessed by BLI. S309 (VH: SEQ ID NO.:105; VL: SEQ ID NO.:168) with wild-type Fc and S309 N55Q (VH: SEQ ID NO.:113; VL: SEQ ID NO.:168) bearing MLNS or MLNS+ GAALIE Fc mutations were assessed. REGN10987 and REGN10933 were included as comparators. Briefly, antibodies were diluted in kinetics buffer at 3 μg/ml and loaded on Protein-A sensors for 75 seconds. After a short equilibration step in kinetics buffer, loaded sensors were moved in wells containing the RBD variants at 5 μg/ml in kinetics buffer and association was recorded during 3 minutes. Dissociation of the complex was performed in kinetics buffer for 3 minutes.

Neutralization of S309 antibodies against SARS-CoV-2 variants was assessed using MLV pseudovirus and Vero-E6 target cells expressing TMPRSS2. S309 (VH: SEQ ID NO.: 105; VL: SEQ ID NO.:168) with wild-type Fc and S309 N55Q (VH: SEQ ID NO.:113; VL: SEQ ID NO.:168) bearing MLNS (Sotrovimab) or MLNS+ GAALIE (VIR-7832) Fc mutations were assessed. REGN10987, REGN10933, and the combination of REGN10987+REGN10933, were also assessed. S309 antibodies (S309, sotrovimab, and VIR-7832) neutralized the SARS-Cov-2 variants.

Sotrovimab was assessed for in vitro activity against SARS-CoV-2 live virus variants of concern from the United Kingdom (UK) (B.1.1.7), South Africa (SA) (B.1.351) and Brazil (P.1). Sotrovimab exhibited an average fold change in EC₅₀ of 3.0 or less against each variant.

Sotrovimab was also assessed in a pseudotyped virus assay for neutralization activity against other SARS-CoV-2 variants of concern. Results are presented in Table 18.

TABLE 18

| | | Average Fold Change |
|---|---|---|
| SARS-CoV-2 | | in EC₅₀ Compared to |
| Variant Name | Variants in Tested Spike Sequence | Relative Wild-Type <3.0 |
| B.1.1.7 (UK) | H69-, V70-, Y144-, N501Y, A570D, D614G, | Y |
| | P681H, T716I, S982A, D1118H | |
| B.1.1.7 + E484K | H69-, V70-, Y144-, E484K, N501Y, A570D, | Y |
| | D614G, P681H, T716I, S982A, D1118H | |
| B.1.351 (South | L18F, D80A, D215G, R246I, K417N, E484K, | Y |
| Africa) | N501Y, D614G, A701V | |
| P.1 (Brazil) | D138Y, D614G, E484K, H655Y, K417T, L18F, | Y |
| | N501Y, P26S, R190S, T1027I, T20N, V1176F | |
| B.1.427/B.1.429 | D614G, L452R, S13I, W152C | Y |
| (California) | | |
| B.1.617 (India) | T95I, G142D, E154K, L452R, E484Q, D614G, | Y |
| | P681R, | |

Using a mAb (VIR-7832 aka S309 N55Q MNLS_GAALIE) with identical variable regions and SARS-CoV-2 spike protein binding characteristics as sotrovimab but also containing the immune-modulating modifications G236A/A330L/I332E, resistance selection studies, studies examining generation of variants conferring reduced susceptibility and studies characterizing effector functions were performed with sotrovimab and VIR-7832. No viral breakthrough was observed when virus was passaged for 10 passages (34 days) in the presence of fixed concentration of antibody even at the lowest concentration tested (˜10×EC50) suggesting a high barrier to resistance. Forcing the emergence of resistance variants through an increasing concentration selection method identified E340A as a sotrovimab mAb resistance mutant (MARM). In vitro pseudotyped virus experiments examining currently circulating epitope polymorphisms demonstrate that amino acid substitutions at P337 (T/L/H/R) and E340 (A/K/G) confer reduced susceptibility to sotrovimab. Notably, E340 and P337 are >99.99% conserved among available SARS-CoV-2 sequences. In addition, pseudotyped virus experiments demonstrate that sotrovimab retains activity against spike variants that confer reduced susceptibility to bamlanivimab, imdevimab and/or casivirimab. sotrovimab binds FcTR in a manner consistent with human IgG and demonstrates the potential for antibody-dependent cellular cytotoxicity and antibody-dependent cellular phagocytosis based on in vitro studies. In combination in vitro studies with remdesivir or bamlanivimab, sotrovimab showed additivity with the nucleoside inhibitor, and no antagonism was observed with either combination.

In more recent pseudotyped virus-like particle in vitro assessments, sotrovimab retains activity against the UK (Alpha, B.1.1.7; 2.30-fold change in EC50 value); South Africa (Beta, B.1.351; 0.60-fold change in EC50 value); Brazil (Gamma, P.1; 0.35-fold change in EC50 value); California (Epsilon, B.1.427/B.1.429; 0.70-fold change in EC50 value); New York (Iota, B.1.526; 0.6-fold change in EC50 value), India (Kappa, B.1.617.1; 0.7-fold change in EC50 value, Delta, B.1.617.2; 1-fold changed in EC50 value, Delta Plus [AY.1; 1.1-fold change in EC50 value and AY.2; 1.3-fold change in EC50 value]) and Peru (Lambda, C.37; 1.5-fold change in EC50 value) variant spike proteins.

Microneutralization data from authentic SARS-CoV-2 variant virus also indicate that sotrovimab retains activity against the UK (3-fold change in EC50 value), South Africa (1.2-fold change in EC50 value) and Brazil (1.6-fold change in EC50 value) variants.

### Example 50

### PK Results from Cynomolgus Monkey Studies

Following a single IV administration of sotrovimab at 5 mg/kg in cynomolgus monkeys, there was limited distribution of sotrovimab outside the vascular space, consistent with other IgGs. Consistent with the addition of the half-life extending LS modification, sotrovimab had a long half-life of 17.7 days and low clearance (3.87 mL/day/kg). No marked gender differences in PK parameters were observed.

A biodistribution study in cynomolgus monkeys was conducted to investigate uptake of sotrovimab into various tissues, including respiratory tract by full body Positron Emission Tomography and Computed Tomography (PET/CT) imaging and to quantitatively determine tissue to blood ratios. Following a single 5 mg/kg IV administration (containing approximately 2.5 mCi 89-Zr sotrovimab), tissue to blood ratios on Day 3 in respiratory tract tissues were determined, including nasal cavity (0.29), pharynx (0.20), larynx (0.23), trachea (0.16), pulmonary bronchi (0.41) and lung (0.25 including air space and 0.70 excluding air space).

### Example 51

### Clinical and PK Results from Adult Clinical Studies

As of 31 Mar. 2021, approximately 1350 participants have been randomized to either sotrovimab (500 mg dose) or placebo in two clinical studies: 1057 participants in a study evaluating sotrovimab for the treatment of non-hospitalized individuals with mild to moderate COVID-19 (COMET-ICE [NCT04545060]) and 300 participants in a study that evaluated sotrovimab for the treatment of individuals hospitalized with COVID-19 (NCT04501978]).

COMET-ICE, is a seamless first-in-human (FIH) Phase II/III study assessing the safety and efficacy of a single 500 mg IV dose of sotrovimab for the early treatment of COVID-19 in non-hospitalized participants at high risk for progression and subsequent hospitalization. Participants were randomized in a 1:1 ratio to sotrovimab or placebo. COMET-ICE started with a lead-in phase (N=21) in August 2020 to assess safety and tolerability. An IDMC met 23 Sep. 2020 to review unblinded safety data after the 20th participant from the lead-in cohort completed Day 15 (1 participant was withdrawn).

There were no deaths or SAEs reported up to this IDMC review. The IDMC recommended the study to proceed with the expansion-phase to enroll additional participants across each treatment group (˜1300 participants total). The IDMC subsequently met on 10 Mar. 2021 for a planned interim analysis, with review of data from 583 participants. There was an 85% reduction in the primary endpoint of hospitalization or death in the sotrovimab arm versus the placebo arm (p=0.002). The IDMC recommended that the study halt enrollment on the basis of overwhelming efficacy [Vir Biotechnology, 2021]. There have been no safety concerns identified at the IDMC reviews conducted to date.

Enrollment into COMET-ICE was closed on 11 Mar. 2021, participants continue to be followed-up to Week 24. Results from the COMET-ICE study at the Day 29 Analysis data cut-off (DCO; 27 Apr. 2021) indicate that sotrovimab is a highly efficacious treatment for participants with mild-to-moderate COVID-19 who are at risk of progressing to severe disease, meeting an unmet medical need. A review of COMET-ICE efficacy data based on the intent-to-treat (Day 29) population (N=1057) who received either sotrovimab or placebo demonstrated that the primary efficacy endpoint was met.

Treatment with sotrovimab resulted in a significant reduction in the proportion of participants with mild/moderate COVID-19 progressing to greater than 24 hours hospitalization or death in the sotrovimab arm when compared with placebo through Day 29 by 79% (adjusted relative risk reduction; p<0.001).

In vitro neutralization data using a SARS-CoV-2 pseudotyped virus are currently available for 13 of the 17 unique variants detected in the sotrovimab epitope. Of the variants with available data, sotrovimab effectively neutralized epitope variants at most amino acid positions tested with fold changes in half maximal effective concentration (EC50)<3-fold (range: 0.70 to 1.72). The variants E340A and E340K resulted in significant EC50 shifts (>100-fold) indicating reduced susceptibility to sotrovimab in vitro.

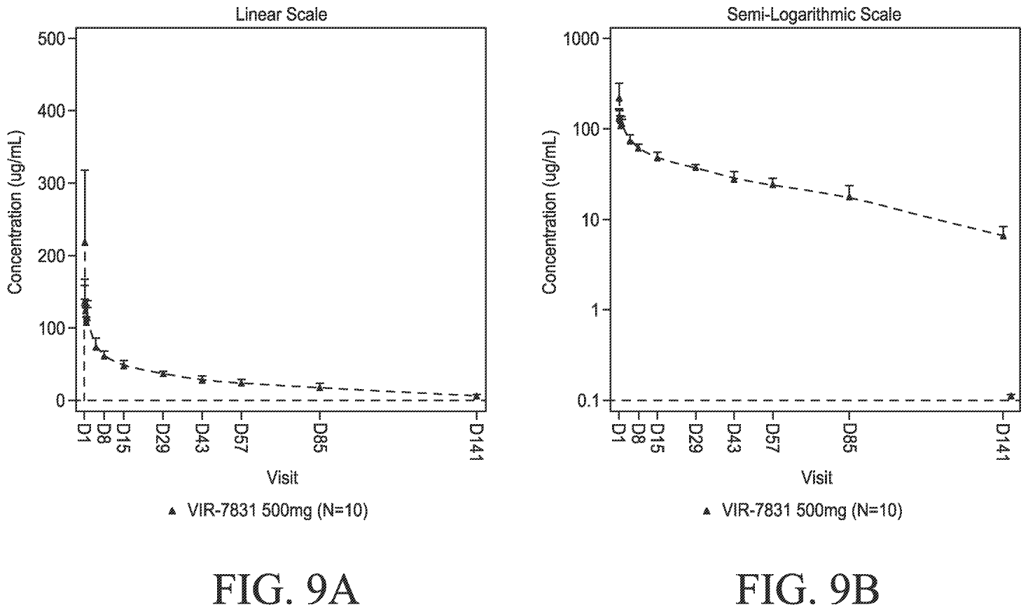

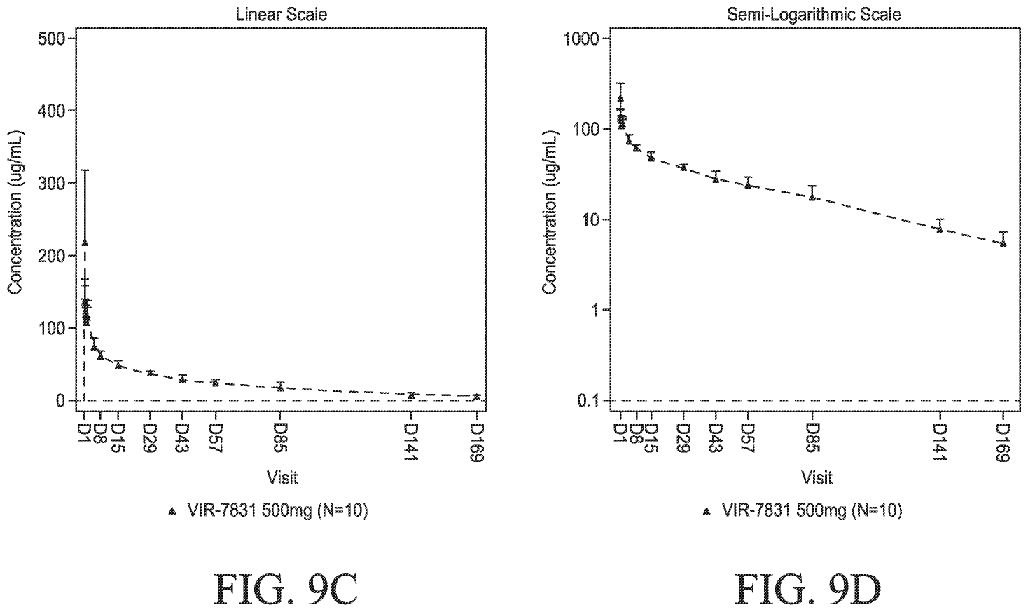

Limited nucleotide sequencing data from a total of 539 COMET-ICE participants indicated that 36 participants (16 treated with placebo and 20 treated with sotrovimab) carried the B.1.1.7 (Alpha, UK origin) variant. Four participants (2 treated with placebo and 2 treated with sotrovimab) carried the N501Y substitution. Thirty-one participants (19 treated with placebo and 12 treated with sotrovimab) carried the B.1.427/B.1.429 (Epsilon, California origin) variant. Eight additional participants carried the L452R substitution (6 treated with placebo and 2 treated with sotrovimab). Eleven participants carried the P.1 (Gamma, Brazil origin) variant (3 treated with placebo and 8 treated with sotrovimab). Three participants carried the B.1.526 (Iota, New York origin) variant with the E484K substitution (2 treated with placebo and 1 treated with sotrovimab), while 9 participants (4 treated with placebo and 5 treated with sotrovimab) carried the S477N substitution that has been associated with the B.1.526 (Iota, New York origin) variant. Additionally, 10 participants carried the E484K substitution (4 treated with placebo and 6 treated with sotrovimab), 2 carried the S494P substitution (1 treated with placebo and 1 treated with sotrovimab), and 3 carried the S494P substitution with the N501Y substitution (2 treated with placebo and 1 treated with sotrovimab). Two participants in the group receiving sotrovimab (1 carrying the B.1.427/B.1.429 [Epsilon, California origin] variant and 1 carrying the B.1.1.7 [Alpha, UK origin] variant) progressed to hospitalization. Four participants in the placebo group (2 carrying the E484K substitution, 1 carrying the P.1 [Gamma, Brazil origin] variant, and 1 carrying the B.1.1.7 [Alpha, UK origin] variant) progressed to hospitalization. None of the participants with currently available baseline sequences carried the full complement of substitutions characteristic The PK of a 500 mg IV dose of sotrovimab administered via a 1 hour IV infusion was evaluated in COMET-ICE. There were 10 participants in the sotrovimab arm in the Lead-in Phase of this study. One participant discontinued early due to withdrawal of consent following infusion with sotrovimab. Complete serum PK from 9 participants (sotrovimab) in the Lead-in phase of COMET-ICE is therefore available. The mean maximum observed concentration (Cmax) of 500 mg sotrovimab was 219 μg/mL following a 1 hour IV infusion. The mean serum level on Day 29 is 37.2 μg/mL. The mean clearance (CL) and volume of distribution at steady state (Vss) were 125 mL/day and 8.1 L, respectively. The median half-life was 48.8 days. The mean PK profile and parameters are presented in FIGS. 9A-D and Table 19.

Partial sparse serum PK through study Day 29 from 363 participants in the COMET-ICE Expansion shows the mean serum concentration of sotrovimab on study Day 29 was 25.8 μg/mL.

TABLE 19
Sotrovimab PK Parameters Following a 500
mg IV Dose: COMET-ICE Lead-in Phase

| | Dose | |
|---|---|---|
| Parameter | 500 mg(N = 9^{a}) | |
| Cₘₐₓ, μg/mL | 219 | (45.5) |
| Tₘₐₓ, day | 0.04 | (0.04, 0.05) |
| Cₗₐₛₜ, μg/mL | 5.41 | (37.2) |
| Tₗₐₛₜ, day | 161 | (160, 167) |
| AUC_{D 1-29}, day*μg/mL | 1529 | (9.6) |
| AUCₗₐₛₜ, day*μg/mL | 3714 | (14.5) |
| AUC % Extrapolated | 9.4 | (37.9) |
| AUC_{inf}, day*μg/mL | 4116 | (16.9) |
| CL (mL/day) | 125 | (17.9) |
| V_{z}, L | 8.76 | (15.7) |
| Vₛₛ, L | 8.1 | (11.1) |
| t_{1/2}, day | 48.8 | (37.8, 59.4) |

Abbreviations: AUC = area under the curve; AUClast = area under the serum concentration-time curve from time zero to time of last measurable concentration; AUC % Extrapolated = area under the plasma concentration-time curve extrapolated from time to infinity as a percentage of total AUC; AUCD 1-29 = area under the serum concentration-time curve from Day 1 to Day 29; AUCinf = area under the serum concentration-time curve from time zero to infinity; Clast = last measurable serum concentration; Cmax = maximum observed concentration; CL = clearance; t½ = terminal elimination half-life; tmax = time to reach Cmax; tlast = time of the last quantifiable concentration; Vss = volume of distribution at steady state; Vz = apparent volume of distribution during terminal phase.
Note:
Parameters are reported as mean (% CV) except for Tmax, Tlast, and t½, which are presented as median (min, max).
Data is based on 1-hour infusion time.
^{a}N = 8 for AUCD 1-29 as participant 10016 was missing all PK samples prior to Study Day 5.

### Example 52

### Phase IIb Study of Pharmacokinetics, Pharmacodynamics, and Safety of Single-Dose Sotrovimab in High-Risk Pediatric Participants with Mild to Moderate Covid-19

An open-label, non-comparator, multicenter study is performed to assess pharmacokinetics (PK), pharmacodynamics (PD; viral load) and safety following a single intravenous or intramuscular dose of sotrovimab in pediatric participants with mild to moderate COVID-19 at high risk of disease progression.

Abbreviations: ADA=anti-drug antibodies; AEs=adverse events; AESI=adverse events of special interest; anti-N=anti-nucleocapsid; AUCinf=area under the serum concentration time curve from time zero to infinity; Cmax=maximum observed concentration; CL=clearance; ECGs=electrocardiograms; F=bioavailability; SAEs=serious adverse events; t1/2=terminal elimination

### Primary Objectives

1. To evaluate the pharmacokinetics by IV or IM administration of sotrovimab in children from birth to <18 years.
Endpoints: i. Body weight-adjusted serum clearance of sotrovimab; ii. Serum PK of sotrovimab administered by IM injection or IV infusion (PK parameters may include Cmax, Tmax, AUCinf, t1/2, Vz, CL, F)
Other Estimand Attributes: i. Population: PK (all participants in Cohort A/Cohort B who are exposed to study intervention and who had at least 1 non-missing PK assessment [non-quantifiable values will be considered as non-missing values]); ii. Summary measure: PK model parameters
2. To evaluate the safety and tolerability of sotrovimab by IV or IM administration Endpoints: Incidence of adverse events (AEs), serious adverse events (SAEs), and adverse events of special interest (AESI) through Day 29 and Week 36 Other Estimand Attributes: i. Population: Safety (all participants who are exposed to study intervention in Cohort A/Cohort B); ii. Summary measure: Counts and percentages

### Secondary Objectives:

1. To evaluate disease progression following IV or IM administration of sotrovimab Endpoints: i. Progression of COVID-19 through Day 29 as defined by need for attended medical visit* or escalation to higher level of medical care or death; *An attended medical visit includes visit to a hospital emergency room for management of illness or hospitalization for acute management of illness; ii. Development of severe and/or critical respiratory COVID-19 as manifested by requirement for supplemental oxygen through Day 29*; *For participants who require oxygen or respiratory support for premorbid conditions, disease progression is defined as any sustained (>24 hours) increase in the level or method of oxygen support required Other Estimand Attributes: i. Population: Safety; ii. Summary measure: Counts and percentages
2. To characterize the effect of IV or IM administration of sotrovimab on SARS-CoV-2 viral load in respiratory tract samples among participants infected with SARS-CoV-2 Endpoints: Change from baseline in viral load in nasal secretions measured by qRT-PCR at Day 5, Day 8, and Day 11
Other Estimand Attributes: i. Population: Virology (all participants who are exposed to study intervention in Cohort A/Cohort B and have a quantifiable SARS-CoV-2 viral load measurement at baseline); ii. Summary measure: Arithmetic mean

### Other Safety Objectives:

1. To evaluate the safety and tolerability of sotrovimab following IV or IM administration.
Endpoint: Change from baseline in laboratory parameters, and vital signs
Other Estimand Attributes: i. Population: Safety; ii. Summary measure: Means Endpoint: i. Clinically significant change from baseline in ECGs; ii. Occurrence of disease-related events and/or disease-related outcomes (not classified as AEs) through Day 29; iii.; and occurrence of multisystem inflammatory syndrome in children (MIS-C) through Day 29 and Week 36
Other Estimand Attributes: i. Population: Safety; ii. Summary measure: Counts and percentages

### Exploratory Objectives

1. To evaluate the incidence of mortality
Endpoint: Incidence of all-cause mortality at Week 36
Other Estimand Attributes: i. Population: Safety; ii. Summary measure: Count and percentage
2. To monitor SARS-CoV-2 resistant mutants against sotrovimab
Endpoints: i. SARS-CoV-2 resistance mutants to sotrovimab at baseline; ii. Emergence of viral resistance mutants to sotrovimab by SARS-CoV-2
Other Estimand Attributes: i. Population: Virology; ii. Summary measure: Count and percentage
3. Evaluate the incidence of respiratory coinfection with SARS-CoV-2
Endpoint: Detection of respiratory pathogen in nasal secretion at Day 1
Other Estimand Attributes: i. Population: Safety; ii. Summary measure: Count and percentage
4. To assess the immunogenicity of sotrovimab
Endpoint: Incidence and titers (if applicable) of serum anti-drug antibodies (ADA) to sotrovimab
Other Estimand Attributes: i. Population: Safety; ii. Summary measure: Count and percentage
5. To assess the effects of sotrovimab on SARS-CoV-2 antibodies in participants >2 years of age
Endpoint: Incidence and titers (if applicable) of anti-nucleocapsid (anti-N) SARS-CoV-2 antibodies at Day 1 and Day 29 for participants >2 years of age
Other Estimand Attributes: i. Population: Safety; ii. Summary measure: Count and percentage
6. To assess COVID-19 symptom change over time
Endpoint: COVID-19 symptom severity assessment
Other Estimand Attributes: i. Population: Safety; ii. Summary measure: Count and percentage
Endpoints for all objectives will be assessed separately for IV and IM administration.

### Overall Design:

This study is a Phase 2b open-label, non-comparator, multi-center study to evaluate PK, safety, and PD of IM or IV administration of sotrovimab in pediatric participants aged from birth to <18 years with mild/moderate COVID-19 at high risk of disease progression.

This study includes 2 cohorts (Cohort A and Cohort B). Participants in Cohort A receive IV sotrovimab and participants in Cohort B receive sotrovimab via IM injections. The decision to enroll participants in Cohort A or Cohort B is per investigator discretion when both Cohorts enroll a given age group simultaneously. Cohort B initiation will be contingent upon 1) acceptable PK and safety from adolescents aged 12 to <18 years in cohort A and 2) data from COMET-TAIL supporting the efficacy of IM dosing in adults. The exposure-response relationship has not yet been established for sotrovimab in adults following IM administration. This will be determined after the read-out from the COMET-TAIL study and will be used to support initiation of IM administration and enrollment in Cohort B.

Participants are enrolled into the following age bands for both Cohort A and Cohort B:
Adolescents aged 12 to less than 18 years
Children aged 6 to less than 12 years
Children aged 2 to less than 6 years
Birth to less than 2 years

### Brief Summary

The purpose of the study is to assess the pharmacokinetics (PK), safety, and pharmacodynamics (PD) of sotrovimab administered via intravenous (IV) infusion or intramuscular (IM) injection in pediatric participants (aged from birth to <18 years) with mild/moderate COVID-19 at high risk of disease progression. Participants are enrolled in one of two cohorts (Cohort A or Cohort B). Participants in Cohort A receive IV sotrovimab and participants in Cohort B receive sotrovimab via an IM injection.
Study Duration: 36 weeks
Treatment Duration: A single dose of sotrovimab is administered by IV infusion or administered intramuscularly on Day 1.
Visit frequency: Participants are screened at the study site. Participants are dosed at 10 the study site on the same day as Screening or within 2 days after Screening. The day of dosing is referred to as Day 1. Participants have a home visit or return to the study site on Days 3, 5, 8, 11, 14, 22, and 29, as well as on Week 12 and Week 36, after dosing for follow-up visits. COVID-19 disease progression is monitored daily from Day 1 through Day 14 and on Day 22 and Day 29 (monitoring is via a phone call on days when a home visit or clinic visit is not scheduled). Additionally, a weekly phone call is conducted starting at Week 5 on weeks when there is not a scheduled clinic/home visit. Participants in both Cohorts have nasal mid-turbinate swabs for virology and resistance analyses and have blood drawn for assessment of PK, anti-drug antibodies (ADA), and for safety assessments during the study Participants >2 years of age will also have blood drawn for assessment of anti-nucleocapsid and anti-spike SARS-CoV-2 antibodies.

### Number of Participants:

Cohort A and Cohort B each enroll approximately 36 participants; therefore, a total of approximately 72 participants are enrolled in this study. The following number of participants are enrolled in each age band for each Cohort:
Adolescents aged 12 to less than 18 years: approximately 6 participants
Children aged 6 to less than 12 years: approximately 12 participants
Children aged 2 to less than 6 years: approximately 12 participants
Birth to less than 2 years: approximately 6 participants

Recruitment starts with Cohort A as follows:
The Cohort A 2 to <18 years age bands is recruited simultaneously.
The Cohort A birth to <2 years age band is recruited if deemed appropriate by the JSRT and IDMC after review of safety data once half of Cohort A participants 2 to <18 years (i.e., 15 participants) complete Day 29.

Recruitment in Cohort B occurs as follows:
Cohort B starts recruitment if deemed appropriate after review of PK data through Day 29 from all adolescents in the Cohort A 12 to <18 years age band (i.e., 6 participants) and after the efficacy of IM dosing is confirmed in adults. Safety data through Day 29 from all Cohort A 12 to <18 years age band participants are also reviewed by the JSRT and the IDMC prior to initiating Cohort B recruitment. Following favorable review, the Cohort B 2 to <18 years age bands are recruited simultaneously.
The Cohort B birth to <2 years age band is recruited if deemed appropriate by the JSRT and IDMC after review of safety data once half of Cohort B participants 2 to <18 years (i.e., 15 participants) complete Day 29.

The decision to enroll participants in Cohort A or Cohort B is per investigator discretion when both Cohorts enroll a given age group simultaneously. By using the Interactive Response Technology (IRT) system, the dispensation is monitored to ensure that the target number of participants in each cohort and in each age band receive IV infusion or IM injection.

Dosing is performed within 2 days of the Screening assessment. Dosing must occur ≤7 days from onset of COVID-19 symptoms. Screening can occur on the same day as dosing. Eligible participants are treated with a single IM or IV dose of sotrovimab on Day 1 and followed for up to 36 weeks.

### Inclusion Criteria:

Participants are eligible to be included in the study only if all of the following criteria apply:

### Age

1. Participant must be 32 weeks estimated gestational age (EGA), day of life (DOL) 0 to <18 years of age inclusive, at either the time of participant's signed assent (if age-appropriate) or parent(s)/legally authorized representative signing the informed consent.

### Type of Participant and Disease Characteristics

2. Participants with mild-moderate COVID-19, as defined by:
   A positive SARS-CoV-2 test result by any validated qRT-PCR or other nucleic acid amplification test (NAAT) [FDA, 2021a]; AND
   SpO₂≥94% on room air*; AND
   Have one or more of the following symptoms: fever, chills, cough, sore throat, malaise, headache, joint or muscle pain, change in smell or taste, vomiting, diarrhea, shortness of breath, poor appetite or poor feeding, nasal congestion/runny nose, lethargy; AND
   Less than or equal to 7 days from onset of symptoms to dosing day (Day 1).
*Note: Some participants may be on baseline oxygen supplementation or respiratory support (CPAP, BiPAP, or ventilator-dependence) prior to contracting COVID-19. Others, such as participants with some unrepaired congenital heart disease, may have baseline SPO₂ that is <94% on room air prior to contracting COVID-19. If a participant can maintain his/her baseline SpO₂ while on his/her baseline oxygen supplementation (including room air) and baseline respiratory support (modality, pressures, and frequency of use), he/she may be included in the study.
3. Participants at risk of disease progression with at least one of the following criteria:
   Age <1 year
   Diabetes mellitus
   Genetic or metabolic diseases: Pompe Disease, Mucopolysaccharidoses, glycogen storage diseases, fatty acid oxidation disorders, maple syrup urine disease, organic acidemias
   Obesity (as defined as body mass index (kg/m2) ≥95th percentile for age and sex based on local growth charts for children ≥2 years of age [or if not available based on the CDC or WHO growth charts respectively])
   Cardiovascular disease: congenital heart disease, hypertension, cardiomyopathy, heart failure
   Sickle cell disease
   Pulmonary disease: moderate to severe asthma, chronic lung disease, obstructive sleep apnea, Cystic Fibrosis
   Neurologic disease: seizure disorder, global developmental delay, cerebral palsy, or structural brain defect/malformation
   Immunosuppressed: primary immunodeficiency (e.g., Severe Combined Immunodeficiency), HIV infection with CD4+ count <200 cells/mm3, solid organ or bone marrow transplant, long-term use of systemic corticosteroids (defined by either ≥0.5 mg/kg/day by body weight or ≥20 mg/day prednisone equivalents [whichever is the lower dose of the two] taken for ≥2 weeks, immunosuppressive biologic agents (e.g., rituximab), and disease-modifying anti-rheumatic drugs (e.g., azathioprine, methotrexate, leflunomide).
   Baseline medical complexity (gastrostomy- or jejunostomy-dependence, parenteral nutrition dependence, tracheostomy-dependence, baseline oxygen requirement, use of CPAP/BiPAP/ventilator support).

### Weight

4. Body weight with the range stated below:
   Preterm newborn infants and term newborn infants: ≥2 kg
   Children 2 years to <18 years:
   Body mass index (BMI) ≥5th percentile for age based on local growth charts (or if not available based on the CDC or WHO growth charts).

### Sex and Contraceptive Barrier Requirements

5. Male and/or female (according to their reproductive organs and functions assigned by chromosomal complement) [FDA, 2016].
   Contraception and barriers as well as pregnancy testing is required for females only as appropriate for the age and sexual activity of pediatric participants and as required by local regulations regarding the methods of contraception for those participating in clinical studies.
   A female participant is eligible to participate if she is not breastfeeding and is either:
   Premenarcheal or
   Not pregnant as confirmed by a negative pregnancy test (serum or highly sensitive urine as required by local regulations) at Screening, before the first dose of study intervention, if of reproductive potential. Conducted again at Week 36 or the Early Withdrawal visit.
   If a urine test cannot be confirmed as negative (e.g., an ambiguous result), a serum pregnancy test is required. In such cases, the participant must be excluded from participation if the serum pregnancy result is positive.
   Additional serum or high sensitivity urine pregnancy tests may be performed, as determined necessary by the investigator or required by local regulation, to establish the absence of pregnancy at any time during the participant's participation in the study.

Females of childbearing potential commit to using a contraceptive method that is highly effective, with a failure rate of <1% during the study intervention period and for at least 36 weeks after the last dose of study intervention. The investigator evaluates potential for contraceptive method failure (e.g., noncompliance, recently initiated) in relationship to the first dose of study intervention.

The investigator is responsible for review of medical history, menstrual history, and recent sexual activity to decrease the risk for inclusion of a female with an early undetected pregnancy.

Note: If the childbearing potential changes after start of the study (e.g., a premenarcheal female participant experiences menarche) or the risk of pregnancy changes (e.g., a female participant who is not heterosexually active becomes active), the participant discusses this with the investigator, who determines if a female participant must begin a highly effective method of contraception. If reproductive status is questionable, additional evaluation should be considered.

### Informed Consent and Assent

6. The investigator, or a person designated by the investigator, obtains written informed consent from each study participant's legal guardian and the participant's assent, when applicable, before any study-specific activity is performed (unless a waiver of informed consent has been granted by an Institutional Review Board [IRB]/Ethics Committee [EC]). All legal guardians are fully informed, and participants are informed to the fullest extent possible, about the study in language and terms they are able to understand.
7. The participant capable of providing signed and dated written assent signs and dates a written assent form (age appropriate) and the parent/guardian signs and dates a written informed consent form (ICF) for study participation prior to the initiation of any study-related activities.

### Other

8. A legal guardian or primary caregiver is available to help the study-site personnel ensure follow-up; support the participant to attended assessment days according to the SoA (e.g., able to comply with scheduled visits, treatment plan, laboratory tests, and other study procedures); consistently and consecutively available to provide information on the participant using the rating scales during the scheduled study visits; accurately and reliably dispense study intervention as directed.
9-1. A legal guardian or primary caregiver is able to accurately maintain the child's take-home record, including items of general health.

In one alternative, Dosing will be based on weight/age bands as set forth in Table 20.

TABLE 20

| | |
|---|---|
| Weight- or age-based dosing bands | Sotrovimab dose(volume) |

For participants under 2 years of age, weight-based dosing is used

| | | |
|---|---|---|
| 2 kg to <4 kg | 62.5 mg | (1 mL) |
| ≥4 kg | 125 mg | (2 mL) |

For participants ≥2 years of age, weight-based dosing is not used

| | | |
|---|---|---|
| 2 to <6 yr | 187.5 mg | (3 mL) |
| 6 to <12 yr | 250 mg | (4 mL) |
| 12 to <18 yr | 375 mg | (6 mL) |
| Reference (Adult) | 500 mg | (8 mL) |

Intravenous sotrovimab is administered undiluted using a syringe pump for participants under 2 years of age, and diluted in 40 mL saline for participants >2 years of age. Because endotoxin specification limits for sotrovimab exceeds allowable levels for infants weighing <1.88 kg, infants <2 kg are excluded from the study.

Intramuscular sotrovimab is administered in dorsogluteal, ventrogluteal, or anterolateroal thigh (<2 years old), or deltoid muscle (location is per participant/legally authorized representative [LAR] preference and the clinical discretion of the investigator) without any saline dilution.

After IV infusion or IM injection of sotrovimab, participants are monitored for 2 hours. Local injection site tolerability is also monitored during the 2 hours post-dose. Throughout the study, a JSRT and IDMC review safety data from all participants enrolled in either Cohort. After two-thirds of participants are dosed in any of the age bands in either Cohort (Cohort A or Cohort B), the JSRT reviews safety data from that age band, and, if there are no safety concerns, a recommendation may be made by the JSRT to reduce the post-dose Day 1 monitoring observation time to 1 hour for that same age band within the same Cohort.

All participants in Cohort A and Cohort B are actively monitored on an outpatient basis for 36 weeks after dosing (i.e., Week 36). Monitoring occurs by home or clinic visits and phone calls throughout the 36 week period.
9-2. Alternatively

Dosing will be performed within 2 days of the Screening assessment. Dosing must occur ≤7 days from onset of COVID-19 symptoms. Screening can occur on the same day as dosing. Eligible participants will be treated with a single IM or IV dose of sotrovimab on Day 1 and followed for up to 36 weeks. Dosing will be based on weight in each age band as set forth in Table 21.

TABLE 21

| Age | Weight Band | Dose (mg) | Volume (mL) |
|---|---|---|---|
| 0 to <2 years | 2 to <5 kg | 62.5 | 1 |
| | 5 to <15 kg | 125 | 2 |
| | 15 to <40 kg | 250 | 4 |
| 2 to <6 years | 5 to <15 kg | 125 | 2 |
| | 15 to <40 kg | 250 | 4 |
| 6 to <12 years | 15 to <40 kg | 250 | 4 |
| | ≥40 kg | 500 | 8 |
| 12 to <18 years | 15 to <40 kg | 250 | 4 |
| | ≥40 kg | 500 | 8 |

Intravenous sotrovimab will be administered undiluted using a syringe pump for participants <15 kg, and diluted in 40 mL saline for participants >15 kg. Because endotoxin specification limits for sotrovimab exceeds allowable levels for infants weighing <1.88 kg, infants <2 kg will be excluded from the study. Intramuscular sotrovimab may be administered in dorsogluteal, anterolateral thigh, or deltoid muscles. Location of IM injections will be per participant/legally authorized representative [LAR] preference and the clinical discretion of the investigator.

After IV infusion or IM injection of sotrovimab, participants will be monitored for 2 hours. Local injection site tolerability will also be monitored during the 2 hours post-dose. Throughout the study, a JSRT and IDMC will review safety data from all participants enrolled in either Cohort. After half of participants are dosed in either Cohort (Cohort A or Cohort B), the JSRT will review safety data and, if there are no safety concerns, a recommendation may be made by the JSRT to reduce the post-dose Day 1 monitoring and observation time to 1 hour within the same Cohort.

All participants in Cohort A and Cohort B will be actively monitored on an outpatient basis for 36 weeks after dosing (i.e., Week 36). Monitoring will occur by home or clinic visits and phone calls throughout the 36 week period.

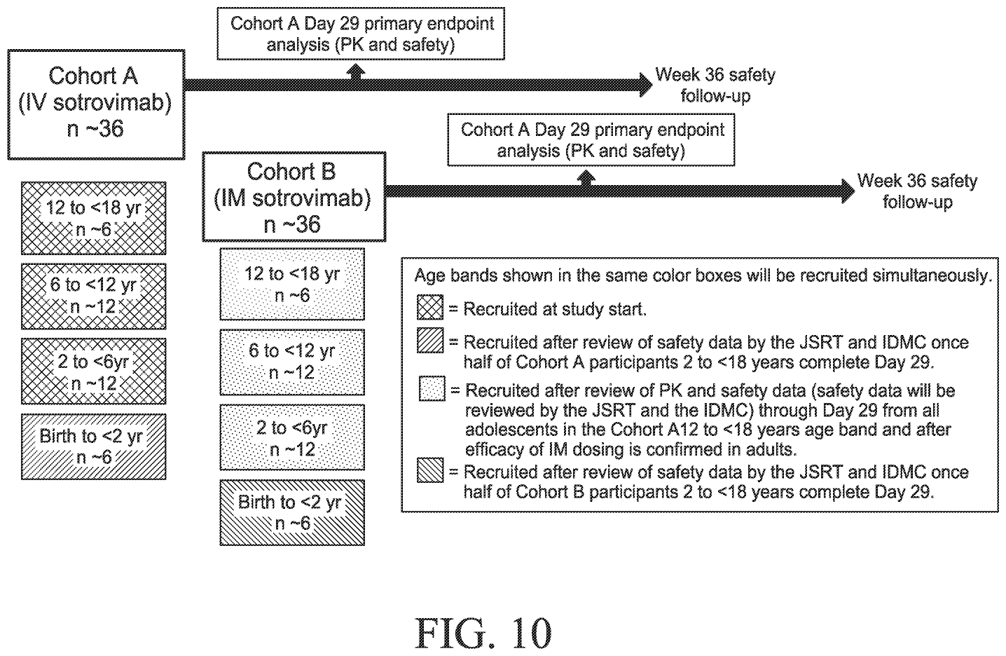

A summary of the clinical study is provided in FIG. 10.

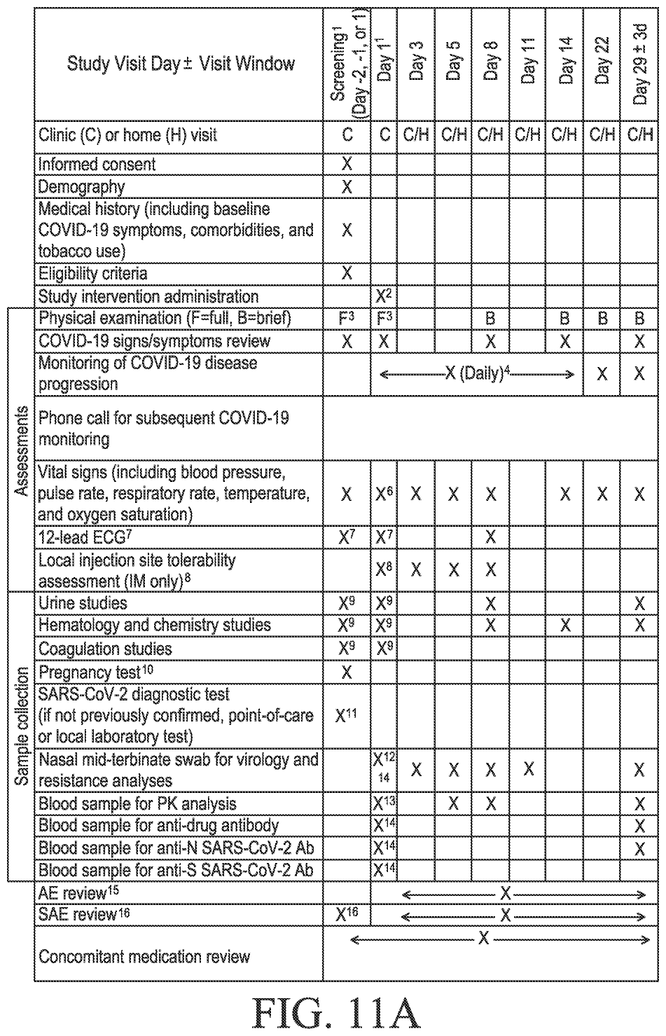

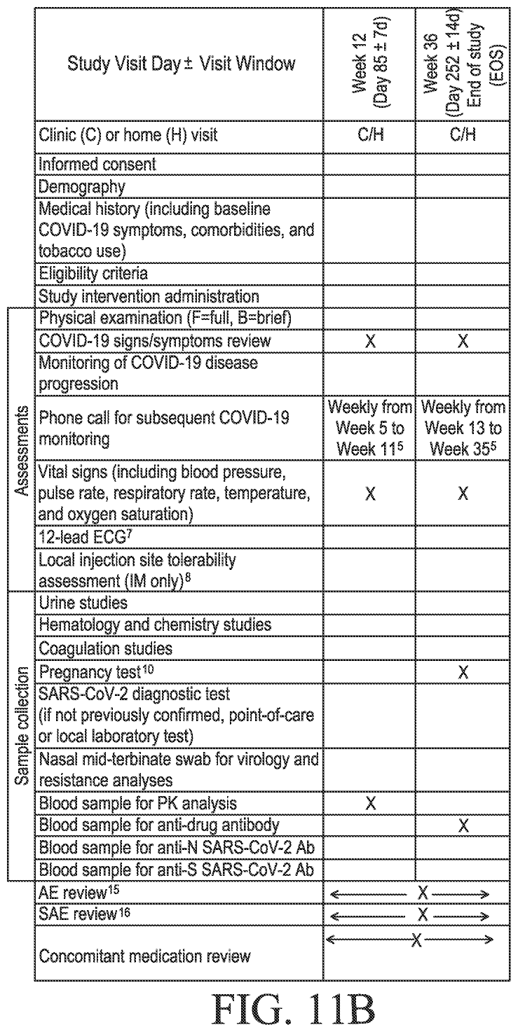

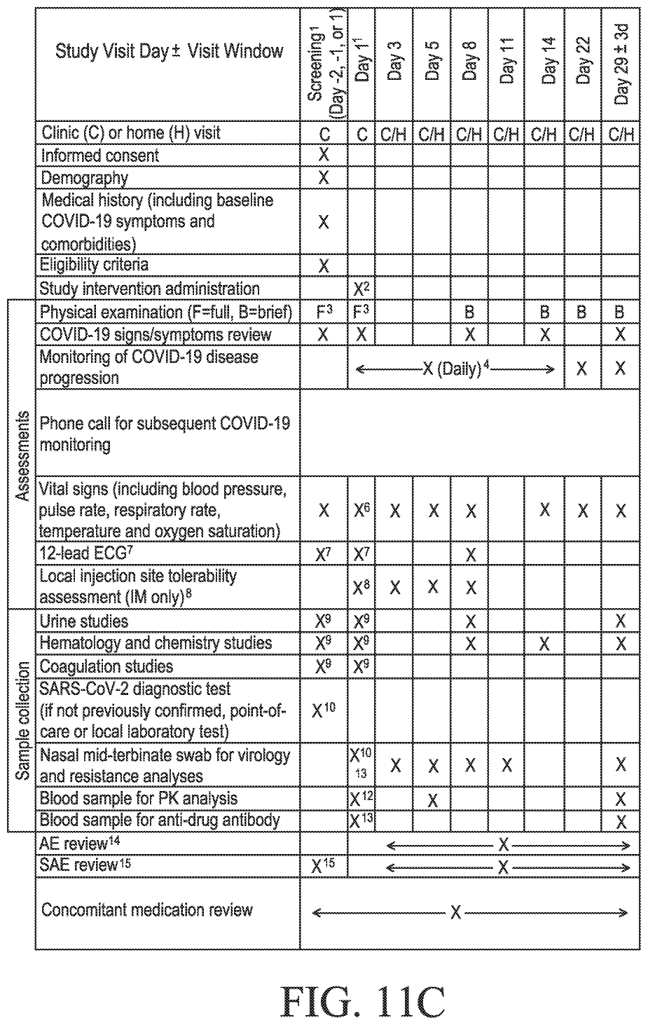

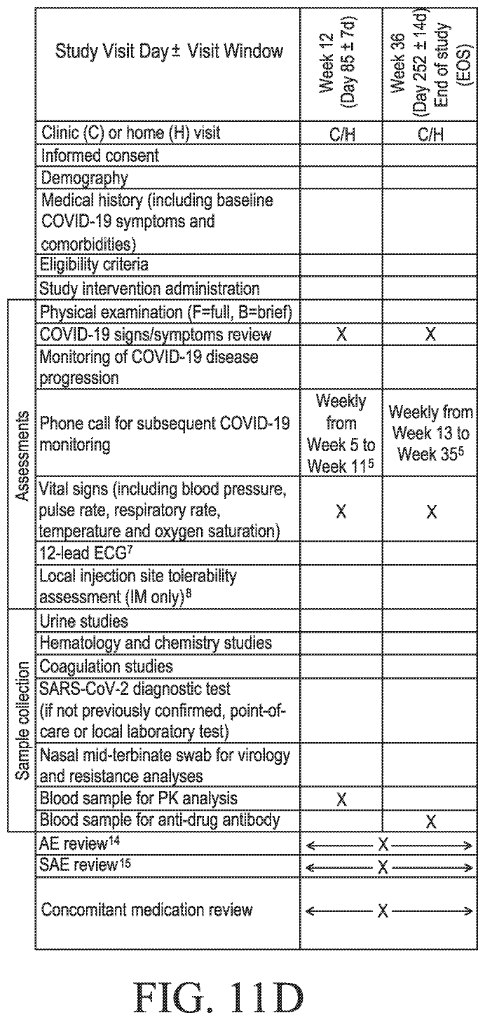

FIGS. 11A-11B and FIGS. 11C-11D provide a timeline of activities to be conducted during the study. FIGS. 11A-B and associated guidelines apply to study participants 2 year of age or older.

The following guidelines are also used in conjunction with the timeline of FIGS. 11A-11B. Notes: in case of early discontinuation (ED) or withdrawal (EW), all Week 36 activities should be performed.
1. All Screening procedures must be completed within 2 days prior to dosing. When possible, Screening and dosing (Day 1) can be done on the same day. Contact information for secondary contacts will be collected at Screening and information about health care providers/facilities may also be collected at Screening.
2. Dosing will be performed ≤7 days from the onset of symptoms. Participants will be monitored for 2 hours after dosing. If the Joint Safety Review Team (JSRT) recommends reducing to 1 hour monitoring, participants will be monitored for approximately 1 hour post-dose.
3. Including height, weight, and body mass index (BMI). The full physical examination only needs to be performed once if Screening and dosing occur on the same day.
4. Daily assessment will be via a phone call when a clinic or home visit is not scheduled.
5. Weekly phone call from Week 5 to Week 11 and Week 13 to Week 35 to assess for the incidence and severity of subsequent COVID 19 illness, if any. Weekly assessments must be performed within ±1 day of the scheduled phone call.
6. Record Day 1 vital signs within 1 hour prior to dosing. Vital signs will be monitored every 15 minutes over the 30 minute IV infusion. Vital signs will also be monitored at approximately 30 minutes, 1 hour, and 2 hours (IM or IV infusion) after dosing. If the post-dose monitoring is reduced to 1 hour per JSRT recommendation: Record Day 1 vital signs within 1 hour prior to dosing. Vital signs will be monitored every 15 minutes over the 30 minute IV infusion. Vital signs will also be monitored at approximately 30 minutes and 1 hour after dosing (IM or IV infusion).
7. At Screening, triplicate ECGs will be performed. A single 12-lead ECG will be performed prior to dosing on Day 1, within 30 minutes of end of dosing on Day 1, and on Day 8. If Screening and dosing occur on the same day, only the one set of triplicate ECGs is required prior to dosing (an additional pre-dosing single ECG is not required). Additional ECGs will be done as clinically needed.
8. On Day 1, local injection site tolerability assessment will be performed at approximately 1 hour and 2 hours post-dose. If JSRT recommends reducing monitoring time, Day 1 assessment will be 1 hour post-dose. All injection site reactions need to be followed by the principal investigator (PI) to resolution.
9. On Day 1, sample collection will occur pre-dose. Screening/Day 1 hematology, coagulation, chemistry, and urinalysis only need to be performed once if Screening and dosing occur on the same day. For IM administration, the results of hematology and coagulation laboratory results must be received and reviewed prior to dosing.
10. For women of child-bearing potential: serum or highly sensitive urine pregnancy test, as required by local guidelines.
11. Documentation of laboratory-confirmed SARS-CoV-2 infection via any validated qRT-PCR or other nucleic acid amplification test (NAAT) from any respiratory specimen collected ≤7 days prior to study entry must be confirmed at Screening. If not available, or previous qRT-PCR test was negative, a SARS CoV-2 qRT-PCR or other NAAT must be performed at Screening to confirm eligibility.
12. Day 1 samples will also be evaluated for SARS-CoV-2 characterization (e.g., identification of variants).
13. On Day 1, Cohort A participants will have a single PK sample collected at the end of infusion. Cohort B participants will not have any PK samples collected on Day 1.
14. Day 1 sample collection is pre-dose only.
15. AEs, including MIS-C, will be collected until Week 36 (EOS) during site visits and phone calls.
16. All SAEs will be collected from dose administration and only SAEs considered related to study participation or a GSK product will be collected from signing informed consent. SAEs will be collected during all site visits and phone calls. Note: all SAEs must be reported within 24 hours.

The following guidelines are also used in conjunction with the timeline of FIGS. 11C-11D. FIGS. 11C-11D and associated guidelines apply to study participants less than 2 years of age. Notes: In case of early discontinuation (ED) or withdrawal (EW), all Week 36 activities should be performed.
1. All Screening procedures must be completed within 2 days prior to dosing. When possible, Screening and dosing (Day 1) can be done on the same day. Contact information for secondary contacts will be collected at Screening and information about health care providers/facilities may also be collected at Screening.
2. Dosing will be performed ≤7 days from the onset of symptoms. Participants will be monitored for 2 hours after dosing. If the Joint Safety Review Team (JSRT) recommends reducing to 1 hour monitoring, Participants will be monitored for approximately 1 hour post-dose.
3. Including height, weight, and body mass index (BMI). The full physical examination only needs to be performed once if Screening and dosing occur on the same day.
4. Daily assessment will be via a phone call when a clinic or home visit is not scheduled.
5. Weekly phone call from Week 5 to Week 11 and Week 13 to Week 35 to assess for the incidence and severity of subsequent COVID 19 illness, if any. Weekly assessments must be performed within ±1 day of the scheduled phone call.
6. Record Day 1 vital signs within 1 hour prior to dosing. Vital signs will be monitored every 15 minutes over the 30 minute IV infusion. Vital signs will also be monitored at approximately 30 minutes, 1 hour, and 2 hours (IM or IV infusion) after dosing. If the post-dose monitoring is reduced to 1 hour per JSRT recommendation: Record Day 1 vital signs within 1 hour prior to dosing. Vital signs will be monitored every 15 minutes over the 30 minute IV infusion. Vital signs will also be monitored at approximately 30 minutes and 1 hour after dosing (IM or IV infusion).
7. At Screening, triplicate ECGs will be performed. A single 12-lead ECG will be performed prior to dosing on Day 1, within 30 minutes of end of dosing on Day 1, and on Day 8. If Screening and dosing occur on the same day, only the one set of triplicate ECGs is required prior to dosing (an additional pre-dosing single ECG is not required). Additional ECGs will be done as clinically needed.
8. On Day 1, local injection site tolerability assessment will be performed at approximately 1 hour and 2 hours post-dose. If JSRT recommends reducing monitoring time, Day 1 assessment will be 1 hour post-dose. All injection site reactions need to be followed by the principal investigator (PI) to resolution.
9. On Day 1, sample collection will occur pre-dose. Screening/Day 1 hematology, coagulation, chemistry, and urinalysis only need to be performed once if Screening and dosing occur on the same day. For IM administration the results of hematology and coagulation laboratory results must be received and reviewed prior to dosing.
10. Documentation of laboratory-confirmed SARS-CoV-2 infection via any validated qRT-PCR or other nucleic acid amplification test (NAAT) from any respiratory specimen collected ≤7 days prior to study entry must be confirmed at Screening. If not available, or previous qRT-PCR test was negative, a SARS-CoV-2 qRT-PCR or other NAAT must be performed at Screening to confirm eligibility.
11. Day 1 samples will also be evaluated for SARS-CoV-2 characterization (e.g., identification of variants).
12. On Day 1, Cohort A participants will have a single PK sample collected at the end of infusion. Cohort B participants will not have any PK samples collected on Day 1.
13. Day 1 sample collection is pre-dose only.
14. AEs (Section 10.3) including MIS-C will be collected until Week 36 (EOS) during site visits and phone calls.
15. All SAEs will be collected from dose administration and only SAEs considered related to study participation or a GSK product will be collected from signing informed consent. SAEs will be collected during all site visits and phone calls. Note: all SAEs must be reported within 24 hours.

### Example 53

### Pediatric Investigation Plan for Treatment of Covid-19

Three studies are performed on pediatric subjects.

### Study 1

Open-label, non-comparator, multicenter study to describe the pharmacokinetics (PK), pharmacodynamics (viral load) and safety following a single intravenous or intramuscular dose of sotrovimab in pediatric participants with mild to moderate COVID-19 at high risk of progression.

### Study Design Features and Main Objectives:

1. To assess the PK of sotrovimab in serum
2. To evaluate the safety and tolerability of sotrovimab

### Study Population and Subset:

Male and female infants, children and adolescents from birth to less than 18 years of age (including premature infants born at least at 32 weeks gestational age) with mild to moderate COVID-19 at high risk of progression

Number of Study Participants by Pediatric Subset (e.g. Age, Sex, Severity, or Stage):

At least 38 pediatric participants evaluable for the primary analysis, divided in the following age groups:
Adolescents from 12 to less than 18 years of age: at least 6
Children from 6 to less than 12 years of age: at least 12
Children from 2 to less than 6 years of age: at least 12
Infants and toddlers from birth to less than 2 years of age (including premature infants born at least at 32 weeks gestational age): at least 8

Recruitment is based on descending age groups, with age range from 6 years to less than 18 years recruited first.

### Study Duration for Participants:

Treatment duration: Single dose sotrovimab on Day 1
Follow-up duration (part of completion of this study): planned for at least 24 weeks

### Dosage, Treatment Regimen and Route of Administration:

Single dose sotrovimab, intravenous use (IV) or intramuscular (IM) use. A minimum of 10 participants are dosed via IV administration and a minimum of 10 participants are dosed via IM injection across all age groups. The remainder of participants are dosed via IV or IM administration per investigator discretion.

Dosing is based on allometric principles, with preferred doses minimizing differences in predicted pediatric exposure compared with adult AUC (0-28d) by and (if necessary) by weight band.

IM dosing may allow greater access to mAb treatment without the requirement of dedicated infusion centers, as is often required for IV administration

### Doses Between 100 mg and 500 mg:

Adolescents with a body mass >40 kg and aged 12 to 18 years: fixed dose of 500 mg
Children with a body mass between 10 kg and 40 kg and aged from 1 year to less than 12 years: fixed dose of between 200 mg and 250 mg
Children with a body mass <10 kg and aged below 1 year: fixed dose of 100 mg OR

### Doses Between 100 mg and 350 mg:

Adolescents aged 12 to 18 years: fixed dose of 350 mg
Children aged 6 to 12 years: fixed dose of 225 mg
Children aged 2 to 6 years: fixed dose of 150 mg
Children aged below 2 years: fixed dose of 100 mg
   Primary Endpoints with Time Points of Assessments:

### Pharmacokinetics:

Clearance (CL), Volume of distribution (Vdss), Cmax, C(28d), AUC(0-28d), AUC(0-inf). At least 5 samples taken at different timepoints between 0 and 84 days.

### Safety:

Treatment-emergent adverse events (TEASs)
Serious AEs
The incidence of MIS-C will be assessed as an AE/SAE
Infusion related reactions
Anti-drug antibodies (ADA) and antibody dependent enhancement (ADE)
   Main Secondary Endpoints with Time(s) of Assessment:

Proportion of participants who have progression of COVID-19 through Day 29 as defined by need for any type of attended medical visit or escalation to higher level of care for those already in medical care. Development of severe and/or critical respiratory COVID-19 as manifested by requirement for supplemental oxygen through Day 29. For participants requiring oxygen or respiratory support at baseline, disease progression is defined as any sustained (>24 hours) increase in the level or method of oxygen support required.

Change from baseline to day 7 in viral load as measured by quantitative reverse transcriptase polymerase chain reaction (qRT-PCR) in respiratory tract samples

### Exploratory Endpoint:

The incidence of Multisystem inflammatory syndrome in children (MIS-C) will be assessed as an AE/SAE.

### Statistical Plan Including Study Conduct and Analysis:

PK: descriptive statistics, estimation of body weight-adjusted clearance and volume of distribution compared with adults, estimation of AUC(028d), comparison of C(28d) with adults.

Safety: the analysis of the primary safety endpoint is assessed in the Safety Population (all participants who received sotrovimab) and focuses on safety data during the study for all age cohorts. Safety data is summarized descriptively and change from baseline is determined for select safety endpoints with PK samples.

Proportion of participants with progression of COVID-19 through Day 29: Descriptive Statistics

### Virology: Descriptive Statistics

### Inclusion Criteria:

1. Laboratory confirmed SARS-CoV-2 infection (by PCR or other nucleic acid test)
2. Children hospitalized and requiring medical care for COVID-19
3. Children with mild to moderate COVID-19 at risk of disease progression with at least one of the following criteria:
Asthma requiring daily medication or chronic lung disease
Diabetes
Genetic, neurologic or metabolic conditions
Sickle cell disease
Congenital heart disease
Immunosuppression (weakened immune system due to certain medical conditions or being on medications that weaken the immune system)
Medical complexity (e.g. tracheostomy, ventilator-support, or gastrostomy tube)
Obesity (defined in Study 1 as body mass index (kg/m²) ≥percentile for age and sex based on local growth charts)

### Study 2

Population PK (PopPK) model for dosing prediction and conformation in pediatric patients for 32 weeks gestational age (at birth) to less than 18 years of age. Used for dose finding.

### Model Type: Minimal Physiologically Based PK Model

### Data to be Used to Build Model:

Adult data: dense and sparse PK data from single dose sotrovimab 500 mg IV
Pediatric data from all participants from birth to less than 18 years of age

Data are used to define and identify a target efficacious exposure, defined as plasma sotrovimab C(28d) and AUC(0-28d) in keeping with the primary efficacy endpoint determined on Day 29.

Additional data on viral load levels are also used to confirm target engagement and evaluate exposure-response.
Model methodology and software: Population PK analysis using appropriate software.
Covariates: Age; Weight; Additional covariates may be tested, including body surface area, sex, and race
Model qualification: Standard model goodness of fit evaluation, including Visual Predictive Checks using boot-strap re-estimation methods with propagation of parameter uncertainty

### Study 3

PK bridging and extrapolation of safety and virology data to support the use of sotrovimab for the treatment of mild, moderate COVID-19 disease in children from 32 weeks gestational age (at birth) to less than 18 years of age.

### Study Objectives:

To provide justification for:
Pediatric dosing via extrapolation/PL bridging based on the similarity of the underlying disease process and the exposure-response to sotrovimab in adults and pediatric participants
Extrapolation of accumulated adult safety and immunogenicity data across all pediatric age groups
Extrapolation of virologic effect assumptions in all pediatric age groups based on adult data

Target population: Children from 32 weeks gestational age (at birth) to less than 18 years of age.

### Methodology:

To present data supporting the assumption that the efficacy of treatment is likely to be similar across all pediatric age groups based on confirmation of comparative C(d28) and AUC(0-28d) exposure by age/body weight band, together with confirmation of target engagement, as measured by viral load decline (based on if there is a demonstrable correlation between viral load decline and clinical efficacy in the adult population). Spare sampled (log 10-transformed) viral load data will be compared with adult reference using Bayesian Dynamic Borrowing.

The present a summary of safety and immunogenicity data from adults and children across all age groups using descriptive statistics to support safety.

Study Population and Subset Definition (Incl. Stratification):
Adults: patients with mild, early COVID-19 disease with less than 5 days' symptoms) with high risk for disease progression and patients with mild to moderate COVID-19
Children: patients from birth to less than 18 years of age

### Number of Study Participants by Pediatric Subset (e.g., Age, Sex, Stratum): Same as in Study 1.

Extrapolation: Data to be used to build the model: Adult: dense and sparse PK data from single dose sotrovimab 500 mg IV and IM from studies COMET-ICE (214367) and COMET-PEAK (216912).

Covariates for Pharmacokinetic analysis: Age, weight and body mass index (BMI) as covariates of sotrovimab exposure allowing adjustment for both body weight and obesity.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Patent Application No. 63/239,888, filed Sep. 1, 2021, and U.S. Patent Application No. 63/286,966, filed Dec. 7, 2021, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

The following references are incorporated by reference herein in their entireties.

### REFERENCES

Coronavirus Resource Center. (https://coronavirus.jhu.edu.)
Angulo F J, Finelli L, Swerdlow D L. Estimation of U S SARS-CoV-2 infections, symptomatic infections, hospitalizations, and deaths using seroprevalence surveys. JAMA Network Open 2021; 4:e2033706-e.
Arvin A M, Fink K, Schmid M A, et al. A perspective on potential antibody-dependent enhancement of SARS-CoV-2. Nature 2020; 584:353-63.
Baxter L T, Zhu H, Mackensen D G, Jain R K. Physiologically based pharmacokinetic model for specific and nonspecific monoclonal antibodies and fragments in normal tissues and human tumour xenografts in nude mice. Cancer Res. 1994 Mar. 15; 54(6):1517-1528.
Cathcart A L, Havenar-Daughton C, Lempp F A, et al. The dual function monoclonal antibodies VIR-7831 and VIR-7832 demonstrate potent in vitro and in vivo activity against SARS-CoV-2. bioRxiv 2021. doi:10.1101/2021.03.09.434607. Epub ahead of print.
Cariou B, Hadjadj S, Wargny M, et al. Phenotypic characteristics and prognosis of inpatients with COVID-19 and diabetes: the CORONADO study. Diabetologia 2020; 63:1500-15.
CDC COVID-19 Response Team. Severe outcomes among patients with coronavirus disease 2019 (COVID-19)—United States, February 12-Mar. 16, 2020. MMWR Morb Mortal Wkly Rep 2020; 69:343-6.
Chen P, Nirula A, Heller B, et al. SARS-CoV-2 neutralizing antibody LY-CoV555 in outpatients with Covid-19. N Engl J Med 2021; 384:229-37.
Chen R E, Zhang X, Case J B, et al. Resistance of SARS-CoV-2 variants to neutralization by monoclonal and serum-derived polyclonal antibodies. Nat Med 2021; 27(4):717-26.
Covell D G, Barbet J, Holton O D, Black C D, Parker R J, Weinstein I N. Pharmacokinetics of monoclonal immunoglobulin G1, F(ab′)2, and Fab′ in mice. Cancer Res.1986 August; 46(8):3969-78.
COVID-19: developing drugs and biological products for treatment or prevention. May 2020. (Accessed Feb. 19, 2021, at www.fda.gov/meida/137926/download.)
Datta-Mannan A. Mechanisms Influencing the Pharmacokinetics and Disposition of Monoclonal Antibodies and Peptides. Drug Metab Dispos. 2019 October; 47(10):1100-1110.
Deng R, Iyer S, Theil F P, Mortensen D L, Fielder P J, Prabhu S. Projecting human pharmacokinetics of therapeutic antibodies from nonclinical data: what have we learned? MAbs. 2011 January-February; 3(1):61-6. doi: 10.4161/mabs.3.1.13799. Epub 2011 Jan. 1. PMID: 20962582; PMCID: PMC3038012.
Duan K, Liu B, Li C, Zhang H, Yu T, Qu J, et al. Effectiveness of convalescent plasma therapy in severe COVID-19 patients. Proc Natl Acad Sci USA. 2020; 117(17):9490-6.
Focosi D, Maggi F. Neutralising antibody escape of SARS-CoV-2 spike protein: risk assessment for antibody-based Covid-19 therapeutics and vaccines. Rev Med Virol 2021. doi:10.1002/rmv.2231. Epub ahead of print.
Gaudinski M R, Coates E E, Houser K V, et al. Safety and pharmacokinetics of the Fc-modified HIV-1 human monoclonal antibody VRCO1L S: a phase 1 open-label clinical trial in healthy adults. PLoS Med 2018; 15:e1002493.
Hope. Multiscale imaging of therapeutic antibody distribution and localisation. Presented at 2019 HIV & HBV Cure Forum; July 20-21; Mexico City, Mexico Huang C, Wang Y, Li X, et al. Clinical features of patients infected with 2019 novel coronavirus in Wuhan, China. Lancet 2020; 395:497-506.
Hwang I K, Shih W J, De Cani J S. Group sequential designs using a family of type I error probability spending functions. Stat Med 1990; 9:1439-45. <<https://clinicaltrials.gov/ct2/show/NCT04545060>> <<https://clinicaltrials.gov/ct2/show/NCT04033406?term=VIR-2482&draw=2&rank=1 (VIR-2482 against influenza)>>
Ko S Y, Pegu A, Rudicell R S, et al. Enhanced neonatal Fc receptor function improves protection against primate SHIV infection. Nature 2014; 514:642-5.
Lempp F A, Soriaga L, Montiel-Ruiz M, et al. Membrane lectins enhance SARS-CoV-2 infection and influence the neutralizing activity of different classes of antibodies. bioRxiv 2021. doi:10.1101/2021.04.03.438258. Epub ahead of print.
Levey A S, Bosch J P, Lewis J B, Greene T, Rogers N, Roth D. A more accurate method to estimate glomerular filtration rate from serum creatinine: a new prediction equation. Modification of Diet in Renal Disease Study Group. Ann Intern Med 1999; 130:461-70.
Liang W, Liang H, Ou L, et al. Development and validation of a clinical risk score to predict the occurrence of critical illness in hospitalized patients with COVID-19. JAMA Intern Med 2020; 180:1081-9.
Lim J J, Derby M A, Zhang Y, Deng R, Larouche R, Anderson M, Maia M, Carrier S, Pelletier I, Girard J, Kulkarni P, Newton E, Tavel J A. A Phase 1, Randomized, Double-Blind, Placebo-Controlled, Single-Ascending-Dose Study To Investigate the Safety, Tolerability, and Pharmacokinetics of an Anti-Influenza B Virus Monoclonal Antibody, MHAB5553A, in Healthy Volunteers. Antimicrob Agents Chemother. 2017 Jul. 25; 61(8):e00279-17. doi: 10.1128/AAC.00279-17. PMID: 28559255; PMCID:PMC5527589.
Liu H, Wei P, Zhang Q, et al. 501Y.V2 and 501Y.V3 variants of SARS-CoV-2 lose binding to Bamlanivimab in vitro. bioRxiv 2021. doi:10.1101/2021.02.16.431305. Epub ahead of print.
Lobo E D, Hansen R J, Balthasar J P. Antibody pharmacokinetics and pharmacodynamics. J Pharm Sci. 2004 November; 93(11):2645-68
McCallum M, Bassi J, Marco A, et al. SARS-CoV-2 immune evasion by variant B.1.427/B.1.429. bioRxiv 2021. doi:10.1101/2021.03.31.437925. Epub ahead of print.
Petrilli C M, Jones S A, Yang J, et al. Factors associated with hospital admission and critical illness among 5279 people with coronavirus disease 2019 in New York City: prospective cohort study. BMJ 2020; 369:m1966.
Pinto D, Park Y J, Beltramello M, et al. Cross-neutralization of SARS-CoV-2 by a human monoclonal SARS-CoV antibody. Nature 2020; 583:290-5.
Reese H, Iuliano A D, Patel N N, et al. Estimated incidence of COVID-19 illness and hospitalization—United States, February-September, 2020. Clin Infect Dis 2020. doi:10.1093/cid/ciaa1780. Epub ahead of print.
Tada T, Dcosta B M, Zhou H, Vaill A, Kazmierski W, Landau N R. Decreased neutralization of SARS-CoV-2 global variants by therapeutic anti-spike protein monoclonal antibodies. bioRxiv 2021. doi:10.1101/2021.02.18.431897. Epub ahead of print.
Starr T N, Greaney A J, Dingens A S, Bloom J D. Complete map of SARS-CoV-2 RBD mutations that escape the monoclonal antibody LY-CoV555 and its cocktail with LY-CoV016. Cell Rep Med 2021; 2:100255.
VIR-7831 Investigator's Brochure, Edition 1, 28 Jul. 2020.
Wang P, Nair M S, Liu L, et al. Antibody resistance of SARS-CoV-2 variants B.1.351 and B.1.1.7. Nature 2021. doi:10.1038/s41586-021-03398-2. Epub ahead of print.
Weinreich D M, Sivapalasingam S, Norton T, et al. REGN-COV2, a neutralizing antibody cocktail, in outpatients with Covid-19. N Engl J Med 2021; 384:238-51.
Zalevsky J, Chamberlain A K, Horton H M, et al. Enhanced antibody half-life improves in vivo activity. Nat Biotechnol 2010; 28:157-9.

1. (canceled)

2. A method of treating a SARS-CoV-2 infection in a pediatric subject, the method comprising administering to the pediatric subject an effective amount of (1) a SARS-CoV-2 neutralizing antibody or antigen-binding fragment, or (2) a composition comprising (a) the antibody or antigen-binding fragment and (b) a pharmaceutically acceptable excipient, carrier, or diluent,
wherein the antibody or antigen-binding fragment comprises:
(A)(i) a heavy chain variable domain (VH) that comprises: the complementarity determining region (CDR)H1 amino acid sequence set forth in SEQ ID NO.:106, the CDRH2 amino acid sequence set forth in SEQ ID NO.:121, and the CDRH3 amino acid sequence set forth in SEQ ID NO.:108; and (ii) a light chain variable domain (VL) that comprises: the CDRL1 amino acid sequence set forth in SEQ ID NO.:169, the CDRL2 amino acid sequence set forth in SEQ ID NO.:170, and the CDRL3 amino acid sequence set forth in SEQ ID NO.:171;
(B)(i) a heavy chain variable domain (VH) that comprises the CDRH1, CDRH2, and CDRH3, of the amino acid sequence set forth in SEQ ID NO: 113 or 105, optionally as determined by IMGT; and (ii) and a light chain variable region that comprises the CDRL1, CDRL2, and CDRL3 of the amino acid sequence set forth in SEQ ID NO: 168; or
(C)(i) a heavy chain variable domain (VH) that comprises or consists of the amino acid sequence set forth in SEQ ID NO: 113 or 105; and (ii) a light chain variable region that comprises or consists of the amino acid sequence set forth in SEQ ID NO: 168.

3. The method of claim 2, wherein the method comprises administering to the pediatric subject only a single dose of the antibody or antigen-binding fragment or the composition.

4. The method of claim 2, wherein (1) the VH comprises the amino acid sequence set forth in SEQ ID NO.:113 and the VL comprises the amino acid sequence set forth in SEQ ID NO.:168, or (2) the VH comprises the amino acid sequence set forth in SEQ ID NO.:105 and the VL comprises the amino acid sequence set forth in SEQ ID NO.:168.

5. The method of claim 2, wherein the antibody or antigen-binding fragment is an IgG1 isotype.

6. The method of claim 2, wherein the antibody or antigen-binding fragment further comprises an Fc polypeptide or fragment thereof which comprises the following mutations, wherein the numbering of amino acid residues in the Fc polypeptide is according to the EU numbering system:
(i) M428L/N434S; or
(ii) M428L/N434S/G236A/A330L/I332E.

7. The method of claim 2, wherein the antibody or antigen-binding fragment comprises:
(1) the CH1-CH3 amino acid sequence of SEQ ID NO.:173 or 265 and the CL amino acid sequence of SEQ ID NO.:174; or
(2) the CH1-CH3 amino acid sequence of SEQ ID NO.:175 or 266 and the CL amino acid sequence of SEQ ID NO.:174.

8. (canceled)

9. The method of claim 2, wherein the antibody or antigen-binding fragment comprises a heavy chain polypeptide and a light chain polypeptide, wherein:
(1)(i) the heavy chain polypeptide comprises the VH amino acid sequence set forth in SEQ ID NO.:113 and aCH1-CH3 amino acid sequence set forth in SEQ ID NO.:173 or 265; and
(ii) the light chain comprises the VL amino acid sequence set forth in SEQ ID NO.:168 and a CL amino acid sequence set forth in SEQ ID NO.:174; or
(2)(i) the heavy chain polypeptide comprises the VH amino acid sequence set forth in SEQ ID NO.:113 and aCH1-CH3 amino acid sequence set forth in SEQ ID NO.:175 or 266; and
(ii) the light chain comprises the VL amino acid sequence set forth in SEQ ID NO.:168 and a CL amino acid sequence set forth in SEQ ID NO.:174.

10. (canceled)

11. The method of claim 2, wherein the pediatric subject:
(i) is aged less than two years, but at least 32 weeks gestational age at birth;
(ii) is aged two years to less than eighteen years,
(iii) is aged two years to less than six years;
(iv) is aged six years to less than twelve years;
(v) is aged twelve years to less than eighteen years;
(vi) has mild to moderate COVID-19;
(vii) has had fewer than seven days since onset of symptoms;
(viii) has had a positive reverse-transcriptase-polymerase-chain-reaction or antigen SARS-CoV-2 test result;
(ix) has any one or more of: age less than one year, diabetes mellitus, Pompe Disease, Mucopolysaccharidoses, glycogen storage diseases, fatty acid oxidation disorders, maple syrup urine disease, organic acidemias, obesity (body mass index (kg/m2) ≥95th percentile for age and sex based on CDC or WHO growth charts for children ≥2 years of age), congenital heart disease, hypertension, cardiomyopathy, heart failure, sickle cell disease, moderate to severe asthma, chronic lung disease, obstructive sleep apnea, Cystic Fibrosis, seizure disorder, global developmental delay, cerebral palsy, structural brain defect/malformation, primary immunodeficiency, HIV infection with CD4+ count <200 cells/mm³, solid organ or bone marrow transplant, long-term use of systemic corticosteroids (defined by either ≥0.5 mg/kg/day by body weight or ≥20 mg/day prednisone equivalents [whichever is the lower dose of the two] taken for ≥2 weeks), immunosuppressive biologic agents, disease-modifying anti-rheumatic drugs, gastrostomy- or jejunostomy-dependence, parenteral nutrition dependence, tracheostomy-dependence, baseline oxygen requirement, use of CPAP/BiPAP/ventilator support, or other baseline medical complexity;
(x) is a preterm newborn infant or term newborn infant weighing 2 kg or more; or
(xi) has or is any combination of (i)-(x).

12. The method of claim 2, comprising administering the antibody, antigen-binding fragment, or composition to the pediatric subject intravenously.

13. The method of claim 2, comprising administering the antibody, antigen-binding fragment, or composition to the pediatric subject intramuscularly by deltoid, gluteal, or thigh injection.

14. The method claim 2, wherein the method comprises administering the antibody or antigen-binding fragment to the pediatric subject at a dose of up to 62.5 mg, up to 100 mg, up to 125 mg, up to 150 mg, up to 187.5 mg, up to 200 mg, up to 225 mg, up to 250 mg, up to 300 mg, up to 350 mg, up to 375 mg, up to 400 mg, up to 450 mg, or up to 500 mg.

15. The method of claim 2, wherein the method comprises administering:
(i) 62.5, 125, 187.5, 250, or 375 mg of the antibody or antigen-binding fragment to the pediatric subject;
(ii) 100 mg, a range from 200 mg to 250 mg, or 500 mg of the antibody or antigen-binding fragment to the pediatric subject;
(iii) 100 mg, 150 mg, 225 mg, or 350 mg of the antibody or antigen-binding fragment to the pediatric subject;
(iv)
(a) administering 62.5 mg of the antibody or antigen-binding fragment to the pediatric subject, wherein the pediatric subject is aged less than two years, but at least 32 weeks gestational age at birth, and weighs at least 2 kg but less than 4 kg;
(b) administering 125 mg of the antibody or antigen-binding fragment to the pediatric subject, wherein the pediatric subject is aged less than two years, but at least 32 weeks gestational age at birth, and weighs at least 4 kg;
(c) administering 187.5 mg of the antibody or antigen-binding fragment to the pediatric subject, wherein the pediatric subject is aged two years to less than six years;
(d) administering 250 mg of the antibody or antigen-binding fragment to the pediatric subject, wherein the pediatric subject is aged six years to less than twelve years; and/or
(e) administering 375 mg of the antibody or antigen-binding fragment to the pediatric subject, wherein the pediatric subject is aged twelve years to less than eighteen years;
(v)
(a) administering 100 mg of the antibody or antigen-binding fragment to the pediatric subject, wherein the pediatric subject is aged less than one year, but at least 32 weeks gestational age at birth;
(b) administering a range from 200 and 250 mg of the antibody or antigen-binding fragment to the pediatric subject, wherein the pediatric subject is aged at least one year to less than twelve years; and/or
(c) administering 500 mg of the antibody or antigen-binding fragment to the pediatric subject, wherein the pediatric subject is aged at least twelve years to less than eighteen years; (vi)
(a) administering 100 mg of the antibody or antigen-binding fragment to the pediatric subject, wherein the pediatric subject is aged less than two years, but at least 32 weeks gestational age at birth;
(b) administering 150 mg of the antibody of antigen-binding fragment to the pediatric subject, wherein the pediatric subject is aged at least two years to less than six years;
(c) administering 225 mg of the antibody or antigen-binding fragment to the pediatric subject, wherein the pediatric subject is aged at least six years to less than twelve years; and/or
(d) administering 350 mg of the antibody or antigen-binding fragment to the pediatric subject, wherein the pediatric subject is aged at least twelve years to less than eighteen years; or
(vii) administering 500 mg of the antibody or antigen-binding fragment to the pediatric subject, wherein the pediatric subject is aged at least twelve years and weights more than 40 kg.

16. The method of claim 2, wherein the pediatric subject has a PCR or other nucleic acid amplification test-confirmed SARS-CoV-2 infection.

17. The method of claim 2, wherein the pediatric subject has SpO₂ ≥94% in room air and one or more of: fever, chills, cough, sore throat, malaise, headache, joint or muscle pain, change in smell or taste, vomiting, diarrhea, shortness of breath, poor appetite or poor feeding, nasal congestion/runny nose, and lethargy, but does not have hypoxemia (O₂ saturation ≤93% on room air or PaO₂/FiO₂<300) requiring oxygen supplementation for more than 1 day, require ≥4 L/min oxygen supplementation or equivalent, respiratory failure requiring at least one of invasive mechanical ventilation or ECMO, shock, or multi-organ dysfunction/failure.

18. The method of claim 2, wherein the pediatric subject has had a positive SARS-CoV-2 test result, has oxygen saturation ≥94% on room air, has COVID-19 symptoms, and is less than or equal to 7 days from onset of symptoms.

19. The method of claim 18, wherein the pediatric subject has one or more of: age less than one, diabetes mellitus, Pompe Disease, Mucopolysaccharidoses, glycogen storage diseases, fatty acid oxidation disorders, maple syrup urine disease, organic acidemias, obesity, congenital heart disease, hypertension, cardiomyopathy, heart failure, sickle cell disease, moderate to severe asthma, chronic lung disease, obstructive sleep apnea, Cystic Fibrosis, seizure disorder, global developmental delay, cerebral palsy, structural brain defect/malformation, primary immunodeficiency, HIV infection with CD4+ count <200 cells/mm3, solid organ or bone marrow transplant, long-term use of systemic corticosteroids, immunosuppressive biologic agents, or disease-modifying anti-rheumatic drugs, gastrostomy- or jejunostomy-dependence, parenteral nutrition dependence, tracheostomy-dependence, baseline oxygen requirement, use of CPAP/BiPAP/ventilator support, or other baseline medical complexity.

20. The method of claim 2, wherein the pediatric subject is receiving or has received, for SARS-CoV-2:
i) convalescent plasma therapy,
ii) remdesivir, sofosbuvir, acyclovir, zidovudine, molnupiravir, nirmatrelvir, ritonavir, or favipiravir, or brequinar, dexamethasone, tocilizumab, etesevimab, lopinavir, leronlimab, bebtelovimab, casirivimab, imdevimab, or any combination thereof, or
both i) and ii).

21-22. (canceled)

23. A kit comprising:
a liquid composition comprising an anti-SARS-CoV-2 antibody or antigen binding fragment comprising:
(A)(i) a heavy chain variable domain (VH) that comprises: the complementarity determining region (CDR)H1 amino acid sequence set forth in SEQ ID NO.:106, the CDRH2 amino acid sequence set forth in SEQ ID NO.:121, and the CDRH3 amino acid sequence set forth in SEQ ID NO.:108; and (ii) a light chain variable domain (VL) that comprises: the CDRL1 amino acid sequence set forth in SEQ ID NO.:169, the CDRL2 amino acid sequence set forth in SEQ ID NO.:170, and the CDRL3 amino acid sequence set forth in SEQ ID NO.:171;
(B)(i) a heavy chain variable domain (VH) that comprises the CDRH1, CDRH2, and CDRH3, of the amino acid sequence set forth in SEQ ID NO: 113 or 105, optionally as determined by IMGT; and (ii) and a light chain variable region that comprises the CDRL1, CDRL2, and CDRL3 of the amino acid sequence set forth in SEQ ID NO: 168, optionally as determined by IMGT; or
(C)(i) a heavy chain variable domain (VH) that comprises or consists of the amino acid sequence set forth in SEQ ID NO: 113 or 105; and (ii) a light chain variable region that comprises or consists of the amino acid sequence set forth in SEQ ID NO: 168; and
instructions for use in treating a SARS-CoV-2 infection in a pediatric subject by administering to the pediatric subject an effective amount of the SARS-CoV-2 neutralizing antibody or antigen-binding fragment.

24. The kit of claim 23, wherein the kit comprises the antibody or antigen-binding fragment in a composition further comprising a pharmaceutically acceptable excipient, carrier, or diluent, and the instructions for use are include instructions for administering a therapeutically effective amount of the composition.

25. The kit of claim 23, wherein the antibody or antigen-binding fragment comprises a heavy chain polypeptide and a light chain polypeptide, wherein:
(1)(i) the heavy chain polypeptide comprises the VH amino acid sequence set forth in SEQ ID NO.:113 and aCH1-CH3 amino acid sequence set forth in SEQ ID NO.:173 or 265; and
(ii) the light chain comprises the VL amino acid sequence set forth in SEQ ID NO.:168 and a CL amino acid sequence set forth in SEQ ID NO.:174; or
(2)(i) the heavy chain polypeptide comprises the VH amino acid sequence set forth in SEQ ID NO.:113 and aCH1-CH3 amino acid sequence set forth in SEQ ID NO.:175 or 266; and
(ii) the light chain comprises the VL amino acid sequence set forth in SEQ ID NO.:168 and a CL amino acid sequence set forth in SEQ ID NO.:174.
